SEQUENCE LISTING (98 sequences)

SEQ ID NO: 1 - DNA, 1320 bp, Psilocybe cubensis
  source 1..1320: mol_type = genomic DNA
atgcaggtgatacccgcgtgcaactcggcagcaataagatcactatgtcctactcccgagtcttttagaaacatgggatggctctctgtcagcgatgcggtctacagcgagttcataggagagttggctacccgcgcttccaatcgaaattactccaacgagttcggcctcatgcaacctatccaggaattcaaggctttcattgaaagcgacccggtggtgcaccaagaatttattgacatgttcgagggcattcaggactctccaaggaattatcaggaactatgtaatatgttcaacgatatctttcgcaaagctcccgtctacggagaccttggccctcccgtttatatgattatggccaaattaatgaacacccgagcgggcttctctgcattcacgagacaaaggttgaaccttcacttcaaaaaacttttcgatacctggggattgttcctgtcttcgaaagattctcgaaatgttcttgtggccgaccagttcgacgacagacattgcggctggttgaacgagcgggccttgtctgctatggttaaacattacaatggacgcgcatttgatgaagtcttcctctgcgataaaaatgccccatactacggcttcaactcttacgacgacttctttaatcgcagatttcgaaaccgagatatcgaccgacctgtagtcggtggagttaacaacaccaccctcatttctgctgcttgcgaatcactttcctacaacgtctcttatgacgtccagtctctcgacactttagttttcaaaggagagacttattcgcttaagcatttgctgaataatgaccctttcaccccacaattcgagcatgggagtattctacaaggattcttgaacgtcaccgcttaccaccgatggcacgcacccgtcaatgggacaatcgtcaaaatcatcaacgttccaggtacctactttgcgcaagccccgagcacgattggcgaccctatcccggataacgattacgacccacctccttaccttaagtctcttgtctacttctctaatattgccgcaaggcaaattatgtttattgaagccgacaacaaggaaattggcctcattttccttgtgttcatcggcatgaccgaaatctcgacatgtgaagccacggtgtccgaaggtcaacacgtcaatcgtggcgatgacttgggaatgttccatttcggtggttcttcgttcgcgcttggtctgaggaaggattgcagggcagagatcgttgaaaagttcaccgaacccggaacagtgatcagaatcaacgaagtcgtcgctgctctaaaggcttag

SEQ ID NO: 2 - protein, 439 aa, Psilocybe cubensis
MQVIPACNSAAIRSLCPTPESFRNMGWLSVSDAVYSEFIGELATRASNRNYSNEFGLMQPIQEFKAFIESDPVVHQEFIDMFEGIQDSPRNYQELCNMFNDIFRKAPVYGDLGPPVYMIMAKLMNTRAGFSAFTRQRLNLHFKKLFDTWGLFLSSKDSRNVLVADQFDDRHCGWLNERALSAMVKHYNGRAFDEVFLCDKNAPYYGFNSYDDFFNRRFRNRDIDRPVVGGVNNTTLISAACESLSYNVSYDVQSLDTLVFKGETYSLKHLLNNDPFTPQFEHGSILQGFLNVTAYHRWHAPVNGTIVKIINVPGTYFAQAPSTIGDPIPDNDYDPPPYLKSLVYFSNIAARQIMFIEADNKEIGLIFLVFIGMTEISTCEATVSEGQHVNRGDDLGMFHFGGSSFALGLRKDCRAEIVEKFTEPGTVIRINEVVAALKA

SEQ ID NO: 3 - DNA, 2155 bp, Psilocybe cubensis
  source 1..2155: mol_type = genomic DNA
atgatcgctgtactattctccttcgtcattgcaggatgcatatactacatcgtttctcgtagagtgaggcggtcgcgcttgccaccagggccgcctggcattcctattcccttcattgggaacatgtttgatatgcctgaagaatctccatggttaacatttctacaatggggacgggattacagtctgtcttgccgcgttgacttctaatatatgaacagctaatatattgtcagacaccgatattctctacgtggatgctggagggacagaaatggttattcttaacacgttggagaccattaccgatctattagaaaagcgagggtccatttattctggccggtgagctgatgttgagttttttgcaattgaatttgtggtcacacgtttccagacttgagagtacaatggtcaacgaacttatggggtgggagtttgacttagggttcatcacatacggcgacaggtggcgcgaagaaaggcgcatgttcgccaaggagttcagtgagaagggcatcaagcaatttcgccatgctcaagtgaaagctgcccatcagcttgtccaacagcttaccaaaacgccagaccgctgggcacaacatattcgccagtaagtactacttgaggaaaatagcgtacgcttcgctgaccggtccgtacatcaaagtcagatagcggcaatgtcactggatattggttatggaattgatcttgcagaagacgacccttggctggaagcgacccatttggctaatgaaggcctcgccatagcatcagtgccgggcaaattttgggtcgattcgttcccttctcgtgagcatccttcttctatgtaggaagggaaggagtctaacaagtgttagtaaaataccttcctgcttggttcccaggtgctgtcttcaagcgcaaagcgaaggtctggcgagaagccgccgaccatatggttgacatgccttatgaaactatgaggaaattagcagttagtcaaatgcgttctccccgtattttttcaatactctaacttcagctcacagcctcaaggattgactcgtccgtcgtatgcttcagctcgtctgcaagccatggatctcaacggtgaccttgagcatcaagaacacgtaatcaagaacacagccgcagaggttaatgtcggtaagtcaaaagcgtccgtcggcaattcaaaattcaggcgctaaagtgggtcttctcaccaaggtggaggcgatactgtaaggatttctcaatcgttagagtataagtgttctaatgcagtacatactccaccaaccagactgtctctgctatgtctgcgttcatcttggccatggtgaagtaccctgaggtccagcgaaaggttcaagcggagcttgatgctctgaccaataacggccaaattcctgactatgacgaagaagatgactccttgccatacctcaccgcatgtatcaaggagcttttccggtggaatcaaatcgcacccctcgctataccgcacaaattaatgaaggacgacgtgtaccgcgggtatctgattcccaagaacactctagtcttcgcaaacacctggtgaggctgtccattcattcctagtacatccgttgccccactaatagcatcttgataacagggcagtattaaacgatccagaagtctatccagatccctctgtgttccgcccagaaagatatcttggtcctgacgggaagcctgataacactgtacgcgacccacgtaaagcggcatttggctatggacgacgaaattggtaagtgcgctttcagaacccccccttccgttgactagtgccatgcgcgcatacaatatcgctattgatctgatataacttccctgcggcatttattttggcattcctttagtcccggaattcatctagcgcagtcgacggtttggattgcaggggcaaccctcttatcagcgttcaatatcgagcgacctgtcgatcagaatgggaagcccattgacataccggctgattttactacaggattcttcaggtagctaatttccgtctttgtgtgcataatacccctaacgacgcacgtttacctttttgtaaagacacccagtgcctttccagtgcaggtttgttcctcgaacagagcaagtctcacagtcggtatccggaccctga

SEQ ID NO: 4 - protein, 508 aa, Psilocybe cubensis
MIAVLFSFVIAGCIYYIVSRRVRRSRLPPGPPGIPIPFIGNMFDMPEESPWLTFLQWGRDYNTDILYVDAGGTEMVILNTLETITDLLEKRGSIYSGRLESTMVNELMGWEFDLGFITYGDRWREERRMFAKEFSEKGIKQFRHAQVKAAHQLVQQLTKTPDRWAQHIRHQIAAMSLDIGYGIDLAEDDPWLEATHLANEGLAIASVPGKFWVDSFPSLKYLPAWFPGAVFKRKAKVWREAADHMVDMPYETMRKLAPQGLTRPSYASARLQAMDLNGDLEHQEHVIKNTAAEVNVGGGDTTVSAMSAFILAMVKYPEVQRKVQAELDALTNNGQIPDYDEEDDSLPYLTACIKELFRWNQIAPLAIPHKLMKDDVYRGYLIPKNTLVFANTWAVLNDPEVYPDPSVFRPERYLGPDGKPDNTVRDPRKAAFGYGRRNCPGIHLAQSTVWIAGATLLSAFNIERPVDQNGKPIDIPADFTTGFFRHPVPFQCRFVPRTEQVSQSVSGP

SEQ ID NO: 5 - DNA, 1089 bp, Psilocybe cubensis
  source 1..1089: mol_type = genomic DNA
atggcgttcgatctcaagactgaagacggcctcatcacatatctcactaaacatctttctttggacgtcgacacgagcggagtgaagcgccttagcggaggctttgtcaatgtaacctggcgcattaagctcaatgctccttatcaaggtcatacgagcatcatcctgaagcatgctcagccgcacatgtctacggatgaggattttaagataggtgtagaacgttcggtttacgaataccaggctatcaagctcatgatggccaatcgggaggttctgggaggcgtggatggcatagtttctgtgccagaaggcctgaactacgacttagagaataatgcattgatcatgcaagatgtcgggaagatgaagacccttttagattatgtcaccgccaaaccgccacttgcgacggatatagcccgccttgttgggacagaaattggggggttcgttgccagactccataacataggccgcgagaggcgagacgatcctgagttcaaattcttctctggaaatattgtcggaaggacgacttcagaccagctgtatcaaaccatcatacccaacgcagcgaaatatggcgtcgatgaccccttgctgcctactgtggttaaggaccttgtggacgatgtcatgcacagcgaagagacccttgtcatggcggacctgtggagtggaaatattcttctccagttggaggagggaaacccatcgaagctgcagaagatatatatcctggattgggaactttgcaagtacggcccagcgtcgttggacctgggctatttcttgggtgactgctatttgatatcccgctttcaagacgagcaggtcggtacgacgatgcggcaagcctacttgcaaagctatgcgcgtacgagcaagcattcgatcaactacgccaaagtcactgcaggtattgctgctcatattgtgatgtggaccgactttatgcagtgggggagcgaggaagaaaggataaattttgtgaaaaagggggtagctgcctttcacgacgccaggggcaacaacgacaatggggaaattacgtctaccttactgaaggaatcatccactgcgtaa

SEQ ID NO: 6 - protein, 309 aa, Psilocybe cubensis
MHIRNPYRTPIDYQALSEAFPPLKPFVSVNADGTSSVDLTIPEAQRAFTAALLHRDFGLTMTIPEDRLCPTVPNRLNYVLWIEDIFNYTNKTLGLSDDRPIKGVDIGTGASAIYPMLACARFKAWSMVGTEVERKCIDTARLNVVANNLQDRLSILETSIDGPILVPIFEATEEYEYEFTMCNPPFYDGAADMQTSDAAKGFGFGVGAPHSGTVIEMSTEGGESAFVAQMVRESLKLRTRCRWYTSNLGKLKSLKEIVGLLKELEISNYAINEYVQGSTRRYAVAWSFTDIQLPEELSRPSNPELSSLF

SEQ ID NO: 7 - DNA, 930 bp, Psilocybe cubensis
  source 1..930: mol_type = genomic DNA
atgcatatcagaaatccttaccgtacaccaattgactatcaagcactttcagaggccttccctcccctcaagccatttgtgtctgtcaatgcagatggtaccagttctgttgacctcactatcccagaagcccagagggcgttcacggccgctcttcttcatcgtgacttcgggctcaccatgaccataccagaagaccgtctgtgcccaacagtccccaataggttgaactacgttctgtggattgaagatattttcaactacacgaacaaaaccctcggcctgtcggatgaccgtcctattaaaggcgttgatattggtacaggagcctccgcaatttatcctatgcttgcctgtgctcggttcaaggcatggtctatggttggaacagaggtcgagaggaagtgcattgacacggcccgcctcaatgtcgtcgcgaacaatctccaagaccgtctctcgatattagagacatccattgatggtcctattctcgtccccattttcgaggcgactgaagaatacgaatacgagtttactatgtgtaaccctccattctacgacggtgctgccgatatgcagacttcggatgctgccaaaggatttggatttggcgtgggcgctccccattctggaacagtcatcgaaatgtcgactgagggaggtgaatcggctttcgtcgctcagatggtccgtgagagcttgaagcttcgaacacgatgcagatggtacacgagtaacttgggaaagctgaaatccttgaaagaaatagtggggctgctgaaagaacttgagataagcaactatgccattaacgaatacgttcaggggtccacacgtcgttatgccgttgcgtggtctttcactgatattcaactgcctgaggagctttctcgtccctctaaccccgagctcagctctcttttctag

SEQ ID NO: 8 - protein, 309 aa, Psilocybe cubensis; identical to SEQ ID NO: 6

SEQ ID NO: 9 - DNA, 3042 bp, Psilocybe cubensis
  source 1..3042: mol_type = genomic DNA
atgccttccagtcaccctcacattactcatcgctatcgggttccttcgagtgacgaccatgaacgtatatctgctctgttcttgggtcccaaagcagaaaatgccgcatttctccagcaatggttgaccacggtcgtcgcacagcaaaaggctgcccgcgatgcatacttcccggatgacaatgcttttattactacagacatgcaaacttcccccgcctttgctcagactactaaagtaatcgcctccaatctcaccgaattattgactgcactcggtgaaaggtcgattcctttcttctcacctcggtacagcggccatatgtctgtggaccaaagtctacctgccattctcggattcttatcgaccacattttataatcctaacaatgttgccttcgaggctagtccattcacgaccctcatcgaggaagaagttggcttgcaactctctgaaatgctgggttataatcggctaaataacaccgagaaacctctcgcctggggacatattgcatcaggtggaactgttgcaaacttggaagcgatgtgggcggcgcgaaacctcaagttttaccctctctcactccgtgatgcttcagccgaaggcgcagagatggaattcattcgtgacacattctccgtcaaaacctgtgttggtgacaaaaaattattaaaggattgcagcccatgggaactcctcaatttgcatgtttctactatcttagacatgcccgaccgtctgcacgacgagtacaatatttcacctcagttcctcgaaaaggttatgcgaaagtatatcatccagtctaccaacaaagacacgttgatgcagcgttggggacttacccaacaacctgtcgttttatccccgagcacaaaccattattcctggccaaaggctgcagctgtgctcggtattggctcagacaaccttcgcaacgtcccagtagacatccaagcccacatggacataaacgaactcgatcgtatgttaaaaatttgcttggacgaggagacgccagtatatcaagtagttgctgttatcggtaccaccgaagagggcggtgtcgatcgcattacggagatcctgaagctgcgccaaaagtatgaagctttggggctgtcttttgccatccatgcagatgctgcttggggaggctattttgcaaccatgctacccaaagatacattgggtcgaaaccggactaggcttcccaaagaggacactacctcgggctttgtccctcacgtcggtctgcgcgaggagagcgcgttacaactcagccatataaagtatgccgattctattactatcgacccgcacaaggcaggctatgttccttaccccgctggggcactctgttatcgcgacggaagaatgaggtacctgcttacatggtccgcgccctaccttgcccaaggcaacgagggccaaagtatcggaatatacgggatcgaaggaagcaaacctggtgcagcagcatccgcggtattcatggcgcacgaaaccattggcctgactccttctggatacgggaaccttcttggccaggcaatgtttacatgtcgccgatacgctgctcactggtctgcaatgtcaacggatactaccagtttcactgtcaccccgttcaatcctatccctgctgacatcgaccccaacgctgaccccgcaaaggtcgaagagcaaaaacagttcatcagagatcgtatcttgttcaaatcgaacgaggaaatatacaacgattctgaggctatggaactcttgcaccaacttgggtccgatctcaatatcaacgttttcgcatgcaacttccgcgaccgcgataataatctcaacaccgacgtcgaggaagccaactggctcaataaccgtattttccaacgcttttctgttacaagtgctgaggagaacccattggaaacgccattcttcctcagctcaactacattgaaacaatccgaatacggcgtctgcgcaaccgaagtaaagagacgcatgggacttgttggtgaccaggatgttatagtcctgaggaacgtcgttatgtctccatttactacaacgaacgactttgtgggaactctggcaaacaccttccaaaagatcgttgaggaggaggtcgagtatgcacggatccgcaacgatatgaaacctagcattcacaccttccttcttcatggttcaggagagcaatactatcttgtccacaccccaacgatccatatggccagcggccgtcgccaaatcatcctttcagtaaatgttgaaggccaagttcggcaggcgatacatgcccatgaaagagttgaagcagtgattgtacataacactgtgcccctccgccttgacgaaatcgttgacggaggatcatttgacggcatactcaccatcggaaagaggaaaactagtttcaaagtgaagatttcaaacattaaagtagtcaagaagcgctctctgatgactgaggacctggaatctgcgtacccatcgttgatgccattctatttctacgggactcaaggacacgctcatctcgaccatgtcattactgtcgttcctaacatccatctgagtgctggcgaaatacagtacaaattcgacgacgaggtgtcaagcgaggacctcgccaagggcctcattgttgttgctgagaacgtacacgaggcatccatgcagcccttcccgctcatgaaagatttcaagatcaccaaccaattcttcttcagctccgggcaaatactccgcgtcaaagtgtacagagatccatacccggcatcgacaatggatcccatccctctccacgacatcaagaaccagcccgtcgtgacacaaggcaccatcacgctcgtcggaaatatttacgtcgattctgatgcgctcaacgtcgcttccgagcctactgccgacgaagacgcggcgcatgttcctcacgctcgcaacatgtacggcgagatgaccgctggaacgatcaaaggctggcaaaacgctgttcgtcatttccacaacaaattggagactgttgctccgacgaagtag

SEQ ID NO: 10 - protein, 1013 aa, Psilocybe cubensis
MPSSHPHITHRYRVPSSDDHERISALFLGPKAENAAFLQQWLTTVVAQQKAARDAYFPDDNAFITTDMQTSPAFAQTTKVIASNLTELLTALGERSIPFFSPRYSGHMSVDQSLPAILGFLSTTFYNPNNVAFEASPFTTLIEEEVGLQLSEMLGYNRLNNTEKPLAWGHIASGGTVANLEAMWAARNLKFYPLSLRDASAEGAEMEFIRDTFSVKTCVGDKKLLKDCSPWELLNLHVSTILDMPDRLHDEYNISPQFLEKVMRKYIIQSTNKDTLMQRWGLTQQPVVLSPSTNHYSWPKAAAVLGIGSDNLRNVPVDIQAHMDINELDRMLKICLDEETPVYQVVAVIGTTEEGGVDRITEILKLRQKYEALGLSFAIHADAAWGGYFATMLPKDTLGRNRTRLPKEDTTSGFVPHVGLREESALQLSHIKYADSITIDPHKAGYVPYPAGALCYRDGRMRYLLTWSAPYLAQGNEGQSIGIYGIEGSKPGAAASAVFMAHETIGLTPSGYGNLLGQAMFTCRRYAAHWSAMSTDTTSFTVTPFNPIPADIDPNADPAKVEEQKQFIRDRILFKSNEEIYNDSEAMELLHQLGSDLNINVFACNFRDRDNNLNTDVEEANWLNNRIFQRFSVTSAEENPLETPFFLSSTTLKQSEYGVCATEVKRRMGLVGDQDVIVLRNVVMSPFTTTNDFVGTLANTFQKIVEEEVEYARIRNDMKPSIHTFLLHGSGEQYYLVHTPTIHMASGRRQIILSVNVEGQVRQAIHAHERVEAVIVHNTVPLRLDEIVDGGSFDGILTIGKRKTSFKVKISNIKVVKKRSLMTEDLESAYPSLMPFYFYGTQGHAHLDHVITVVPNIHLSAGEIQYKFDDEVSSEDLAKGLIVVAENVHEASMQPFPLMKDFKITNQFFFSSGQILRVKVYRDPYPASTMDPIPLHDIKNQPVVTQGTITLVGNIYVDSDALNVASEPTADEDAAHVPHARNMYGEMTAGTIKGWQNAVRHFHNKLETVAPTK

SEQ ID NO: 11 - DNA, 2103 bp, Psilocybe cubensis
  source 1..2103: mol_type = genomic DNA
atggaggctatcaaaaaggtttttgagaacaaaaaggcggagggcattcctgtgttggtgacctttgttactgcaggatatcctcgtcccgaagatactgttcccatcttgctggccatggaggccggtggtgctgatatcatcgagcttggtatgccattttcagacccaattgcagatggtcctgtcatccaggaaacgaacacaatcgccgttgcaaaccaggtagattataccactgttctcggacaacttcgggaagcccgcaaacaagggctcaaggcacccgttcttctgatgggatattataaccccatattggcttacggagaagacagatctattcaagatgcggctgaagctggagccaatgggtttattatggtcgaccttccacccgaggaggctgtcgcttttcgagagaaatgtatcaaatccaacctctcatatgttcctctaattgcaccctcaacgactctgtcgcgtataaagttcctctcaacaattgcagacacgttcatctatgtcgtgtctaaaatgggaaccaccggatcctcagagaaggttgccatgaataacgcccttcccaccatcatcgatcgtattcgcgagtacgctgaagttcctttagcagtcggatttggagtcgccactcgggctcacttcaactacgtcgccgattccggtgctgatggtgtcgttattggcaccaaactcgttaacgttattaaagagtcaccgcaaggggaagcacccaaaaatgttgaggcatactgccgtgagatgagccaaaagggagaaacaaatcgcgtcaaatctccaccaactgcccgtgctgccagctccgaatcaattcctgttgttgttccttctgttctccccgcacgtttcggagaattcggaggacaatacgttcccgaagctcttgtcgattgtctggttgaactagaagaagctcacaaatctgccatggctgatcctgaattccagaaggaactacaatcgcatgccggatatgcaaatcgtccttcacaaatatacctcgccgaaaatctcaccaaggatgctgggggtgcaaatatttggttgaaacgtgaagatttgaaccacacaggttcccacaaaatcaataacgctttgggacaaattctgcttgcccggagaatcggaaagaccagaattatcgcagaaacaggtgccggccagcatggtgttgcaacagcgactgtttgcgctaagtttggaatggaatgtgttatctacatgggcgcagaagatgtgcgacggcaagctctaaatgtattcaggattgagatgctaggagcaaaagttgttcctgttacttcaggatcatgcacattgaaggacgctgtaaacgaggccttccgtgactgggtgacaaacctttctacgacgcattatttggttggctctgtaattggacctcatcccttccccaccattgtccgagatttccaaaaggtcattggtcaagagatcaaggctcagatgttggccgcccgcggcaaacttcctgatgtcgtcgtcgcttgtgttggtggaggaagcaatgctatcggtacgttctatgattttattggcgacaagagtgtacgtctagttggggtggaagcaggaggagaaggtattgacggagaccgacatagcgccacactttcgatggggcaaccgggagtacttcacggtgttagaacatatattctacaagacaaggccggtcaaatcatcgagacgcactcaatcagcgctggattggattatcccggcgttggaccagaacatgcttggctaaaggactctaaaagagcagaatatgttgtcgccacagacgaagaagcacttcgcggtttccgtatgctaacacaaagggagggaattattcctgcccttgaatcttcccatgcgatctgggaggctgtcaggattgcccgcaccatgtcgaaggaccaggatcttgttgtgtgtttgtctggccgaggtgataaagacgttgagcaaatttctcaacttcttcccaagtgggcggatattctagactggcatgtttcttcccatgccgttggacacacaacaaaattctaa

SEQ ID NO: 12 - protein, 700 aa, Psilocybe cubensis [sequence omitted]

SEQ ID NO: 13 - DNA, 1599 bp, Streptomyces rugosporus
  source 1..1599: mol_type = genomic DNA
atggaaaggcggaagcgtgagcgtcttggctcactcggccgaccgaccaaaaaggagctccgcatgatccgatctgtggtgatcgtgggtggtggcacggcgggctggatgaccgcctcctacctcaaggccgccttcgacgaccgcatcgacgtaacgctcgtggagtcagggaacgtcaggcggatcggggtcggcgaagcgaccttcagcacggtccgccacttcttcgactacctgggcctcgacgagcgcgagtggctgccccgctgcgccggcggctacaagctcggcatccgcttcgagaactggagcgagccgggcgagtacttctaccacccgttcgagcgcctgcgcgtcgtcgacggcttcaacatggccgagtggtggctcgcggtcggcgaccgcaggacgtccttcagcgaggcctgctatctcacgcaccggctgtgcgaggccaagcgggcgccccgcatgctcgacggctcgctcttcgcctcccaggtggacgagtcgctcggccgctcgaccctggccgagcagcgcgcccagttcccgtacgcctaccacttcgacgccgacgaggtcgcccgctacctgtcggagtacgccatcgcccgcggcgtccgccacgtggtcgacgacgtgcagcacgtcggccaggacgagcgcggctggatcagcggcgtccacaccaagcagcacggcgagatcagcggcgacctgttcgtcgactgcaccggcttccgcggcctgctcatcaaccagacgctgggcggcaggttccagtccttctccgacgtgctgcccaacaaccgggcggtcgcgctgcgcgtcccgcgggagaacgacgaggacatgcggccgtacacgacggcgaccgcgatgagcgccggctggatgtggacgatcccgctgttcaagcgcgacggcaacggctacgtctactccgacgagttcatctcgccggaggaggccgagcgcgagctgcggtccaccgtcgcccccggccgcgacgacctggaggccaaccacatccagatgcggatcggcaggaacgagcgcacctggatcaacaactgcgtcgccgtcggcctgtccgccgccttcgtcgagccgctggagtcgaccggcatcttcttcatccagcacgccatcgagcagctcgtgaagcacttccccggcgagcgctgggacccggtgctgatcagcgcgtacaacgagcgcatggcgcacatggtcgacggggtcaaggagttcctcgtcctccactacaagggcgcccagcgcgaggacaccccctactggaaggccgccaagaccagggccatgcccgacggcctcgcccgcaagctggagctgtccgcctcccacctgctggacgagcagacgatctacccctactaccacggcttcgagacctattcgtggatcaccatgaacctcggtctcggcatcgtgcccgagcggccgcgtcccgcgctcctgcacatggacccggcgcccgcgctggccgagttcgaacggctcaggcgcgagggcgacgagctgatcgccgccctgcccagctgctacgagtacctcgccagcatccaatga

SEQ ID NO: 14 - protein, 532 aa, Streptomyces rugosporus
MERRKRERLGSLGRPTKKELRMIRSVVIVGGGTAGWMTASYLKAAFDDRIDVTLVESGNVRRIGVGEATFSTVRHFFDYLGLDEREWLPRCAGGYKLGIRFENWSEPGEYFYHPFERLRVVDGFNMAEWWLAVGDRRTSFSEACYLTHRLCEAKRAPRMLDGSLFASQVDESLGRSTLAEQRAQFPYAYHFDADEVARYLSEYAIARGVRHVVDDVQHVGQDERGWISGVHTKQHGEISGDLFVDCTGFRGLLINQTLGGRFQSFSDVLPNNRAVALRVPRENDEDMRPYTTATAMSAGWMWTIPLFKRDGNGYVYSDEFISPEEAERELRSTVAPGRDDLEANHIQMRIGRNERTWINNCVAVGLSAAFVEPLESTGIFFIQHAIEQLVKHFPGERWDPVLISAYNERMAHMVDGVKEFLVLHYKGAQREDTPYWKAAKTRAMPDGLARKLELSASHLLDEQTIYPYYHGFETYSWITMNLGLGIVPERPRPALLHMDPAPALAEFERLRREGDELIAALPSCYEYLASIQ

SEQ ID NO: 15 - DNA, 1518 bp, Kutzneria sp. 744
  source 1..1518: mol_type = genomic DNA
atgaccgccgcctacctgaagaccgcgttcggcgaccggctttcgatcaccgtcgtggaatcgagcagaatcgggaccatcggcgtcggtgaggcgaccttcagcgacatccagcacttcttccagttcctcaacctccgggaacaggactggatgccggcctgcaacgccacctacaagctgggcatccgcttcgagaactggcggcatgtcggccatcatttctaccagccgttcgagcagatccgcccggtctacggctttccgctgaccgactggtggctgcacgacgcgccgaccgaccgcttcgacaccgactgcttcgtcatgcccaacctgtgcgaggccgggcgcagcccgcgccacctcgacggcacgctggccgacgaggacttcgtggaggagggcgacgagctcgccaaccgcaccatgtccgagcaccagggcaagtcgcagttcccgtacgcctaccacttcgaggcggcgctgctggccaagttcctcaccgggtacgcggtggaccgcggcgtcgagcacgtcgtcgacgacgtcctggacgtgcggctggaccagcgcgggtggatcgagcacgtggtcaccgccgagcacggcgagatccacggcgacctgttcgtcgactgcaccggtttccggggactgctgctgaacaaggcgctcggtgtgccgttcgtgtcctatcaggacactctgcccaacgacagcgcggtcgccctccaggtcccgctggacatgcagcggcgcggcatcgtgccgaacaccacggccaccgcgcgggaggccggctggatctggaccatcccgctgttcggtcgggtcggcaccgggtacgtctacgccaaggactacctctcgcccgaggaggccgagcgcacgctgcgcgagttcgtcggcccggcggccgcggacgtggaggccaaccacatccgcatgcggatcggccgcagccaggagtcctggcggaacaactgcgtggccatcggcctgtccagcggcttcgtcgagccgctggagtcgaccgggatcttcttcatccaccacgccatcgagcagctggtgaagcacttcccggcggccgactggaacccgaagtcccgggacatgtacaactccgcggtcgcgcacgtgatggacggcatccgcgagttcctggtgatccactaccggggcgcggcgcgggccgacaaccagtactggcgcgacaccaagacccgcccgctgccggacggcctggccgagcgcatcgagtgctggcagacgcagctgccggacaccgagacgatctacccgtactaccacgggctgccgccctactcctacatgtgcatcctgatgggcggcggggccatccggacgccggcctcggccgcgttggccctgaccgaccagggggccgcgcagaaggagttcgccgccgtccgggaccgggccgcgcagctgcgcgacacgctgcccagccactacgagtacctcgcgcggatgcgtggtctggatgtctga

SEQ ID NO: 16 - protein, 505 aa, Kutzneria sp. 744
MTAAYLKTAFGDRLSITVVESSRIGTIGVGEATFSDIQHFFQFLNLREQDWMPACNATYKLGIRFENWRHVGHHFYQPFEQIRPVYGFPLTDWWLHDAPTDRFDTDCFVMPNLCEAGRSPRHLDGTLADEDFVEEGDELANRTMSEHQGKSQFPYAYHFEAALLAKFLTGYAVDRGVEHVVDDVLDVRLDQRGWIEHVVTAEHGEIHGDLFVDCTGFRGLLLNKALGVPFVSYQDTLPNDSAVALQVPLDMQRRGIVPNTTATAREAGWIWTIPLFGRVGTGYVYAKDYLSPEEAERTLREFVGPAAADVEANHIRMRIGRSQESWRNNCVAIGLSSGFVEPLESTGIFFIHHAIEQLVKHFPAADWNPKSRDMYNSAVAHVMDGIREFLVIHYRGAARADNQYWRDTKTRPLPDGLAERIECWQTQLPDTETIYPYYHGLPPYSYMCILMGGGAIRTPASAALALTDQGAAQKEFAAVRDRAAQLRDTLPSHYEYLARMRGLDV

SEQ ID NO: 17 - DNA, 1572 bp, Streptomyces toxytrinici
  source 1..1572: mol_type = genomic DNA
atgaacacgagaaatccggacaaggtcgtcatcgtcggcggcggcacggcgggatggatgaccgcctcgtacctgaagaaggcgttcggcgagcgcgtgtccgtgaccctcgtcgaatcgggcaccatcggcaccgtcggcgtcggcgaggcgaccttcagcgacatccggcacttcttcgaattcctcgacctgcgggaggaggaatggatgccggcctgcaacgccacgtacaagctggccgtccgcttccaggactggcagcgccccggccaccacttctaccacccgttcgagcagatgcgctccgtagacgggttccccctgaccgactggtggctgcagaacgggccgaccgaccggttcgacagggactgcttcgtgatggcgtcgctgtgcgacgcggggcgcagcccgcgctatctcaacggttccctgctgcagcaggagttcgacgagcgcgcggaggagccggcggggctcaccatgtccgagcaccagggcaagacgcagttcccctacgcctaccacttcgaggcggccctgctcgccgaattcctttctgggtactccaaggaccgcggcgtgaagcacgtggtcgacgaggtgctggaggtgaagctcgacgaccgcggatggatcagccacgttgtcaccaaggagcacggcgacatcggcggcgacctgttcgtcgactgcaccggtttccgcggcgtactgctgaaccaggcgctgggtgtgcccttcgtctcctaccaggacaccctgcccaatgacagcgcggtcgccctccaggtgccgctcgacatggaggcgcggggcatcccgccgtacacccgggccaccgccaaggaggccgggtggatctggacgatcccgctgatcggccggatcggcaccgggtacgtgtacgccaaggactactgctcgccggaggaggccgagcgcaccctgcgcgagttcgtcggtccggaggccgccgacgtcgaggccaaccacatccgcatgcggatcgggcgcagcgagcagtcctggaagaacaactgcgtcgcgatcgggctctccagtggcttcgtcgaacccctcgagtcgaccggcatcttcttcatccaccacgccatcgagcagttggtgaagcacttcccggcgggcgactggcacccgcagctgcgcgccggctacaactcggccgtcgccaacgtcatggacggggtccgggagttcctcgtcctgcactacctgggcgccgcgcgcaacgacacccggtactggaaggacaccaagacccgggcggttccggatgccctggcggagcgcatcgagcgctggaaggtgcagctgcccgactccgagaacgtcttcccctactaccacggcctgccgccgtactcgtacatggccatcctgctcgggaccggcgcgatcgggctgcgaccgtcgccggcgctcgcactggccgacccggcggcggcggagaaggagttcaccgcgatccgggacagggcgcggttcctggtcgacaccctgccgagccagtacgagtacttcgccgcgatggggcagcgtgtctga

SEQ ID NO: 18 - protein, 523 aa, Streptomyces toxytrinici
MNTRNPDKVVIVGGGTAGWMTASYLKKAFGERVSVTLVESGTIGTVGVGEATFSDIRHFFEFLDLREEEWMPACNATYKLAVRFQDWQRPGHHFYHPFEQMRSVDGFPLTDWWLQNGPTDRFDRDCFVMASLCDAGRSPRYLNGSLLQQEFDERAEEPAGLTMSEHQGKTQFPYAYHFEAALLAEFLSGYSKDRGVKHVVDEVLEVKLDDRGWISHVVTKEHGDIGGDLFVDCTGFRGVLLNQALGVPFVSYQDTLPNDSAVALQVPLDMEARGIPPYTRATAKEAGWIWTIPLIGRIGTGYVYAKDYCSPEEAERTLREFVGPEAADVEANHIRMRIGRSEQSWKNNCVAIGLSSGFVEPLESTGIFFIHHAIEQLVKHFPAGDWHPQLRAGYNSAVANVMDGVREFLVLHYLGAARNDTRYWKDTKTRAVPDALAERIERWKVQLPDSENVFPYYHGLPPYSYMAILLGTGAIGLRPSPALALADPAAAEKEFTAIRDRARFLVDTLPSQYEYFAAMGQRV

SEQ ID NO: 19 - DNA, 1596 bp, Streptomyces albogriseolus
  source 1..1596: mol_type = genomic DNA
atggacaatcgaatcaagacagtcgtgatcctgggcggcggcaccgccggctggatgactgcggcgtatctcggcaaggcgctgcagaacacggtgaagatcgtggtcctggaggcgcccaccatcccgcggatcggtgtgggcgaagccaccgtccccaacctgcagcgcgcgttcttcgactacctcggcatccccgaggaggagtggatgcgcgagtgcaacgccagttacaagatggcggtgaagttcatcaactggcgcacaccgggggagggcagcccggatccccggacgctcgacgacggtcacaccgacaccttccaccaccccttcggactgctgccgtccgccgaccagataccgctctcccactactgggcggccaagcggctccagggcgagaccgacgagaacttcgacgaggcgtgtttcgccgacaccgcgatcatgaacgccaagaaggcgccccgcttcctcgacatgcggcgggcgaccaactacgcctggcacttcgacgccagcaaggtcgccgcgttcctgcgcaacttcgccgtcaccaagcaggcggtcgagcacgtcgaggacgagatgaccgaggtcctcaccgacgaacggggcttcatcaccgccctgcgcaccaagtcgggacggatcctgcagggtgacctcttcgtcgactgctccggcttccgcgggctgctgatcaacaaggccatggaggagccgttcatcgacatgagcgaccacctcctgtgcaacagcgcggtcgccacggccgtaccgcacgacgacgagaagaacggtgtcgagccgtacacctcctcgatcgccatggaggccggctggacctggaagattcccatgctgggccggttcggcagcggtcacgtctactccgaccacttcgccacccaggacgaggccaccctggccttctcgaagctgtggggcctcgacccggacaacaccgagttcaaccacgtccgcttccgggtcggccgcaaccgcagggcctgggtgcgcaactgcgtcagcgtcggcctcgcgtcgtgcttcgtggagccgctggagtccagcggcatctacttcatctacgcggccatccacatgctggccaagcacttccccgacaagaccttcgacaaggtgctcgtcgaccggttcaaccgggagatcgaggagatgttcgacgacacccgggacttcctccaggcgcactactacttctcgccgcgcgtcgacacgccgttctggcgggcgaacaaggaactgaagctggccgactccatcaaggacaaggtcgagacgtaccgggccgggctgccggtcaacctgccggtcaccgacgaggggacctactacggcaacttcgaggccgagttccggaacttctggaccaacggcagctactactgcatcttcgccggcctcggcctgatgccgcgcaacccgctgcccgcgctcgcctacaagccgcagtcgatcgcggaggccgagctgctgttcgccgacgtcaagcgcaagggggacaccctggtcgagtcgctgccgtcgacctatgacctgctgcgtcagttgcacggtgcgtcgtga

SEQ ID NO: 20 - protein, 531 aa, Streptomyces albogriseolus
MDNRIKTVVILGGGTAGWMTAAYLGKALQNTVKIVVLEAPTIPRIGVGEATVPNLQRAFFDYLGIPEEEWMRECNASYKMAVKFINWRTPGEGSPDPRTLDDGHTDTFHHPFGLLPSADQIPLSHYWAAKRLQGETDENFDEACFADTAIMNAKKAPRFLDMRRATNYAWHFDASKVAAFLRNFAVTKQAVEHVEDEMTEVLTDERGFITALRTKSGRILQGDLFVDCSGFRGLLINKAMEEPFIDMSDHLLCNSAVATAVPHDDEKNGVEPYTSSIAMEAGWTWKIPMLGRFGSGHVYSDHFATQDEATLAFSKLWGLDPDNTEFNHVRFRVGRNRRAWVRNCVSVGLASCFVEPLESSGIYFIYAAIHMLAKHFPDKTFDKVLVDRFNREIEEMFDDTRDFLQAHYYFSPRVDTPFWRANKELKLADSIKDKVETYRAGLPVNLPVTDEGTYYGNFEAEFRNFWTNGSYYCIFAGLGLMPRNPLPALAYKPQSIAEAELLFADVKRKGDTLVESLPSTYDLLRQLHGAS

SEQ ID NO: 21 - DNA, 1590 bp, unidentified
  source 1..1590: mol_type = genomic DNA; note = BorH halogenase
atggacaaccgcatcaaccggatcgtcatcctcggcggcgggaccgcgggatggatgaccgcctcctacctggcgaaggcgctcggtgacaccgtcacgatcaccctgctggaagcaccggcgatcggccggatcggggtcggcgaggccaccgtgcccaacctgcagcgcgtgttcttcgacttcctcggcctccgcgaggaggagtggatgccggagtgcaacgccgcgttcaagacggccgtgaagttcatcaactggcgtacccccgggcccggcgaggccaaggcccgcacgatcgacggccggccggaccacttctaccacccgttcgggctgctgcccgagcacggccaggtgccgctgtcgcactactgggcctacaaccgggcggccggcaccaccgacgagccgttcgactacgcctgtttcgccgagaccgccgccatggacgcggtccgcgcgcccaagtggctcgacgggcggcccgccacccggtatgcctggcacttcgacgcccacctggtcgccgagttcctgcgccggcacgccaccgagcggctcaacgtggagcacgtgcagggtgagatgcagcaggtgctgcgcgacgagcggggcttcatcaccgccctgcgcacggtcgagggccgggacctcgagggtgacctgttcatcgactgctccggcttccgcggcctgctgatcaacaaggccatggaggagcccttcatcgacatgaacgaccagctgctgtgcaaccgcgcggtggccacggcgatcaagcacgacgacgacgcgcacggggtcgagccgtacacctcggcgatcgccatgcggtccggctggagctggaagatcccgatgctgggccgcttcgggaccggctacgtctactccagccggttcgccgagaaggacgaggccaccctcgacttctgccggatgtggggcctggacccggagaacaccccgctcaaccaggtggccttccgggtcggccgcaaccgccgggcctgggtgaagaactgcgtgagcatcggcctggcgtcgtgcttcctggagccactggagtccaccggcatctacttcatcaccgcggccatctaccagctgactcagcacttcccggaccgcaccttcgcgctcgcgttgagcgacgcgttcaaccacgagatcgaagccatgttcgacgacacccgggacttcatccaggcgcacttctacgtgtcgccgcgcaccgacacgccgttctggaaggcgaacaaggacctgcacctgcccgagcagatgcgcgagaagatcgcgatgtacaaggccggcctgccgatcaacgcgccggtcaccgacgagtcgacctactacggcaggttcgaggccgagttccgcaacttctggaccaacggcagctactactgcatcttcgcgggcctcgggctgcgcccggacaacccgctgccgatgctgcgacaccgtccggagcaggtccgcgaggcgcaggcgctgttcgccggggtgaaggacaagcagcgggagttggtggagacgctgccgagcaacctggagttcctgcgcagcctgcacggcaagtag

SEQ ID NO: 22 - protein, 529 aa, unidentified
  source 1..529: note = BorH halogenase
MDNRINRIVILGGGTAGWMTASYLAKALGDTVTITLLEAPAIGRIGVGEATVPNLQRVFFDFLGLREEEWMPECNAAFKTAVKFINWRTPGPGEAKARTIDGRPDHFYHPFGLLPEHGQVPLSHYWAYNRAAGTTDEPFDYACFAETAAMDAVRAPKWLDGRPATRYAWHFDAHLVAEFLRRHATERLNVEHVQGEMQQVLRDERGFITALRTVEGRDLEGDLFIDCSGFRGLLINKAMEEPFIDMNDQLLCNRAVATAIKHDDDAHGVEPYTSAIAMRSGWSWKIPMLGRFGTGYVYSSRFAEKDEATLDFCRMWGLDPENTPLNQVAFRVGRNRRAWVKNCVSIGLASCFLEPLESTGIYFITAAIYQLTQHFPDRTFALALSDAFNHEIEAMFDDTRDFIQAHFYVSPRTDTPFWKANKDLHLPEQMREKIAMYKAGLPINAPVTDESTYYGRFEAEFRNFWTNGSYYCIFAGLGLRPDNPLPMLRHRPEQVREAQALFAGVKDKQRELVETLPSNLEFLRSLHGK

SEQ ID NO: 23 - DNA, 1602 bp, Kutzneria sp. 744
  source 1..1602: mol_type = genomic DNA
atggatgacaatcgaattcggagcatccttgtccttggcggcggcacggccggctggatgtccgcctgctacctgagcaaggcgctcgggcccggcgtcgaggtcaccgtgctcgaggcgccctccatctcgcgcatccgggtcggcgaggccaccattcccaacctgcacaaggtcttcttcgacttcctgggcatcgccgaggacgagtggatgcgggagtgcaacgccagctacaaggccgcggtccggttcgtcaactggcggacgccgggcgacggccaggccacgccgcggcggcgtccggacggccgccccgaccacttcgaccacctgttcggccagctgcccgagcacgagaacctgccgctgtcgcagtactgggcgcaccggcgcctcaacggcctgaccgacgaacccttcgaccgctcctgctacgtgcagcccgagctgctggaccgcaagctctcgccgaggttgatggacggcacgaaactggccagctacgcctggcacttcgacgccgacctggtggccgacttcctctgccggttcgccgtgcagaagctgaacgtgacccacgtccaggacgtgttcacgcatgccgacctcgaccagcgcggccacatcacggccgtcaacaccgagtccggccgcacgctggccgccgacctgttcatcgactgcagcggcttccgcagcgtgctcatgggcaaggtcatgcaggagccgttcctggacatgagcaagcacctgctcaacgaccgcgcggtggcgctgatgctcccgcacgacgacgagaaggtgggcatcgagccgtacacctcgtcgctggccatgcggtcgggctggtcgtggaagatcccgctgctcggccggttcggctccggctacgtctactccagccagttcacctcccaggacgaggcggccgaggagctctgccgcatgtgggacgtcgacccggcggagcagacgttcaacaacgtccggttccgggtcggccgcagccgccgggcctgggtgcgcaactgcgtcgccatcggcgtgtccgccatgttcgtggagccgctggagtcgaccggcctgtacttcagttacgcctcgctctaccagctggtgaagcacttcccggacaagcggttccggccgatcctggccgaccggttcaaccgcgaggtggcgaccatgtacgacgacacccgcgacttcctccaggcgcacttcagcctgtcgccgcgtgacgactccgagttctggcgggcctgcaaggagctgccgttcgcggacgggttcgccgagaaggtcgagatgtacagggccgggctgccggtcgaactgccggtcaccatcgacgacgggcactactacggcaatttcgaggccgagttccgcaacttctggaccaactcgaactactactgcatcttcgccgggctcggtttcctgcccgagcatccgctgccggtgctcgaattccgcccggaggccgtcgatcgcgcggagccggtgttcgccgcggtgcgccggcgcacggaggagctggtcgccaccgccccgaccatgcaggcctacctgcggcgcctgcaccagggcacgtag

SEQ ID NO: 24 - protein, 533 aa, Kutzneria sp. 744
MDDNRIRSILVLGGGTAGWMSACYLSKALGPGVEVTVLEAPSISRIRVGEATIPNLHKVFFDFLGIAEDEWMRECNASYKAAVRFVNWRTPGDGQATPRRRPDGRPDHFDHLFGQLPEHENLPLSQYWAHRRLNGLTDEPFDRSCYVQPELLDRKLSPRLMDGTKLASYAWHFDADLVADFLCRFAVQKLNVTHVQDVFTHADLDQRGHITAVNTESGRTLAADLFIDCSGFRSVLMGKVMQEPFLDMSKHLLNDRAVALMLPHDDEKVGIEPYTSSLAMRSGWSWKIPLLGRFGSGYVYSSQFTSQDEAAEELCRMWDVDPAEQTFNNVRFRVGRSRRAWVRNCVAIGVSAMFVEPLESTGLYFSYASLYQLVKHFPDKRFRPILADRFNREVATMYDDTRDFLQAHFSLSPRDDSEFWRACKELPFADGFAEKVEMYRAGLPVELPVTIDDGHYYGNFEAEFRNFWTNSNYYCIFAGLGFLPEHPLPVLEFRPEAVDRAEPVFAAVRRRTEELVATAPTMQAYLRRLHQGT

SEQ ID NO: 25 - DNA, 1617 bp, Pseudomonas fluorescens
  source 1..1617: mol_type = genomic DNA
atgaacaagccgatcaagaatatcgtcatcgtgggcggcggtactgcgggctggatggccgcctcgtacctcgtccgggccctccaacagcaggcgaacattacgctcatcgaatctgcggcgatccctcggatcggcgtgggcgaagcgaccatcccaagtttgcagaaggtgttcttcgatttcctcgggataccggagcgggaatggatgccccaagtgaacggcgcgttcaaggccgcgatcaagttcgtgaattggagaaagtctcccgacccctcgcgcgacgatcacttctaccatttgttcggcaacgtgccgaactgcgacggcgtgccgcttacccactactggctgcgcaagcgcgaacagggcttccagcagccgatggagtacgcgtgctacccgcagcccggggcactcgacggcaagctggcaccgtgcctgtccgacggcacccgccagatgtcccacgcgtggcacttcgacgcgcacctggtggccgacttcttgaagcgctgggccgtcgagcgcggggtgaaccgcgtggtcgatgaggtggtggacgttcgcctgaacaaccgcggctacatctccaacctgctcaccaaggaggggcggacgctggaggcggacctgttcatcgactgctccggcatgcgggggctcctgatcaatcaggcgctgaaggaacccttcatcgacatgtccgactacctgctgtgcgacagcgcggtcgccagcgccgtgcccaacgacgacgcgcgcgatggggtcgagccgtacacctcctcgatcgccatgaactcgggatggacctggaagattccgatgctgggccggttcggcagcggctacgtcttctcgagccatttcacctcgcgcgaccaggccaccgccgacttcctcaaactctggggcctctcggacaatcagccgctcaaccagatcaagttccgggtcgggcgcaacaagcgggcgtgggtcaacaactgcgtctcgatcgggctgtcgtcgtgctttctggagcccctggaatcgacggggatctacttcatctacgcggcgctttaccagctcgtgaagcacttccccgacacctcgttcgacccgcggctgagcgacgctttcaacgccgagatcgtccacatgttcgacgactgccgggatttcgtccaagcgcactatttcaccacgtcgcgcgatgacacgccgttctggctcgcgaaccggcacgacctgcggctctcggacgccatcaaagagaaggttcagcgctacaaggcggggctgccgctgaccaccacgtcgttcgacgattccacgtactacgagaccttcgactacgaattcaagaatttctggttgaacggcaactactactgcatctttgccggcttgggcatgctgcccgaccggtcgctgccgctgttgcagcaccgaccggagtcgatcgagaaagccgaggcgatgttcgccagcatccggcgcgaggccgagcgtctgcgcaccagcctgccgacaaactacgactacctgcggtcgctgcgtgacggcgacgcggggctgtcgcgcggccagcgtgggccgaagctcgcagcgcaggaaagcctgtag

SEQ ID NO: 26 - protein, 538 aa, Pseudomonas fluorescens
MNKPIKNIVIVGGGTAGWMAASYLVRALQQQANITLIESAAIPRIGVGEATIPSLQKVFFDFLGIPEREWMPQVNGAFKAAIKFVNWRKSPDPSRDDHFYHLFGNVPNCDGVPLTHYWLRKREQGFQQPMEYACYPQPGALDGKLAPCLSDGTRQMSHAWHFDAHLVADFLKRWAVERGVNRVVDEVVDVRLNNRGYISNLLTKEGRTLEADLFIDCSGMRGLLINQALKEPFIDMSDYLLCDSAVASAVPNDDARDGVEPYTSSIAMNSGWTWKIPMLGRFGSGYVFSSHFTSRDQATADFLKLWGLSDNQPLNQIKFRVGRNKRAWVNNCVSIGLSSCFLEPLESTGIYFIYAALYQLVKHFPDTSFDPRLSDAFNAEIVHMFDDCRDFVQAHYFTTSRDDTPFWLANRHDLRLSDAIKEKVQRYKAGLPLTTTSFDDSTYYETFDYEFKNFWLNGNYYCIFAGLGMLPDRSLPLLQHRPESIEKAEAMFASIRREAERLRTSLPTNYDYLRSLRDGDAGLSRGQRGPKLAAQESL

SEQ ID NO: 27 - DNA, 1593 bp, Lentzea areocolonigenes
  source 1..1593: mol_type = genomic DNA
atgtccggcaagattgacaagatcctcatcgtcggcggcggcaccgccggatggatggccgcgtcctatctcggcaaggccctgcagggcaccgcggacatcacactgctgcaggcacccgacatcccgacgctcggggtcggcgaggccacgatccccaatctgcagacggcgttcttcgacttcctcggaatccccgaggacgagtggatgcgggagtgcaacgcgagctacaaggtcgccatcaagttcatcaactggcgcaccgcgggcgaggggacgtccgaggcccgcgagctcgacggagggcccgaccacttctaccactccttcggtctgctcaagtaccacgagcagattccgctgtcgcactactggttcgaccgttcgtaccgggggaagaccgtcgagccgttcgactacgcctgctacaaggaacccgtcatcctcgacgccaacaggtcaccgcgcaggctcgacggttccaaggtgacgaactacgcgtggcacttcgacgcgcacctcgtcgccgacttcctgcgccggttcgccaccgagaagctcggcgtgcgccacgtcgaggaccgcgtcgagcacgtccagcgcgacgccaacggcaacatcgagtcggttcgcacggcaacggggcgtgtcttcgatgccgacctcttcgtcgactgctcgggcttccgcgggctgctgatcaacaaggcgatggaggagcccttcctcgacatgagcgatcacctgctcaacgacagcgccgtcgccacccaggtgccgcacgacgacgacgcgaacggtgtggaaccgttcacctcggcgatcgccatgaagtcgggctggacgtggaagatcccgatgctcggcaggttcggcaccgggtacgtctactcgagccggttcgccaccgaggacgaggcggtgcgcgagttctgcgagatgtggcacctcgacccggagacccagcccctcaacaggatccggttccgggtcggccgcaaccggcgcgcgtgggtcggcaactgcgtcagcatcggcacgtcgtcgtgcttcgtggaaccactggagtcgacgggcatctacttcgtctacgccgcgctgtaccagctggtgaagcacttccccgacaagagcctcaaccccgtgctgaccgccaggttcaaccgcgagatcgagacgatgttcgacgacacgcgcgacttcatccaggcgcacttctacttctcgccgcgcacggacaccccgttctggagggccaacaaggagctgcgcctggcggacggcatgcaggagaagatcgacatgtaccgcgcgggcatggcgatcaacgcgcccgcgtccgacgacgcccagctctactacggcaacttcgaggaggagttccgcaacttctggaacaacagcaactactactgcgtgctggccggcctcggtctggtgcccgacgcaccctcaccacgcctggcgcacatgccacaggcgacggagtcggtggacgaggtcttcggcgccgtcaaggaccggcagcggaacctgctcgagaccctgccgagcctccacgagttcctgaggcaacagcacggccgctga

SEQ ID NO: 28 - protein, 530 aa, Lentzea areocolonigenes
MSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMAINAPASDDAQLYYGNFEEEFRNFWNNSNYYCVLAGLGLVPDAPSPRLAHMPQATESVDEVFGAVKDRQRNLLETLPSLHEFLRQQHGR

SEQ ID NO: 29 - protein, 516 aa, Streptomyces albus
MLNNVVIVGGGTAGWMTASYFKAAFGERINITLVESGNIGAVGVGEATFSDIRHFFDFLGLKEKDWMPACNATYKLAVRFENWREEGHYFYHPFEQMRSVNGFPLADWWLREKPTDRFDKDCFVMPSIIDAGLSPRHLDGSLIDQPFVEGAEEMQGLTMSEHQGKTQFPYAYQFEAALLAKYLTTYSVERGVKHIVDDVQQVNLDERGWIRNVTTAEHGEIGGDLFIDCTGFRGLLLNKALEEPFISFQDTLPNDSAVALQVPMDMERRGILPCTTATAQDAGWIWTIPLMGRVGTGYVYAKDYISPEEAERTLREFVGPAAADAEANHIKMRVGRSENSWVKNCVAIGLSSGFVEPLESTGIFFIHHAIEQLAKNFPGEDWNPVQRNLYNDSVAHVMDGVREFLVLHYVAAKRSDTQYWRDTKTRKIPDALAERIEKWKTQLPDTETVFPYYHGLPPYSYHCVLLGMGGIDVKPSPALALGDSAAAEREFAEIREKTRHLTSVLPKAYDYFSRMR

SEQ ID NO: 30 - protein, 530 aa, Lentzea aerocologinenes
MSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMAINAPASDDAQLYWGNFEEEFRNFWNNSNYYCVLAGLGLVPDAPSPRLAHMPQATESVDEVFGAVKDRQRNLLETLPSLHEFLRQQHGR

SEQ ID NO: 31 - protein, 530 aa, Lentzea aerocologinenes
MSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMAINAPASDDAQLYYGNFEEEFRNFWNNSSYYCVLAGLGLVPDAPSPRLAHMPQATESVDEVFGAVKDRQRNLLETLPSLHEFLRQQHGR

SEQ ID NO: 32 - protein, 530 aa, Lentzea aerocologinenes
MSGKIDKILIVGGGTAGWMAASYLGKALQGTADITLLQAPDIPTLGVGEATMPNLQTAFFDFLGIPEDEWMRECNASYKVAIKFINWRTAGEGTSEARELDGGPDHFYHPFGLLKYHEQIPLSHYWFDRLYRGKTVEPFDYACYKEPVILDANRSPRRLDGSKVTSYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSFNPVLTARFNREIETMFDDTRDFIQAHFYFSPRTDTPFWRANKELRLADGMQEKIDMYRAGMAINAPAPDDAQLYWGNFEEEFRNLWNNSSYYCVLAGLGLVPDAPSPRLAHMPRATESVDEVFGAVKDQQRNLLETLPSLHEFLRQQHGR

SEQ ID NO: 33 - protein, 530 aa, Lentzea aerocologinenes
MSGKIDKILIVGGGTAGWMAASYLGKALQGTADITLLQAPDIPTLGVGEATHPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSFNPVLTARFNREIETMFDDTRDFIQAHFYFSPRTDTPFWRANKELRLADGMQEKIDMYRAGMAINAPASDDAQLYYGNFEEEFRNCWNNSSYYCVLAGLGLVPDAPSPRLAHMPRATESVDEVFGAVKDQQRNLLETLPSLHEFLRQQHGR

SEQ ID NO: 34 - protein, 530 aa, Lentzea aerocologinenes
MSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFSLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMAINAPASDDAQLYYGNFEEEFRNFWNNSSYYCVLAGLGLVPDAPSPRLAHMPQATESVDEVFGAVKDRQRNLLETLPSLHEFLRQQHGR

SEQ ID NO: 35 - protein, 530 aa, Lentzea aerocologinenes
MSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMVINAPASDDAQLYYGNFEEEFRNFWNNSNYYCVLAGLGLVPDAPSPRLAHMPQATESVDEVFGAVKDRQRNLLETLPSLHEFLRQQHGR

SEQ ID NO: 36 - DNA, 1446 bp, Bacillus atrophaeus
  source 1..1446: mol_type = genomic DNA
atgatgtctgaaaatttgcaattgtcagctgaagaaatgagacaattgggttaccaagcagttgatttgatcatcgatcacatgaaccatttgaagtctaagccagtttcagaaacaatcgattctgatatcttgagaaataagttgactgaatctatcccagaaaatggttcagatccaaaggaattgttgcatttcttgaacagaaacgtttttaatcaaattacacatgttgatcatccacatttcttggcttttgttccaggtccaaataattacgttggtgttgttgcagatttcttggcttctggttttaatgtttttccaactgcatggattgctggtgcaggtgctgaacaaatcgaattgactacaattaattggttgaaatctatgttgggttttccagattcagctgaaggtttatttgtttctggtggttcaatggcaaatttgacagctttgactgttgcaagacaggctaagttgaacaacgatatcgaaaatgctgttgtttacttctctgatcaaacacatttctcagttgatagagcattgaaggttttaggttttaaacatcatcaaatctgtagaatcgaaacagatgaacatttgagaatctctgtttcagctttgaagaaacaaattaaagaagatagaactaagggtaaaaagccattctgtgttattgcaaatgctggtactacaaattgtggtgctgttgattctttgaacgaattagcagatttgtgtaacgatgaagatgtttggttgcatgctgatggttcttatggtgctccagctatcttgtctgaaaagggttcagctatgttgcaaggtattcatagagcagattctttgactttagatccacataagtggttgttccaaccatacgatgttggttgtgttttgatcagaaactctcaatatttgtcaaagacttttagaatgatgccagaatacatcaaggattcagaaactaacgttgaaggtgaaattaatttcggtgaatgtggtatcgaattgtcaagaagattcagagctttgaaggtttggttgtcttttaaagttttcggtgttgctgcttttagacaagcaatcgatcatggtatcatgttagcagaacaagttgaagcatttttgggtaaagcaaaagattgggaagttgttacaccagctcaattgggtatcgttacttttagatacattccatctgaattggcatcaacagatactattaatgaaattaataagaaattggttaaggaaatcacacatagaggtttcgctatgttatctactacagaattgaaggaaaaggttgttattagattgtgttcaattaatccaagaactacaactgaagaaatgttgcaaatcatgatgaagattaaagcattggctgaagaagtttctatttcatacccatgtgttgctgaataa

SEQ ID NO: 37 - protein, 481 aa, Bacillus atrophaeus
MMSENLQLSAEEMRQLGYQAVDLIIDHMNHLKSKPVSETIDSDILRNKLTESIPENGSDPKELLHFLNRNVFNQITHVDHPHFLAFVPGPNNYVGVVADFLASGFNVFPTAWIAGAGAEQIELTTINWLKSMLGFPDSAEGLFVSGGSMANLTALTVARQAKLNNDIENAVVYFSDQTHFSVDRALKVLGFKHHQICRIETDEHLRISVSALKKQIKEDRTKGKKPFCVIANAGTTNCGAVDSLNELADLCNDEDVWLHADGSYGAPAILSEKGSAMLQGIHRADSLTLDPHKWLFQPYDVGCVLIRNSQYLSKTFRMMPEYIKDSETNVEGEINFGECGIELSRRFRALKVWLSFKVFGVAAFRQAIDHGIMLAEQVEAFLGKAKDWEVVTPAQLGIVTFRYIPSELASTDTINEINKKLVKEITHRGFAMLSTTELKEKVVIRLCSINPRTTTEEMLQIMMKIKALAEEVSISYPCVAE

SEQ ID NO: 38 - DNA, 1473 bp, Ruminococcus gnayus
  source 1..1473: mol_type = genomic DNA
atgtctcaagtgatcaaaaaaaagagaaacactttcatgatcggtaccgaatatatattgaactctacccaattagaagaggctattaaatctttcgtccatgatttctgtgccgaaaagcacgaaattcacgaccaaccagttgtcgttgaagctaaggaacaccaagaagataagattaagcaaatcaaaattccagaaaagggtcgtccagtcaatgaagttgtttctgaaatgatgaacgaagtctacagatacagaggtgatgccaaccatccaagattcttctctttcgttccaggtccagcttcctctgtctcctggttgggtgacatcatgacttctgcttacaacatccacgctggtggttctaaattggccccaatggttaactgtatcgaacaagaagtcttgaaatggctcgcaaagcaagttggtttcaccgaaaaccctggtggtgttttcgtttccggtggttctatggctaacattactgctttgacagccgctagagacaacaagcttaccgatattaacttgcacttgggtactgcttacatttccgaccaaacccactcttcagttgctaagggtttacgtatcattggtatcactgactccagaatcagaagaattccaactaactcccacttccaaatggacaccactaagttggaagaagctatcgaaaccgacaagaagtccggttacatcccatttgttgttatcggtactgccggtaccaccaacactggttccattgacccattgactgaaatttccgctttgtgtaagaagcatgacatgtggttccacattgatggtgcctacggtgcttcagttttgttgtctccaaagtacaagtctttgttaacaggtaccggtttggctgactctatctcttgggatgctcacaagtggttatttcaaacttacggttgtgctatggttttggtcaaggatattagaaacttgtttcattccttccacgtcaatccagaatacttgaaggatctggaaaacgacatcgataacgttaacacctgggacatcggcatggaattgaccagaccagccagaggtttgaagttgtggttgactttgcaagttctaggttctgatcttattggttctgccattgaacacggtttccaattggctgtctgggctgaagaagccttgaatccaaagaaggactgggaaattgtctctcctgctcaaatggctatgatcaacttcagatatgctccaaaggacttgaccaaggaagaacaagatatcttgaacgaaaagatctctcaccgtattttggaatccggttacgctgctatcttcactaccgtcctaaacggtaagactgtcttgagaatctgcgctattcacccagaagctactcaagaagacatgcaacacaccattgatttgttggaccaatacggtagagaaatctacactgaaatgaagaaggcttag

SEQ ID NO: 39 - protein, 490 aa, Ruminococcus gnayus
MSQVIKKKRNTFMIGTEYILNSTQLEEAIKSFVHDFCAEKHEIHDQPVVVEAKEHQEDKIKQIKIPEKGRPVNEVVSEMMNEVYRYRGDANHPRFFSFVPGPASSVSWLGDIMTSAYNIHAGGSKLAPMVNCIEQEVLKWLAKQVGFTENPGGVFVSGGSMANITALTAARDNKLTDINLHLGTAYISDQTHSSVAKGLRIIGITDSRIRRIPTNSHFQMDTTKLEEAIETDKKSGYIPFVVIGTAGTTNTGSIDPLTEISALCKKHDMWFHIDGAYGASVLLSPKYKSLLTGTGLADSISWDAHKWLFQTYGCAMVLVKDIRNLFHSFHVNPEYLKDLENDIDNVNTWDIGMELTRPARGLKLWLTLQVLGSDLIGSAIEHGFQLAVWAEEALNPKKDWEIVSPAQMAMINFRYAPKDLTKEEQDILNEKISHRILESGYAAIFTTVLNGKTVLRICAIHPEATQEDMQHTIDLLDQYGREIYTEMKKA

SEQ ID NO: 40 - DNA, 1482 bp, Streptomyces griseofuscus
  source 1..1482: mol_type = genomic DNA
atgaagccagctgaagctaaaccacctcacatggaccatgatactttcagatccttgggtcaccaagctattgactggattgctgattactggcaacacttggctgacagaccagttgctccattggttgaaccaggtactgttagagcccaattgcctaccactccaccagagtacggtgaagacttcccagctttgttatctgacttggaaagaattgtcttgccaggcttgttgcactggcaacacccaagattcttcggttacttcccagctaacgcttccggtccagccgttttagctgaattattatctgctggtctaggtgtccaaggtatgaactggaacacttctccagcttgtaccgaagttgaacaacaaatgttggactggttcgttcatttgttagggttgccagaacacttcagaggtggtggtgttatccaagacactgcctcctccgccgttttggtcgctctattgactgctttgcacagagcttctgctggtagaaccagagatcacggtactggtgaatgtggttacagggtttacctaaccgctgaaactcactccgctgctagaaaggctgccgtcatcaccggtttgggtttgcgtgctgtttgtgaagtcgcaactgatgctgacggtgctatggatgccgctgacttggccgctcgtattcaagccgatttggctgccggtttgaccccattgatggttgttgctacccgtggtaccacctctgatttgtccttcgatccattggaagacatcggtccagtctgtagaagacatggtgtctggttacacgttgacgctgcttatgctggtgttgctgcagtgtgcgatgaattgcattgggttaatgatggtgttagatacgctgattcttactgtactaacccacacaagtggttgttgaccaacttcgactgtgatttattatgggttgccgacccagaagctttggtcaacgctttgtccgttttgcctgcttacttgagaaacccagcttctgaatcaggtagagttactgactacagacactggcaagttccattgggcagaagattcagagccttgaagttgtggtctgtcttaagatggtacggtgccgaaggtttgagatctcacatcagaactggtgtcagacacgccagattgttcgctgacctggtcagagaagatgaccgtttcaccttgttggcgccagctactttgggtttggtcactttccgtcacgctggttctgacaccgacaacagaaacttgttgcacgctatcaacgccgaaggtacaacttttttaactcatagtgaaaagaacggtacctttttcttgagatttgctaccggtggtacccacactgaagaccatcacgtccacgaagcttggagagctgtccaaaatgccgtccaaaacgccgtcccaagagcccaacatgccaccgatggtactgccgatgctttgaacggttaa

SEQ ID NO: 41 - protein, 493 aa, Streptomyces griseofuscus
MKPAEAKPPHMDHDTFRSLGHQAIDWIADYWQHLADRPVAPLVEPGTVRAQLPTTPPEYGEDFPALLSDLERIVLPGLLHWQHPRFFGYFPANASGPAVLAELLSAGLGVQGMNWNTSPACTEVEQQMLDWFVHLLGLPEHFRGGGVIQDTASSAVLVALLTALHRASAGRTRDHGTGECGYRVYLTAETHSAARKAAVITGLGLRAVCEVATDADGAMDAADLAARIQADLAAGLTPLMVVATRGTTSDLSFDPLEDIGPVCRRHGVWLHVDAAYAGVAAVCDELHWVNDGVRYADSYCTNPHKWLLTNFDCDLLWVADPEALVNALSVLPAYLRNPASESGRVTDYRHWQVPLGRRFRALKLWSVLRWYGAEGLRSHIRTGVRHARLFADLVREDDRFTLLAPATLGLVTFRHAGSDTDNRNLLHAINAEGTTFLTHSEKNGTFFLRFATGGTHTEDHHVHEAWRAVQNAVQNAVPRAQHATDGTADALNG

SEQ ID NO: 42 - DNA, 1482 bp, Ceriporiopsis subvermispora
  source 1..1482: mol_type = genomic DNA
atggatattgaagcttttagaaaagcaggttaccaagctatcgatagaatttgtgactactactactctttgcaaaatagaccagttgttccatcagttcaaccaggttatttgttagatgcattaccagattctccaccagaacaaggtgaagatttcactgttattgctgatgattaccaaaagtacatcttgccaggtttgacacattggcaacatccatctttctttgcatactttccaacagcttgtacttttgaaggtattttgggtgacttgtattctacatcaactgcaaatccaggttttaattggttggcttctccagcatgtactgaattagaaatggttgttatggattggtctgctaagttgttaggtttgtcagaacatttcttgcattcttcaggtaaaggtggtggtgttattcaaactacagcatcagaattggctttagttgttgttgttgctgcaagagaaagatacttgagaattcatccagatgctaaggcagatgaattggttatatatactacaactcaaacacattctttgggtgttaaagctggtttagtttttggtatggaatgtagagcattggaagttaaagctgaagatgcttacgcattaagaggtgcaactttgaagtcagctttagaagaagatgaaaagagaggtaaaagaccttttattttggttgcaacagttggtacaacttcttcaggtgctattgatagattggatgaaattggtcaagtttctgaagattatccatcattatggattcatgttgatgctgcatgggcaggtgttactttggcttgtccagaatacagaggtacagctcaattagaaaacatcaacgcttatgcaacatctttcggtactaacttccataagtggggtttggttaactttgatgctgcattgttgtgggttaaggatagaaaggatttgacagatgctttagatgttactccagaattcttgagaacaaaacaaggtgacgctggtgcagttgttgattttagaaattggcatttgggtttgggtagaagattcagatctttgaaggtttggttcgttttgagatcatacggtgttgaaggttttagaaactacatcagacaaggtattaaattgaatgaacattttacttctttaattagagcttctttggatttttcattagttacagcaccatcattcgctttgacagtttttagattgactccagctggtgcatctttgactggttcagaattgaacgaattaaacagagctttctacgcaagattatcttcaagacatgatatcatgttgacacaaactgttttgaacggtgttttctgtatcagatttgcagttggtgctgcaagaactcaacaagaacatattgatacagcttgggatttgttacaacaagaaggtgctgttgcagttcaagaatacatgcaaaagatttctaaggatgttcaataa

SEQ ID NO: 43 - protein, 493 aa, Ceriporiopsis subvermispora
MDIEAFRKAGYQAIDRICDYYYSLQNRPVVPSVQPGYLLDALPDSPPEQGEDFTVIADDYQKYILPGLTHWQHPSFFAYFPTACTFEGILGDLYSTSTANPGFNWLASPACTELEMVVMDWSAKLLGLSEHFLHSSGKGGGVIQTTASELALVVVVAARERYLRIHPDAKADELVIYTTTQTHSLGVKAGLVFGMECRALEVKAEDAYALRGATLKSALEEDEKRGKRPFILVATVGTTSSGAIDRLDEIGQVSEDYPSLWIHVDAAWAGVTLACPEYRGTAQLENINAYATSFGTNFHKWGLVNFDAALLWVKDRKDLTDALDVTPEFLRTKQGDAGAVVDFRNWHLGLGRRFRSLKVWFVLRSYGVEGFRNYIRQGIKLNEHFTSLIRASLDFSLVTAPSFALTVFRLTPAGASLTGSELNELNRAFYARLSSRHDIMLTQTVLNGVFCIRFAVGAARTQQEHIDTAWDLLQQEGAVAVQEYMQKISKDVQ

SEQ ID NO: 44 - DNA, 1254 bp, Clostridium sporogenes
  source 1..1254: mol_type = genomic DNA
atgaagttctggagaaagtacacacaacaagaaatggatgaaaagattactgaatctttggaaaagactttgaactacgataacactaagacaatcggtattccaggtactaagttggatgatacagttttctatgatgatcattctttcgttaagcattcaccatacttgagaacttttattcaaaacccaaaccatatcggttgtcatacttatgataaggctgatatcttgttcggtggtacattcgatatcgaaagagaattaatccaattgttagcaatcgatgttttgaacggtaacgatgaagaatttgatggttacgttactcaaggtggtacagaagctaacatccaagcaatgtgggtttacagaaactacttcaagaaagaaagaaaggctaagcatgaagaaatcgctatcatcacttcagcagatacacattactctgcatacaaaggttcagatttgttgaacatcgatattattaaggttccagttgatttttattcaagaaaaattcaagaaaatacattggattcaattgttaaagaagctaaagaaattggtaaaaagtacttcatcgttatctctaacatgggtactacaatgtttggttcagttgatgatccagatttgtacgctaacatcttcgataagtacaatttggaatacaaaattcatgttgatggtgcatttggtggttttatatatccaattgataataaggaatgtaaaactgatttctctaataagaacgtttcttcaatcacattagatggtcataagatgttgcaagctccatacggtactggtatcttcgtttcaagaaagaatttgatccataacactttgacaaaggaagcaacttacatcgaaaatttggatgttacattgtctggttcaagatctggttcaaatgctgttgcaatttggatggttttagcttcttatggtccatacggttggatggaaaagattaataagttgagaaatagaactaaatggttgtgtaagcaattgaacgatatgagaattaaatattacaaagaagattcaatgaatattgttacaattgaagaacaatatgttaataaggaaatcgctgaaaagtactttttagttccagaagttcataacccaactaacaactggtacaagatcgttgttatggaacatgttgaattggatatcttgaactctttggtttacgatttgagaaagtttaataaggaacatttgaaggcaatgtaa

SEQ ID NO: 45 - protein, 417 aa, Clostridium sporogenes
MKFWRKYTQQEMDEKITESLEKTLNYDNTKTIGIPGTKLDDTVFYDDHSFVKHSPYLRTFIQNPNHIGCHTYDKADILFGGTFDIERELIQLLAIDVLNGNDEEFDGYVTQGGTEANIQAMWVYRNYFKKERKAKHEEIAIITSADTHYSAYKGSDLLNIDIIKVPVDFYSRKIQENTLDSIVKEAKEIGKKYFIVISNMGTTMFGSVDDPDLYANIFDKYNLEYKIHVDGAFGGFIYPIDNKECKTDFSNKNVSSITLDGHKMLQAPYGTGIFVSRKNLIHNTLTKEATYIENLDVTLSGSRSGSNAVAIWMVLASYGPYGWMEKINKLRNRTKWLCKQLNDMRIKYYKEDSMNIVTIEEQYVNKEIAEKYFLVPEVHNPTNNWYKIVVMEHVELDILNSLVYDLRKFNKEHLKAM

SEQ ID NO: 46 - DNA, 1443 bp, Homo sapiens
  source 1..1443: mol_type = genomic DNA
atgaatgcatctgaattcagaagaagaggtaaagaaatggttgattacatggctaactacatggaaggtattgaaggtagacaagtttatccagatgttgaaccaggttacttgagaccattaattccagctgcagctccacaagaaccagatacatttgaagatattattaacgatgttgaaaagattatcatgccaggtgttacacattggcattcaccatatttctttgcatactttccaactgcttcttcatatccagcaatgttggctgatatgttatgtggtgcaattggttgtattggtttttcttgggcagcttcaccagcttgtacagaattggaaactgttatgatggattggttaggtaaaatgttggaattaccaaaggcatttttgaacgaaaaggctggtgaaggtggtggtgttattcaaggttctgcatcagaagctacattagttgctttgttagcagctagaactaaagttattcatagattgcaagcagcttctccagaattaacacaagcagctatcatggaaaaattggttgcatattcttcagatcaagctcattcttcagttgaaagagctggtttgatcggtggtgttaagttgaaggcaatcccatctgatggtaattttgctatgagagcatcagctttgcaagaagcattagaaagagataaagcagctggtttgattccatttttcatggttgctactttaggtactacaacttgttgttctttcgataatttgttagaagttggtccaatttgtaataaggaagatatttggttgcatgttgatgcagcttacgcaggttcagcttttatttgtccagaattcagacatttgttgaacggtgttgaattcgctgattcttttaatttcaatccacataaatggttgttagttaacttcgattgttcagcaatgtgggttaagaaaagaacagatttgactggtgcttttagattagatccaacatatttgaagcattctcatcaagattcaggtttgatcactgattacagacattggcaaattccattgggtagaagattcagatctttgaagatgtggttcgtttttagaatgtacggtgttaagggtttgcaagcttacatcagaaagcatgttcaattgtctcatgaattcgaatcattagttagacaagatccaagattcgaaatctgtgttgaagttattttgggtttagtttgtttcagattgaagggttctaataaggttaacgaagcattgttgcaaagaattaattcagctaagaaaattcatttggttccatgtcatttgcgtgataagttcgttttgagattcgctatttgttcaagaactgttgaatcagcacatgttcaaagagcttgggaacatatcaaggaattggcagctgatgttttgagagctgaaagagaataa

SEQ ID NO: 47 - protein, 480 aa, Homo sapiens
MNASEFRRRGKEMVDYMANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 48 - DNA, 1521 bp, Ophiorrhiza pumila
  source 1..1521: mol_type = genomic DNA
atgggttctatctcagaaaactgtgatgattctatttcattagctgcaccttttagaccattggaaccagaagaattcagaaagcaagctcatgttatggttgatttcatcgcagattactacaagaacattgaaaattatccagttttgtcacaagttgaaccaggttatttgaaaaatagattgccagaaactgctccacatttgccagaatctttcgaaacaatcttgaaggatattaagaaagatatcgttcctggtatgactaattggttgtcaccaaatttctttgcttattttccagcaactgtttcttcagctgcatttgttggtgaaatgttgtgtacaggttttaattctgttggttttaattggttagcttcaccagcatctacagaattggaaatggttgttattgattggttagctaacatgttgaagttgccaaagtcttttatgtttcatggtactggtggtggtgttattcaaggtactacatctgaagctatcttgtgtacattgattgctgcaagagatggtgctttggaaaagattggtatggaaaacgttggtaaattagttgtttacggttcagatcaaactcattctttctttcaaaagacttgtaaggttgctggtatcttcccatgtaacatcaagttgatcccaactacaagagaagataacttctctatgtcaccaatcgcattgagagaacaaattgaagctgatgttgcagatggtttagttccaattttcttgtgtactacagttggtactacatcaactgctgcaattgatccagtttctgaagttgctaaggttgcaaacgatttcaatatttgggttcatgttgatgctgcttatgctggttctgcatgtatttgtccagaattcagacaatacttagatggtatcgaattggttgattctttttcattgtctccacataagtggttgttgtgtttcttggattgttgttgtttgtggttgaagaaaccacatttgatggttaaggctttgtcaactaacccagaatatttgagaaataagagatctgaattcgatggtgttgttgatttcaaggattggcaaatcggtacaggtagaagattcaaagctttgagattatggttggttatgagatcatacggtgttgaaaatttgaagagacatatcttgtctgatgttcaaatggctaagatgttcgaaggtttagttaaatctgatccaagattcgaaatcatcgttccaagagctttcgcattagtttgtttcagattgaaccctggtaaaggttatgatgatgaaatcgataaggaaatcttgaataaggaattgttagatttgattaattcaacaggtagagcttacatgactcatacaaaagcaggtggtatctatatgttgagatttgctgttggtactacattgactgaagaacatcatgtttacgctgcatgggaattgattaaagaatgtacagatgcatctttgactaaaacaaatattattgaataa

SEQ ID NO: 49 - protein, 506 aa, Ophiorrhiza pumila
MGSISENCDDSISLAAPFRPLEPEEFRKQAHVMVDFIADYYKNIENYPVLSQVEPGYLKNRLPETAPHLPESFETILKDIKKDIVPGMTNWLSPNFFAYFPATVSSAAFVGEMLCTGFNSVGFNWLASPASTELEMVVIDWLANMLKLPKSFMFHGTGGGVIQGTTSEAILCTLIAARDGALEKIGMENVGKLVVYGSDQTHSFFQKTCKVAGIFPCNIKLIPTTREDNFSMSPIALREQIEADVADGLVPIFLCTTVGTTSTAAIDPVSEVAKVANDFNIWVHVDAAYAGSACICPEFRQYLDGIELVDSFSLSPHKWLLCFLDCCCLWLKKPHLMVKALSTNPEYLRNKRSEFDGVVDFKDWQIGTGRRFKALRLWLVMRSYGVENLKRHILSDVQMAKMFEGLVKSDPRFEIIVPRAFALVCFRLNPGKGYDDEIDKEILNKELLDLINSTGRAYMTHTKAGGIYMLRFAVGTTLTEEHHVYAAWELIKECTDASLTKTNIIE

SEQ ID NO: 50 - DNA, 1545 bp, Oryza sativa
  source 1..1545: mol_type = genomic DNA
atgggttcattagatacaaatccaactgctttttctgcatttccagctggtgaaggtgaaacttttcaaccattgaacgcagatgatgttagatcatatttgcataaggctgttgatttcatctctgattactacaaatcagttgaatctatgccagttttgccaaacgttaagccaggttatttgcaagatgaattaagagcatcaccaccaacttactctgctccattcgatgttacaatgaaggaattgagatcttcagttgttcctggtatgactcattgggcatctccaaatttctttgctttctttccatcaacaaattctgctgcagctattgctggtgacttgattgcatcagctatgaatacagttggttttacttggcaagcttctccagcagctactgaaatggaagttttggcattagattggttggctcaaatgttgaatttgccaacttcttttatgaacagaactggtgaaggtagaggtactggtggtggtgttattttgggtactacatctgaagctatgttggttacattagttgcagctagagatgcagctttgagaagatctggttcagatggtgttgcaggtttgcatagattagctgtttatgcagctgatcaaacacattcaactttctttaaggcatgtagattggctggttttgatccagcaaatattagatctattccaacaggtgctgaaactgattatggtttagatccagcaagattgttagaagctatgcaagcagatgctgatgcaggtttagttccaacatacgtttgtgcaactgttggtactacatcttcaaatgctgttgatccagttggtgctgttgcagatgttgcagctagatttgcagcttgggttcatgttgatgcagcttacgcaggttcagcttgtatttgtccagaattcagacatcatttggatggtgttgaaagagttgattctatctcaatgtctccacataagtggttgatgacatgtttagattgtacttgtttgtacgttagagatacacatagattgactggttctttagaaactaacccagaatacttgaaaaatcatgcttcagattctggtgaagttacagatttgaaagatatgcaagttggtgttggtagaagattcagaggtttgaagttgtggatggttatgagaacttatggtgttgctaagttgcaagaacatatcagatcagatgttgctatggcaaaagtttttgaagatttggttagaggtgacgatagattcgaagttgttgttccaagaaacttcgctttggtttgtttcagaattagagctggtgcaggtgcagctgcagctacagaagaagatgcagatgaagctaacagagaattgatggaaagattgaataagactggtaaagcatacgttgctcatacagttgttggtggtagatttgttttgagatttgcagttggttcttcattacaagaagaacatcatgttagatctgcttgggaattgattaagaaaactacaactgaaatgatgaactaa

SEQ ID NO: 51 - protein, 514 aa, Oryza sativa
MGSLDTNPTAFSAFPAGEGETFQPLNADDVRSYLHKAVDFISDYYKSVESMPVLPNVKPGYLQDELRASPPTYSAPFDVTMKELRSSVVPGMTHWASPNFFAFFPSTNSAAAIAGDLIASAMNTVGFTWQASPAATEMEVLALDWLAQMLNLPTSFMNRTGEGRGTGGGVILGTTSEAMLVTLVAARDAALRRSGSDGVAGLHRLAVYAADQTHSTFFKACRLAGFDPANIRSIPTGAETDYGLDPARLLEAMQADADAGLVPTYVCATVGTTSSNAVDPVGAVADVAARFAAWVHVDAAYAGSACICPEFRHHLDGVERVDSISMSPHKWLMTCLDCTCLYVRDTHRLTGSLETNPEYLKNHASDSGEVTDLKDMQVGVGRRFRGLKLWMVMRTYGVAKLQEHIRSDVAMAKVFEDLVRGDDRFEVVVPRNFALVCFRIRAGAGAAAATEEDADEANRELMERLNKTGKAYVAHTVVGGRFVLRFAVGSSLQEEHHVRSAWELIKKTTTEMMN

SEQ ID NO: 52 - DNA, 1494 bp, Oryza sativa
  source 1..1494: mol_type = genomic DNA
atggaaggtgttggtggtggtggtggtggtgaagaatggttgagaccaatggatgcagaacaattaagagaatgtggtcatagaatggttgatttcgttgcagattactacaagtcaatcgaagcttttccagttttgtctcaagttcaaccaggttatttgaaagaagttttgccagattcagctccaagacaaccagatactttagattctttgttcgatgatatccaacaaaagattatcccaggtgttacacattggcaatcaccaaactacttcgcatactacccatctaattcttctactgctggtttcttgggtgaaatgttgtctgctgcttttaatatcgttggtttttcatggatcacttctccagctgcaacagaattggaagttattgttttagattggttcgcaaagatgttgcaattgccatcacaattcttatctactgctttgggtggtggtgttattcaaggtacagcttcagaagcagttttggttgcattgttagctgcaagagatagagctttgaagaaacatggtaaacattctttagaaaaattggttgtttacgcatcagatcaaactcattctgctttacaaaaagcatgtcaaatcgctggtattttctctgaaaacgttagagttgttattgctgattgtaataagaactacgctgttgcaccagaagcagtttcagaagctttgtctatcgatttgtcttcaggtttgatcccatttttcatttgtgcaacagttggtactacatcttcatctgctgttgatccattaccagaattgggtcaaatcgctaagtcaaacgatatgtggttccatatcgatgctgcttatgctggttctgcatgtatttgtccagaatacagacatcatttgaatggtgttgaagaagcagattcttttaatatgaatgctcataagtggttcttgactaacttcgattgttcattgttgtgggttaaggatagatcatttttgatccaatcattatctacaaatccagaattcttgaaaaataaggcttcacaagcaaactctgttgttgatttcaaggattggcaaatcccattgggtagaagattcagatcattgaagttgtggatggttttgagattgtacggtgttgataatttgcaatcttacatcagaaagcatatccatttggctgaacatttcgaacaattgttgttgtcagattcaagattcgaagttgttactccaagaacattttcattggtttgtttcagattagttccaccaacttctgatcatgaaaacggtagaaagttgaactacgatatgatggatggtgttaattcatctggtaaaattttcttgtcacatacagttttgtctggtaaattcgttttgagatttgctgttggtgcaccattaactgaagaaagacatgttgatgctgcatggaagttgttgagagatgaagctacaaaggttttaggtaaaatggtttaa

SEQ ID NO: 53 - protein, 497 aa, Oryza sativa
MEGVGGGGGGEEWLRPMDAEQLRECGHRMVDFVADYYKSIEAFPVLSQVQPGYLKEVLPDSAPRQPDTLDSLFDDIQQKIIPGVTHWQSPNYFAYYPSNSSTAGFLGEMLSAAFNIVGFSWITSPAATELEVIVLDWFAKMLQLPSQFLSTALGGGVIQGTASEAVLVALLAARDRALKKHGKHSLEKLVVYASDQTHSALQKACQIAGIFSENVRVVIADCNKNYAVAPEAVSEALSIDLSSGLIPFFICATVGTTSSSAVDPLPELGQIAKSNDMWFHIDAAYAGSACICPEYRHHLNGVEEADSFNMNAHKWFLTNFDCSLLWVKDRSFLIQSLSTNPEFLKNKASQANSVVDFKDWQIPLGRRFRSLKLWMVLRLYGVDNLQSYIRKHIHLAEHFEQLLLSDSRFEVVTPRTFSLVCFRLVPPTSDHENGRKLNYDMMDGVNSSGKIFLSHTVLSGKFVLRFAVGAPLTEERHVDAAWKLLRDEATKVLGKMV

SEQ ID NO: 54 - DNA, 1944 bp, Xenorhabdus doucetiae
  source 1..1944: mol_type = genomic DNA
atgacaaagccattccatccaaatttgaacgttgatgcattgtttttaggtccaaagtctgaaaacgctgttttctttagagaaatgatggattatgcagtttctgaacacatgcattggagatcaggttttcatccagaagattctacattggttacttcagttgatagatacgaacataactacagagaaacattgtacagaactgaaggtattttgaatcaattatctgctaagttgaaaaattcttcaatcccatttttctctccaagatacttaggtcatatttcttcagatactttgatggtttctaatttggcttatgttatggctatgatgtacaacccaaacaactgttcttatgaagcttcaccaactacaactgaattggaattagaatctggtttggatttgtgtagaatgttcggttacaatccacaacaatcttggggtcatattacatcaggtggtactgttgcaaattacgaaggtttgtgggttgctagaaatttgaagacattgtctttcgcaatctcacaacatccaaaggctaaggatttgatcgaacataagactccaaagcaattgaaaaatatgccaacatctgaattgttagatttgatcactgaattacaaaagagaggtttgttcgaagaaatcagagatatgacatgtagaggtactggtgttaaggctcaaattttaggtaaattgttagttccacaatctaagcattattcatggatgaaagctgcagatatcttcggtatcggtcaagaaaacatcatcccattgccagttaatgatcattaccaaacagatgttgctcaaatgagaaagatcactttcggtttgattgaacaaggtgaaccaattttagcaatgattgctgttgttggtacaactgaagttggtgcaatcgatagaatcgatgaagttattgctttaagaaaagaatgtgaagaaagatacggtgaatctttctacatccatgttgatgctgcttatgctggttacgcttgttcaatgttgttgaacgaacagggtgaattcgttgaatacgatgaattggcaaactaccatagatctgctggtattatgccagaaaatatttcatggccaaaaccagaagtttaccaatcttttaaagctttgaaagatgttgattcaattacagttgatccacataaggttggttttattcaatatgctgcaggtgcaatttgtatgaaggataagagaattttggaattaatttcttcacatgctgcatacgtttttgaaggtcatgctgataagacatctactaaaaatagaggtattttgggttcttcaattatggaaggttctaaatcaggtgcaacagctgcagctttgtgggcagctcatagattgttaccattgaacatcaacggttacggtaaagttattgcagctggtatcgttactgcacaatctttgttgaataagttggctaatttgccaccaatcgaaatcggtaaacatagattcgaagttcatatttttccatcaccagatttccacatgatcaacttcacttttaaagaagttggtaacgaatctttgtcaaaccataacgctttgaataagagattgtatgaattatgttcttactcagcaggtagagcttacacaaatgatttcttgacatcttcaactattttagattacaaggaatacggtgacactccttggcatcatgctcaaaaatgtggtttttcaagatcagaatgggaaaaagttagaaacatctatgttttgagagcagctgttatgacatactgtttgagagatgaagaacatttcgaagaattctgggaacaattgcaagctatcttcgttaagaaattgactcaaatcgttgatgaagaagataagaaagcaagattgagattagaaccagatgttccttttattggttaa

SEQ ID NO: 55 - protein, 647 aa, Xenorhabdus doucetiae
MTKPFHPNLNVDALFLGPKSENAVFFREMMDYAVSEHMHWRSGFHPEDSTLVTSVDRYEHNYRETLYRTEGILNQLSAKLKNSSIPFFSPRYLGHISSDTLMVSNLAYVMAMMYNPNNCSYEASPTTTELELESGLDLCRMFGYNPQQSWGHITSGGTVANYEGLWVARNLKTLSFAISQHPKAKDLIEHKTPKQLKNMPTSELLDLITELQKRGLFEEIRDMTCRGTGVKAQILGKLLVPQSKHYSWMKAADIFGIGQENIIPLPVNDHYQTDVAQMRKITFGLIEQGEPILAMIAVVGTTEVGAIDRIDEVIALRKECEERYGESFYIHVDAAYAGYACSMLLNEQGEFVEYDELANYHRSAGIMPENISWPKPEVYQSFKALKDVDSITVDPHKVGFIQYAAGAICMKDKRILELISSHAAYVFEGHADKTSTKNRGILGSSIMEGSKSGATAAALWAAHRLLPLNINGYGKVIAAGIVTAQSLLNKLANLPPIEIGKHRFEVHIFPSPDFHMINFTFKEVGNESLSNHNALNKRLYELCSYSAGRAYTNDFLTSSTILDYKEYGDTPWHHAQKCGFSRSEWEKVRNIYVLRAAVMTYCLRDEEHFEEFWEQLQAIFVKKLTQIVDEEDKKARLRLEPDVPFIG

SEQ ID NO: 56 - DNA, 1509 bp, Camptotheca acuminata
  source 1..1509: mol_type = genomic DNA
atgggttctttggattcaaactacgatactgaatctccagcttcagttggtcaattcaatccattagatccagaagaattcagaaagcaagctcattgtatcgttgatttcatcgcagattactacaagaacattgaatcttatccagttttgtcacaagttgatccaggttacagacattcaagattgggtaaaaatgctccatacagatctgaaccattcgaatcaatcttgaaggatgttcaaaaggatatcatccctggtatgactcattggatgtctccaaatttctttgctcattttccagcaactgtttcttcagctgcatttgttggtgaaatgttgtgtacatgtttcaactctgttggttttaattggttagcatcaccagctgcaacagaattggaaatggttgttattgattggttagctaacatgttgaagttgccaaagtcttttatgttttcaggtactggtggtggtgttttgcaaggtactacatctgaagctatcttgtgtacattgattgctgcatcaccaatgcatttcgaaatcgttggtgttaagacttctacatcattcgttgtttatggttctgatcaaactcattcaacatacgctaaagcatgtaaattagcaggtattttgccatgtaacatcagatctattccaactacagctgattcaaacttctctgtttcaccattgttgttgagaagagctattgaagcagataaagctgcaggtatggttccattgtatatttgtgctacagttggtactacatctactacagcaattgatccattatcttcattggctgatgttgcaaatgattatggtgtttggtttcatgttgatgctgcatacgctggttctgcatgtatttgtccagaattcagacattatttggatggtatcgaaagagcagattctttgtcattgtctccacataagtggttgttgtcttacttagattgttgttgtttgtgggttaagtcaccatctttattggttaaggctttatcaactgatccagaatatttgaaaaatcaaccatcagaatctaagtcagttgttgattacaaggattggcaagttggtacaggtagaagattcaaagctttgagattgtggttcgttatgagatcttatggtgttgcaaatttgcaatcacatatcagaactgatgttcaaatggctaagatgttcgaaggtttcgttaagtctgatccaagattcgaaattttggttccaagagttttctctttggtttgtttcagattgaacccaatctctggttcagatccaactggtacagaagctttgaacagaaagttattggattgggttaattctacaggtagagtttatatgactcatacaaaagttggtggtatctatatgttgagatttgctgttggtgcaactttaacagaaaagagacatgtttcttcagcttggaagttgattaaagaaggtgcagatgttttgttgaaggaagattaa

SEQ ID NO: 57 - protein, 502 aa, Camptotheca acuminata
MGSLDSNYDTESPASVGQFNPLDPEEFRKQAHCIVDFIADYYKNIESYPVLSQVDPGYRHSRLGKNAPYRSEPFESILKDVQKDIIPGMTHWMSPNFFAHFPATVSSAAFVGEMLCTCFNSVGFNWLASPAATELEMVVIDWLANMLKLPKSFMFSGTGGGVLQGTTSEAILCTLIAASPMHFEIVGVKTSTSFVVYGSDQTHSTYAKACKLAGILPCNIRSIPTTADSNFSVSPLLLRRAIEADKAAGMVPLYICATVGTTSTTAIDPLSSLADVANDYGVWFHVDAAYAGSACICPEFRHYLDGIERADSLSLSPHKWLLSYLDCCCLWVKSPSLLVKALSTDPEYLKNQPSESKSVVDYKDWQVGTGRRFKALRLWFVMRSYGVANLQSHIRTDVQMAKMFEGFVKSDPRFEILVPRVFSLVCFRLNPISGSDPTGTEALNRKLLDWVNSTGRVYMTHTKVGGIYMLRFAVGATLTEKRHVSSAWKLIKEGADVLLKED

SEQ ID NO: 58 - DNA, 792 bp, Oryctolagus cuniculus
  source 1..792: mol_type = genomic DNA
atggaaggtggttttactggtggtgacgaataccaaaagcatttcttgccaagagattacttaaacacatactactcattccaatctggtccatcaccagaagctgaaatgttgaagtttaatttggaatgtttgcataagactttcggtccaggtggtttgcaaggtgacactttaattgatattggttctggtccaacaatctatcaagttttggctgcatgtgaatcttttaaagatatcactttgtcagattttacagatagaaatagagaagaattggctaaatggttgaagaaagaaccaggtgcatacgattggactccagctttgaagttcgcatgtgaattagaaggcaactctggtagatggcaagaaaaagctgaaaagttgagagcaacagttaagagagttttgaagtgtgatgctaatttgtcaaatccattaactccagttgttttgccaccagcagattgtgttttgacattgttagctatggaatgtgcatgttgttctttggatgcttatagagctgcattgagaaatttggcatcattgttaaaaccaggtggtcatttggttactacagttactttgcaattatcttcatacatggttggtgaaagagaattttcttgtgttgctttggaaaaagaagaagttgaacaagctgttttagatgcaggtttcgatatcgaacaattgttgtactctccacaatcttactcagcttctacagcaccaaatagaggtgtttgtttcttggttgctagaaagaaaccaggttcataa

SEQ ID NO: 59 - protein, 263 aa, Oryctolagus cuniculus
MEGGFTGGDEYQKHFLPRDYLNTYYSFQSGPSPEAEMLKFNLECLHKTFGPGGLQGDTLIDIGSGPTIYQVLAACESFKDITLSDFTDRNREELAKWLKKEPGAYDWTPALKFACELEGNSGRWQEKAEKLRATVKRVLKCDANLSNPLTPVVLPPADCVLTLLAMECACCSLDAYRAALRNLASLLKPGGHLVTTVTLQLSSYMVGEREFSCVALEKEEVEQAVLDAGFDIEQLLYSPQSYSASTAPNRGVCFLVARKKPGS

SEQ ID NO: 60 - DNA, 1131 bp, Hordeum vulgare
  source 1..1131: mol_type = genomic DNA
atggataagatctctgcaccatttttctctggtacttcaccagctgcagcttcagttgctggtgttgatgaagatgatagattgtgtttccaagctcaagaattgatgttcgcatacaacatctctatggttttgagagcagctattcaattaggtttgttagatgctttgtcagcagctggtggtaaagcattgactccaaacgaattagttgaaaacgttgaaacatcttcaaataaggctgaagcagctgcagctgttgatagaattttgagatatttgtcttgtttcaacgttgttacttgttcttcagaagcagctggtccagatggtacattagttagaagatacactacaggtccattgtgtagatggttaactaaagatagaggtgacggtacattatctccatttgctgtttttgttgttgatccagatcatttgtttccttggcatcatattgcagaagctgttactgctggtggtccatcagcattcgaaagaacacaaaagtggccatactacgaatacatgggtaaaaatcaaagattgggtactttgttcgataacgcaatggctcaacattctgttattttggttacaaagatgttagaaagattcaaaggttttgatggtgttcaaagattagttgatgttggtggtggtactggttctacattgggtatgatcacttcaaagtacaagcacatgacaggtattaattacgatttgccacatgttattgctcaaggtttgccattaccaggtgttgaacatgttgcaggtgacatgtacgaatctattccaactggtgacgctgttttgttacaatggattacattgatgttgaacgatgatgaattcgttaagatcttgtcaaattgtcataatgctttgccaaaagatggtaaagttattgttgttgatggtattttaccagaaaacccagattcttcattgactgcaagagatgcttttacattagatatcatcatgttcgttttgtttaaaggtgcaaagcaaagaactgaaaaggaattcgcaagattagctaagcaagcaggttttactggtggtattaagaaaacttatattttctttaatttttacgctttggaattcactaaataa

SEQ ID NO: 61 - protein, 376 aa, Hordeum vulgare
MDKISAPFFSGTSPAAASVAGVDEDDRLCFQAQELMFAYNISMVLRAAIQLGLLDALSAAGGKALTPNELVENVETSSNKAEAAAAVDRILRYLSCFNVVTCSSEAAGPDGTLVRRYTTGPLCRWLTKDRGDGTLSPFAVFVVDPDHLFPWHHIAEAVTAGGPSAFERTQKWPYYEYMGKNQRLGTLFDNAMAQHSVILVTKMLERFKGFDGVQRLVDVGGGTGSTLGMITSKYKHMTGINYDLPHVIAQGLPLPGVEHVAGDMYESIPTGDAVLLQWITLMLNDDEFVKILSNCHNALPKDGKVIVVDGILPENPDSSLTARDAFTLDIIMFVLFKGAKQRTEKEFARLAKQAGFTGGIKKTYIFFNFYALEFTK

SEQ ID NO: 62 - DNA, 549 bp, Streptomyces griseofuscus
  source 1..549: mol_type = genomic DNA
atgaacaccttcagaacagccactgccagagacatacctgatgtagcagcaactcttacggaagccttcgcaactgatccacccacgcagtgggtgttccccgacggtactgccgccgtcagcaggttctttacacatgttgcagatagggttcacacggccggtggtattgttgagctactaccagacagagccgccatgattgcattgccaccacacgtgaggctgccaggagaagctgccgacggaaggcaggcggaaattcagagaaggctggcagacaggcacccgctgacacctcactactacctgctgttttacggagttagaacggcacaccagggttcgggattgggcggaagaatgctggccagattaactagcagagctgatagggacagggtgggtacatatactgaggcatccacctggcgtggcgctagactgatgctgagacatggattccatgctacaaggccactaagattgccagatggacccagcatgtttccactttggagagatccaatccatgatcattctgattag

SEQ ID NO: 63 - protein, 182 aa, Streptomyces griseofuscus
MNTFRTATARDIPDVAATLTEAFATDPPTQWVFPDGTAAVSRFFTHVADRVHTAGGIVELLPDRAAMIALPPHVRLPGEAADGRQAEIQRRLADRHPLTPHYYLLFYGVRTAHQGSGLGGRMLARLTSRADRDRVGTYTEASTWRGARLMLRHGFHATRPLRLPDGPSMFPLWRDPIHDHSD

SEQ ID NO: 64 - DNA, 1170 bp, Thermotoga maritima
  source 1..1170: mol_type = genomic DNA
atgaaaggatatttcggaccatacggtggccagtacgtaccagaaatattaatgggtgccttagaggagttagaggcagcatacgaggagattatgaaggatgagagcttctggaaggagttcaacgatctactgagggattacgcaggcagaccaacgccattgtactttgccaggagattgtctgagaagtacggcgcccgtgtttacttgaagcgtgaggatctgctgcacactggagcacacaagataaataacgctatcggacaggttttattggccaaattaatggggaagacacgtatcatagccgagacgggagctgggcagcatggagtcgctactgctaccgctgctgccctgttcggaatggaatgtgtgatctacatgggtgaagaggacacaatcagacagaagttgaacgtggagcgtatgaaattattaggggctaaagttgtccctgttaagtctggcagtaggaccttgaaggatgcgatagacgaggctttgagagactggattactaatttacagacaacatattatgttatcggatctgttgttggtccccacccttacccaattatcgtaaggaatttccagaaggttatcggtgaggagaccaagaagcaaataccagaaaaggaaggtcgtttgccagactatatagttgcctgcgtaggcggcggtagcaatgccgcaggtatattttacccattcatagactctggagtaaagctgataggtgttgaggcaggtggcgagggattggagacaggtaaacacgcagcctcgttattaaagggtaaaattggctatttacatggatcgaagacctttgttctacaagatgactggggtcaagtccaagtgagccattcggtgtcagctggtcttgactattcaggagtaggacctgagcatgcttattggagagagacagggaaggttctgtacgacgcagtgactgacgaagaggctttggacgcatttatagagttatcaagactagagggcattatacccgctttagagtcatcgcatgctctagcatatttgaagaagataaatataaaaggtaaggttgtggtggtcaacctatcagggagaggggataaagacctggagtcagtcttaaaccatccatacgtgagagaaagaattagatga

SEQ ID NO: 65 - protein, 389 aa, Thermotoga maritima
MKGYFGPYGGQYVPEILMGALEELEAAYEEIMKDESFWKEFNDLLRDYAGRPTPLYFARRLSEKYGARVYLKREDLLHTGAHKINNAIGQVLLAKLMGKTRIIAETGAGQHGVATATAAALFGMECVIYMGEEDTIRQKLNVERMKLLGAKVVPVKSGSRTLKDAIDEALRDWITNLQTTYYVIGSVVGPHPYPIIVRNFQKVIGEETKKQIPEKEGRLPDYIVACVGGGSNAAGIFYPFIDSGVKLIGVEAGGEGLETGKHAASLLKGKIGYLHGSKTFVLQDDWGQVQVSHSVSAGLDYSGVGPEHAYWRETGKVLYDAVTDEEALDAFIELSRLEGIIPALESSHALAYLKKINIKGKVVVVNLSGRGDKDLESVLNHPYVRERIR

SEQ ID NO: 66 - DNA, 1167 bp, Pyrococcus furiosus
  source 1..1167: mol_type = genomic DNA
atgtggttcggtgagtttggtggacaatatgtgccagagactttagtgggtcctcttaaggaattggaaaaggcatataaaaggttcaaggacgatgaggagttcaacaggcaactaaactattatttgaagacatgggccggtagaccaacgcccttgtattatgctaagaggttaactgaaaagattggcggcgcgaaagtgtatctgaaaagagaagacctagttcatggtggagcacacaagacaaataatgccattggacaagcactattggcaaagctaatgggtaaaactagattgatagctgagacaggagcgggtcaacatggggtcgcgacagcgatggctggtgcactactggggatgaaggtagatatttacatgggtgctgaggacgttgagcgtcagaaactaaatgtcttcaggatgaagctattaggtgccaatgttatacctgtaaattctggctcaagaacactaaaggacgccttcgacgaggctcttagagactgggttgccactttcgagtatactcattacttgatcggttcagtggttggaccacatccatacccaaccatcgttagggactttcagagcgtgattggtagagaggctaaggcacagatcttagaagcagagggacagctacctgacgtcatagttgcctgcgtcggcggtggctctaacgcaatgggtatattctatccattcgttaatgacaagaaggttaaattagtaggagtcgaagctggcggaaaggggttagagtcgggtaaacactcagcaagcttaaatgcaggacaggtaggggtgtcccacggcatgttgtcgtatttcttgcaagacgaggaaggtcagataaagccaagtcattcaattgctccaggccttgaccaccccggtgttggtccagagcacgcttacttaaagaagattcaaagggccgagtacgtcgctgtaacagacgaagaggcattgaaagctttccatgagctatccagaactgaggggattatacccgcccttgagtctgcccatgctgtggcgtacgccatgaagttagctaaagagatgtcccgtgacgaaatcatcattgtaaatctatcagggagaggagacaaggatttggacattgtattgaaggcaagcggaaatgtttga

SEQ ID NO: 67 - protein, 388 aa, Pyrococcus furiosus
MWFGEFGGQYVPETLVGPLKELEKAYKRFKDDEEFNRQLNYYLKTWAGRPTPLYYAKRLTEKIGGAKVYLKREDLVHGGAHKTNNAIGQALLAKLMGKTRLIAETGAGQHGVATAMAGALLGMKVDIYMGAEDVERQKLNVFRMKLLGANVIPVNSGSRTLKDAFDEALRDWVATFEYTHYLIGSVVGPHPYPTIVRDFQSVIGREAKAQILEAEGQLPDVIVACVGGGSNAMGIFYPFVNDKKVKLVGVEAGGKGLESGKHSASLNAGQVGVSHGMLSYFLQDEEGQIKPSHSIAPGLDHPGVGPEHAYLKKIQRAEYVAVTDEEALKAFHELSRTEGIIPALESAHAVAYAMKLAKEMSRDEIIIVNLSGRGDKDLDIVLKASGNV

SEQ ID NO: 68 - DNA, 1167 bp, Pyrococcus furiosus
  source 1..1167: mol_type = genomic DNA
atgtggtttggggaattcggtggtcaatacgtgccagaaaccttagttgggcctttaaaggagcttgagaaagcttacaaaagatttaaggacgacgaggagtttaataggcaattaaactattacttgaagacttgggcaggtagaccaacaccactttattacgcgaagagacttacagagaagatcggtggcgctaaggtttatttgaagcgtgaggatttggttcatggtggagcacataagacaaacaatgccatcgggcaagccttgttggccaaattgatgggtaagactaggttgatagctggcacaggtgccggccagcatggcgttgcaacagcaatggctggtgctcttttagggatgaaggtggatatctatatgggcgccgaggacgttgaaagacaaaagcttaacgtattcagaatgaagttattaggtgcgaatgttattccagtgaattcaggttccaggacgctgaaggacgcaatagacgaagccttgagagactgggttgctacttttgagtacacacattatttgattggttccgtggttggaccccacccgtaccctacaatagttagagattttcaatctgttatcggtagagaggcgaaggctcaaattccagaggccgagggacagctacccgatgttatagtcgcctgcgttggaggaggatcaaatgcgatgggaatcttctacccttttgtaaatgataagaaagttaagttagtcggagttgaggccggtggtaaagggctagaaagcggcaaacactcggcatctcttaatgccggtcaagtaggagtaagtcacgggatgttatcttacttcttgcaggatgaggaaggtcaaattaagccatctcattctatagccccaggattagactatcccggtgttgggcctgagcacgcttatctgaagaagatacagcgtgctgagtacgttgcagtaactgatgaagaggctttgaaggcttttcacgagttatcaagaactgagggaataattcctgccttggaatcggcacatgcggttgcctacgccatgaagcttgctaaggaaatgtcaagagacgaaatcattatagtgaatttatcgggaaggggcgataaggacttggacattgttttaaaagcaagtggcaatgtataa

SEQ ID NO: 69 - protein, 388 aa, Pyrococcus furiosus
MWFGEFGGQYVPETLVGPLKELEKAYKRFKDDEEFNRQLNYYLKTWAGRPTPLYYAKRLTEKIGGAKVYLKREDLVHGGAHKTNNAIGQALLAKLMGKTRLIAGTGAGQHGVATAMAGALLGMKVDIYMGAEDVERQKLNVFRMKLLGANVIPVNSGSRTLKDAIDEALRDWVATFEYTHYLIGSVVGPHPYPTIVRDFQSVIGREAKAQIPEAEGQLPDVIVACVGGGSNAMGIFYPFVNDKKVKLVGVEAGGKGLESGKHSASLNAGQVGVSHGMLSYFLQDEEGQIKPSHSIAPGLDYPGVGPEHAYLKKIQRAEYVAVTDEEALKAFHELSRTEGIIPALESAHAVAYAMKLAKEMSRDEIIIVNLSGRGDKDLDIVLKASGNV

SEQ ID NO: 70 - DNA, 1173 bp, Pyrococcus furiosus
  source 1..1173: mol_type = genomic DNA
atgtggtttggtgagtttggtggtcagtatgtgcctgagacgctagttgggccactgaaggagttagagaaagcgtacaagagattcaaagatgacgaggagtttaaccgtcagttgaattattatcttaagacgtgggctggtaggccgacccctctatactacgctaagcgtttaaccgaaaagataggtggcgccaagatctatctgaagagagaggacctagtccacggcggggctcacaaaactaacaacgcaattggtcaggcaccattagcaaagttaatgggcaaaacacgtctaattgccgaaactggtgctggtcaacatggagttgctacggcaatggcgggcgcactgttaggtatgaaagtggatatctatatgggagcagaagacgtggagagacagaagatgaacgtgttccgtatgaaactattgggagctaatgttatcccggttaactctggctcaagaactgcaaaggacgccataaacgaagctttgagggattgggaagccacattcgagtacacacactaccttataggctcagtcgtaggcccgcatccctaccccaccatagttcgtgacttccagagtgttattggccgtgaggccaaggcacagatcttggaagctgaaggtcaattgcctgacgtaatagttgcatgtgtgggtggcggttctaacgctatgggtatattctaccccttcgtgaacgataagaaagtaaagctagttggagtggaagcaggtggcaaagggttggagtcgggcaagcactcagcctcattaaacgcaggtcaggtaggcgttctgcacggtatgttatcctatttcttacaggacgaggaaggccaaatcaaaccatcgcactcaatcgcacctggtttagattacccaggagtcggtcccgagcacgcatatttaaagaaaatccagagagcagagtacgtcacggtgacggatgaggaagcccttaaagcgtttcacgagctgtcgaggaccgagggcataatacctgctcttgaaagtgcccacgcagttgcctatgctatgaagttggccaaggagatgagtagagacgagattatcatagtaaatctgtcaggcagaggtgacaaggatcttgacatagtccttaaggcgagcggtaatgttttggagtaa

SEQ ID NO: 71 - protein, 390 aa, Pyrococcus furiosus
MWFGEFGGQYVPETLVGPLKELEKAYKRFKDDEEFNRQLNYYLKTWAGRPTPLYYAKRLTEKIGGAKIYLKREDLVHGGAHKTNNAIGQAPLAKLMGKTRLIAETGAGQHGVATAMAGALLGMKVDIYMGAEDVERQKMNVFRMKLLGANVIPVNSGSRTAKDAINEALRDWEATFEYTHYLIGSVVGPHPYPTIVRDFQSVIGREAKAQILEAEGQLPDVIVACVGGGSNAMGIFYPFVNDKKVKLVGVEAGGKGLESGKHSASLNAGQVGVLHGMLSYFLQDEEGQIKPSHSIAPGLDYPGVGPEHAYLKKIQRAEYVTVTDEEALKAFHELSRTEGIIPALESAHAVAYAMKLAKEMSRDEIIIVNLSGRGDKDLDIVLKASGNVLE

SEQ ID NO: 72 - DNA, 1173 bp, Pyrococcus furiosus
  source 1..1173: mol_type = genomic DNA
atgtggttcggagagttcggcgggcagtacgtcccagaaacgctagtcggcccactgaaggagctggagaaagcatacaaaagattcaaggatgacgaggagttcaacaggcaattaaactattacctaaagacctgggctggtaggcccacccctttatactatgcaaaacgtttaacggagaaaatcggtggcgcaaaagtgtacttgaagagagaagacttagtccacggtggtgctcataaaactaacaacgctatagggcaggcacttttagctaagttgatgggtaaaacgaggttgattgcgggtaccggtgccggtcaacatggcgttgctacggcaatggcgggagccctattgggcatgaaggttgacatttatatgggcgctgaggacgtggaacgtcagaaactgaacgtctatcgtatgaaattgcttggtgccaacgttataccagtcaattccggctcgcgtacggttaaagacgctttcgatgaggcactatgtgatagagttgccactttcgaatacacccactatcttattggtactgtctggggtccacatccgtatccaactattgtaagggacttccagactgttatcggaagagaggcaaaggcccaaatattagaggctgagggaaggttacccgacgccattgttgcatgcgtaggcggtggtagtaacgcaatgggtattttctatcccttcgttaacgataagaaggttaagttagtgggtgtcgaggccggtggtaaggggcttgagtccggtaaacattccgcatctttgaatgctgggcaagtaggtgtcagtcatggaatgttatcatactttctacaggacgaggaaggacagataaaaccttctcattctatagcgccaggtcttgaccaccctggcgttggaccagagcatgcttatttgaagaagatccagagagctgaatatgtagctgttacggatgaggaagctctaaaggcgttccacgagttaagtaggactgagggaattataccggcattagagtctgcccacgctgtagcttacgcaatgaagctggccaaagagatgagccgtgacgagatcatcatcgtaaacttgtctggcagaggcgataaggacctagacatcgtattaaaggcctcggggaacgtcctagagtag

SEQ ID NO: 73 - protein, 390 aa, Pyrococcus furiosus
MWFGEFGGQYVPETLVGPLKELEKAYKRFKDDEEFNRQLNYYLKTWAGRPTPLYYAKRLTEKIGGAKVYLKREDLVHGGAHKTNNAIGQALLAKLMGKTRLIAGTGAGQHGVATAMAGALLGMKVDIYMGAEDVERQKLNVYRMKLLGANVIPVNSGSRTVKDAFDEALCDRVATFEYTHYLIGTVWGPHPYPTIVRDFQTVIGREAKAQILEAEGRLPDAIVACVGGGSNAMGIFYPFVNDKKVKLVGVEAGGKGLESGKHSASLNAGQVGVSHGMLSYFLQDEEGQIKPSHSIAPGLDHPGVGPEHAYLKKIQRAEYVAVTDEEALKAFHELSRTEGIIPALESAHAVAYAMKLAKEMSRDEIIIVNLSGRGDKDLDIVLKASGNVLE

SEQ ID NO: 74 - DNA, 6347 bp, Saccharomyces cerevisiae
  source 1..6347: mol_type = genomic DNA
gtatccggctgttccttcatagccctttcaatgaacgttgcagccctttgaagattggccattttgtcaggactcgagcctgacagttggaccaacgcaactttaattttttgtgaaagaatcttcgaagcactcatactggcgatcttcacgccctcctgctattacaaaagctgtgtttttacaagaatcaaattaagttagcaagatattatacaacattattgataatttcaatatcgtgttcgtacctgatgacgtatctgtgcattgataaggcccgcatggtttcagaaagcagagcggaacgattccaaattagtggccttgtgctttgcatgtcaattgtgttaccttcagctcgtggatttgttttatcaatacacagtctacagtcaagaattttttttatcaaattttgcgttcgagcgtataaaatagccgctgtagctacttaagttcctgttcagcgatagtttttttccatcacacgtactatggcaattaagtcctcagcgagctcgcatggaatgcgtgcgatgagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacggaatgcgtgcgattcaggcgtagtctggaacgtcgtatgggtatggaccagagacggattgagaaacttgttcggttcttgggacgaatctacattggaatggaactgggtgcctgaagaaaccagtggtgaaatcagctgggatgtcaattggcttaccgttttggtcaactggtctttcaatgttgaaagcagacaacaaagtagcaccggcaatccaaacagtagattgagctaggtgaatacctgggcagtttcttctaccgtaaccgaaagcagcctttcttgggtctctaacagtgttgtctggtttaccatcaggacccaagtatctttctggacggaaaacggatggatctggatagacttctgggtcattcaaaactgcccaggtgttagcaaaaaccaatgtgttctttggaatcaaataacctctgtaaacatcatccttcatcaatttatgagggatggctaatggagcaatttggttccatctgaataattccttgatacaagcggtcaaatatgggagtgagtcgtcttcctcatcgtagtcagggatttgaccgttgttggtcaaagcatccaattcagcttgaacctttctttgcacttctgggtatttaaccatggccaatatgaaagcggacatagcagagacggtagtgtcgccaccaccaacgttaacttcagcagcagtgttcttgataacatgttcttggtgttctaaatcaccgttcaagtccatagcttgtaatctagcagaagcgtaagatggtctggttagaccttgtggagccaactttctcatagtttcgtatggcatgtcaaccatgtggtcagcggcttctctccagaccttggccttacgcttgaagacggcacctgggaaccaagctggcaagtacttcaaagatgggaaggagtcaacccagaacttacctggaacactagcaatggccaaaccttcgttggctaagtgggtagcttctaaccatgggtcatcttcggctagatcgataccgtaaccaatgtccaaagacatagcagcgatttgatgtctgatgtgttgagcccaccggtccggagtcttggtcaattgttgaaccaattggtgagcagccttgacttgagcgtgtctgaattgcttgatacccttttcagagaattccttagcaaacattcttctttcttctctccaacggtcaccgtaagtgataaaacccagatcaaattcccaacccatcaattcattgaccatagtggattccaatctaccggagtagatggaaccacgtttttccaagagatccgtgatagtttccaaggtgtttaaaatgaccatttcggtaccaccagcatctacgtacaagatatcagtgttgtagtcacgaccccattgcaagaaagtcaaccatggagattcttctggcatgtcgaacatgttaccaataaatgggattggaattcctggtggaccaggtggcaatctggatctacgaactcttctggagacgatgtagtaaatacaaccagcaatgacgaaagagaacaagacagcaatcattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggcttctagttcttccgatgtcttcgttttgggtctaggtgttgttttggctgccttgtatatcttcagagaccaattattcgctgcttctaagccaaaggtggctccagtttccactacgaagcctgccaacggttccgctaacccaagagacttcatcgccaagatgaaacaaggtaagaagagaatcgtaatcttctacggttctcaaactggtaccgctgaagaatatgctattcgtttggctaaggaagctaagcaaaagttcggtctagcctccttggtttgtgatccagaagaatacgattttgaaaagttggaccaattgccagaagattctattgctttcttcgtcgttgctacctatggtgaaggtgaacctacagacaacgctgtccaattgttgcaaaacttgcaagatgaaagcttcgaattctcctctggtgagagaaagttgtcaggtttgaagtacgttgtttttggtctgggtaacaagacctacgaacattacaacctcattgggagaactgttgacgctcaattggccaagatgggtgctatcagaatcggtgaaagaggtgaaggtgatgatgacaagtccatggaagaagactacttggaatggaaggatggtatgtgggaagcgtttgccactgctatgggtgttgaagaaggtcaaggtggtgactccgctgatttcgtcgtttccgaattggaatctcacccaccagaaaaggtttaccaaggtgaattttctgctagagctttaaccaaaaccaagggtattcacgacgctaagaatccttttgctgctccaattgcggttgctagagaattgttccaatctgttgtcgatagaaactgtgtccacgtcgaattcaacattgaaggctctggtatcacctatcaacacggtgaccacgttggtttgtggccattgaatccagatgttgaagtcgaacggttgttgtgtgttttaggtttagctgaaaagagagatgctgtcatctccattgaatccttagacccggctttggctaaggttccattcccagtcccaactacttacggtgctgtgttgagacactacattgacatctctgctgtcgccggtagacaaatcttgggtactttgtccaaattcgctccaaccccagaagctgaagctttcttgagaaacttgaacactaacaaggaagaataccacaacgtcgtcgctaacggttgtttgaaattgggtgaaattttgcaaatcgctaccggtaacgacattactgtcccaccaactactgccaacaccaccaaatggccaattccattcgacatcattgtttctgccatcccaagattgcaaccaagatactactctatctcttcttccccaaaaattcatccaaacaccatccacgctaccgttgttgtgctcaaatacgaaaacgttccaaccgaaccaatcccaagaaagtgggtttacggtgtcggtagtaacttcttgttgaatttaaagtacgctgttaacaaggaaccagttccatacatcactcaaaatggcgaacaaagagtcggtgtcccggaatacttgattgctggtccacgtggttcttacaagactgaatctttctacaaggctccaatccatgttagacgttctactttccgtttgccaaccaacccaaagtctccagtcatcatgattggtccaggtactggtgtcgccccattcagaggcttcgttcaagaaagagttgccttggccagaagatccatcgaaaagaacggtcctgactctttggctgactggggtcgtatttccttgttctacggttgtagaagatccgacgaagacttcttgtacaaggacgaatggccacaatacgaagctgagttgaagggtaagttcaagttgcactgtgctttctccagacaaaactacaagccagacggttctaagatttacgtccaagatttgatctgggaagacagagaacacattgccgatgccatcttaaacggtaagggttacgtctacatctgcggtgaagctaagtccatgtctaaacaagttgaagaagttctagccaagatcttgggcgaagccaaaggtggttccggtccagttgaaggtgttgctgaagtcaagttactgaaggaacggtccagattgatgttggatgtctggtctgaacaaaagttaatttctgaagaagatttggaatgaatcgcgtgcattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatcgcgtcccaattcgccctatagtgagtcgtattacgcgcgctcactggccgtcgttttacaacgtcgtgactgggaaaaccctggcgttacccctgcaggactagtgctgaggcattaatagtttactcaattcttgaagccaatttgtacaattccccattagagtcaaataaaaggatgcctcacggaggtatgttacccgcgctatttcacatggctcattgaattagaggtggaatttggtgtaccctcccctcctcatctgatgaagtagtgatccgacaattcttaaaagttgtagacattacttttaccaccaactaagttgtatttatattgctacccttatccttttatatctaactagcgctcataaggttggggcaatactaaaactgtgttcttattcaactcattaaatacgtggcagtacgtaccctattagaaacaataggaaacagcagagtcggaagaagccaaatgccagatttgaagtccaaaaccttgtcaagccaatctttgggagcggctattcctccagaaattgtgtaccaaatacttacataccagtttagggatttgttaagaaatgaccatccaggtacggcagaaa

SEQ ID NO: 75 - DNA, 4792 bp, Saccharomyces cerevisiae
  source 1..4792: mol_type = genomic DNA
caatctggcggcttgagttctcaacatgttttattttttacttatattgctggtagggtaaaaaaatataactcctaggaataggttgtctatatgtttttgtcttgcttctataattgtaacaaacaaggaaagggaaaatactgggtgtaaaagccattgagtcaagttaggtcatcccttttatacaaaatttttcaattttttttccaagattcttgtacgattaattattttttttttgcgtcctacagcgtgatgaaaatttcgcctgctgcaagatgagcgggaacgggcgaaatgtgcacgcgcacaacttacgaaacgcggatgagtcactgacagccaccgcagaggttctgactcctactgagctctattggaggtggcagaaccggtaccggaggaggccgctataaccggtttgaatttattgtcacagtgtcacatcagcattaagtcctcagcgagctcgcatggaatgcgtgcgatgagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacggaatgcgtgcgattcaggtggagtccaaacccaacaaaggatttggaattggcttaccggcagtggaggattcctttagcaacgtggaagtgatttcaccgttgtcgttgttacctctagcatcgtggaaagcagcaacacccttcttaacaaagttgattctttcttcttcagaaccccattgcatgaagtcagtccacataacaatgtgagcggcgataccagcggtaaccttggcgtagttgatggaatgcttggaagtacgggcgtaagattgcaagtaagcttgtctcatggttgtaccaacttgttcgtcttggaatctgctaatcaagtaacagtcacccaagaagtaacccaaatccaatgaagctggaccgtacttacacaattcccagtctaagatgtagatcttttgcaacttagatgggttaccttcttcaagttgcaacaagatgttcccagaccacaagtcagccatgaccaaagtttcttcggagtgcataacatcgtcaactagatccttgacaacagttggcaacaatggatcatcgacgccgtatttagcggcgtttgggataatagtttggtacaattggtcagaggtggttctaccgacaatgttaccagagaagaacttgaattctgggtcgtctcttctttctctacctatgttgtgcaatctggcgacgaaaccaccaatctcggtaccaaccaatctagcaatatcggtagccaaaggtggcttagcagtaacgtagtctaataaggtcttcattttaccgacatcttgcataatcaaagcattgttttccaagtcatagttgagaccttctggaacagagacaataccatcaacaccacccaaaacttctctgttagccatcatcaacttgatagcttggtattcgtagacagaacgttcaacaccgattttgaaatcttcatcagtagacatgtgtggttgagcgtgcttcaaaatgatagaagtgtgaccttggtatggagcgttcaatttaatacgccaggtgacgttaacgaaaccaccggataatctcttgacaccagaagtgtcaacatctaaagacaaatgcttggtcaaataggtgattaaaccgtcttcagtcttcaagtcaaaggccattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgcatatcagaaacccatatagaactccaattgactaccaagctttgtctgaagctttcccaccattgaagccatttgtttccgttaacgctgatggtacctcctcagttgacttgaccattccagaagcccaaagagcttttaccgctgcccttttgcacagagacttcggcttgactatgactatcccagaagatcgtttgtgtccaaccgttccaaacagattgaactacgttttgtggattgaagacattttcaactacaccaacaagactttgggtttatctgacgaccgtccaatcaagggtgttgatatcggtaccggtgcttctgccatttacccaatgttggcttgcgccagattcaaggcttggtccatggttggtactgaagttgaaagaaagtgtatcgacactgctagattaaacgttgttgctaacaacttgcaagatcgtctatccatcttggaaacctctattgacggtccaattttagtcccaattttcgaagctaccgaagaatacgaatacgaattcaccatgtgtaacccacctttctacgatggtgccgctgacatgcaaactagcgatgctgcaaagggttttggtttcggtgtcggtgctccacactctggtacagtcatcgaaatgtctactgaaggtggtgaatccgctttcgtggctcaaatggttagagaatctcttaagttgagaaccagatgtagatggtacacttctaacttaggtaagttgaaatctttgaaggaaattgtcggtttgttgaaggaattagaaatctctaattacgccatcaacgaatatgtccaaggttccactagaagatacgctgtcgcttggagtttcactgatatccaattgccagaagaattgtccagaccatctaatcctgaattgtcctctttgttcgactacaaggatgacgatgacaaatgaatcgcgtgcattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatcgcgtacccaattcgccctatagtgagtcgtattacgcgcgctcactggccgtcgttttacaacgtcgtgactgggaaaaccctggcgttacccctgcaggactagtgctgaggcattaatgcaactcagaagtttgacagcaagcaagttcatcattcgaactagccttattgttttagttcagtgacagcgaactgccgtactcgatgctttatttctcacggtagagcggaagaacagataggggcagcgtgagaagagttagaaagtaaatttttatcacgtctgaagtattcttattcataggaaattttgcaaggttttttagctcaataacgggctaagttatataaggtgttcacgcgattttcttgttatgtatacctcttctctgaggaatggtactactgtcctgatgtaggctccttaaattggtgggcaagaataacttatcgatattttgtatattggtcttggagttcaccacgtaatgcctgtttaagaccatcagttaactctagtattatttggtcttggctactggccgtttgctattattcaagtcttttgtgccttcccgtcgggtaagggagttatttagggatacagaatctaacgaaaactaaatctcaatgattaactctatttaatccttttttgaaaggcaaaagaggtcccttgttcacttacaacgttcttagccaaattcgcttatcacttactacttcacgatatacagaagtaaaaacatataaaaagatgtctgtttgtttagccatcacaaaaggtatcgcag

SEQ ID NO: 76 - DNA, 9145 bp, Saccharomyces cerevisiae
  source 1..9145: mol_type = genomic DNA
tgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaggatccatgatgaagttctggagaaagtacacacaacaagaaatggatgaaaagattactgaatctttggaaaagactttgaactacgataacactaagacaatcggtattccaggtactaagttggatgatacagttttctatgatgatcattctttcgttaagcattcaccatacttgagaacttttattcaaaacccaaaccatatcggttgtcatacttatgataaggctgatatcttgttcggtggtacattcgatatcgaaagagaattaatccaattgttagcaatcgatgttttgaacggtaacgatgaagaatttgatggttacgttactcaaggtggtacagaagctaacatccaagcaatgtgggtttacagaaactacttcaagaaagaaagaaaggctaagcatgaagaaatcgctatcatcacttcagcagatacacattactctgcatacaaaggttcagatttgttgaacatcgatattattaaggttccagttgatttttattcaagaaaaattcaagaaaatacattggattcaattgttaaagaagctaaagaaattggtaaaaagtacttcatcgttatctctaacatgggtactacaatgtttggttcagttgatgatccagatttgtacgctaacatcttcgataagtacaatttggaatacaaaattcatgttgatggtgcatttggtggttttatatatccaattgataataaggaatgtaaaactgatttctctaataagaacgtttcttcaatcacattagatggtcataagatgttgcaagctccatacggtactggtatcttcgtttcaagaaagaatttgatccataacactttgacaaaggaagcaacttacatcgaaaatttggatgttacattgtctggttcaagatctggttcaaatgctgttgcaatttggatggttttagcttcttatggtccatacggttggatggaaaagattaataagttgagaaatagaactaaatggttgtgtaagcaattgaacgatatgagaattaaatattacaaagaagattcaatgaatattgttacaattgaagaacaatatgttaataaggaaatcgctgaaaagtactttttagttccagaagttcataacccaactaacaactggtacaagatcgttgttatggaacatgttgaattggatatcttgaactctttggtttacgatttgagaaagtttaataaggaacatttgaaggcaatgcatcatcatcatcatcattaaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcactacataagaacacctttggtggagggaacatcgttggtaccattgggcgaggtggcttctcttatggcaaccgcaagagccttgaacgcactctcactacggtgatgatcattcttgcctcgcagacaatcaacgtggagggtaattctgctagcctctgcaaagctttcaagaaaatgcgggatcatctcgcaagagagatctcctactttctccctttgcaaaccaagttcgacaactgcgtacggcctgttcgaaagatctaccaccgctctggaaagtgcctcatccaaaggcgcaaatcctgatccaaacctttttactccacgcacggcccctagggcctctttaaaagcttgaccgagagcaatcccgcagtcttcagtggtgtgatggtcgtctatgtgtaagtcaccaatgcactcaacgattagcgaccagccggaatgcttggccagagcatgtatcatatggtccagaaaccctatacctgtgtggacgttaatcacttgcgattgtgtggcctgttctgctactgcttctgcctctttttctgggaagatcgagtgctctatcgctaggggaccaccctttaaagagatcgcaatctgaatcttggtttcatttgtaatacgctttactagggctttctgctctgtcatctttgccttcgtttatcttgcctgctcattttttagtatattcttcgaagaaatcacattactttatataatgtataattcattatgtgataatgccaatcgctaagaaaaaaaaagagtcatccgctaggtggaaaaaaaaaaatgaaaatcattaccgaggcataaaaaaatatagagtgtactagaggaggccaagagtaatagaaaaagaaaattgcgggaaaggactgtgttatgacttccctgactaatgccgtgttcaaacgatacctggcagtgactcctagcgctcaccaagctcttaaaacgggaatttatggtgcactctcagtacacgcgccagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttttgcggccgc

SEQ ID NO: 77 - DNA, 499 bp, Saccharomyces cerevisiae
  source 1..499: mol_type = genomic DNA
gtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 78 - DNA, 500 bp, Saccharomyces cerevisiae
  source 1..500: mol_type = genomic DNA
acagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 79 - DNA, 500 bp, Saccharomyces cerevisiae
  source 1..500: mol_type = genomic DNA
gctggattgagctgaatggtgccaggtcgaggctgggagggagactaactcgaaagtgacgaagactcgaaaattaaaaaaaaagatactgcagaaggcaagattgagaatggagtaaaggcagcgtgggtcccctgtggaaaccgcagttttcctgcgccaagtggtaccggtgcgagtgcagcaattaatctctcgatattttcttagtatctctttttatataagaatatattttggaattggtaatgcttatcttcaatagtttcttagttgaatgcacacttaagagcaaattggccaaggagttcttcgttcgctttaatttatttcctggttattgtcaatttattcatcccatctccccaggatagaagaaattagtgtaattttgctgacaatacattttaacgacgataacaataatagcaattaaataaaatagcactaccaccactccactgctcgttagctatttctgtaaaataaataaaaagatc

SEQ ID NO: 80 - DNA, 501 bp, Saccharomyces cerevisiae
  source 1..501: mol_type = genomic DNA
attcgcgctatctcgatttctacctatatagttaatctctgtacaaaaacaatctttccaactatccattaatcatagtatattatcagcgtcggcgattttaccacgcttgacaaaagccgcgggcgggattcctgtgggtagtggcaccggcagttaatctaatcaaaggcgcttgaaggaagagatagataatagaacaaagcaatcgccgctttggacggcaaatatgtttatccattggtgcggtgattggatatgatttgtctccagtagtataagcaagcgccagatctgtttactgtaaaattaagtgagtaatctcgcgggatgtaatgatttaagggaatctggttcaggttttcacatatatttgtatataaggccatttgtaatttcaatagttttaggatttttccttctcccaaaatactcacttactgtgttacattacagaaagaacagacaagaaaccgtcaataagaaatataactaagaaca

SEQ ID NO: 81 - DNA, 27 bp, synthetic construct
  misc_feature 1..27: note = Synthetic HA epitope tag polypeptide
  source 1..27: mol_type = other DNA
tacccatacgacgttccagactacgcc

SEQ ID NO: 82 - protein, 9 aa, synthetic construct
  REGION 1..9: note = Synthetic HA epitope tag polypeptide
YPYDVPDYA

SEQ ID NO: 83 - DNA, 33 bp, synthetic construct
  misc_feature 1..33: note = Synthetic c-MYC epitope tag polypeptide
  source 1..33: mol_type = other DNA
gaacaaaagttaatttctgaagaagatttggaa

SEQ ID NO: 84 - protein, 10 aa, synthetic construct
  REGION 1..10: note = Synthetic c-MYC epitope tag polypeptide
EQKLISEEDL

SEQ ID NO: 85 - DNA, 24 bp, synthetic construct
  misc_feature 1..24: note = Synthetic FLAG epitope tag polypeptide
  source 1..24: mol_type = other DNA
gactacaaggatgacgatgacaaa

SEQ ID NO: 86 - protein, 8 aa, synthetic construct
  REGION 1..8: note = Synthetic FLAG epitope tag polypeptide
DYKDDDDK

SEQ ID NO: 87 - DNA, 42 bp, synthetic construct
  misc_feature 1..42: note = Synthetic V5 epitope tag polypeptide
  source 1..42: mol_type = other DNA
ggtaagccaattccaaatcctttgttgggtttggactccacc

SEQ ID NO: 88 - protein, 14 aa, synthetic construct
  REGION 1..14: note = Synthetic V5 epitope tag polypeptide
GKPIPNPLLGLDST

SEQ ID NO: 89 - DNA, 18 bp, synthetic construct
  misc_feature 1..18: note = Synthetic HIS epitope tag polypeptide
  source 1..18: mol_type = other DNA
catcatcatcatcatcat

SEQ ID NO: 90 - protein, 6 aa, synthetic construct
  REGION 1..6: note = Synthetic HIS epitope tag polypeptide
HHHHHH

SEQ ID NO: 91 - DNA, 7882 bp, synthetic construct
  misc_feature 1..7882: note = pMM1 vector
  source 1..7882: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggatcttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatcttagctagccgcggtaccaagctggtggatcctttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacagcggccgcaaaagatctgagctcttaattaacaattcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctccaattcagctggcgtaatagcgaagaggcccgcaccgatcgcccttcccaacagttgcgcagcctgaatggcgaattcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctggcgcgtgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgtt

SEQ ID NO: 92 - DNA, 3810 bp, synthetic construct
  misc_feature 1..3810: note = pCDM4 vector
  source 1..3810: mol_type = other DNA
gctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagcctaggatcgagatcgatctcgatcccgcgaaattaatacgactcactataggggaattgtgagcggataacaattcccctctagaaataattttgtttaactttaagaaggagatatacatatggcagatctcaattggatatcggccggccacgcgatcgctgacgtcggtaccctcgagtctggtaaagaaaccgctgctgcgaaatttgaacgccagcacatggactcgtctactagtcgcagcttaattaacctaaactgctgccaccgctgagcaataactagcataaccccttggggcctctaaacgggtcttgaggggttttttgctagcgaaaggaggagtcgacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagccagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggccc

SEQ ID NO: 93 - DNA, 5203 bp, synthetic construct
  misc_feature 1..5203: note = pETM6 vector
  source 1..5203: mol_type = other DNA
ggggaattgtgagcggataacaattcccctctagaaataattttgtttaactttaagaaggagatatacatatggcagatctcaattggatatcggccggccacgcgatcgctgacgtcggtaccctcgagtctggtaaagaaaccgctgctgcgaaatttgaacgccagcacatggactcgtctactagtcgcagcttaattaacctaaactgctgccaccgctgagcaataactagcataaccccttggggcctctaaacgggtcttgaggggttttttgctagcgaaaggaggagtcgactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagcctaggatcgagatcgatctcgatcccgcgaaattaatacgactcactata

SEQ ID NO: 94 - DNA, 5369 bp, synthetic construct
  misc_feature 1..5369: note = pET28a(+) vector
  source 1..5369: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 95 - DNA, 2187 bp, Psilocybe cubensis
  source 1..2187: mol_type = genomic DNA
atggcttctagttcttccgatgtcttcgttttgggtctaggtgttgttttggctgccttgtatatcttcagagaccaattattcgctgcttctaagccaaaggtggctccagtttccactacgaagcctgccaacggttccgctaacccaagagacttcatcgccaagatgaaacaaggtaagaagagaatcgtaatcttctacggttctcaaactggtaccgctgaagaatatgctattcgtttggctaaggaagctaagcaaaagttcggtctagcctccttggtttgtgatccagaagaatacgattttgaaaagttggaccaattgccagaagattctattgctttcttcgtcgttgctacctatggtgaaggtgaacctacagacaacgctgtccaattgttgcaaaacttgcaagatgaaagcttcgaattctcctctggtgagagaaagttgtcaggtttgaagtacgttgtttttggtctgggtaacaagacctacgaacattacaacctcattgggagaactgttgacgctcaattggccaagatgggtgctatcagaatcggtgaaagaggtgaaggtgatgatgacaagtccatggaagaagactacttggaatggaaggatggtatgtgggaagcgtttgccactgctatgggtgttgaagaaggtcaaggtggtgactccgctgatttcgtcgtttccgaattggaatctcacccaccagaaaaggtttaccaaggtgaattttctgctagagctttaaccaaaaccaagggtattcacgacgctaagaatccttttgctgctccaattgcggttgctagagaattgttccaatctgttgtcgatagaaactgtgtccacgtcgaattcaacattgaaggctctggtatcacctatcaacacggtgaccacgttggtttgtggccattgaatccagatgttgaagtcgaacggttgttgtgtgttttaggtttagctgaaaagagagatgctgtcatctccattgaatccttagacccggctttggctaaggttccattcccagtcccaactacttacggtgctgtgttgagacactacattgacatctctgctgtcgccggtagacaaatcttgggtactttgtccaaattcgctccaaccccagaagctgaagctttcttgagaaacttgaacactaacaaggaagaataccacaacgtcgtcgctaacggttgtttgaaattgggtgaaattttgcaaatcgctaccggtaacgacattactgtcccaccaactactgccaacaccaccaaatggccaattccattcgacatcattgtttctgccatcccaagattgcaaccaagatactactctatctcttcttccccaaaaattcatccaaacaccatccacgctaccgttgttgtgctcaaatacgaaaacgttccaaccgaaccaatcccaagaaagtgggtttacggtgtcggtagtaacttcttgttgaatttaaagtacgctgttaacaaggaaccagttccatacatcactcaaaatggcgaacaaagagtcggtgtcccggaatacttgattgctggtccacgtggttcttacaagactgaatctttctacaaggctccaatccatgttagacgttctactttccgtttgccaaccaacccaaagtctccagtcatcatgattggtccaggtactggtgtcgccccattcagaggcttcgttcaagaaagagttgccttggccagaagatccatcgaaaagaacggtcctgactctttggctgactggggtcgtatttccttgttctacggttgtagaagatccgacgaagacttcttgtacaaggacgaatggccacaatacgaagctgagttgaagggtaagttcaagttgcactgtgctttctccagacaaaactacaagccagacggttctaagatttacgtccaagatttgatctgggaagacagagaacacattgccgatgccatcttaaacggtaagggttacgtctacatctgcggtgaagctaagtccatgtctaaacaagttgaagaagttctagccaagatcttgggcgaagccaaaggtggttccggtccagttgaaggtgttgctgaagtcaagttactgaaggaacggtccagattgatgttggatgtctggtcttga

SEQ ID NO: 96 - protein, 728 aa, Psilocybe cubensis
MASSSSDVFVLGLGVVLAALYIFRDQLFAASKPKVAPVSTTKPANGSANPRDFIAKMKQGKKRIVIFYGSQTGTAEEYAIRLAKEAKQKFGLASLVCDPEEYDFEKLDQLPEDSIAFFVVATYGEGEPTDNAVQLLQNLQDESFEFSSGERKLSGLKYVVFGLGNKTYEHYNLIGRTVDAQLAKMGAIRIGERGEGDDDKSMEEDYLEWKDGMWEAFATAMGVEEGQGGDSADFVVSELESHPPEKVYQGEFSARALTKTKGIHDAKNPFAAPIAVARELFQSVVDRNCVHVEFNIEGSGITYQHGDHVGLWPLNPDVEVERLLCVLGLAEKRDAVISIESLDPALAKVPFPVPTTYGAVLRHYIDISAVAGRQILGTLSKFAPTPEAEAFLRNLNTNKEEYHNVVANGCLKLGEILQIATGNDITVPPTTANTTKWPIPFDIIVSAIPRLQPRYYSISSSPKIHPNTIHATVVVLKYENVPTEPIPRKWVYGVGSNFLLNLKYAVNKEPVPYITQNGEQRVGVPEYLIAGPRGSYKTESFYKAPIHVRRSTFRLPTNPKSPVIMIGPGTGVAPFRGFVQERVALARRSIEKNGPDSLADWGRISLFYGCRRSDEDFLYKDEWPQYEAELKGKFKLHCAFSRQNYKPDGSKIYVQDLIWEDREHIADAILNGKGYVYICGEAKSMSKQVEEVLAKILGEAKGGSGPVEGVAEVKLLKERSRLMLDVWS

SEQ ID NO: 97 - DNA, 5203 bp, synthetic construct
  misc_feature 1..5203: note = pETM6-H10 vector
  source 1..5203: mol_type = other DNA
gaagaattgtgagcggataacaattcccctctagaaataattttgtttaactttaagaaggagatatacatatggcagatctcaattggatatcggccggccacgcgatcgctgacgtcggtaccctcgagtctggtaaagaaaccgctgctgcgaaatttgaacgccagcacatggactcgtctactagtcgcagcttaattaacctaaactgctgccaccgctgagcaataactagcataaccccttggggcctctaaacgggtcttgaggggttttttgctagcgaaaggaggagtcgactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagcctaggatcgagatcgatctcgatcccgcgaaattaatacgactcactacg

SEQ ID NO: 98 - DNA, 3777 bp, synthetic construct
  misc_feature 1..3777: note = pRSM3 vector
  source 1..3777: mol_type = other DNA
cgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagcctaggatcgagatcgatctcgatcccgcgaaattaatacgactcactataggggaattgtgagcggataacaattcccctctagaaataattttgtttaactttaagaaggagatatacatatggcagatctcaattggatatcggccggccacgcgatcgctgacgtcggtaccctcgagtctggtaaagaaaccgctgctgcgaaatttgaacgccagcacatggactcgtctactagtcgcagcttaattaacctaaactgctgccaccgctgagcaataactagcataaccccttggggcctctaaacgggtcttgaggggttttttgctagcgaaaggaggagtcgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgcagcgctcttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcc

(19) United States
(12) Patent Application Publication
(10) Pub. No.: US 2024/0299427 A1
(43) Pub. Date: Sep. 12, 2024

(54) HALOGENATED PSILOCYBIN DERIVATIVES AND METHODS OF USING

(71) Applicant: Enveric Biosciences Canada Inc., Calgary (CA)

(72) Inventors: Jillian M. Hagel, Calgary (CA); Peter J. Facchini, Calgary (CA)

(21) Appl. No.: 18/655,793

(22) Filed: May 6, 2024

Related U.S. Application Data
(63) Continuation of application No. 17/903,080, filed on Sep. 6, 2022, now Pat. No. 11,998,557.
Application No. 17/903,080 is a continuation of application No. PCT/CA2021/051209, filed on Sep. 1, 2021.
(60) Provisional application No. 63/073,104, filed on Sep. 1, 2020.

(51) Int. Cl. A61K 31/675; A61K 31/4045; C12N 9/02; C12N 9/10; C12N 9/12; C12N 15/52; C12P 17/10

(52) U.S. Cl. CPC A61K 31/675; A61K 31/4045; C12N 9/0083; C12N 9/1007; C12N 9/1205; C12N 15/52; C12P 17/10

(57) ABSTRACT

Disclosed are novel halogenated psilocybin derivative compounds and pharmaceutical and recreational drug formulations containing the same. The compounds may be produced using a biosynthetic system which comprises cells comprising a psilocybin biosynthetic enzyme complement.

### RELATED APPLICATION

This application is a continuation of U.S. application Ser. No. 17/903,080 filed Sep. 6, 2022 which is a continuation of PCT Application No. PCT/CA2021/051209 filed Sep. 1, 2021, which claims the benefit of U.S. Provisional Application No. 63/073,104 filed Sep. 1, 2020; the entire contents of which are hereby incorporated by reference.

### INCORPORATION OF SEQUENCE LISTING

A computer readable form of the Sequence Listing “29664-P62051 US03_SequenceListing.xml” (216,574 bytes), submitted via Patent Center and created on May 6, 2024, is herein incorporated by reference.

### FIELD OF THE DISCLOSURE

The compositions and methods disclosed herein relate to a chemical compound known as psilocybin. Furthermore, the compositions and methods disclosed herein relate in particular to halogenated forms of psilocybin.

### BACKGROUND OF THE DISCLOSURE

The following paragraphs are provided by way of background to the present disclosure. They are not however an admission that anything discussed therein is prior art or part of the knowledge of a person of skill in the art.

The biochemical pathways in the cells of living organisms may be classified as being part of primary metabolism, or as being part of secondary metabolism. Pathways that are part of a cell's primary metabolism are involved in catabolism for energy production or in anabolism for building block production for the cell. Secondary metabolites, on the other hand, are produced by the cell without having an obvious anabolic or catabolic function. It has long been recognized that secondary metabolites can be useful in many respects, including as therapeutic compounds.

Psilocybin, for example, is a secondary metabolite that is naturally produced by certain mushrooms which taxonomically can be classified as belonging the Basidiomycota division of the fungi kingdom. Mushroom species which can produce psilocybin include species belonging to the genus Psilocybe, such as Psilocybe azurescens, Psilocybe semilanceata, Psilocybe serbica, Psilocybe mexicana, and Psilocybe cyanescens, for example. The interest of the art in psilocybin is well established. Thus, for example, psilocybin is a psychoactive compound and is therefore used as a recreational drug. Furthermore, psilocybin is used as a research tool in behavioral and neuro-imaging studies in psychotic disorders, and has been evaluated for its clinical potential in the treatment of mental health conditions (Daniel, J. et al. Mental Health Clin/, 2017; 7(1): 24-28), including to treat anxiety in terminal cancer patients (Grob, C. et al. Arch. Gen. Psychiatry, 2011, 68(1) 71-78) and to alleviate symptoms of treatment-resistant depression (Cathart-Harris, R. L. et al. Lancet Psychiatry, 2016, 3: 619-627).

Although the toxicity of psilocybin is low, adverse side effects, including, for example, panic attacks, paranoia and psychotic states, sometimes together or individually referred to as “a bad trip”, are not infrequently experienced by recreational psilocybin users.

There exists therefore a need in the art for improved psilocybin compounds.

### SUMMARY OF THE DISCLOSURE

The following paragraphs are intended to introduce the reader to the more detailed description, not to define or limit the claimed subject matter of the present disclosure.

In one aspect, the present disclosure relates to psilocybin and derivative compounds.

In another aspect, the present disclosure relates to halogenated psilocybin derivative compounds and methods of making and using these compounds.

Accordingly, in one aspect, the present disclosure provides, in accordance with the teachings herein, in at least one embodiment, a chemical compound or salts thereof having the formula (I):

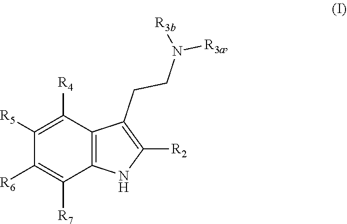

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, hydrogen atom or an alkyl or O-alkyl group, and wherein R_{3A} and R_{3B} are a hydrogen atom, an alkyl group, an aryl group, or an acyl group.

In at least one embodiment, in an aspect, the halogen atom can be a bromine atom.

In at least one embodiment, in an aspect, the halogen atom can be a chlorine atom.

In at least one embodiment, in an aspect, the halogen atom can be a fluorine atom.

In at least one embodiment, in an aspect, the halogen atom can be an iodine atom.

In at least one embodiment, in an aspect, R₂ can be a halogen atom and R₅, R₆ and R₇ can each be a hydrogen atom or an alkyl group or O-alkyl group, and R₄ can be a phosphate group, a hydrogen atom or an alkyl group or O-alkyl group.

In at least one embodiment, in an aspect, R₄ can be a halogen atom and R₂, R₅, R₆ and R₇ can each be a hydrogen atom or an alkyl group or O-alkyl group.

In at least one embodiment, in an aspect, R₅ can be a halogen atom and R₂, R₆ and R₇ can each be a hydrogen atom or an alkyl group or O-alkyl group, and R₄ can be a phosphate group, a hydrogen atom or an alkyl group or O-alkyl group.

In at least one embodiment, in an aspect, R₆ can be a halogen atom and R₂, R₅ and R₇ can each be a hydrogen atom or an alkyl group or O-alkyl group, and R₄ can be a phosphate group, a hydrogen atom or an alkyl group or O-alkyl group.

In at least one embodiment, in an aspect, R₇ can be a halogen atom and R₂, R₅ and R₆ can each be a hydrogen atom or an alkyl group or O-alkyl group, and R₄ can be a phosphate group, a hydrogen atom or an alkyl group or O-alkyl group.

In at least one embodiment, in an aspect, at least two of R₂, R₄, R₅, R₆ or R₇ can be a halogen atom.

In at least one embodiment, in an aspect, the at least of two of R₂, R₄, R₅, R₆ or R₇ that are halogen atoms can be at least two identical halogen atoms.

In at least one embodiment, in an aspect, the at least of two of R₂, R₄, R₅, R₆ or R₇ that are halogen atoms can at least two non-identical halogen atoms.

In at least one embodiment, in an aspect, R₂ and R₄ can be a halogen atom, and R₅, R₆ and R₇ can be a hydrogen atom or an alkyl group or O-alkyl group.

In at least one embodiment, in an aspect, R₂ and R₅ can be a halogen atom, R₆ and R₇ can be a hydrogen atom or an alkyl group or O-alkyl group, and R₄ can be a hydrogen atom, a phosphate group or an alkyl group or O-alkyl group.

In at least one embodiment, in an aspect, R₂ and R₆ can be a halogen atom, R₅ and R₇ can be a hydrogen atom or an alkyl group or O-alkyl group, and R₄ can be a hydrogen atom, a phosphate group or an alkyl group or O-alkyl group.

In at least one embodiment, in an aspect, R₂ and R₇ can be a halogen atom, R₅ and R₆ can be a hydrogen atom or an alkyl group or O-alkyl group, and R₄ can be a hydrogen atom, a phosphate group or an alkyl group or O-alkyl group.

In at least one embodiment, in an aspect, R₄ and R₅ can be a halogen atom, and R₂, R₆ and R₇ can be a hydrogen atom or an alkyl group or O-alkyl group.

In at least one embodiment, in an aspect, R₄ and R₆ can be a halogen atom, and R₂, R₅ and R₇ can be a hydrogen atom or an alkyl group or O-alkyl group.

In at least one embodiment, in an aspect, R₄ and R₇ can be a halogen atom, and R₂, R₅ and R₆ can be a hydrogen atom or an alkyl group or O-alkyl group.

In at least one embodiment, in an aspect, R₅ and R₆ can be a halogen atom, R₂ and R₇ can be a hydrogen atom or an alkyl group or O-alkyl group, and R₄ can be a hydrogen atom, a phosphate group or an alkyl group or O-alkyl group.

In at least one embodiment, in an aspect, R₅ and R₇ can be a halogen atom, R₂ and R₆ can be a hydrogen atom or an alkyl group or O-alkyl group, and R₄ can be a hydrogen atom, a phosphate group or an alkyl group or O-alkyl group.

In at least one embodiment, in an aspect, R₆ and R₇ can be a halogen atom, R₂ and R₅ can be a hydrogen atom or an alkyl group or O-alkyl group, and R₄ can be a hydrogen atom, a phosphate group or an alkyl group or O-alkyl group.

In at least one embodiment, in an aspect, R₄ when it is not halogenated can be a hydrogen atom.

In at least one embodiment, in an aspect, R₄ when it is not halogenated can be an alkyl group.

In at least one embodiment, in an aspect, R₄ when it is not halogenated can be an O-alkyl group.

In at least one embodiment, in an aspect, R₄ when it is not halogenated can be a phosphate group.

In at least one embodiment, in an aspect, three, four or all five of R₂, R₄, R₅, R₆ or R₇ can be a halogen atom.

In at least one embodiment, in an aspect, the three, four, or five of R₂, R₄, R₅, R₆ or R₇ that are halogen atoms can all be identical halogen atoms.

In at least one embodiment, in an aspect, the three, four or five of R₂, R₄, R₅, R₆ or R₇ that are halogen atoms, can include at least two non-identical halogen atoms.

In at least one embodiment, in an aspect, the chemical compound can be selected from the group consisting of compounds having formulas (II); (III); (IV); (V); (VI); (VII): (VIII); (IX); (X); (XI); (XII); (XIII); (XIV); (XV); (XVI); and (XVII):

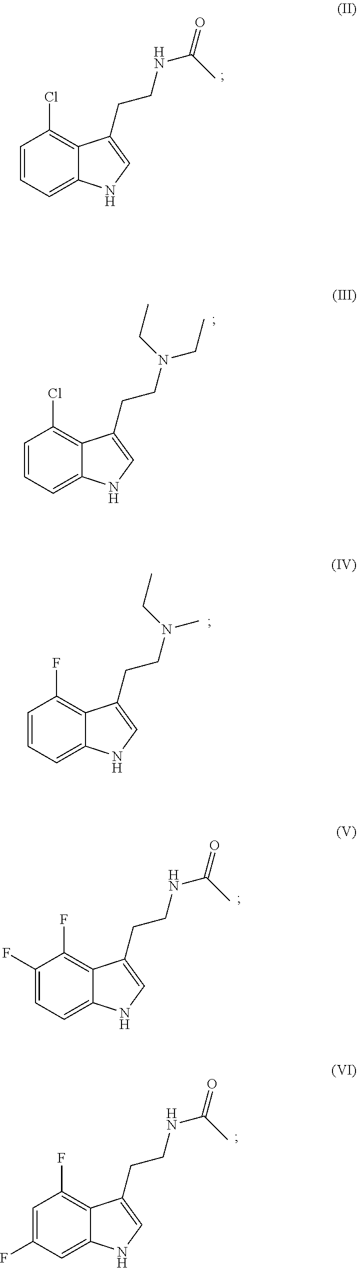 [SMILES: CC(=O)NCCc1c[nH]c2cccc(Cl)c12 ; CCN(CC)CCc1c[nH]c2cccc(Cl)c12 ; CCN(C)CCc1c[nH]c2cccc(F)c12 ; CC(=O)NCCc1c[nH]c2ccc(F)c(F)c12 ; CC(=O)NCCc1c[nH]c2cc(F)cc(F)c12]

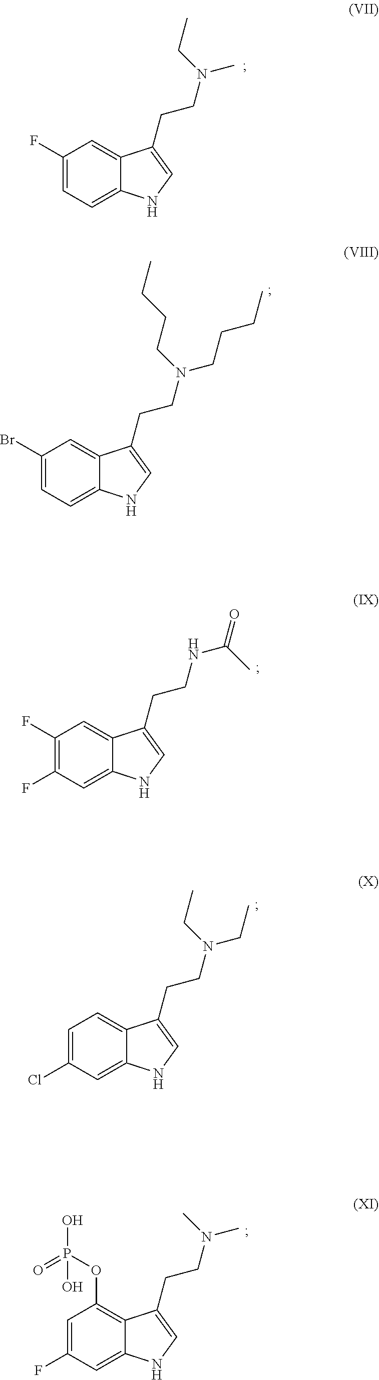 [SMILES: CCN(C)CCc1c[nH]c2ccc(F)cc12 ; CCCCN(CCCC)CCc1c[nH]c2ccc(Br)cc12 ; CC(=O)NCCc1c[nH]c2cc(F)c(F)cc12 ; CCN(CC)CCc1c[nH]c2cc(Cl)ccc12 ; CN(C)CCc1c[nH]c2cc(F)cc(OP(=O)(O)O)c12]

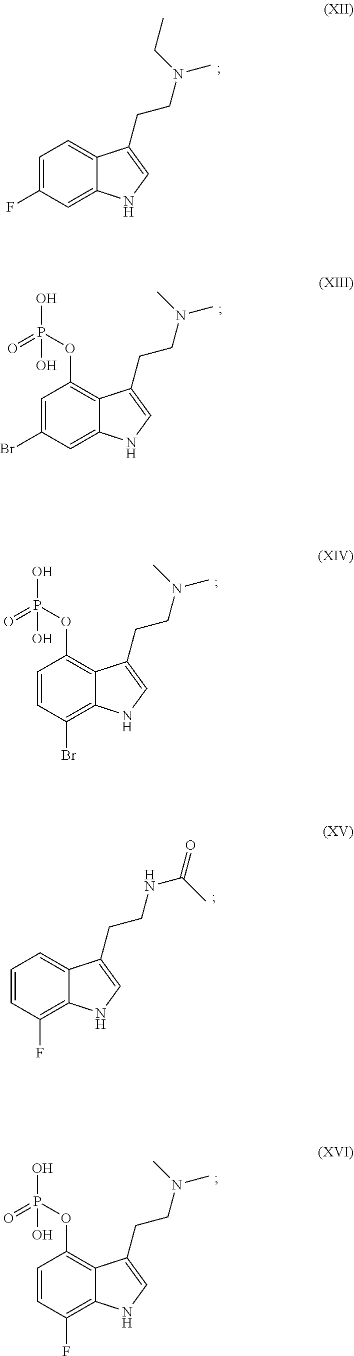 [SMILES: CCN(C)CCc1c[nH]c2cc(F)ccc12 ; CN(C)CCc1c[nH]c2cc(Br)cc(OP(=O)(O)O)c12 ; CN(C)CCc1c[nH]c2c(Br)ccc(OP(=O)(O)O)c12 ; CC(=O)NCCc1c[nH]c2c(F)cccc12 ; CN(C)CCc1c[nH]c2c(F)ccc(OP(=O)(O)O)c12]

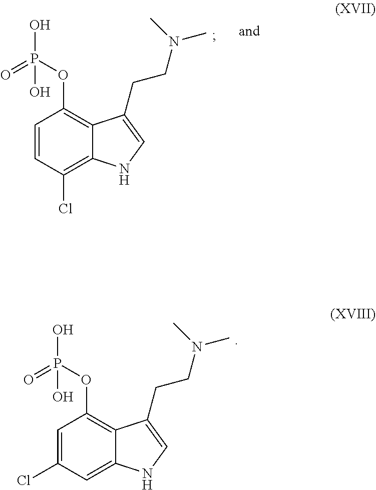 [SMILES: CN(C)CCc1c[nH]c2c(Cl)ccc(OP(=O)(O)O)c12 ; CN(C)CCc1c[nH]c2cc(Cl)cc(OP(=O)(O)O)c12]

In at least one embodiment, the chemical compound is at least about 95% (w/w) pure.

In another aspect, the present disclosure relates to pharmaceutical and recreational drug formulations comprising halogenated psilocybin derivatives. Accordingly, in one aspect, the present disclosure provides in at least one aspect, a pharmaceutical or recreational drug formulation comprising an effective amount of a chemical compound or salts thereof having the formula (I):

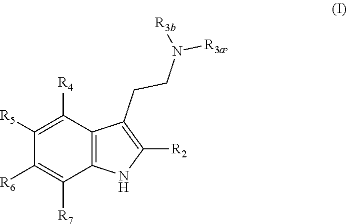

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein R_{3A} and R_{3B} are a hydrogen atom, an alkyl group, an aryl group, or an acyl group, together with a pharmaceutically acceptable excipient, diluent or carrier.

In another aspect, the present disclosure relates to methods of treatment of psychiatric disorders. Accordingly, the present disclosure further provide a method for treating a psychiatric disorder, the method comprising administering to a subject in need thereof a pharmaceutical formulation comprising a chemical compound or salts thereof having the formula (I):

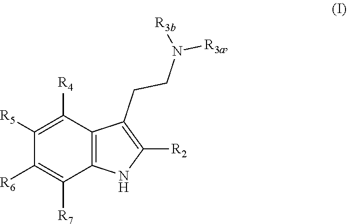

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein R_{3A} and R_{3B} are a hydrogen atom, an alkyl group, an aryl group, or an acyl group, wherein the pharmaceutical formulation is administered in an effective amount to treat the psychiatric disorder in the subject.

In at least one embodiment, in an aspect the disorder can be a 5-HT_{2A} receptor mediated disorder.

In at least one embodiment, in an aspect, a dose can be administered of about 0.001 mg to about 5,000 mg.

In another aspect, the present disclosure relates to methods of making halogenated psilocybin derivatives. Accordingly, in one aspect, the present disclosure provides in at least one aspect, a method of making a halogenated psilocybin derivative the method comprising:
(a) contacting a halogenated psilocybin precursor compound with a host cell comprising a psilocybin biosynthetic enzyme complement; and
(b) growing the host cell to produce a halogenated psilocybin derivative or salts thereof having the formula (I):

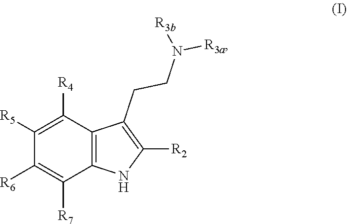

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein R_{3A} and R_{3B} are a hydrogen atom, an alkyl group, an aryl group, or an acyl group.

In at least one embodiment, in an aspect, the halogenated psilocybin precursor compound can be contacted with the host cell by including the psilocybin precursor compound in a growth medium for the host cell.

In at least one embodiment, in an aspect, the halogenated psilocybin precursor compound can be formed by contacting the host cell with a non-halogenated psilocybin precursor compound and a halogen, the host cell further comprising a halogenase capable of halogenating the non-halogenated psilocybin compound and forming the halogenated psilocybin precursor compound.

In at least one embodiment, in an aspect, R₂ can be a halogen atom and R₅, R₆ and R₇ can each be a hydrogen atom, and R₄ can be a phosphate group, a hydrogen atom or an alkyl group or O-alkyl group.

In at least one embodiment, in an aspect, R₄ can be a halogen atom and R₂, R₅, R₆ and R₇ can each be a hydrogen atom or an alkyl group or O-alkyl group.

In at least one embodiment, in an aspect, R₅ can be a halogen atom and R₂, R₆ and R₇ can each be a hydrogen atom or an alkyl group or O-alkyl group, and R₄ can be a phosphate group, a hydrogen atom or an alkyl group or O-alkyl group.

In at least one embodiment, in an aspect, R₆ can be a halogen atom and R₂, R₅ and R₇ can each be a hydrogen atom or an alkyl group or O-alkyl group, and R₄ can be a phosphate group, a hydrogen atom or an alkyl group or O-alkyl group.

In at least one embodiment, in an aspect, R₇ can be a halogen atom and R₂, R₅ and R₆ can each be a hydrogen atom or an alkyl group or O-alkyl group, and R₄ can be a phosphate group, a hydrogen atom or an alkyl group or O-alkyl group.

In at least one embodiment, in an aspect, at least two of R₂, R₄, R₅, R₆ or R₇ can be a halogen atom.

In at least one embodiment, in an aspect, the at least of two of R₂, R₄, R₅, R₆ or R₇ that are halogen atoms can be two identical halogen atoms.

In at least one embodiment, in an aspect, the at least of two of R₂, R₄, R₅, R₆ or R₇ that are halogen atoms can be two non-identical halogen atoms.

In at least one embodiment, in an aspect, R₂ and R₄ can be a halogen atom, and R₅, R₆ and R₇ can be a hydrogen atom or an alkyl group or O-alkyl group.

In at least one embodiment, in an aspect, R₂ and R₅ can be a halogen atom, R₆ and R₇ can be a hydrogen atom or an alkyl group or O-alkyl group, and R₄ can be a hydrogen atom, an alkyl group or O-alkyl group or a phosphate group.

In at least one embodiment, in an aspect, R₂ and R₆ can be a halogen atom, R₅ and R₇ can be a hydrogen atom or an alkyl group or O-alkyl group, and R₄ can be a hydrogen atom, an alkyl group or O-alkyl group or a phosphate group.

In at least one embodiment, in an aspect, R₂ and R₇ can be a halogen atom, R₅ and R₆ can be a hydrogen atom or an alkyl group or O-alkyl group, and R₄ can be a hydrogen atom, an alkyl group or O-alkyl group or a phosphate group.

In at least one embodiment, in an aspect, R₄ and R₅ can be a halogen atom, and R₂, R₆ and R₇ can be a hydrogen atom or an alkyl group or O-alkyl group.

In at least one embodiment, in an aspect, R₄ and R₆ can be a halogen atom, and R₂, R₅ and R₇ can be a hydrogen atom or an alkyl group or O-alkyl group.

In at least one embodiment, in an aspect, R₄ and R₇ can be a halogen atom, and R₂, R₅ and R₆ can be a hydrogen atom or an alkyl group or O-alkyl group.

In at least one embodiment, in an aspect, R₅ and R₆ can be a halogen atom, R₂ and R₇ can be a hydrogen atom or an alkyl group or O-alkyl group, and R₄ can be a hydrogen atom, an alkyl group or O-alkyl group or a phosphate group.

In at least one embodiment, in an aspect, R₅ and R₇ can be a halogen atom, R₂ and R₆ can be a hydrogen atom or an alkyl group or O-alkyl group, and R₄ can be a hydrogen atom, an alkyl group or O-alkyl group or a phosphate group.

In at least one embodiment, in an aspect, R₆ and R₇ can be a halogen atom, R₂ and R₅ can be a hydrogen atom or an alkyl group or O-alkyl group, and R₄ can be a hydrogen atom, an alkyl group or O-alkyl group or a phosphate group.

In at least one embodiment, in an aspect, R₄ when it is not halogenated can be a hydrogen atom.

In at least one embodiment, in an aspect, R₄ when it is not halogenated can be an alkyl group.

In at least one embodiment, in an aspect, R₄ when it is not halogenated can be an O-alkyl group.

In at least one embodiment, in an aspect, R₄ when it is not halogenated can be a phosphate group.

In at least one embodiment, in an aspect, the halogenated psilocybin derivative can be a derivative selected from the group consisting of derivatives having formulas (II); (III); (IV); (V); (VI); (VII): (VIII); (IX); (X); (XI); (XII); (XIII); (XIV); (XV); (XVI); and (XVII):

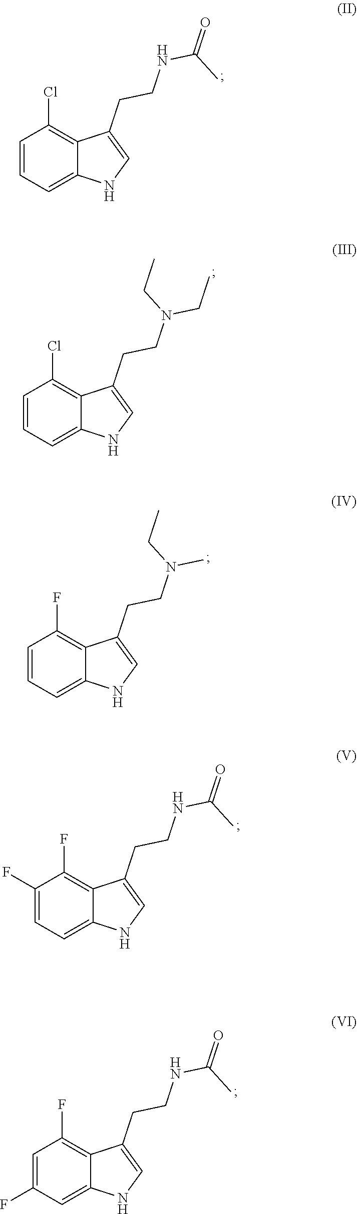 [SMILES: CC(=O)NCCc1c[nH]c2cccc(Cl)c12 ; CCN(CC)CCc1c[nH]c2cccc(Cl)c12 ; CCN(C)CCc1c[nH]c2cccc(F)c12 ; CC(=O)NCCc1c[nH]c2ccc(F)c(F)c12 ; CC(=O)NCCc1c[nH]c2cc(F)cc(F)c12]

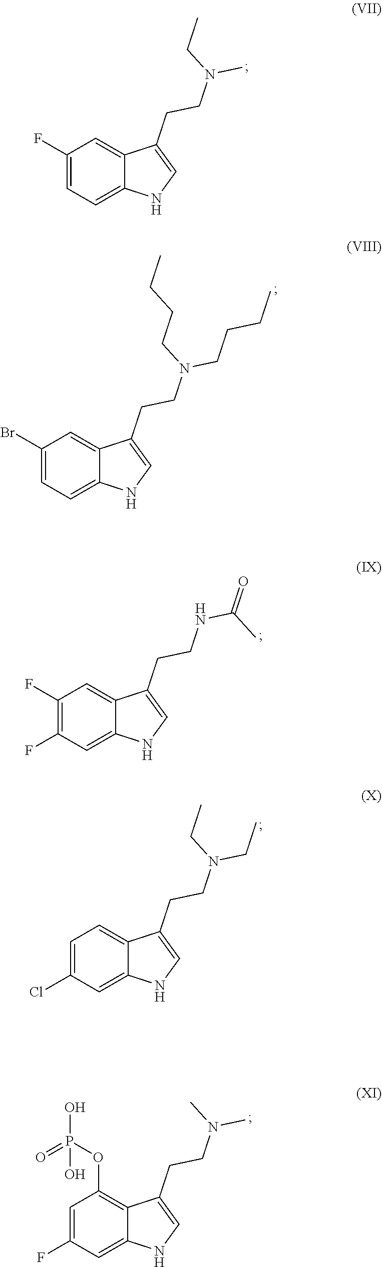 [SMILES: CCN(C)CCc1c[nH]c2ccc(F)cc12 ; CCCCN(CCCC)CCc1c[nH]c2ccc(Br)cc12 ; CC(=O)NCCc1c[nH]c2cc(F)c(F)cc12 ; CCN(CC)CCc1c[nH]c2cc(Cl)ccc12 ; CN(C)CCc1c[nH]c2cc(F)cc(OP(=O)(O)O)c12]

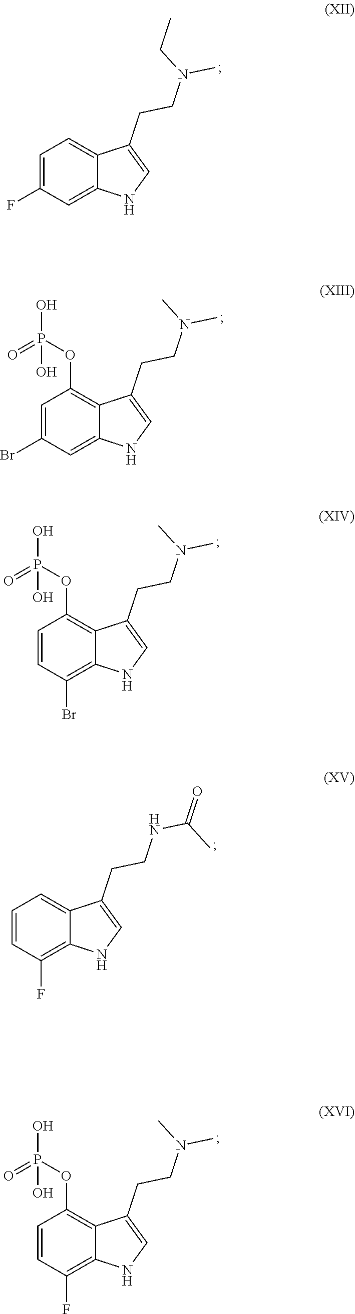 [SMILES: CCN(C)CCc1c[nH]c2cc(F)ccc12 ; CN(C)CCc1c[nH]c2cc(Br)cc(OP(=O)(O)O)c12 ; CN(C)CCc1c[nH]c2c(Br)ccc(OP(=O)(O)O)c12 ; CC(=O)NCCc1c[nH]c2c(F)cccc12 ; CN(C)CCc1c[nH]c2c(F)ccc(OP(=O)(O)O)c12]

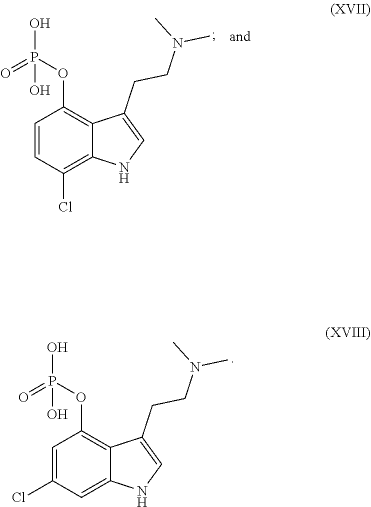 [SMILES: CN(C)CCc1c[nH]c2c(Cl)ccc(OP(=O)(O)O)c12 ; CN(C)CCc1c[nH]c2cc(Cl)cc(OP(=O)(O)O)c12]

In at least one embodiment, in an aspect, the halogenated psilocybin precursor compound can be selected from the group of halogenated compounds consisting of halogenated tryptophan, halogenated tryptamine, halogenated 4-hydroxytryptamine, halogenated 4-hydroxy-indole, halogenated 4-hydroxytryptophan, halogenated norbaeocystin, and halogenated baeocystin.

In at least one embodiment, in an aspect, the psilocybin biosynthetic enzyme complement can comprise at least one enzyme encoded by a nucleic acid selected from:
(a) SEQ.ID NO: 1, SEQ.ID NO: 3, SEQ.ID NO: 5, SEQ.ID NO: 7, SEQ.ID NO: 9, SEQ.ID NO 11, SEQ.ID NO: 36, SEQ.ID NO: 38, SEQ.ID NO: 40, SEQ. ID NO: 42, SEQ.ID NO: 44, SEQ.ID NO: 46, SEQ.ID NO: 48, SEQ.ID NO: 50, SEQ.ID NO: 52, SEQ.ID NO: 54, SEQ. ID NO: 56, SEQ.ID NO: 58, SEQ.ID NO: 60, SEQ.ID NO: 64, SEQ.ID NO: 66, SEQ.ID NO: 68, SEQ. ID NO: 70, and SEQ.ID NO: 72;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 2, SEQ.ID NO: 4, SEQ.ID NO: 6, SEQ.ID NO: 8, SEQ.ID NO: 10, SEQ.ID NO 12, SEQ.ID NO: 37, SEQ.ID NO: 39, SEQ.ID NO: 41, SEQ. ID NO: 43, SEQ.ID NO: 45, SEQ.ID NO: 47, SEQ.ID NO: 49, SEQ.ID NO: 51, SEQ.ID NO: 53, SEQ.ID NO: 55, SEQ. ID NO: 57, SEQ.ID NO: 59, SEQ.ID NO: 61, SEQ.ID NO: 65, SEQ.ID NO: 67, SEQ.ID NO: 69, SEQ. ID NO: 71, and SEQ.ID NO: 73;
(f) a nucleic acid sequence that encodes a functional variant of any one of the amino acid sequences set forth in SEQ.ID NO: 2, SEQ.ID NO: 4, SEQ.ID NO: 6, SEQ.ID NO: 8, SEQ.ID NO: 10, SEQ.ID NO 12, SEQ.ID NO: 37, SEQ.ID NO: 39, SEQ.ID NO: 41, SEQ. ID NO: 43, SEQ.ID NO: 45, SEQ.ID NO: 47, SEQ.ID NO: 49, SEQ.ID NO: 51, SEQ.ID NO: 53, SEQ.ID NO: 55, SEQ. ID NO: 57, SEQ.ID NO: 59, SEQ.ID NO: 61, SEQ.ID NO: 65, SEQ.ID NO: 67, SEQ.ID NO: 69, SEQ. ID NO: 71, and SEQ.ID NO: 73; and
(g) a nucleic acid sequence that hybridizes under stringent conditions to any one of the nucleic acid sequences set forth in (a), (b), (c), (d), (e) or (f).

In at least one embodiment, in an aspect, the halogenase can be encoded by a nucleic acid selected from:
(a) SEQ.ID NO: 13, SEQ.ID NO: 15, SEQ. ID NO: 17, SEQ.ID NO: 19, SEQ. ID NO: 21, SEQ.ID NO: 23, SEQ.ID NO: 25, and SEQ.ID NO: 27;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 14, SEQ.ID NO: 16, SEQ. ID NO: 18, SEQ.ID NO: 20, SEQ. ID NO: 22, SEQ.ID NO: 24, SEQ.ID NO: 26, SEQ.ID NO: 28, SEQ.ID NO: 29, SEQ.ID NO: 30, SEQ.ID NO: 31, SEQ.ID NO: 32. SEQ.ID NO: 33, SEQ.ID NO: 34, and SEQ.ID NO: 35;
(f) a nucleic acid sequence that encodes a functional variant of any one of the amino acid sequences set forth in SEQ.ID NO: 14, SEQ.ID NO: 16, SEQ. ID NO: 18, SEQ.ID NO: 20, SEQ. ID NO: 22, SEQ.ID NO: 24, SEQ.ID NO: 26, SEQ.ID NO: 28, SEQ.ID NO: 29, SEQ.ID NO: 30, SEQ.ID NO: 31, SEQ.ID NO: 32. SEQ.ID NO: 33, SEQ.ID NO: 34, and SEQ.ID NO: 35; and
(g) a nucleic acid sequence that hybridizes under stringent conditions to any one of the nucleic acid sequences set forth in (a), (b), (c), (d), (e) or (f).

In at least one embodiment, in an aspect, the host cell can further comprise an acetyl transferase capable of acetylating R₃ₐ or R_{3b}, wherein the cell comprises a compound having chemical formula (I):

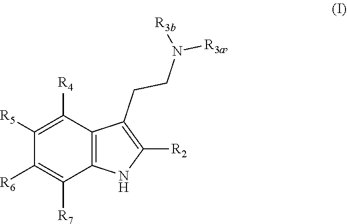

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein at least one of R_{3A} and R_{3B} is a hydrogen atom,
to form a compound having the chemical formula (XIX):

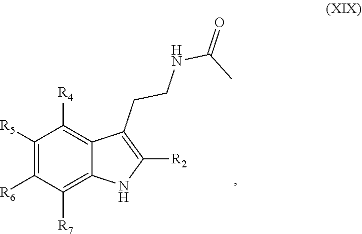

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group.

In at least one embodiment, the acetylase can be encoded by a nucleic acid selected from:
(a) SEQ.ID NO: 62;
(b) a nucleic acid sequence that is substantially identical the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having an amino acid sequences set forth in SEQ.ID NO: 63;
(f) a nucleic acid sequence that encodes a functional variant of the amino acid sequence set forth in SEQ.ID NO: 63; and
(g) a nucleic acid sequence that hybridizes under stringent conditions to any one of the nucleic acid sequences set forth in (a), (b), (c), (d), (e) or (f).

In at least one embodiment, in an aspect,
(a) the halogenated psilocybin precursor compound can be a halogenated indole, and the a psilocybin biosynthetic enzyme complement can comprise (i) a nucleic acid encoding TrpB selected from SEQ. ID NO: 12, SEQ. ID NO: 65, SEQ.ID NO: 67, SEQ.ID NO: 69, SEQ.ID NO: 71, and SEQ.ID NO: 73, or a nucleic acid sequence encoding a polypeptide sequence substantially similar thereto; (ii) a nucleic acid encoding PsiD selected from SEQ.ID NO: 2, SEQ.ID NO: 10, SEQ.ID NO: 37, SEQ.ID NO: 39, SEQ.ID NO: 41, SEQ.ID NO: 43, SEQ.ID NO: 45, SEQ.ID NO: 47, SEQ.ID NO: 49, SEQ.ID NO: 51, SEQ.ID NO: 53, SEQ.ID NO: 55, and SEQ.ID NO: 57, or a nucleic acid sequence encoding a polypeptide sequence substantially similar thereto; (iii) a nucleic acid encoding PsiK encoded by SEQ.ID NO: 6, or a nucleic acid sequence encoding a polypeptide sequence substantially similar thereto; and (iv) a nucleic acid encoding PsiM selected from SEQ.ID NO: 8, SEQ.ID NO: 59 and SEQ.ID NO: 61 or a nucleic acid sequence encoding a polypeptide sequence substantially similar thereto; or
(b) the halogenated psilocybin precursor compound can be a halogenated tryptophan, and the psilocybin biosynthetic enzyme complement can comprise (i) a nucleic acid encoding PsiD selected from SEQ.ID NO: 2, SEQ.ID NO: 10, SEQ.ID NO: 37, SEQ.ID NO: 39, SEQ.ID NO: 41, SEQ.ID NO: 43, SEQ.ID NO: 45, SEQ.ID NO: 47, SEQ.ID NO: 49, SEQ.ID NO: 51, SEQ.ID NO: 53, SEQ.ID NO: 55, and SEQ.ID NO: 57, or a nucleic acid sequence encoding a polypeptide sequence substantially similar thereto; (ii) a nucleic acid encoding PsiK encoded by SEQ.ID NO: 6, or a nucleic acid sequence encoding a polypeptide sequence substantially similar thereto; and (iii) a nucleic acid encoding PsiM selected from SEQ. ID NO: 8, SEQ.ID NO: 59 and SEQ.ID NO: 61 or a nucleic acid sequence encoding a polypeptide sequence substantially similar thereto; or
(c) the halogenated psilocybin precursor compound can be a halogenated tryptamine, and the psilocybin biosynthetic enzyme complement can comprise (i) a nucleic acid encoding PsiK encoded by SEQ.ID NO: 6, or a nucleic acid sequence encoding a polypeptide sequence substantially similar thereto; and (ii) and a nucleic acid encoding PsiM selected from SEQ.ID NO: 8, SEQ.ID NO: 59 and SEQ.ID NO: 61 or a nucleic acid sequence encoding a polypeptide sequence substantially similar thereto; or
(d) the halogenated psilocybin precursor compound can be a halogenated norbaeocystin or a halogenated baeocystin and the psilocybin biosynthetic enzyme complement can comprise a nucleic acid encoding PsiM selected from SEQ.ID NO: 8, SEQ.ID NO: 59 and SEQ.ID NO: 61 or a nucleic acid sequence encoding a polypeptide sequence substantially similar thereto; or
(e) the halogenated psilocybin precursor compound can be a halogenated tryptophan, and the psilocybin biosynthetic enzyme complement can comprise (i) a nucleic acid encoding PsiD selected from SEQ.ID NO: 2, SEQ.ID NO: 10, SEQ.ID NO: 37, SEQ.ID NO: 39, SEQ.ID NO: 41, SEQ.ID NO: 43, SEQ.ID NO: 45, SEQ.ID NO: 47, SEQ.ID NO: 49, SEQ.ID NO: 51, SEQ.ID NO: 53, SEQ.ID NO: 55, and SEQ.ID NO: 57, or a nucleic acid sequence encoding a polypeptide sequence substantially similar thereto; (ii) a nucleic acid encoding PsiH encoded by SEQ.ID NO: 4, or a nucleic acid sequence encoding a polypeptide sequence substantially similar thereto; (iii) a nucleic acid encoding PsiK encoded by SEQ.ID NO: 6, or a nucleic acid sequence encoding a polypeptide sequence substantially similar thereto; and (iv) and a nucleic acid encoding PsiM selected from SEQ.ID NO: 8, SEQ.ID NO: 59 and SEQ.ID NO: 61 or a nucleic acid sequence encoding a polypeptide sequence substantially similar thereto; or
(f) the halogenated psilocybin precursor compound can be, a halogenated tryptamine, and psilocybin biosynthetic enzyme complement can comprise (i) a nucleic acid encoding PsiH encoded by SEQ.ID NO: 4, or a nucleic acid sequence encoding a polypeptide sequence substantially similar thereto; (ii) a nucleic acid encoding PsiK encoded by SEQ.ID NO: 6, or a nucleic acid sequence encoding a polypeptide sequence substantially similar thereto; and (iii) and a nucleic acid encoding PsiM selected from SEQ.ID NO: 8, SEQ.ID NO: 59 and SEQ.ID NO: 61 or a nucleic acid sequence encoding a polypeptide sequence substantially similar thereto.

In at least one embodiment, in an aspect, the method can further include a step comprising isolating the halogenated psilocybin derivative.

In at least one embodiment, in an aspect, the host cell can be a microbial cell.

In at least one embodiment, in an aspect, the host cell can be a bacterial cell or a yeast cell.

In another aspect, the present disclosure provides, in at least one embodiment, a method for modulating a 5-HT_{2A} receptor, the method comprising contacting a 5-HT_{2A} receptor with a chemical compound or salts thereof having the formula (I):

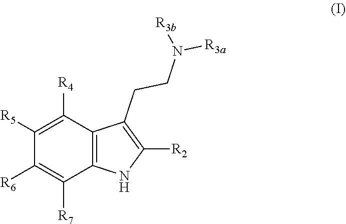

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein R_{3A} and R_{3B} are a hydrogen atom, an alkyl group, an aryl group, or an acyl group under reaction conditions sufficient to thereby modulate receptor activity.

In at least one embodiment, in an aspect, the reaction conditions can be in vitro reaction conditions.

In at least one embodiment, in an aspect, the reaction conditions can be in vivo reaction conditions.

In another aspect, the present disclosure provides, in at least one embodiment, a use of a chemical compound or salts thereof having the formula (I):

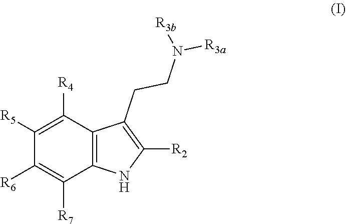

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein R_{3A} and R_{3B} are a hydrogen atom, an alkyl group, an aryl group, or an acyl group in the manufacture of a pharmaceutical or recreational drug formulation.

In at least one embodiment, in an aspect, the manufacture of a pharmaceutical or recreational drug formulation can comprise formulating the chemical compound with an excipient, diluent or carrier.

In at least one embodiment, the manufacture of a pharmaceutical or recreational drug formulation can comprise derivatizing the chemical compound having the formula (I) by substituting the halogen atom with another atom or chemical group.

In another aspect, the present disclosure provides, in at least one embodiment, a use of a chemical compound or salts thereof having the formula (I):

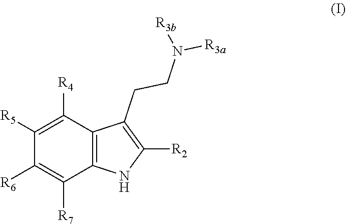

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein R_{3A} and R_{3B} are a hydrogen atom, an alkyl group, an aryl group, or an acyl group together with a diluent, carrier or excipient, as a pharmaceutical or recreational drug formulation.

Other features and advantages will become apparent from the following detailed description. It should be understood, however, that the detailed description, while indicating preferred implementations of the disclosure, are given by way of illustration only, since various changes and modifications within the spirit and scope of the disclosure will become apparent to those of skill in the art from the detailed description.

### BRIEF DESCRIPTION OF THE DRAWINGS

The disclosure is in the hereinafter provided paragraphs described, by way of example, in relation to the attached figures. The figures provided herein are provided for a better understanding of the example embodiments and to show more clearly how the various embodiments may be carried into effect. The figures are not intended to limit the present disclosure.

FIG. 1 depicts the chemical structure of psilocybin.

FIG. 2 depicts a certain prototype structure of a psilocybin precursor compound, namely an indole. Certain carbon and nitrogen atoms may be referred herein by reference to their position within the indole structure, i.e. N₁, C₂, C₃ etc. The pertinent atom numbering is shown.

FIGS. 3A, 3B, 3C, 3D, 3E, 3F and 3G depict the chemical structures of example psilocybin precursor compounds, notably L-tryptophan (FIG. 3A), tryptamine (FIG. 3B), 4-hydroxytryptamine (FIG. 3C), L-4-hydroxytryptophan (FIG. 3D), 4-hydroxyindole (FIG. 3E), norbaeocystin (FIG. 3F), and baeocystin (FIG. 3G).

FIGS. 4A, 4B, 4C, 4D, 4E, 4F, 4G, 4H, 4I, 4J, 4K, 4L, 4M, 4N, 4O, 4P, 4Q and 4R depict the chemical structures of certain example halogenated psilocybin derivative compounds, notably a 2-chloro-4-phospho psilocybin derivative (FIG. 4A); a 2-bromo psilocybin derivative (FIG. 4B); a 4-iodo psilocybin derivative (FIG. 4C); a 4-fluoro psilocybin derivative (FIG. 4D); a 4-phospho-5-fluoro psilocybin derivative (FIG. 4E); a 5-chloro psilocybin derivative (FIG. 4F); a 4-phospho-6-bromo psilocybin derivative (FIG. 4G); a 6-chloro psilocybin derivative (FIG. 4H) a 4-phospho-7-fluoro psilocybin derivative (FIG. 4I); a 7-iodo psilocybin derivative (FIG. 4J); a 2-chloro-4-methyl psilocybin derivative (FIG. 4K); a 4-ethyl-5-bromo psilocybin derivative (FIG. 4L); a 4-methyl-6-fluoro psilocybin derivative (FIG. 4M); a 4-propyl-7-iodo psilocybin derivative (FIG. 4N); a 2-chloro-4-O-methyl psilocybin derivative (FIG. 4K); a 4-O-ethyl-5-bromo psilocybin derivative (FIG. 4L); a 4-O-methyl-6-fluoro psilocybin derivative (FIG. 4M); and a 4-O-propyl-7-iodo psilocybin derivative (FIG. 4N). It is noted that in each of FIGS. 4A, 4B, 4C, 4D, 4E, 4F, 4G, 4H, 4I, 4J, 4K, 4L, 4M, 4N, 4O, 4P, 4Q and 4R R₃ₐ and R_{3b} can be a hydrogen atom, an alkyl group, an aryl group, or an acyl group.

FIGS. 5A, 5B, 5C, 5D, 5E, 5F, 5G, 5H, 5I and 5J depict the chemical structures of certain example halogenated psilocybin derivative compounds, notably a 2,4-di-chloro psilocybin derivative (FIG. 5A); a 2-bromo-5-fluoro-7-methyl psilocybin derivative (FIG. 5B); a 2-chloro-O-methyl-6-bromo psilocybin derivative (FIG. 5C); a 2-fluoro-4-phospho-7-iodo psilocybin derivative (FIG. 5D); a 4,5-di-bromo psilocybin derivative (FIG. 5E); a 4,6-di-chloro psilocybin derivative (FIG. 5F); a 4-chloro-5-fluoro psilocybin derivative (FIG. 5G); a 4-phospho-5-fluoro-6-bromo psilocybin derivative (FIG. 5H) a 4-phospho-5,7-di-fluoro psilocybin derivative (FIG. 5I); and a 6,7-di-iodo psilocybin derivative (FIG. 5J). It is noted that in each of FIGS. 5A, 5B, 5C, 5D, 5E, 5F, 5G, 5H, 5I and 5J R₃ₐ and R_{3b} can be a hydrogen atom, an alkyl group, an aryl group, or an acyl group.

FIGS. 6A, 6B, 6C, 6D, 6E, and 6F depict the chemical structures of certain example halogenated psilocybin derivative compounds, notably a 2,4,5-tri-chloro psilocybin derivative (FIG. 6A); a 2-bromo-5,6-di-fluoro psilocybin derivative (FIG. 6B); a 2-chloro-5-bromo-7-fluoro psilocybin derivative (FIG. 6C); a 4,5-di-bromo-6-chloro psilocybin derivative (FIG. 6D); a 4,5,7-tri-bromo psilocybin derivative (FIG. 6E); and a 4-phospho-5-fluoro-6-chloro-7-iodo psilocybin derivative (FIG. 6F). It is noted that in each of FIGS. 6A, 6B, 6C, 6D, 6E, and 6F R₃ₐ and R_{3b} can be a hydrogen atom, an alkyl group, an aryl group, or an acyl group.

FIGS. 7A, 7B, 7C, 7D, and 7E depict the chemical structures of certain example halogenated psilocybin derivative compounds, notably a 2,4,5,6-tetra-chloro psilocybin derivative (FIG. 7A); a 4,7-di-chloro-5,6-di-fluoro psilocybin derivative (FIG. 7B); a 2-chloro-4-phospho-5-fluoro-6-chloro-7-iodo psilocybin derivative (FIG. 7C); a 2,4,7-tri-bromo-6-chloro psilocybin derivative (FIG. 7D); and a 2-fluoro-4,5,7-tri-bromo psilocybin derivative (FIG. 7E). It is noted that in each of FIGS. 7A, 7B, 7C, 7D, and 7E R₃ₐ and R_{3b} can be a hydrogen atom, an alkyl group, an aryl group, or an acyl group.

FIGS. 8A and 8B depict example biosynthesis pathway relating to the synthesis of a halogenated psilocybin derivatives.

FIGS. 9A and 9B depict example chemical synthesis processes for the synthesis of certain example halogenated psilocybin derivatives, notably the compound denoted as 410B01 (FIG. 9A) and corresponding with a halogenated psilocybin derivative compound having the chemical formula (IV) set forth herein, and the compound denoted as 1 (FIG. 9B) and corresponding with a halogenated psilocybin derivative compound having the chemical formula (VIII) set forth herein.

FIG. 10 depicts a representation of mass spectrometry data in the form of a chromatogram, notably a chromatogram obtained in the performance of an experiment to synthesize an example halogenated psilocybin derivative compound having the chemical formula (XI) set forth herein.

FIG. 11 depicts a representation of mass spectrometry data in the form of a chromatogram, notably a chromatogram obtained in the performance of an experiment to synthesize an example halogenated psilocybin derivative compound having the chemical formula (XIV) set forth herein.

FIG. 12 depicts a representation of mass spectrometry data in the form of a chromatogram, notably a chromatogram obtained in the performance of an experiment to synthesize an example halogenated psilocybin derivative compound having the chemical formula (XVI) set forth herein.

FIG. 13 depicts a representation of mass spectrometry data in the form of a chromatogram, notably a chromatogram obtained in the performance of an experiment to synthesize an example halogenated psilocybin derivative compound having the chemical formula (XVII) set forth herein.

FIG. 14 depicts a graph obtained in the performance of an experimental assay to evaluate the efficacy of an example halogenated psilocybin derivative, notably a cell viability assay involving an example halogenated psilocybin derivative compound having the chemical formula (XV) set forth herein.

FIGS. 15A and 15B depict graphs obtained in the performance of an experimental assay to evaluate the efficacy of an example halogenated psilocybin derivative, notably a 5-HT₂ₐ receptor modulation assay involving +5-HT_{2A} cells and an example halogenated psilocybin derivative compound having the chemical formula (XV) set forth herein (FIG. 15B) and a control (psilocin) (FIG. 15A).

FIGS. 16A and 16B depict graphs obtained in the performance of an experimental assay to evaluate the efficacy of an example halogenated psilocybin derivative, notably a 5-HT₂ₐ receptor modulation assay involving −5-HT_{2A} cells and an example halogenated psilocybin derivative compound having the chemical formula (XV) set forth herein (FIG. 16B) and a control (psilocin) (FIG. 16A).

FIG. 17 depicts a representation of mass spectrometry data in the form of a chromatogram, notably a chromatogram obtained in the performance of an experiment to synthesize an example halogenated psilocybin derivative compound having the chemical formula (II) set forth herein.

FIG. 18 depicts a graph obtained in the performance of an experimental assay to evaluate the efficacy of an example halogenated psilocybin derivative, notably a cell viability assay involving an example halogenated psilocybin derivative compound having the chemical formula (IX) set forth herein.

FIGS. 19A and 19B depict graphs obtained in the performance of an experimental assay to evaluate the efficacy of an example halogenated psilocybin derivative, notably a 5-HT₂ₐ receptor modulation assay involving +5-HT_{2A} cells (FIG. 19A) and −5-HT_{2A} cells (FIG. 19B) and an example halogenated psilocybin derivative compound having the chemical formula (IX) set forth herein.

FIG. 20 depicts a representation of mass spectrometry data in the form of a chromatogram, notably a chromatogram obtained in the performance of an experiment to synthesize an example halogenated psilocybin derivative compound having the chemical formula (VI) set forth herein.

FIG. 21 depicts a graph obtained in the performance of an experimental assay to evaluate the efficacy of an example halogenated psilocybin derivative, notably a cell viability assay involving an example halogenated psilocybin derivative compound having the chemical formula (V) set forth herein.

FIGS. 22A and 22B depict graphs obtained in the performance of an experimental assay to evaluate the efficacy of an example halogenated psilocybin derivative, notably a 5-HT₂ₐ receptor modulation assay involving +5-HT_{2A} cells (FIG. 22A) and −5-HT_{2A} cells (FIG. 22B), and an example halogenated psilocybin derivative compound having the chemical formula (V) set forth herein.

FIG. 23 depicts a representation of mass spectrometry data in the form of a chromatogram, notably a chromatogram obtained in the performance of an experiment to synthesize an example halogenated psilocybin derivative compound having the chemical formula (XIII) set forth herein.

FIG. 24 depicts a graph obtained in the performance of an experimental assay to evaluate the efficacy of an example halogenated psilocybin derivative, notably a cell viability assay involving an example halogenated psilocybin derivative compound having the chemical formula (X) set forth herein.

FIGS. 25A and 25B depict graphs obtained in the performance of an experimental assay to evaluate the efficacy of an example halogenated psilocybin derivative, notably a 5-HT₂ₐ receptor modulation assay involving +5-HT_{2A} cells (FIG. 25A) and −5-HT_{2A} cells (FIG. 25B), and an example halogenated psilocybin derivative compound having the chemical formula (X) set forth herein.

FIG. 26 depicts a graph obtained in the performance of an experimental assay to evaluate the efficacy of an example halogenated psilocybin derivative, notably a cell viability assay involving an example halogenated psilocybin derivative compound having the chemical formula (IV) set forth herein.

FIGS. 27A and 27B depict graphs obtained in the performance of an experimental assay to evaluate the efficacy of an example halogenated psilocybin derivative, notably a 5-HT₂ₐ receptor modulation assay involving +5-HT_{2A} cells (FIG. 27A) and −5-HT_{2A} cells (FIG. 27B), and an example halogenated psilocybin derivative compound having the chemical formula (IV) set forth herein.

FIG. 28 depicts a graph obtained in the performance of an experimental assay to evaluate the efficacy of an example halogenated psilocybin derivative, notably a cell viability assay involving an example halogenated psilocybin derivative compound having the chemical formula (VIII) set forth herein.

FIGS. 29A and 29B depict graphs obtained in the performance of an experimental assay to evaluate the efficacy of an example halogenated psilocybin derivative, notably a 5-HT₂ₐ receptor modulation assay involving +5-HT_{2A} cells (FIG. 29A) and −5-HT_{2A} cells (FIG. 29B), and an example halogenated psilocybin derivative compound having the chemical formula (VIII) set forth herein.

FIG. 30 depicts a representation of mass spectrometry data in the form of a chromatogram, notably a chromatogram obtained in the performance of an experiment to synthesize an example halogenated psilocybin derivative compound having the chemical formula (XVIII) set forth herein.

The figures together with the following detailed description make apparent to those skilled in the art how the disclosure may be implemented in practice.

### DETAILED DESCRIPTION

Various compositions, systems or processes will be described below to provide an example of an embodiment of each claimed subject matter. No embodiment described below limits any claimed subject matter and any claimed subject matter may cover processes, compositions or systems that differ from those described below. The claimed subject matter is not limited to compositions, processes or systems having all of the features of any one composition, system or process described below or to features common to multiple or all of the compositions, systems or processes described below. It is possible that a composition, system or process described below is not an embodiment of any claimed subject matter. Any subject matter disclosed in a composition, system or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

As used herein and in the claims, the singular forms, such “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers.

Various compositions, systems or processes will be described below to provide an example of an embodiment of each claimed subject matter. No embodiment described below limits any claimed subject matter and any claimed subject matter may cover processes, compositions or systems that differ from those described below. The claimed subject matter is not limited to compositions, processes or systems having all of the features of any one composition, system or process described below or to features common to multiple or all of the compositions, systems or processes described below. It is possible that a composition, system or process described below is not an embodiment of any claimed subject matter. Any subject matter disclosed in a composition, system or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

When ranges are used herein for physical properties, such as molecular weight, or chemical properties, such as chemical formulae, all combinations and sub-combinations of ranges and specific embodiments therein are intended to be included. Other than in the operating examples, or where otherwise indicated, all numbers expressing quantities of ingredients or reaction conditions used herein should be understood as modified in all instances by the term “about.” The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context. Furthermore any range of values described herein is intended to specifically include the limiting values of the range, and any intermediate value or sub-range within the given range, and all such intermediate values and sub-ranges are individually and specifically disclosed (e.g. a range of 1 to 5 includes 1, 1.5, 2, 2.75, 3, 3.90, 4, and 5). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only, and is not intended to limit the scope of the present invention, which is defined solely by the claims.

All publications, patents and patent applications are herein incorporated by reference in their entirety to the same extent as if each individual publication, patent or patent application was specifically and individually indicated to be incorporated by reference in its entirety.

### Terms and Definitions

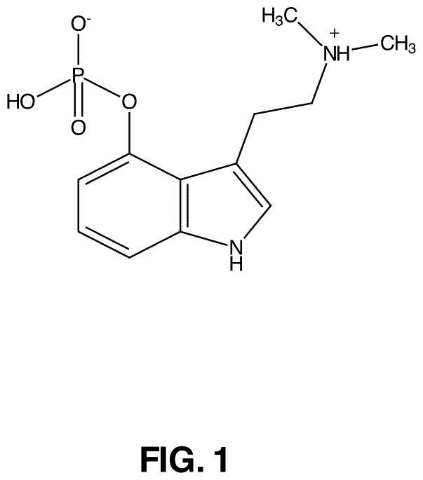

The term “psilocybin”, as used herein, refers to a chemical compound having the structure set forth in FIG. 1, and further includes salts, thereof, such as a sodium salt, a potassium salt, etc.

The term “psilocybin precursor compound”, as used herein, refers to a chemical compound that may serve as a precursor compound in the synthesis or biosynthesis of psilocybin, and includes compounds comprising an indole prototype structure, including, for example, tryptophan, tryptamine, 4-hydroxyindole, 4-hydroxytryptophan, 4-hydroxytryptamine, norbaeocystin, and baeocystin, and further includes halogenated derivatives and salts of any of the foregoing.

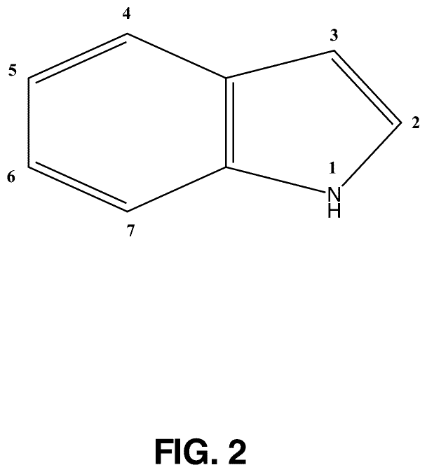

The term “indole prototype structure”, as used herein, refers to the chemical structure shown in FIG. 2. It is noted that specific carbon atoms and a nitrogen atom in the indole prototype structure are numbered. Reference may be made to these carbon and nitrogen numbers herein, for example C₂, C₄, N₁, and so forth. Furthermore, reference may be made to chemical groups attached to the indole prototype structure in accordance with the same numbering, for example R₄ and R₆ reference chemical groups attached to the C₄ and C₆ atom, respectively. In addition, R_{3A} and R_{3B}, in this respect, reference chemical groups extending from the ethyl-amino group extending in turn from the C₃ atom of the prototype indole structure.

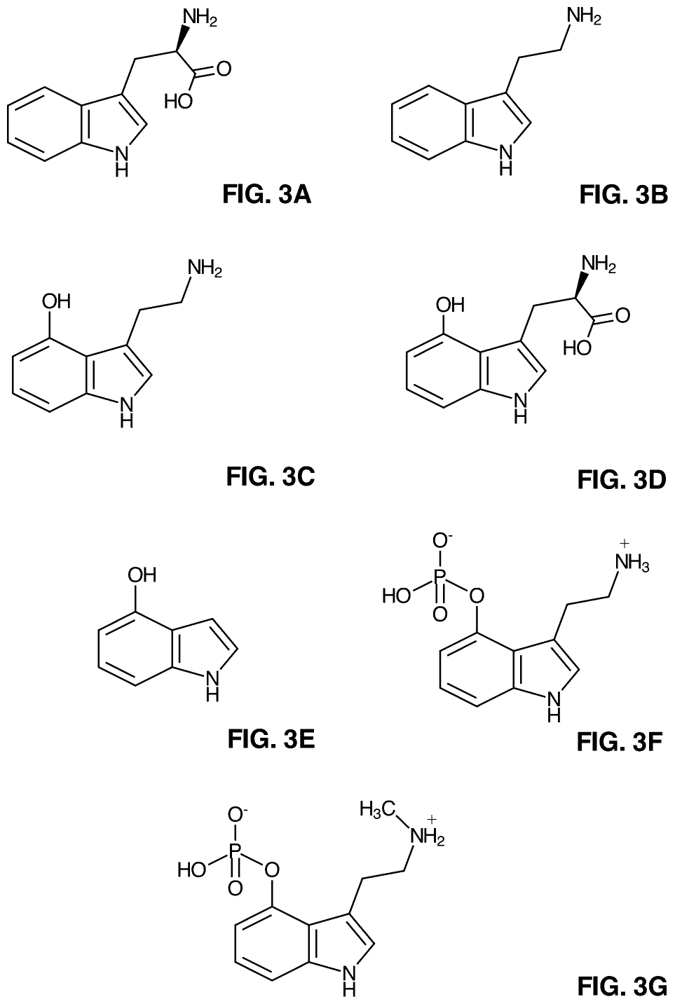

The term “tryptophan”, as used herein, refers to a chemical compound having the structure set forth in FIG. 3A and further includes its D-enantiomeric form (not shown).

The term “tryptamine”, as used herein, refers to a chemical compound having the structure set forth in FIG. 3B.

The term “4-hydroxytryptamine”, as used herein, refers to a chemical compound having the structure set forth in FIG. 3C.

The term “4-hydroxytryptophan”, as used herein, refers to a chemical compound having the structure set forth in FIG. 3D and further includes its D-enantiomeric form (not shown).

The term “4-hydroxyindole”, as used herein, refers to a chemical compound having the structure set forth in FIG. 3E.

The term “norbaeocystin”, as used herein, refers to a chemical compound having the structure set forth in FIG. 3F.

The term “baeocystin”, as used herein, refers to a chemical compound having the structure set forth in FIG. 3G.

The term “phosphate group”, as used herein, is a molecule containing one atom of phosphorus, covalently bound to four oxygen atoms (three single bonds and one double bond). Of the four oxygen atoms one oxygen atom may be a hydroxy group, and one of the non-hydroxylated oxygen atom may be chemically bonded to another entity.

The terms “hydroxy group”, and “hydroxy”, as used herein, refers to a molecule containing one atom of oxygen bonded to one atom of hydrogen, and having the formula —OH. A hydroxy group through its oxygen atom may be chemically bonded to another entity.

The terms “halogen”, “halogenated” and “halo-”, as used herein, refer to the class of chemical elements consisting of fluorine (F), chlorine (Cl), bromine (Br), and iodine (I). Accordingly, halogenated compounds can refer to “fluorinated”, “chlorinated”, “brominated”, or “iodinated” compounds.

The term “halogenated psilocybin derivative”, as used herein, refers to a psilocybin derivative in which a halogen been bonded to psilocybin, or a derivative thereof. Reference may be made to specific carbon atoms of the psilocybin derivative which may be halogenated, for example, 7-halo-psilocybin refers to a halogenated psilocybin in which carbon atom number 7 (as identified in the indole prototype structure) is halogenated, or, similarly, 2-bromo-psilocybin refers to a brominated psilocybin in which carbon atom number 2 (as identified in the indole prototype structure) is brominated, and so on. The term further includes salts of halogenated psilocybin derivatives, such as sodium salt, potassium salt, or a chlorine salt, for example. Salts may be formed by virtue of a charged atom or group included in the psilocybin derivative, notably a charged nitrogen atom, and/or a charged phosphate group.

The term “halogenated psilocybin precursor compound”, as used herein, refers to a psilocybin precursor compound possessing a halogen. Reference may be made to specific carbon atoms of the psilocybin precursor compound which may be halogenated, for example, 6-halo-baeocystin refers to a halogenated baeocystin in which carbon atom number 6 (as identified in the indole prototype structure) is halogenated, or, similarly, 2-chloro-4-hydroxytryptamine refers to a chlorinated 4-hydroxytryptamine in which carbon atom number 2 (as identified in the indole prototype structure) is chlorinated. Furthermore, halogenated psilocybin precursor compounds include, without limitation, halogenated tryptophan, e.g. 7-bromo tryptophan, 6-chloro tryptophan, 5,6-difluoro tryptophan, halogenated 4-hydroxy indole e.g. 4-hydroxy-7-bromo indole, 4-hydroxy-5-chloro indole, 6,7-di-fluoro-indole

The term “alkyl”, as used herein, refers to a straight and/or branched chain, saturated alkyl radical containing from one to “p” carbon atoms (“C₁-Cₚ-alkyl”) and includes, depending on the identity of “p”, methyl, ethyl, propyl, isopropyl n-butyl, s-butyl, isobutyl; t-butyl, 2,2-dimethylbutyl, n-pentyl, 2-methylpentyl, 3-methylpentyl, 4-methylpentyl, n-hexyl and the like, where the variable p is an integer representing the largest number of carbon atoms in the alkyl radical. Alkyl groups further include hydrocarbon groups arranged in a chain having the chemical formula —CₙH₂ₙ₊₁, including, without limitation, methyl groups (—CH₃), ethyl groups (—C₂H₅), propyl groups (—C₃H₇), and butyl groups (—C₄H₉).

The term “O-alkyl”, as used herein, refers to a hydrocarbon group arranged in a chain having the chemical formula —O—CₙH₂ₙ₊₁. O-alkyl groups include, without limitation, O-methyl groups (—O—CH₃), O-ethyl groups (—O—C₂H₅), O-propyl groups (—O—C₃H₇) and O-butyl groups (—O—C₄H₉).

The term “acyl”, as used herein, refers to a carbon atom double bonded to an oxygen and single bonded to an alkyl group. The carbon atom further can be bonded to another entity. An acyl group can be described by the chemical formula: —C(═O)—CₙH₂ₙ₊₁.

The term “aryl”, as used herein, refers to a monocyclic, bicyclic or tricyclic aromatic ring system containing, depending on the number of atoms in the rings, for example, from 6 to 14 carbon atoms (C₆-C₁₄-aryl) or from 6 to 10 carbons (C₆-C₁₀-aryl), and at least 1 aromatic ring and includes phenyl, naphthyl, anthracenyl, 1,2-dihydronaphthyl, 1,2,3,4-tetrahydronaphthyl, fluorenyl, phenanthrenyl, biphenylenyl, indanyl, indenyl and the like,

The term “5-HT_{2A} receptor”, as used herein, refers to a subclass of a family of receptors for the neurotransmitter and peripheral signal mediator serotonin. 5-HT_{2A} receptors can mediate a plurality of central and peripheral physiologic functions of serotonin. Central nervous system effects can include mediation of hallucinogenic effects of hallucinogenic compounds.

The term “modulating 5-HT_{2A} receptors”, as used herein, refers to the ability of a compound disclosed herein to alter the function of 5-HT_{2A} receptors. A 5-HT_{2A} receptor modulator may activate the activity of a 5-HT_{2A} receptor, may activate or inhibit the activity of a 5-HT_{2A} receptor depending on the concentration of the compound exposed to the 5-HT_{2A} receptor, or may inhibit the activity of a 5-HT_{2A} receptor. Such activation or inhibition may be contingent on the occurrence of a specific event, such as activation of a signal transduction pathway, and/or maybe manifest only in particular cell types. The term “modulating 5-HT_{2A} receptors,” also refers to altering the function of a 5-HT_{2A} receptor by increasing or decreasing the probability that a complex forms between a 5-HT_{2A} receptor and a natural binding partner to form a multimer. A 5-HT_{2A} receptor modulator may increase the probability that such a complex forms between the 5-HT_{2A} receptor and the natural binding partner, may increase or decrease the probability that a complex forms between the 5-HT_{2A} receptor and the natural binding partner depending on the concentration of the compound exposed to the 5-HT_{2A} receptor, and or may decrease the probability that a complex forms between the 5-HT_{2A} receptor and the natural binding partner. Furthermore, the term includes allosteric modulation of the receptor 5-HT_{2A}, i.e. modulation of the 5-HT_{2A} receptor through interaction with the 5-HT_{2A} receptor that is topographically different than the orthosteric site recognized by the cell's endogenous agonist, such modulation further including positive allosteric modulation (PAM), negative allosteric modulation (NAM) and silent allosteric modulation (SAM).

The term “5-HT_{2A} receptor-mediated disorder”, as used herein, refers to a disorder that is characterized by abnormal 5-HT_{2A} receptor activity. A 5-HT_{2A} receptor-mediated disorder may be completely or partially mediated by modulating 5-HT_{2A} receptors. In particular, a 5-HT_{2A} receptor-mediated disorder is one in which modulation of 5-HT_{2A} receptors results in some effect on the underlying disorder e.g., administration of a 5-HT_{2A} receptor modulator results in some improvement in at least some of the subjects being treated.

The term “pharmaceutical formulation”, as used herein, refers to a preparation in a form which allows an active ingredient, including a psychoactive ingredient, contained therein to provide effective treatment, and which does not contain any other ingredients which cause excessive toxicity, an allergic response, irritation, or other adverse response commensurate with a reasonable risk/benefit ratio. The pharmaceutical formulation may contain other pharmaceutical ingredients such as excipients, carriers, diluents, or auxiliary agents.

The term “recreational drug formulation”, as used herein, refers to a preparation in a form which allows a psychoactive ingredient contained therein to be effective for administration as a recreational drug, and which does not contain any other ingredients which cause excessive toxicity, an allergic response, irritation, or other adverse response commensurate with a reasonable risk/benefit ratio. The recreational drug formulation may contain other ingredients such as excipients, carriers, diluents, or auxiliary agents.

The term “effective for administration as a recreational drug”, as used herein, refers to a preparation in a form which allows a subject to voluntarily induce a psychoactive effect for non-medical purposes upon administration, generally in the form of self-administration. The effect may include an altered state of consciousness, satisfaction, pleasure, euphoria, perceptual distortion, or hallucination.

The term “effective amount”, as used herein, refers to an amount of an active agent, pharmaceutical formulation or recreational drug formulation, sufficient to induce a desired biological or therapeutic effect, including a prophylactic effect, and further including a psychoactive effect. Such effect can include an effect with respect to the signs, symptoms or causes of a disorder, or disease or any other desired alteration of a biological system. The effective amount can vary depending, for example, on the health condition, injury stage, disorder stage, or disease stage, weight, or sex of a subject being treated, timing of the administration, manner of the administration, age of the subject, and the like, all of which can be determined by those of skill in the art.

The terms “treating” and “treatment”, and the like, as used herein, are intended to mean obtaining a desirable physiological, pharmacological, or biological effect, and includes prophylactic and therapeutic treatment. The effect may result in the inhibition, attenuation, amelioration, or reversal of a sign, symptom or cause of a disorder, or disease, attributable to the disorder, or disease, which includes mental and psychiatric diseases and disorders. Clinical evidence of the prevention or treatment may vary with the disorder, or disease, the subject and the selected treatment.

The term “pharmaceutically acceptable”, as used herein, refers to materials, including excipients, carriers, diluents, or auxiliary agents, that are compatible with other materials in a pharmaceutical or recreational drug formulation and within the scope of reasonable medical judgement suitable for use in contact with a subject without excessive toxicity, allergic response, irritation, or other adverse response commensurate with a reasonable risk/benefit ratio.

The term “psilocybin biosynthetic enzyme complement”, as used herein, refers to one or more polypeptides which alone or together are capable of facilitating the chemical conversion of a psilocybin precursor compound and form another psilocybin precursor compound or psilocybin or a halogenated form thereof. A psilocybin biosynthetic enzyme complement can include, for example, PsiD, PsiH, PsiK, PsiM, Psi-ncAAAD and TrpB.

The term “PsiD” as used herein, refers to any and all enzymes comprising a sequence of amino acid residues which is (i) substantially identical to the amino acid sequences constituting any PsiD polypeptide set forth herein, including, for example, SEQ.ID NO: 2, SEQ.ID NO: 37, SEQ.ID NO: 39, SEQ.ID NO: 41, SEQ.ID NO: 43, SEQ.ID NO: 45, SEQ.ID NO: 47, SEQ.ID NO: 49, SEQ.ID NO: 51, SEQ.ID NO: 53, SEQ.ID NO: 55, and SEQ.ID NO: 57 or (ii) encoded by a nucleic acid sequence capable of hybridizing under at least moderately stringent conditions to any nucleic acid sequence encoding any PsiD set forth herein, but for the use of synonymous codons.

The term “PsiH” as used herein, refers to any and all enzymes comprising a sequence of amino acid residues which is (i) substantially identical to the amino acid sequences constituting any PsiH polypeptide set forth herein, including, for example, SEQ.ID NO: 4, or (ii) encoded by a nucleic acid sequence capable of hybridizing under at least moderately stringent conditions to any nucleic acid sequence encoding any PsiH set forth herein, but for the use of synonymous codons.

The term “PsiK” as used herein, refers to any and all enzymes comprising a sequence of amino acid residues which is (i) substantially identical to the amino acid sequences constituting any PsiK polypeptide set forth herein, including, for example, SEQ.ID NO: 6, or (ii) encoded by a nucleic acid sequence capable of hybridizing under at least moderately stringent conditions to any nucleic acid sequence encoding any PsiK set forth herein, but for the use of synonymous codons.

The term “PsiM” as used herein, refers to any and all enzymes comprising a sequence of amino acid residues which is (i) substantially identical to the amino acid sequences constituting any PsiM polypeptide set forth herein, including, for example, SEQ.ID NO: 8, and SEQ.ID NO: 59 or (ii) encoded by a nucleic acid sequence capable of hybridizing under at least moderately stringent conditions to any nucleic acid sequence encoding any PsiM set forth herein, but for the use of synonymous codons.

The term “Psi-ncAAAD” as used herein, refers to any and all enzymes comprising a sequence of amino acid residues which is (i) substantially identical to the amino acid sequences constituting any Psi-ncAAAD polypeptide set forth herein, including, for example, SEQ.ID NO: 10, or (ii) encoded by a nucleic acid sequence capable of hybridizing under at least moderately stringent conditions to any nucleic acid sequence encoding any Psi-ncAAAD set forth herein, but for the use of synonymous codons.

The term “TrpB” as used herein, refers to any and all enzymes comprising a sequence of amino acid residues which is (i) substantially identical to the amino acid sequences constituting any TrpB polypeptide set forth herein, including, for example, SEQ.ID NO: 12, SEQ.ID NO: 65, SEQ.ID NO: 67, SEQ.ID NO: 69, SEQ.ID NO: 71, and SEQ.ID NO: 73, or (ii) encoded by a nucleic acid sequence capable of hybridizing under at least moderately stringent conditions to any nucleic acid sequence encoding any TrpB set forth herein, but for the use of synonymous codons.

The term “halogenase”, as used herein, refers to any and all enzymes comprising a sequence of amino acid residues which is (i) substantially identical to the amino acid sequences constituting any halogenase polypeptide set forth herein, including, for example, SEQ.ID NO: 14, or (ii) encoded by a nucleic acid sequence capable of hybridizing under at least moderately stringent conditions to any nucleic acid sequence encoding any halogenase set forth herein, but for the use of synonymous codons.

The term “acetyl transferase”, as used herein, refers to any and all enzymes comprising a sequence of amino acid residues which is (i) substantially identical to the amino acid sequences constituting any acetyl transferase polypeptide set forth herein, including, for example, SEQ.ID NO: 63, or (ii) encoded by a nucleic acid sequence capable of hybridizing under at least moderately stringent conditions to any nucleic acid sequence encoding any acetyl transferase set forth herein, but for the use of synonymous codons.

The terms “nucleic acid sequence encoding PsiD”, and “nucleic acid sequence encoding a PsiD polypeptide”, as may be used interchangeably herein, refer to any and all nucleic acid sequences encoding a PsiD polypeptide, including, for example, SEQ.ID NO: 1, SEQ.ID NO: 36, SEQ.ID NO: 38, SEQ.ID NO: 40, SEQ.ID NO: 42, SEQ.ID NO: 44, SEQ.ID NO: 46, SEQ.ID NO: 48, SEQ.ID NO: 50, SEQ.ID NO: 52, SEQ.ID NO: 54, and SEQ.ID NO: 56. Nucleic acid sequences encoding a PsiD polypeptide further include any and all nucleic acid sequences which (i) encode polypeptides that are substantially identical to the PsiD polypeptide sequences set forth herein; or (ii) hybridize to any PsiD nucleic acid sequences set forth herein under at least moderately stringent hybridization conditions or which would hybridize thereto under at least moderately stringent conditions but for the use of synonymous codons.

The terms “nucleic acid sequence encoding PsiH”, and “nucleic acid sequence encoding a PsiH polypeptide”, as may be used interchangeably herein, refer to any and all nucleic acid sequences encoding a PsiH polypeptide, including, for example, SEQ.ID NO: 3. Nucleic acid sequences encoding a PsiH polypeptide further include any and all nucleic acid sequences which (i) encode polypeptides that are substantially identical to the PsiH polypeptide sequences set forth herein; or (ii) hybridize to any PsiH nucleic acid sequences set forth herein under at least moderately stringent hybridization conditions or which would hybridize thereto under at least moderately stringent conditions but for the use of synonymous codons.

The terms “nucleic acid sequence encoding PsiK”, and “nucleic acid sequence encoding a PsiK polypeptide”, as may be used interchangeably herein, refer to any and all nucleic acid sequences encoding a PsiK polypeptide, including, for example, SEQ.ID NO: 5. Nucleic acid sequences encoding a PsiK polypeptide further include any and all nucleic acid sequences which (i) encode polypeptides that are substantially identical to the PsiK polypeptide sequences set forth herein; or (ii) hybridize to any PsiK nucleic acid sequences set forth herein under at least moderately stringent hybridization conditions or which would hybridize thereto under at least moderately stringent conditions but for the use of synonymous codons.

The terms “nucleic acid sequence encoding PsiM”, and “nucleic acid sequence encoding a PsiM polypeptide”, as may be used interchangeably herein, refer to any and all nucleic acid sequences encoding a PsiD polypeptide, including, for example, SEQ.ID NO: 7 SEQ.ID NO: 58 and SEQ.ID NO: 61. Nucleic acid sequences encoding a PsiM polypeptide further include any and all nucleic acid sequences which (i) encode polypeptides that are substantially identical to the PsiM polypeptide sequences set forth herein; or (ii) hybridize to any PsiM nucleic acid sequences set forth herein under at least moderately stringent hybridization conditions or which would hybridize thereto under at least moderately stringent conditions but for the use of synonymous codons.

The terms “nucleic acid sequence encoding Psi-ncAAAD”, and “nucleic acid sequence encoding a Psi-ncAAAD polypeptide”, as may be used interchangeably herein, refer to any and all nucleic acid sequences encoding a PsiD polypeptide, including, for example, SEQ.ID NO: 9. Nucleic acid sequences encoding a Psi-ncAAAD polypeptide further include any and all nucleic acid sequences which (i) encode polypeptides that are substantially identical to the Psi-ncAAAD polypeptide sequences set forth herein; or (ii) hybridize to any Psi-ncAAAD nucleic acid sequences set forth herein under at least moderately stringent hybridization conditions or which would hybridize thereto under at least moderately stringent conditions but for the use of synonymous codons.

The terms “nucleic acid sequence encoding TrpB”, and “nucleic acid sequence encoding a TrpB polypeptide”, as may be used interchangeably herein, refer to any and all nucleic acid sequences encoding a TrpB polypeptide, including, for example, SEQ.ID NO: 11, SEQ.ID NO: 64, SEQ.ID NO: 66, SEQ.ID NO: 68, SEQ.ID NO: 70, and SEQ.ID NO: 72. Nucleic acid sequences encoding a TrpB polypeptide further include any and all nucleic acid sequences which (i) encode polypeptides that are substantially identical to the TrpB polypeptide sequences set forth herein; or (ii) hybridize to any TrpB nucleic acid sequences set forth herein under at least moderately stringent hybridization conditions or which would hybridize thereto under at least moderately stringent conditions but for the use of synonymous codons.

The terms “nucleic acid sequence encoding a halogenase”, and “nucleic acid sequence encoding a halogenase polypeptide”, as may be used interchangeably herein, refer to any and all nucleic acid sequences encoding a halogenase polypeptide, including, for example, SEQ.ID NO: 13. Nucleic acid sequences encoding a halogenase polypeptide further include any and all nucleic acid sequences which (i) encode polypeptides that are substantially identical to the halogenase polypeptide sequences set forth herein; or (ii) hybridize to any halogenase nucleic acid sequences set forth herein under at least moderately stringent hybridization conditions or which would hybridize thereto under at least moderately stringent conditions but for the use of synonymous codons.

The terms “nucleic acid sequence encoding an acetyl transferase”, and “nucleic acid sequence encoding an acetyl transferase polypeptide”, as may be used interchangeably herein, refer to any and all nucleic acid sequences encoding an acetyl transferase polypeptide, including, for example, SEQ.ID NO: 62. Nucleic acid sequences encoding an acetyl transferase polypeptide further include any and all nucleic acid sequences which (i) encode polypeptides that are substantially identical to the acetyl transferase polypeptide sequences set forth herein; or (ii) hybridize to any acetyl transferase nucleic acid sequences set forth herein under at least moderately stringent hybridization conditions or which would hybridize thereto under at least moderately stringent conditions but for the use of synonymous codons.

The terms “nucleic acid”, or “nucleic acid sequence”, as used herein, refer to a sequence of nucleoside or nucleotide monomers, consisting of naturally occurring bases, sugars and intersugar (backbone) linkages. The term also includes modified or substituted sequences comprising non-naturally occurring monomers or portions thereof. The nucleic acids of the present disclosure may be deoxyribonucleic nucleic acids (DNA) or ribonucleic acids (RNA) and may include naturally occurring bases including adenine, guanine, cytosine, thymidine and uracil. The nucleic acids may also contain modified bases. Examples of such modified bases include aza and deaza adenine, guanine, cytosine, thymidine and uracil, and xanthine and hypoxanthine. A sequence of nucleotide or nucleoside monomers may be referred to as a polynucleotide sequence, nucleic acid sequence, a nucleotide sequence or a nucleoside sequence.

The term “polypeptide”, as used herein in conjunction with a reference SEQ.ID NO, refers to any and all polypeptides comprising a sequence of amino acid residues which is (i) substantially identical to the amino acid sequence constituting the polypeptide having such reference SEQ.ID NO, or (ii) encoded by a nucleic acid sequence capable of hybridizing under at least moderately stringent conditions to any nucleic acid sequence encoding the polypeptide having such reference SEQ.ID NO, but for the use of synonymous codons. A sequence of amino acid residues may be referred to as an amino acid sequence, or polypeptide sequence.

The term “nucleic acid sequence encoding a polypeptide”, as used herein in conjunction with a reference SEQ.ID NO, refers to any and all nucleic acid sequences encoding a polypeptide having such reference SEQ.ID NO. Nucleic acid sequences encoding a polypeptide, in conjunction with a reference SEQ.ID NO, further include any and all nucleic acid sequences which (i) encode polypeptides that are substantially identical to the polypeptide having such reference SEQ.ID NO; or (ii) hybridize to any nucleic acid sequences encoding polypeptides having such reference SEQ.ID NO under at least moderately stringent hybridization conditions or which would hybridize thereto under at least moderately stringent conditions but for the use of synonymous codons.

By the term “substantially identical” it is meant that two amino acid sequences preferably are at least 70% identical, and more preferably are at least 85% identical and most preferably at least 95% identical, for example 96%, 97%, 98% or 99% identical. In order to determine the percentage of identity between two amino acid sequences the amino acid sequences of such two sequences are aligned, using for example the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Mol. Biol., 1990:215:403). A particularly preferred method for determining the percentage identity between two polypeptides involves the Clustal W algorithm (Thompson, J D, Higgines, D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89: 10915-10919 using a gap opening penalty of 10 and a gap extension penalty of 0.1, so that the highest order match obtained between two sequences wherein at least 50% of the total length of one of the two sequences is involved in the alignment.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.-16.6 (Log 10 [Na+])+0.41(% (G+C)−600/I), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm (based on the above equation) −5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1.-6.3.6 and in: Sambrook et al., Molecular Cloning, a Laboratory Manual, Cold Spring Harbor Laboratory Press, 1989, Vol. 3.

The term “functional variant”, as used herein in reference to polynucleotides or polypeptides, refers to polynucleotides or polypeptides capable of performing the same function as a noted reference polynucleotide or polypeptide. Thus, for example, a functional variant of the polypeptide set forth in SEQ.ID NO: 2, refers to a polypeptide capable of performing the same function as the polypeptide set forth in SEQ.ID NO: 2. Functional variants include modified a polypeptide wherein, relative to a noted reference polypeptide, the modification includes a substitution, deletion or addition of one or more amino acids. In some embodiments, substitutions are those that result in a replacement of one amino acid with an amino acid having similar characteristics. Such substitutions include, without limitation (i) glutamic acid and aspartic acid; (i) alanine, serine, and threonine; (iii) isoleucine, leucine and valine, (iv) asparagine and glutamine, and (v) tryptophan, tyrosine and phenylalanine. Functional variants further include polypeptides having retained or exhibiting an enhanced psilocybin biosynthetic bioactivity.

The term “chimeric”, as used herein in the context of nucleic acids, refers to at least two linked nucleic acids which are not naturally linked. Chimeric nucleic acids include linked nucleic acids of different natural origins. For example, a nucleic acid constituting a microbial promoter linked to a nucleic acid encoding a plant polypeptide is considered chimeric. Chimeric nucleic acids also may comprise nucleic acids of the same natural origin, provided they are not naturally linked. For example a nucleic acid constituting a promoter obtained from a particular cell-type may be linked to a nucleic acid encoding a polypeptide obtained from that same cell-type, but not normally linked to the nucleic acid constituting the promoter. Chimeric nucleic acids also include nucleic acids comprising any naturally occurring nucleic acids linked to any non-naturally occurring nucleic acids.

The terms “substantially pure” and “isolated”, as used herein, as may be used interchangeably herein describe a compound, e.g., a secondary metabolite, psilocybin or a psilocybin derivative, polynucleotide or a polypeptide, which has been separated from components that naturally accompany it. Typically, a compound is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the compound of interest. Purity can be measured by any appropriate method, e.g., in the case of polypeptides, by chromatography, gel electrophoresis or HPLC analysis.

The term “recovered” as used herein in association with an enzyme, protein, a secondary metabolite or a chemical compound, refers to a more or less pure form of the enzyme, protein, secondary metabolite, or chemical compound.

### General Implementation

As hereinbefore mentioned, the present disclosure relates to psilocybin derivatives. In particular, the present disclosure provides novel halogenated psilocybin derivatives. In general, the herein provided compositions exhibit functional properties which deviate from the functional properties of psilocybin. Thus, for example, the psilocybin derivatives, can exhibit pharmacological properties which deviate from psilocybin. The halogenated psilocybin derivatives in this respect are useful in the formulation of pharmaceutical and recreation drug formulations. Furthermore, the halogenated psilocybin derivatives of the present disclosure may be used as a feedstock material for deriving further psilocybin derivatives. In one embodiment, the halogenated psilocybin derivatives of the present disclosure can conveniently be biosynthetically produced. The practice of this method avoids the extraction of psilocybin from mushrooms and the performance of subsequent chemical reactions to achieve halogenation. Furthermore, the growth of mushrooms can be avoided thus limiting the dependence on climate and weather, and potential legal and social challenges associated with the cultivation of mushrooms containing psychoactive compounds. The method can efficiently yield substantial quantities of halogenated psilocybin derivatives.

In what follows selected embodiments are described with reference to the drawings.

Initially various halogenated psilocybin derivatives will be described. Thereafter methods example methods of using and making the halogenated psilocybin derivatives will be described

In at least one aspect, and in at least one example embodiment, the present disclosure provides a chemical compound or salts thereof having the formula (I):

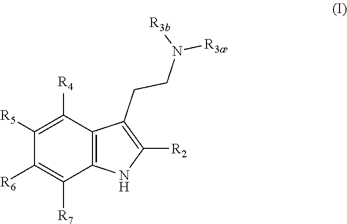

wherein, at least one of R₂, R₄, R₅, R₆ or R₇ is a halogen atom, wherein each non-halogenated R₂, R₅, R₆, or R₇ is a hydrogen atom, wherein R₄ when it is not halogenated is a phosphate group or a hydrogen atom, and wherein R_{3A} and R_{3B} are a hydrogen atom, an alkyl group, an aryl group or an acyl group.

It is noted that in reference to the indole prototype structure shown in FIG. 2 carbon atoms C₂, C₄, C₅, C₆ and C₇ are bonded to R₂, R₄, R₅, R₆ and R₇, respectively. Furthermore, R_{3A} and R_{3B} reference chemical groups extending from the ethyl-amino group extending in turn from carbon atom C₃ of the prototype indole structure.

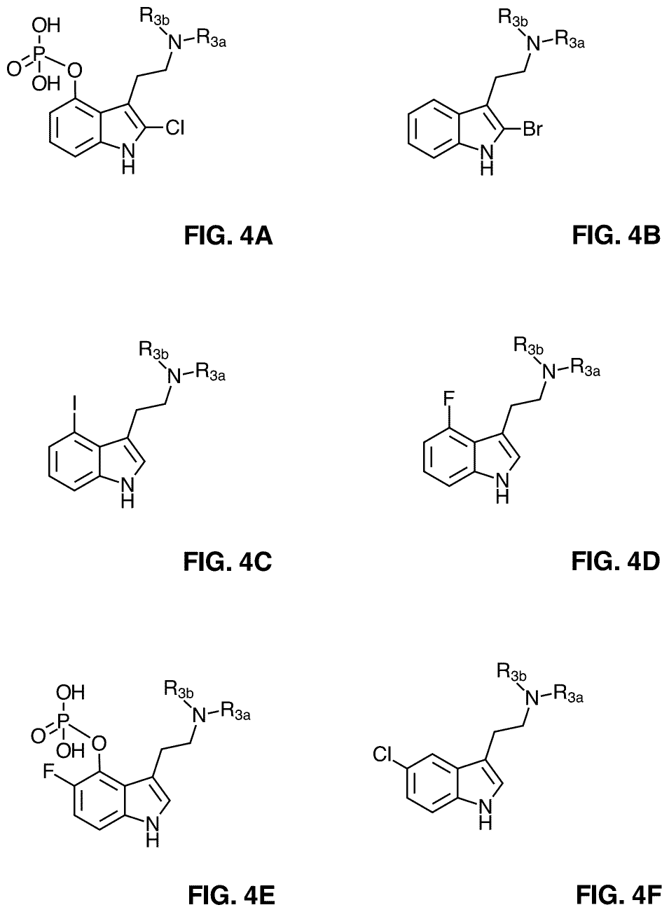

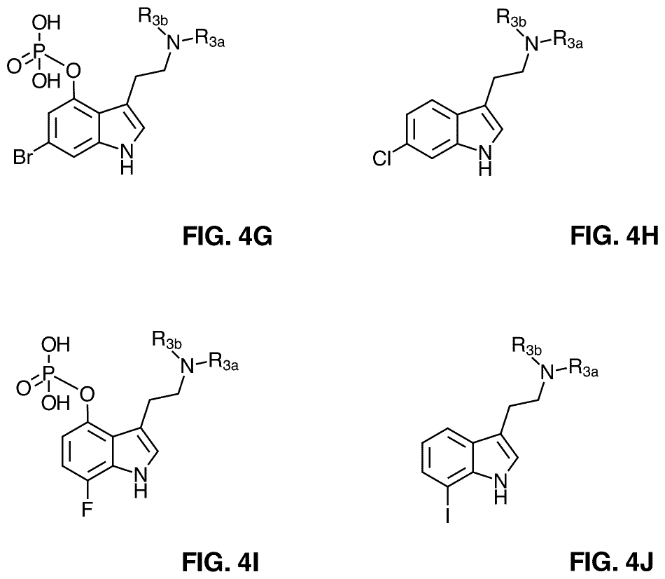

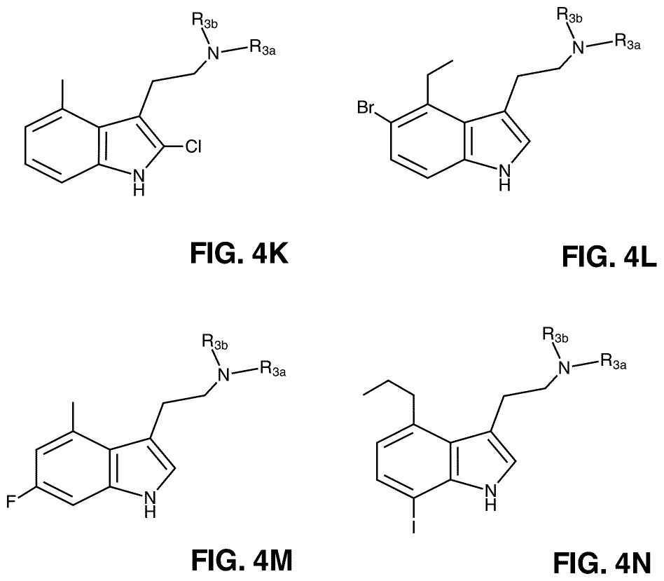

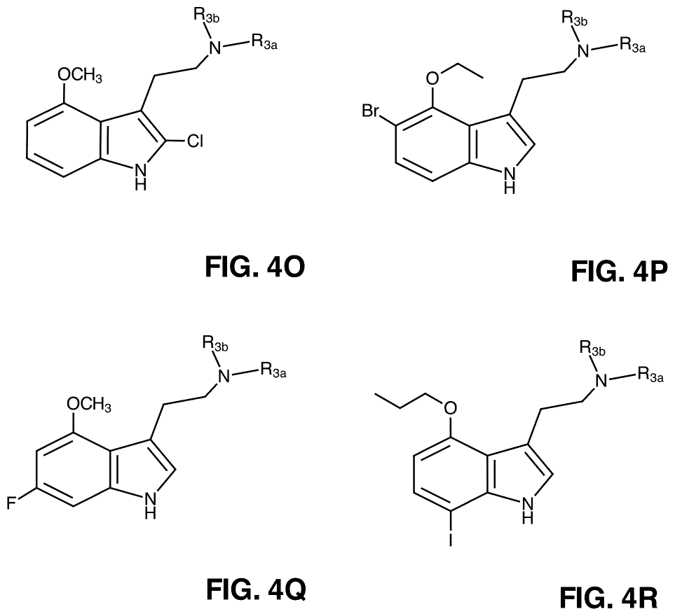

Referring to the chemical compound having formula (I), In one embodiment, one of R₂, R₄, R₅, R₆ and R₇ can be a halogen atom. Thus, in one embodiment, R₂ can be a bromine, chlorine, fluorine or iodine atom, each of R₅, R₆ and R₇ can be a hydrogen atom or an alkyl group or O-alkyl group, and R₄, when it is not a halogen atom, can be a phosphate group, a hydrogen atom or an alkyl group or O-alkyl group (see: the example halogenated psilocybin derivatives shown in FIG. 4A (R₂ is chlorine; R₄ is a phosphate group; R₅, R₆ and R₇ are each a hydrogen atom; R₃ₐ and R_{3b} are a hydrogen atom, an alkyl group, an aryl group, or an acyl group); FIG. 4B (R₂ is bromine; R₄, R₅, R₆ and R₇ are each a hydrogen atom; R₃ₐ and R_{3b} are a hydrogen atom, an alkyl group, an aryl group, or an acyl group; FIG. 4K (R₂ is chlorine; R₄ is a methyl group; R₅, R₆ and R₇ are each a hydrogen atom; R₃ₐ and R_{3b} are a hydrogen atom, an alkyl group, an aryl group, or an acyl group); and FIG. 4O (R₂ is chlorine; R₄ is an O-methyl group; R₅, R₆ and R₇ are each a hydrogen atom; R₃ₐ and R_{3b} are a hydrogen atom, an alkyl group, an aryl group, or an acyl group)).

Continuing to refer to the chemical compound having formula (I), in one embodiment, R₄ can be a bromine, chlorine, fluorine or iodine atom, and each of R₂, R₅, R₆ and R₇ can be a hydrogen atom or an alkyl group or O-alkyl group (see: the example halogenated psilocybin derivatives shown in FIG. 4C (R₄ is iodine; R₂, R₅, R₆ and R₇ are each a hydrogen atom; R₃ₐ and R_{3b} are a hydrogen atom, an alkyl group, an aryl group, or an acyl group); FIG. 4D (R₄ is fluorine; R₂, R₅, R₆ and R₇ are each a hydrogen atom; R₃ₐ and R_{3b} are a hydrogen atom, an alkyl group, an aryl group, or an acyl group))

Continuing to refer to the chemical compound having formula (I), in one embodiment, R₅ can be a bromine, chlorine, fluorine or iodine atom, and each of R₂, R₆ and R₇ can be a hydrogen atom or an alkyl group or O-alkyl group, and R₄ can be a phosphate group, a hydrogen atom or an alkyl group or O-alkyl group (see: the example halogenated psilocybin derivatives shown in FIG. 4E (R₅ is fluorine; R₄ is a phosphate group; R₂, R₆ and R₇ are each a hydrogen atom; R₃ₐ and R_{3b} are a hydrogen atom, an alkyl group, an aryl group, or an acyl group); FIG. 4F (R₅ is chlorine; R₂, R₄, R₆ and R₇ are each a hydrogen atom; R₃ₐ and R_{3b} are a hydrogen atom, an alkyl group, an aryl group, or an acyl group); FIG. 4L (R₅ is bromine; R₄ is an ethyl group; R₂, R₆ and R₇ are each a hydrogen atom; R₃ₐ and R_{3b} are a hydrogen atom, an alkyl group, an aryl group, or an acyl group); and FIG. 4P (R₅ is bromine; R₂, R₆ and R₇ are each a hydrogen atom; R₄ is an O-ethyl group; R₃ₐ and R_{3b} are a hydrogen atom, an alkyl group, an aryl group, or an acyl group)).

Continuing to refer to the chemical compound having formula (I), in one embodiment, R₆ can be a bromine, chlorine, fluorine or iodine atom, and each of R₂, R₅ and R₇ can be a hydrogen atom or an alkyl group or O-alkyl group, and R₄ can be a phosphate group, a hydrogen atom or an alkyl group or O-alkyl group (see: the example halogenated psilocybin derivatives shown in FIG. 4G (R₆ is bromine; R₄ is a phosphate group; R₂, R₅ and R₇ are each a hydrogen atom; R₃ₐ and R_{3b} are a hydrogen atom, an alkyl group, an aryl group, or an acyl group); FIG. 4H (R₆ is chlorine; R₂, R₄, R₅ and R₇ are each a hydrogen atom; R₃ₐ and R_{3b} are a hydrogen atom, an alkyl group, an aryl group, or an acyl group); FIG. 4M (R₆ is fluorine; R₄ is a methyl group; R₂, R₅ and R₇ are each a hydrogen atom; R₃ₐ and R_{3b} are a hydrogen atom, an alkyl group, an aryl group, or an acyl group); and FIG. 4Q (R₆ is fluorine; R₂, R₅ and R₇ are each a hydrogen atom; R₄ is an O-methyl group; R₃ₐ and R_{3b} are a hydrogen atom, an alkyl group, an aryl group, or an acyl group)).

Continuing to refer to the chemical compound having formula (I), in one embodiment, R₇ can be a bromine, chlorine, fluorine or iodine atom, and each of R₂, R₅ and R₆ can be a hydrogen atom or an alkyl group or O-alkyl group, and R₄ can be a phosphate group, a hydrogen atom or an alkyl group or O-alkyl group (see: the example halogenated psilocybin derivatives shown in FIG. 4I (R₇ is fluorine; R₄ is a phosphate group; R₂, R₅ and R₆ are each a hydrogen atom R₃ₐ and R_{3b} are a hydrogen atom, an alkyl group, an aryl group, or an acyl group); FIG. 4J (R₇ is iodine; R₂, R₄, R₅ and R₆ are each a hydrogen atom; R₃ₐ and R_{3b} are a hydrogen atom, an alkyl group, an aryl group, or an acyl group); FIG. 4N (R₇ is iodine; R₄ is a propyl group; R₂, R₅ and R₆ are each a hydrogen atom R₃ₐ and R_{3b} are a hydrogen atom, an alkyl group, an aryl group, or an acyl group); and FIG. 4R (R₇ is iodine; R₂, R₅ and R₆ are each a hydrogen atom; R₄ is an O-propyl group; R₃ₐ and R_{3b} are a hydrogen atom, an alkyl group, an aryl group, or an acyl group)).

Still, continuing to refer to the chemical compound having formula (I), in one embodiment, two of R₂, R₄, R₅, R₆ and R₇ can be a halogen atom, wherein the halogen atoms are independently selected from a bromine, chlorine fluorine or iodine atom, and wherein each non-halogenated R₂, R₅, R₆ and R₇ is a hydrogen atom or an alkyl group or O-alkyl group, and wherein R₄, when it is not a halogen atom, is a phosphate group, a hydrogen atom or an alkyl group or O-alkyl group.

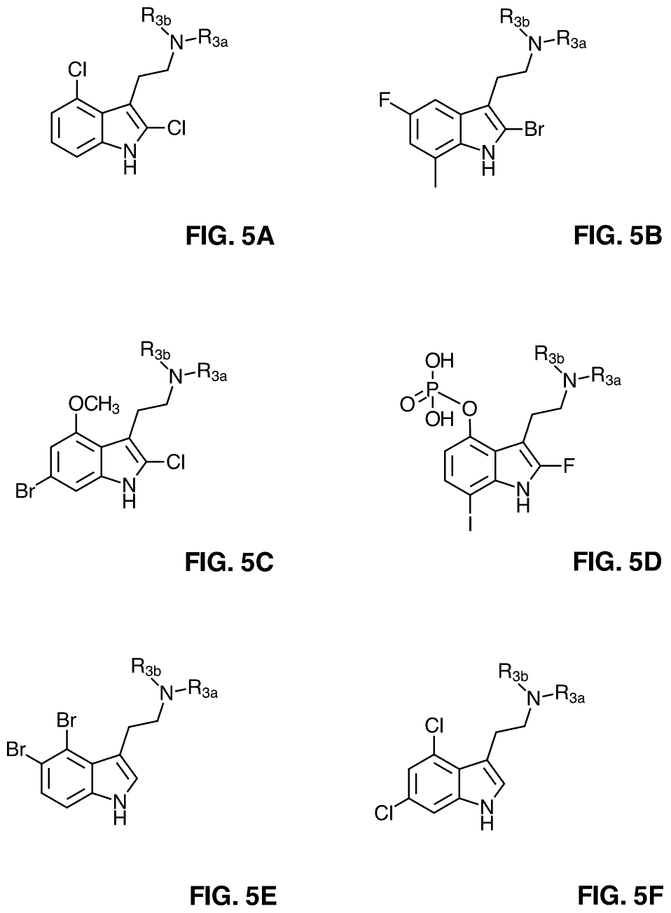

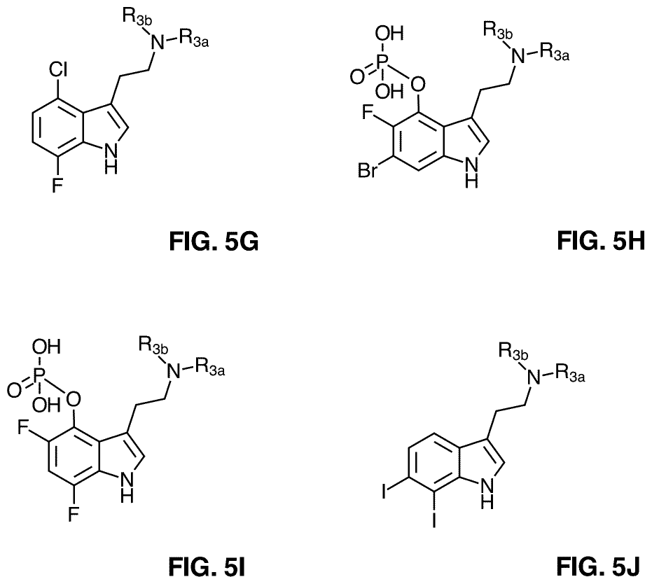

Thus, continuing to refer to the chemical compound having formula (I), in one embodiment, R₂ and R₄ can be halogen atoms, wherein the halogen atoms are independently selected from a bromine, chlorine fluorine or iodine atom, and R₅, R₆ and R₇ can be a hydrogen atom or an alkyl group or O-alkyl group (see: the example halogenated psilocybin derivative shown in FIG. 5A (R₂ is a chlorine atom; R₄ is a chlorine atom; R₅, R₆ and R₇ are each a hydrogen atom; and R₃ₐ and R_{3b} are a hydrogen atom, an alkyl group, an aryl group, or an acyl group)).

Continuing to refer to the chemical compound having formula (I), in one embodiment, R₂ and R₅ can be halogen atoms, wherein the halogen atoms are independently selected from a bromine, chlorine fluorine or iodine atom, and R₆ and R₇ can be a hydrogen atom or an alkyl group or O-alkyl group and R₄ can be a phosphate group, a hydrogen atom or an alkyl group or O-alkyl group (see: the example halogenated psilocybin derivative shown in FIG. 5B (R₂ is a bromine atom; R₅ is a fluorine atom; R₄, and R₆ are each a hydrogen atom and R₇ is a methyl group; and R₃ₐ and R_{3b} are a hydrogen atom, an alkyl group, an aryl group, or an acyl group)).

Continuing to refer to the chemical compound having formula (I), in one embodiment, R₂ and R₆ can be halogen atoms, wherein the halogen atoms are independently selected from a bromine, chlorine fluorine or iodine atom, and R₅ and R₇ can be a hydrogen atom or an alkyl group or O-alkyl group, and R₄ can be a phosphate group, a hydrogen atom or an alkyl group or O-alkyl group (see: the example halogenated psilocybin derivative shown in FIG. 5C (R₂ is a chlorine atom; R₆ is a bromine atom; R₅ and R₇ are each a hydrogen atom; R₄ is an O-methyl group; and R₃ₐ and R_{3b} are a hydrogen atom, an alkyl group, an aryl group, or an acyl group)).

Continuing to refer to the chemical compound having formula (I), in one embodiment, R₂ and R₇ can be halogen atoms, wherein the halogen atoms are independently selected from a bromine, chlorine fluorine or iodine atom, and R₅ and R₆ can be a hydrogen atom or an alkyl group or O-alkyl group and R₄ can be a phosphate group, a hydrogen atom or an alkyl group or O-alkyl group (see: the example halogenated psilocybin derivative shown in FIG. 5D (R₂ is a fluorine atom; R₇ is an iodine atom; R₄ is a phosphate, R₅ and R₆ are each a hydrogen atom; and R₃ₐ and R_{3b} are a hydrogen atom, an alkyl group, an aryl group, or an acyl group)).

Continuing to refer to the chemical compound having formula (I), in one embodiment, R₄ and R₅ can be halogen atoms, wherein the halogen atoms are independently selected from a bromine, chlorine fluorine or iodine atom, and R₂, R₆ and R₇ can be a hydrogen atom or an alkyl group or O-alkyl group (see: the example halogenated psilocybin derivative shown in FIG. 5E (R₄ and R₅ are each bromine atoms; R₂, R₆ and R₇ are each a hydrogen atom; and R₃ₐ and R_{3b} are a hydrogen atom, an alkyl group, an aryl group, or an acyl group)).

Continuing to refer to the chemical compound having formula (I), in one embodiment, R₄ and R₆ can be halogen atoms, wherein the halogen atoms are independently selected from a bromine, chlorine fluorine or iodine atom, and R₂, R₅ and R₇ can be a hydrogen atom or an alkyl group or O-alkyl group (see: the example halogenated psilocybin derivative shown in FIG. 5F (R₄ and R₆ are each chlorine atoms; R₂, R₅ and R₇ are each a hydrogen atom; and R₃ₐ and R_{3b} are a hydrogen atom, an alkyl group, an aryl group, or an acyl group)).

Continuing to refer to the chemical compound having formula (I), in one embodiment, R₄ and R₇ can be halogen atoms, wherein the halogen atoms are independently selected from a bromine, chlorine fluorine or iodine atom, and R₂, R₅ and R₆ can be a hydrogen atom or an alkyl group or O-alkyl group (see: the example halogenated psilocybin derivative shown in FIG. 5G (R₄ is a chlorine atom; R₇ is a fluorine atom; R₂, R₅ and R₆ are each a hydrogen atom; and R₃ₐ and R_{3b} are a hydrogen atom, an alkyl group, an aryl group, or an acyl group)).

Continuing to refer to the chemical compound having formula (I), in one embodiment, R₅ and R₆ can be halogen atoms, wherein the halogen atoms are independently selected from a bromine, chlorine fluorine or iodine atom, and R₂ and R₇ can be a hydrogen atom or an alkyl group or O-alkyl group and R₄ can be a phosphate group, a hydrogen atom or an alkyl group or O-alkyl group (see: the example halogenated psilocybin derivative shown in FIG. 5H (R₅ is a fluorine atom; R₆ is a bromine atom; R₄ is a phosphate group, R₂ and R₇ are each a hydrogen atom; and R₃ₐ and R_{3b} are a hydrogen atom, an alkyl group, an aryl group, or an acyl group)).

Continuing to refer to the chemical compound having formula (I), in one embodiment, R₅ and R₇ can be halogen atoms, wherein the halogen atoms are independently selected from a bromine, chlorine fluorine or iodine atom, and R₂ and R₆ can be a hydrogen atom or an alkyl group or O-alkyl group and R₄ can be a phosphate group, a hydrogen atom or an alkyl group or O-alkyl group (see: the example halogenated psilocybin derivative shown in FIG. 5I (R₅ and R₇ are each fluorine atoms; R₄ is a phosphate group, R₂ and R₆ are each a hydrogen atom; and R₃ₐ and R_{3b} are a hydrogen atom, an alkyl group, an aryl group, or an acyl group)).

Continuing to refer to the chemical compound having formula (I), in one embodiment, R₆ and R₇ can be halogen atoms, wherein the halogen atoms are independently selected from a bromine, chlorine fluorine or iodine atom, and R₂ and R₅ can be a hydrogen atom or an alkyl group or O-alkyl group and R₄ can be a phosphate group, a hydrogen atom or an alkyl group or O-alkyl group (see: the example halogenated psilocybin derivative shown in FIG. 5J (R₆ and R₇ are each iodine atoms; R₂, R₄ and R₅ are each a hydrogen atom; and R₃ₐ and R_{3b} are a hydrogen atom, an alkyl group, an aryl group, or an acyl group)).

In one embodiment, two of R₂, R₄, R₅, R₆ and R₇ can be a halogen atom, and the halogen atoms are identical, the remaining of R₂, R₅, R₆, and R₇ being a hydrogen atom or an alkyl group or O-alkyl group, and R₄ when it is not a halogen atom, being a phosphate group, a hydrogen atom or an alkyl group or O-alkyl group (see: e.g. the halogenated psilocybin derivatives shown in FIGS. 5A, 5E, 5F, 5I and 5J).

In one embodiment, two of R₂, R₄, R₅, R₆ and R₇ can be a halogen atom, and the halogen atoms are non-identical, the remaining of R₂, R₅, R₆, and R₇ being a hydrogen atom or an alkyl group or O-alkyl group, and R₄ when it is not a halogen atom, being a phosphate, a hydrogen atom or an alkyl group or O-alkyl group (see: e.g. the halogenated psilocybin derivatives shown in FIGS. 5B, 5C, 5D, 5G and 5H).

Still continuing to refer to the chemical compound having formula (I), In one further embodiment, three of R₂, R₄, R₅, R₆ and R₇ can be a halogen atom, wherein the halogen atoms are independently selected from a bromine, chlorine fluorine or iodine atom, and wherein the non-halogenated R₂, R₅, R₆, or R₇ is a hydrogen atom or an alkyl group or O-alkyl group, and wherein R₄, when it is not a halogen atom, is a phosphate group, a hydrogen atom or an alkyl group or O-alkyl group.

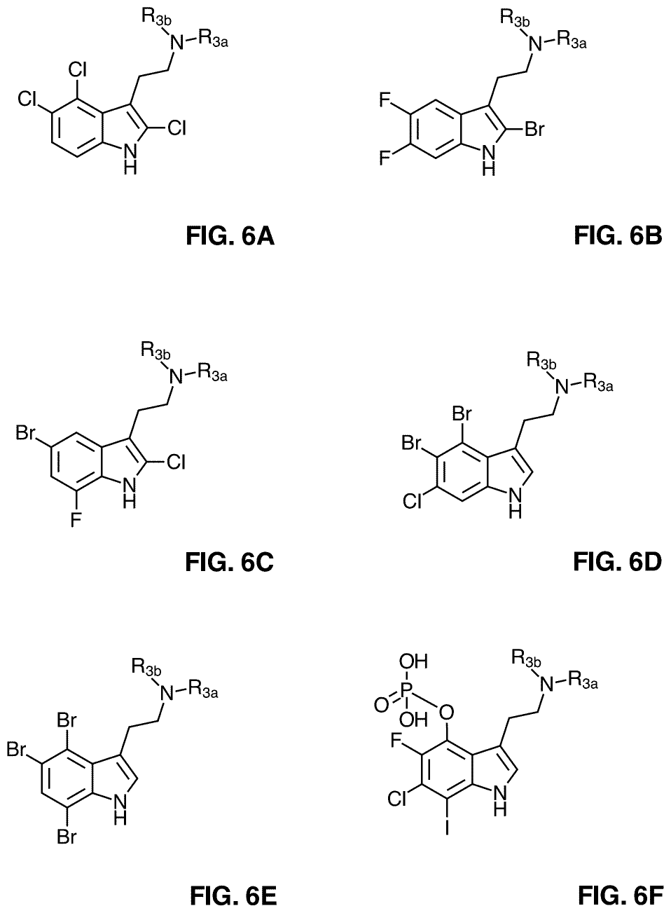

Thus, continuing to refer to the chemical compound having formula (I), in one embodiment R₂, R₄, and R₅ can be halogen atom, wherein the halogen atoms are independently selected from a bromine, chlorine fluorine or iodine atom, and R₆ and R₇ can be a hydrogen atom or an alkyl group or O-alkyl group (see: the example halogenated psilocybin derivative shown in FIG. 6A (R₂, R₄ and R₅ are each chlorine atoms; R₆ and R₇ are each a hydrogen atom; and R₃ₐ and R_{3b} are a hydrogen atom, an alkyl group, an aryl group, or an acyl group)).

Continuing to refer to the chemical compound having formula (I), in one embodiment R₂, R₅, and R₆ can be halogen atom, wherein the halogen atoms are independently selected from a bromine, chlorine fluorine or iodine atom and R₇ can be a hydrogen atom or an alkyl group or O-alkyl group, and R₄ can a phosphate group, a hydrogen atom or an alkyl group or O-alkyl group (see: the example halogenated psilocybin derivative shown in FIG. 6B (R₂ is a bromine atom, R₅ and R₆ are each fluorine atoms; R₄ and R₇ are each a hydrogen atom; and R₃ₐ and R_{3b} are a hydrogen atom, an alkyl group, an aryl group, or an acyl group)).

Continuing to refer to the chemical compound having formula (I), in one embodiment R₂, R₅, and R₇ can be halogen atom, wherein the halogen atoms are independently selected from a bromine, chlorine fluorine or iodine atom and R₆ can be a hydrogen atom or an alkyl group or O-alkyl group, and R₄ can a phosphate group, a hydrogen atom or an alkyl group or O-alkyl group (see: the example halogenated psilocybin derivative shown in FIG. 6C (R₂ is a chlorine atom, R₅ is a bromine atom, and R₇ is a fluorine atoms; R₄ and R₆ are each a hydrogen atom; and R₃ₐ and R_{3b} are a hydrogen atom, an alkyl group, an aryl group, or an acyl group)).

Continuing to refer to the chemical compound having formula (I), in one embodiment R₄, R₅, and R₆ can be halogen atom, wherein the halogen atoms are independently selected from a bromine, chlorine fluorine or iodine atom, and R₂ and R₇ can be a hydrogen atom or an alkyl group or O-alkyl group (see: the example halogenated psilocybin derivative shown in FIG. 6D (R₄ and R₅ are each a bromine atom, R₆ is a chlorine atom; R₂ and R₇ are each a hydrogen atom; and R₃ₐ and R_{3b} are a hydrogen atom, an alkyl group, an aryl group, or an acyl group)).

Continuing to refer to the chemical compound having formula (I), in one embodiment R₄, R₅, and R₇ can be halogen atom, wherein the halogen atoms are independently selected from a bromine, chlorine fluorine or iodine atom, and R₂ and R₆ can be a hydrogen atom or an alkyl group or O-alkyl group (see: the example halogenated psilocybin derivative shown in FIG. 6E (R₄, R₅ and R₇ are each a bromine atom; R₂ and R₆ are each a hydrogen atom; and R₃ₐ and R_{3b} are a hydrogen atom, an alkyl group, an aryl group, or an acyl group)).

Continuing to refer to the chemical compound having formula (I), in one embodiment R₅, R₆, and R₇ can be halogen atom, wherein the halogen atoms are independently selected from a bromine, chlorine fluorine or iodine atom and R₂ can be a hydrogen atom or an alkyl group or O-alkyl group, and R₄ can be a phosphate group, a hydrogen atom or an alkyl group or O-alkyl group (see: the example halogenated psilocybin derivative shown in FIG. 6F (R₅ is a fluorine atom, R₆ is a chlorine atom, and R₇ is an iodine atom; R₄ is a phosphate group; R₂ is a hydrogen atom; and R₃ₐ and R_{3b} are a hydrogen atom, an alkyl group, an aryl group, or an acyl group)).

In one embodiment, three of R₂, R₄, R₅, R₆ and R₇ can be a halogen atom, wherein at least two halogen atoms are identical, the remaining R₂, R₅, R₆, and R₇ being a hydrogen atom or an alkyl group or O-alkyl group, and R₄ when it is not a halogen atom, being a phosphate group, a hydrogen atom or an alkyl group or O-alkyl group (see: e.g. the halogenated psilocybin derivatives shown in FIGS. 6A, 6B and 6E).

In one embodiment, three of R₂, R₄, R₅, R₆ and R₇ can be a halogen atom, and at least two halogen atoms are non-identical, the remaining R₂, R₅, R₆, and R₇ being a hydrogen atom or an alkyl group or O-alkyl group, and R₄ when it is not a halogen atom, being a phosphate group, a hydrogen atom or an alkyl group or O-alkyl group (see: e.g. the halogenated psilocybin derivatives shown in FIGS. 6B, 6C, 6D and 6F).

In one embodiment, three of R₂, R₄, R₅, R₆ and R₇ can be a halogen atom, and all three halogen atoms are identical, the remaining R₂, R₅, R₆, or R₇ being a hydrogen atom or an alkyl group or O-alkyl group, and R₄ when it is not a halogen atom, being a phosphate group, a hydrogen atom or an alkyl group or O-alkyl group (see: e.g. the halogenated psilocybin derivatives shown in FIGS. 6A and 6E).

Still continuing to refer to the chemical compound having formula (I), in one embodiment, four of R₂, R₄, R₅, R₆ and R₇ can be a halogen atom, wherein the halogen atoms are independently selected from a bromine, chlorine fluorine or iodine atom, and wherein the non-halogenated R₂, R₅, R₆, or R₇ is a hydrogen atom or an alkyl group or O-alkyl group, and wherein R₄, when it is not a halogen, is a phosphate group, a hydrogen atom or an alkyl group or O-alkyl group.

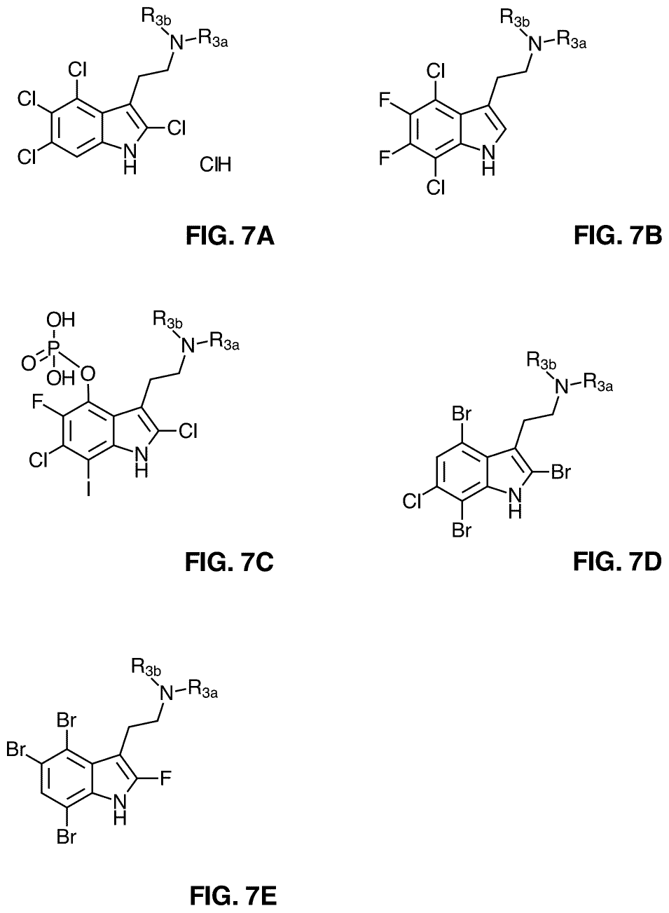

Thus, continuing to refer to the chemical compound having formula (I), in one embodiment R₂, R₄, R₅ and R₆ can be halogen atom, wherein the halogen atoms are independently selected from a bromine, chlorine fluorine or iodine atom, and R₇ can be a hydrogen atom or an alkyl group or O-alkyl group (see: the example halogenated psilocybin derivative shown in FIG. 7A (R₂, R₄, R₅ and R₆ are each chlorine atoms; R₇ is a hydrogen atom; and R₃ₐ and R_{3b} are a hydrogen atom, an alkyl group, an aryl group, or an acyl group)).

Continuing to refer to the chemical compound having formula (I), in one embodiment R₄, R₅, R₆ and R₇ can be halogen atom, wherein the halogen atoms are independently selected from a bromine, chlorine fluorine or iodine atom and R₂ can be a hydrogen atom or an alkyl group or O-alkyl group (see: the example halogenated psilocybin derivative shown in FIG. 7B (R₄ and R₇ are chlorine atoms, R₅ and R₆ are each fluorine atoms; R₂ is a hydrogen atom; and R₃ₐ and R_{3b} are a hydrogen atom, an alkyl group, an aryl group, or an acyl group).

Continuing to refer to the chemical compound having formula (I), in one embodiment R₂, R₅, R₆ and R₇ can be halogen atom, wherein the halogen atoms are independently selected from a bromine, chlorine fluorine or iodine atom and R₄ can be a phosphate group, a hydrogen atom or an alkyl group or O-alkyl group (see: the example halogenated psilocybin derivative shown in FIG. 7C (R₂ and are R₆ are each chlorine atoms, R₅ is a fluorine atom, R₇ is an iodine atoms, R₄ is a phosphate group; and R₃ₐ and R_{3b} are a hydrogen atom, an alkyl group, an aryl group, or an acyl group)).

Continuing to refer to the chemical compound having formula (I), in one embodiment R₂, R₄, R₆ and R₇ can be halogen atom, wherein the halogen atoms are independently selected from a bromine, chlorine fluorine or iodine atom and R₅ can be a hydrogen atom or an alkyl group or O-alkyl group (see: the example halogenated psilocybin derivative shown in FIG. 7D (R₂, R₄ and are R₇ are each bromine atoms, R₆ is a chlorine atom, R₅ is a hydrogen atom; and R₃ₐ and R_{3b} are a hydrogen atom, an alkyl group, an aryl group, or an acyl group)).

In one embodiment R₂, R₄, R₅ and R₇ can be halogen atom, wherein the halogen atoms are independently selected from a bromine, chlorine fluorine or iodine atom and R₆ can be a hydrogen atom or an alkyl group or O-alkyl group (see: the example halogenated psilocybin derivative shown in FIG. 7E (R₄, R₅ and are R₇ are each bromine atoms, R₂ is a fluorine atom, R₆ is a hydrogen atom; and R₃ₐ and R_{3b} are a hydrogen atom, an alkyl group, an aryl group, or an acyl group)).

In one embodiment, four of R₂, R₄, R₅, R₆ and R₇ can be a halogen atom, wherein at least two halogen atoms are identical, the remaining R₂, R₅, R₆, and R₇ being a hydrogen atom or an alkyl group or O-alkyl group, and R₄ when it is not a halogen atom, being a phosphate group, a hydrogen atom or an alkyl group or O-alkyl group (see: e.g. the halogenated psilocybin derivatives shown in FIGS. 7A, 7B, 7C, 7D and 7E).

In one embodiment, four of R₂, R₄, R₅, R₆ and R₇ can be a halogen atom, and at least two halogen atoms are non-identical, the remaining R₂, R₅, R₆, and R₇ being a hydrogen atom or an alkyl group or O-alkyl group, and R₄ when it is not a halogen atom, being a phosphate group, a hydrogen atom or an alkyl group or O-alkyl group (see: e.g. the halogenated psilocybin derivatives shown in FIGS. 7B, 7C, 7D and 7E).

In one embodiment, four of R₂, R₄, R₅, R₆ and R₇ can be a halogen atom, and all four halogen atoms are identical, the remaining R₂, R₅, R₆, or R₇ being a hydrogen atom or an alkyl group or O-alkyl group, and R₄ when it is not a halogen atom, being a phosphate group, a hydrogen atom or an alkyl group or O-alkyl group (see: e.g. the halogenated psilocybin derivative shown in FIG. 7A).

Still continuing to refer to the chemical compound having formula (I), In one embodiment, all five of R₂, R₄, R₅, R₆ and R₇ can be a halogen atom, the halogen selected from bromine, chlorine, fluorine or iodine.

In one embodiment, all five of R₂, R₄, R₅, R₆ and R₇ can be a halogen atom, and at least two halogen atoms are identical.

In one embodiment, all five of R₂, R₄, R₅, R₆ and R₇ can be a halogen atom, and at least three halogen atoms are identical.

In one embodiment, all five of R₂, R₄, R₅, R₆ and R₇ can be a halogen atom, and at least four halogen atoms are identical.

In one embodiment, all five of R₂, R₄, R₅, R₆ and R₇ can be a halogen atom, and all five halogen atoms are identical.

In one embodiment, all five of R₂, R₄, R₅, R₆ and R₇ can be a halogen atom, and the chemical compound comprises at least two non-identical halogen atoms.

In one embodiment, all five of R₂, R₄, R₅, R₆ and R₇ can be a halogen atom, and the chemical compound comprises at least three non-identical halogen atoms.

In one embodiment, all five of R₂, R₄, R₅, R₆ and R₇ can be a halogen atom, and four halogen atoms are non-identical.

Still continuing to refer to the chemical compound having formula (I), it is noted that, in a further aspect hereof, R_{3A} and R_{3B} can be a hydrogen atom, an alkyl group, an aryl group or an acyl group. Thus, for example, R_{3A} and R_{3B} can each be a hydrogen atom, or R_{3A} and R_{3B} can each be an alkyl group, such as a methyl group, ethyl group, propyl group, or longer chain alkyl group, or R_{3A} and R_{3B} can each be an aryl group, such as a phenyl group or a naphthyl group, or R_{3A} and R_{3B} can each be an acyl group, such as an acetyl group. Furthermore, one of R_{3A} and R_{3B} can be a hydrogen atom, and one of R_{3A} and R_{3B} can be an alkyl group, and aryl group, or an acyl group. Furthermore, R_{3A} and R_{3B} can be an aryl group and an alkyl group, an aryl group and an acyl group, or an alkyl group and an acyl group.

Furthermore, in one embodiment, a psilocybin derivative according to the present disclosure can be a chemical compound having the formula (II):

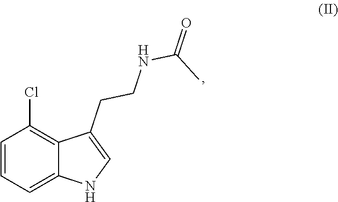 [SMILES: CC(=O)NCCc1c[nH]c2cccc(Cl)c12]

or, a chemical compound having the formula (II), wherein the chlorine is substituted for a bromine, fluorine or iodine.

Furthermore, in one embodiment, a psilocybin derivative according to the present disclosure can be a chemical compound having the formula (III):

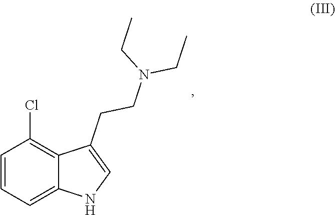 [SMILES: CCN(CC)CCc1c[nH]c2cccc(Cl)c12]

or, a chemical compound having the formula (III), wherein the chlorine is substituted for a bromine, fluorine or iodine.

Furthermore, in one embodiment, a psilocybin derivative according to the present disclosure can be a chemical compound having the formula (IV):

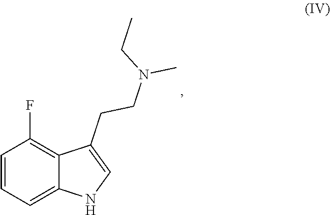 [SMILES: CCN(C)CCc1c[nH]c2cccc(F)c12]

or, a chemical compound having the formula (IV), wherein the fluorine is substituted for a bromine, chlorine or iodine.

Furthermore, in one embodiment, a psilocybin derivative according to the present disclosure can be a chemical compound having the formula (V):

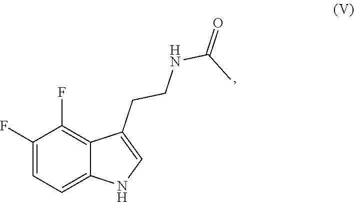 [SMILES: CC(=O)NCCc1c[nH]c2ccc(F)c(F)c12]

or, a chemical compound having the formula (V), wherein one or both of the fluorines are substituted for a bromine, chlorine or iodine.

Furthermore, in one embodiment, a psilocybin derivative according to the present disclosure can be a chemical compound having the formula (VI):

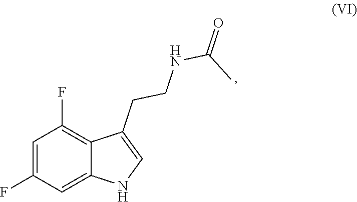 [SMILES: CC(=O)NCCc1c[nH]c2cc(F)cc(F)c12]

or, a chemical compound having the formula (VI), wherein one or both of the fluorines are substituted for a bromine, chlorine or iodine.

Furthermore, in one embodiment, a psilocybin derivative according to the present disclosure can be a chemical compound having the formula (VII):

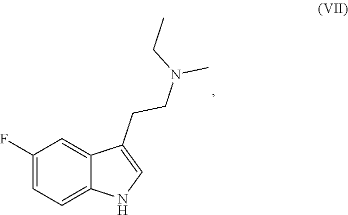 [SMILES: CCN(C)CCc1c[nH]c2ccc(F)cc12]

or, a chemical compound having the formula (VII), wherein the fluorine is substituted for a bromine, chlorine or iodine.

Furthermore, in one embodiment, a psilocybin derivative according to the present disclosure can be a chemical compound having the formula (VIII):

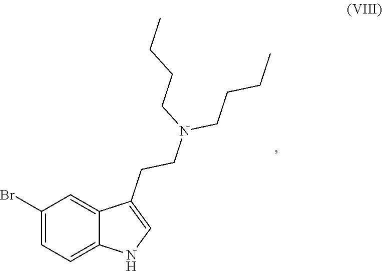 [SMILES: CCCCN(CCCC)CCc1c[nH]c2ccc(Br)cc12]

or, a chemical compound having the formula (VIII), wherein the bromine is substituted for a, chlorine, fluorine or iodine.

Furthermore, in one embodiment, a psilocybin derivative according to the present disclosure can be a chemical compound having the formula (IX):

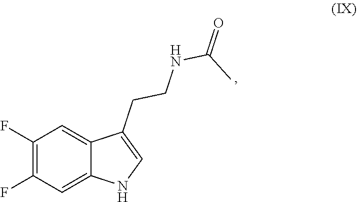 [SMILES: CC(=O)NCCc1c[nH]c2cc(F)c(F)cc12]

or, a chemical compound having the formula (IX), wherein one or both of the fluorines are substituted for a bromine, chlorine or iodine.

Furthermore, in one embodiment, a psilocybin derivative according to the present disclosure can be a chemical compound having the formula (X):

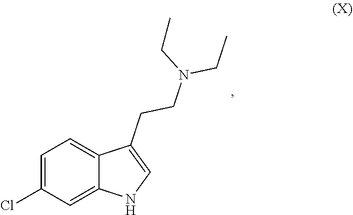 [SMILES: CCN(CC)CCc1c[nH]c2cc(Cl)ccc12]

or, a chemical compound having the formula (X), wherein the chlorine is substituted for a bromine, chlorine or iodine.

Furthermore, in one embodiment, a psilocybin derivative according to the present disclosure can be a chemical compound having the formula (XI):

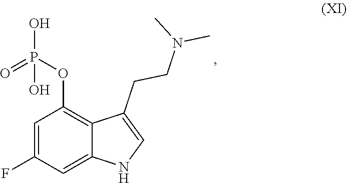 [SMILES: CN(C)CCc1c[nH]c2cc(F)cc(OP(=O)(O)O)c12]

or, a chemical compound having the formula (XI), wherein the fluorine is substituted for a bromine, chlorine or iodine.

Furthermore, in one embodiment, a psilocybin derivative according to the present disclosure can be a chemical compound having the formula (XII):

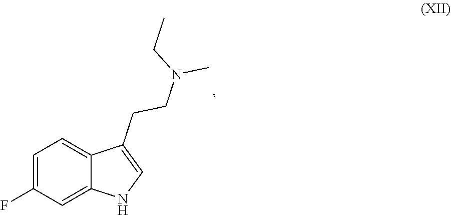 [SMILES: CCN(C)CCc1c[nH]c2cc(F)ccc12]

or, a chemical compound having the formula (XII), wherein the fluorine is substituted for a bromine, chlorine or iodine.

Furthermore, in one embodiment, a psilocybin derivative according to the present disclosure can be a chemical compound having the formula (XIII):

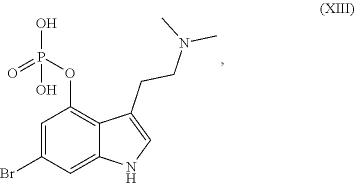 [SMILES: CN(C)CCc1c[nH]c2cc(Br)cc(OP(=O)(O)O)c12]

or, a chemical compound having the formula (XIII), wherein the bromine is substituted for a chlorine, fluorine or iodine.

Furthermore, in one embodiment, a psilocybin derivative according to the present disclosure can be a chemical compound having the formula (XIV):

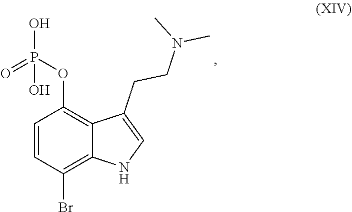 [SMILES: CN(C)CCc1c[nH]c2c(Br)ccc(OP(=O)(O)O)c12]

or, a chemical compound having the formula (XIV), wherein the bromine is substituted for a chlorine, fluorine or iodine.

Furthermore, in one embodiment, a psilocybin derivative according to the present disclosure can be a chemical compound having the formula (XV):

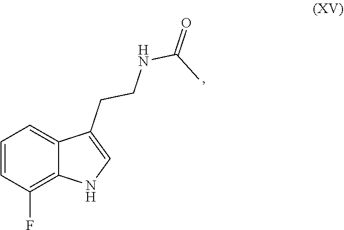 [SMILES: CC(=O)NCCc1c[nH]c2c(F)cccc12]

or, a chemical compound having the formula (XV), wherein the fluorine is substituted for a bromine, chlorine or iodine.

Furthermore, in one embodiment, a psilocybin derivative according to the present disclosure can be a chemical compound having the formula (XVI):

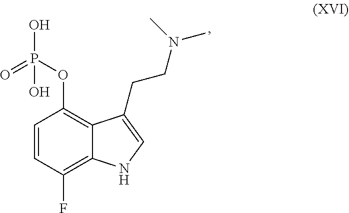 [SMILES: CN(C)CCc1c[nH]c2c(F)ccc(OP(=O)(O)O)c12]

or, a chemical compound having the formula (XV), wherein the fluorine is substituted for a bromine, chlorine or iodine.

Furthermore, in one embodiment, a psilocybin derivative according to the present disclosure can be a chemical compound having the formula (XVII):

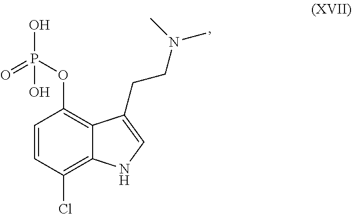 [SMILES: CN(C)CCc1c[nH]c2c(Cl)ccc(OP(=O)(O)O)c12]

or, a chemical compound having the formula (XVII), wherein the fluorine is substituted for a bromine, chlorine or iodine.

Furthermore, in one embodiment, a psilocybin derivative according to the present disclosure can be a chemical compound having the formula (XVII):

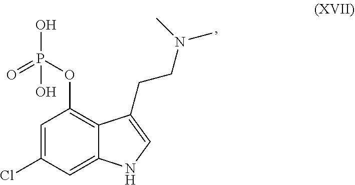 [SMILES: CN(C)CCc1c[nH]c2cc(Cl)cc(OP(=O)(O)O)c12]

or, a chemical compound having the formula (XVII), wherein the fluorine is substituted for a bromine, chlorine or iodine.

Furthermore, it is noted that the halogenated psilocybin derivatives of the present disclosure include salts thereof. Thus, the nitrogen atom of the ethyl-amino group extending in turn from the C₃ atom may be protonated, and the positive charge may be balanced by, for example, chloride or sulfate ions, to thereby form a chloride salt or a sulfate salt. Furthermore, in compounds wherein R₄ is a phosphate group, the phosphate group may be de-protonated, and the negative charge may be balanced by, for example, sodium ions or potassium ions, to thereby form a sodium salt or a potassium salt.

Furthermore, it is noted that when R₄ is a phosphate group, the term halogenated psilocybin derivative also includes compounds having the formula (XVIII):

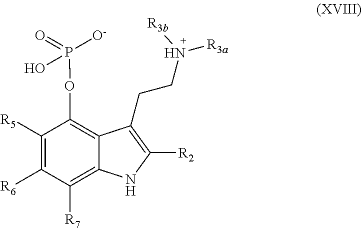

wherein, at least one of R₂, R₅, R₆, or R₇ is a halogen atom or an alkyl group or O-alkyl group, and wherein any R₂, R₅, R₆, or R₇ which are not a halogen is a hydrogen atom, and wherein R_{3A} and R_{3B} are a hydrogen atom, an alkyl group, and aryl group or an acyl group. The term further includes salts, including pharmaceutically acceptable salts, of halogenated psilocybins having the formula (XVIII), such as a sodium salt, a potassium salt etc.

Thus, to briefly recap, the present disclosure provides halogenated psilocybin derivatives. The disclosure provides, in particular, a chemical compound or salts thereof having formula (I):

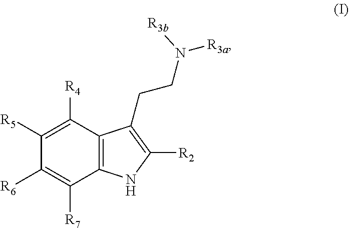

wherein in an aspect, at least one of R₂, R₄, R₅, R₆, or R₇ is a halogen atom. In an aspect, in formula (I), each non-halogenated R₂, R₅, R₆, or R₇ is a hydrogen atom or an alkyl group or O-alkyl group. In a further aspect, in formula (I), R₄ when it is not halogenated is a hydrogen atom, an alkyl group or O-alkyl group, a hydroxy group, or a phosphate group. Yet in a further aspect, R_{3A} and R_{3B} are a hydrogen atom, an alkyl group, an aryl group, or an acyl group.

In one embodiment of the disclosure, a chemical compound or salts thereof having formula (I) is included:

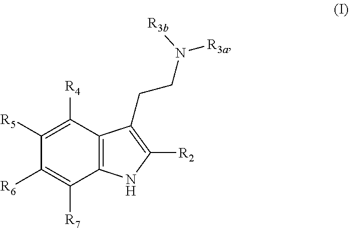

wherein at least one of R₂, R₄, R₅, R₆, or R₇ is a halogen atom, and wherein each non-halogenated R₂, R₅, R₆, or R₇ is a hydrogen atom or an alkyl group or O-alkyl group, wherein R₄ when it is not halogenated is a hydrogen atom, alkyl group or O-alkyl group, a hydroxy group, or a phosphate group, and wherein R_{3A} and R_{3B} are a hydrogen atom, an alkyl group, an aryl group, or an acyl group.

In one embodiment, at least one of R₂, R₄, R₅, R₆, or R₇ is a halogen atom, and wherein each non-halogenated R₂, R₅, R₆, or R₇ is a hydrogen atom or a (C₁-C₂₀)-alkyl group or (C₁-C₂₀)—O-alkyl group. In another embodiment, each non-halogenated R₂, R₅, R₆, or R₇ is a hydrogen atom, a methyl group, ethyl group, a propyl group, an O-methyl group, an O-ethyl group, or an O-propyl group.

In another embodiment, each non-halogenated R₂, R₅, R₆, or R₇ is a hydrogen atom or a (C₁-C₁₀)-alkyl group or (C₁-C₁₀)—O-alkyl group. In another embodiment, each non-halogenated R₂, R₅, R₆, or R₇ is a hydrogen atom, a methyl group, ethyl group, a propyl group, an O-methyl group, an O-ethyl group, or an O-propyl group.

In another embodiment, each non-halogenated R₂, R₅, R₆, or R₇ is a hydrogen atom or a (C₁-C₆)-alkyl group or (C₁-C₆)—O-alkyl group. In another embodiment, each non-halogenated R₂, R₅, R₆, or R₇ is a hydrogen atom, a methyl group, ethyl group, a propyl group, an O-methyl group, an O-ethyl group, or an O-propyl group.

In another embodiment, when R₄ is not halogenated, R₄ is a hydrogen atom, a (C₁-C₂₀)-alkyl group or (C₁-C₂₀)—O-alkyl group, a hydroxy group, or a phosphate group. In another embodiment, when R₄ is not halogenated, R₄ is a hydrogen atom, a (C₁-C₁₀)-alkyl group or (C₁-C₁₀)—O-alkyl group, a hydroxy group, or a phosphate group. In another embodiment, when R₄ is not halogenated, R₄ is a hydrogen atom, a (C₁-C₆)-alkyl group or (C₁-C₆)—O-alkyl group, a hydroxy group, or a phosphate group. In another embodiment, when R₄ is not halogenated, R₄ is a hydrogen atom, a methyl group, an ethyl group, a propyl group, a phosphate group, an O-methyl group, an O-ethyl group, or an O-propyl group.

In another embodiment, R_{3A} and R_{3B} are a hydrogen atom, a (C₁-C₂₀)-alkyl group, a (C₆-C₁₄)-aryl group, or a —C(═O)(C₁-C₂₀)-alkyl group. In another embodiment, R_{3A} and R_{3B} are a hydrogen atom, a (C₁-C₁₀)-alkyl group, a (C₆-C₁₀)-aryl group, or a —C(═O)(C₁-C₁₀)-alkyl group. In another embodiment, R_{3A} and R_{3B} are a hydrogen atom, a (C₁-C₆)-alkyl group, a phenyl group, or a —C(═O)(C₁-C₆)-alkyl group. In another embodiment, R_{3A} and R_{3B} are a hydrogen atom, a methyl group, an ethyl group, a propyl group, a phenyl group, —C(═O)—CH₃, —C(═O)—CH₂CH₃, or —C(═O)—CH₂CH₂CH₃.

In one embodiment of the disclosure, a chemical compound or salts thereof having formula (I) is included:

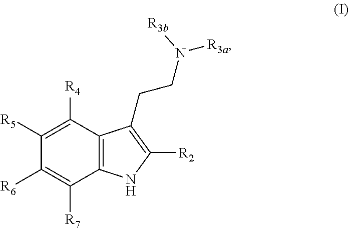

wherein
R₂, R₅, R₆, and R₇ are independently or simultaneously H, an alkyl group or O-alkyl group or a halogen atom, R_{3A} and R_{3B} are a hydrogen atom, an alkyl group, an aryl group, or an acyl group; and R₄ is hydrogen atom, alkyl group or O-alkyl group, a halogen, a hydroxy group, or a phosphate group; wherein at least one of R₂, R₄ R₅, R₆, and R₇ is a halogen.

In one embodiment, R₂, R₅, R₆, and R₇ are independently or simultaneously H, (C₁-C₂₀)-alkyl group or (C₁-C₂₀)—O-alkyl group or a halogen atom. In one embodiment, R₂, R₅, R₆, and R₇ are independently or simultaneously H, (C₁-C₁₀)-alkyl group or (C₁-C₁₀)—O-alkyl group or a halogen atom. In one embodiment, R₂, R₅, R₆, and R₇ are independently or simultaneously H, (C₁-C₆)-alkyl group or (C₁-C₆)—O-alkyl group or a halogen atom. In one embodiment, R₂, R₅, R₆, and R₇ are independently or simultaneously H, methyl, ethyl, propyl, O-methyl, O-ethyl, O-propyl, or a halogen atom.

In one embodiment, R₄ is H, (C₁-C₂₀)-alkyl group or (C₁-C₂₀)—O-alkyl group, a halogen atom or a phosphate group. In one embodiment, R₄ is H, (C₁-C₁₀)-alkyl group or (C₁-C₁₀)—O-alkyl group, a halogen atom or a phosphate group. In one embodiment, R₄ is H, (C₁-C₆)-alkyl group or (C₁-C₆)—O-alkyl group, a halogen atom, a hydroxy group, or a phosphate group. In one embodiment, R₄ is H, methyl, ethyl, propyl, O-methyl, O-ethyl, O-propyl, a halogen atom, a hydroxy group, or a phosphate group.

In another embodiment, R_{3A} and R_{3B} are a hydrogen atom independently or simultaneously a (C₁-C₂₀)-alkyl group, a (C₆-C₁₄)-aryl group, or a —C(═O)(C₁-C₂₀)-alkyl group. In another embodiment, R_{3A} and R_{3B} are independently or simultaneously a hydrogen atom, a (C₁-C₁₀)-alkyl group, a (C₆-C₁₀)-aryl group, or a —C(═O)(C₁-C₁₀)-alkyl group or O-alkyl group. In another embodiment, R_{3A} and R_{3B} are independently or simultaneously a hydrogen atom, a (C₁-C₆)-alkyl group, a phenyl group, or a —C(═O)(C₁-C₆)-alkyl group. In another embodiment, R_{3A} and R_{3B} are independently or simultaneously a hydrogen atom, a methyl group, an ethyl group, a propyl group, a butyl group, a phenyl group, —C(═O)—CH₃, —C(═O)—CH₂CH₃, or —C(═O)—CH₂CH₂CH₃.

The halogenated psilocybin derivatives of the present disclosure may be used to prepare a pharmaceutical or recreational drug formulation. Thus in one embodiment, the present disclosure further provides in another aspect, pharmaceutical and recreational drug formulations comprising halogenated psilocybin derivatives. Accordingly, in one aspect, the present disclosure provides in a further embodiment a pharmaceutical or recreational drug formulation comprising a chemical compound or salts thereof having the formula (I):

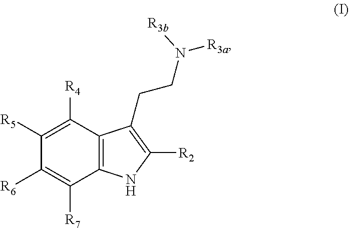

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein R_{3A} and R_{3B} are a hydrogen atom, an alkyl group, an aryl group, or an acyl group, together with a diluent, carrier or excipient. The pharmaceutical or recreational drug formulations may be prepared as liquids, tablets, capsules, microcapsules, nanocapsules, trans-dermal patches, gels, foams, oils, aerosols, nanoparticulates, powders, creams, emulsions, micellar systems, films, sprays, ovules, infusions, teas, decoctions, suppositories, etc. and include a pharmaceutically acceptable salt or solvate of the halogenated psilocybin compound together with an excipient. The term “excipient” as used herein means any ingredient other than the chemical compound of the disclosure. As will readily be appreciated by those of skill in art, the selection of excipient may depend on factors such as the particular mode of administration, the effect of the excipient on solubility of the chemical compounds of the present disclosure and methods for their preparation will be readily apparent to those skilled in the art. Such compositions and methods for their preparation may be found, for example, in “Remington's Pharmaceutical Sciences”, 22nd Edition (Pharmaceutical Press and Philadelphia College of Pharmacy at the University of the Sciences, 2012).

The dose when using the compounds of the present disclosure can vary within wide limits, and as is customary and is known to those of skill in the art, the dose can be tailored to the individual conditions in each individual case. The dose depends, for example, on the nature and severity of the illness to be treated, on the condition of the patient, on the compound employed or on whether an acute or chronic disease state is treated or prophylaxis is conducted, on the mode of delivery of the compound, or on whether further active compounds are administered in addition to the compounds of the present disclosure. Representative doses of the present disclosure include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to about 100 mg, about 0.001 mg to about 50 mg, and about 0.001 mg to about 25 mg. Representative doses of the present disclosure include, but are not limited to, about 0.0001 to about 1,000 mg, about 10 to about 160 mg, about 10 mg, about 20 mg, about 40 mg, about 80 mg, or about 160 mg. Multiple doses may be administered during the day, especially when relatively large amounts are deemed to be needed, for example 2, 3 or 4, doses. Depending on the subject and as deemed appropriate from the patient's physician or care giver it may be necessary to deviate upward or downward from the doses described herein.

The pharmaceutical and drug formulations comprising the halogenated psilocybin derivatives of the present disclosure may be administered orally. Oral administration may involve swallowing, so that the compound enters the gastrointestinal tract, or buccal or sublingual administration may be employed by which the compound enters the blood stream directly from the mouth. Formulations suitable for oral administration include both solid and liquid formulations.

Solid formulations include tablets, capsules (containing particulates, liquids, microcapsules, or powders), lozenges (including liquid-filled lozenges), chews, multi- and nano-particulates, gels, solid solutions, liposomal preparations, microencapsulated preparations, creams, films, ovules, suppositories and sprays.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose.

Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80. When present, surface active agents may comprise from 0.2% (w/w) to 5% (w/w) of the tablet.

Tablets may further contain lubricants such as magnesium stearate, calcium stearate, zinc stearate, sodium stearyl fumarate, and mixtures of magnesium stearate with sodium lauryl sulphate. Lubricants generally comprise from 0.25% (w/w) to 10% (w/w), from 0.5% (w/w) to 3% (w/w) of the tablet.

In addition to the halogenated psilocybin derivative, tablets may contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1% (w/w) to 25% (w/w) or from 5% (w/w) to 20% (w/w) of the dosage form.

Other possible auxiliary ingredients include anti-oxidants, colourants, flavouring agents, preservatives and taste-masking agents.

For tablet dosage forms, depending on the desired effective amount of the chemical compound, the chemical compound of the present disclosure may make up from 1% (w/w) to 80% (w/w) of the dosage form, more typically from 5% (w/w) to 60% (w/w) of the dosage form.

Exemplary tablets contain up to about 80% (w/w) of the chemical compound, from about 10% (w/w) to about 90% (w/w) binder, from about 0% (w/w) to about 85% (w/w) diluent, from about 2% (w/w) to about 10% (w/w) disintegrant, and from about 0.25% (w/w) to about 10% (w/w) lubricant.

The formulation of tablets is discussed in “Pharmaceutical Dosage Forms: Tablets”, Vol. 1-Vol. 3, by CRC Press (2008).

The pharmaceutical and recreational drug formulations comprising the halogenated psilocybin derivatives of the present disclosure may also be administered directly into the blood stream, into muscle, or into an internal organ. Thus, the pharmaceutical and recreational drug formulations can be administered parenterally (for example, by subcutaneous, intravenous, intraarterial, intrathecal, intraventricular, intracranial, intramuscular, or intraperitoneal injection). Parenteral formulations are typically aqueous solutions which may contain excipients such as salts, carbohydrates and buffering agents (in one embodiment, to a pH of from 3 to 9), but, for some applications, they may be more suitably formulated as a sterile non-aqueous solution or as a dried form to be used in conjunction with a suitable vehicle such as sterile water.

Formulations comprising the halogenated psilocybin derivatives of the present disclosure for parenteral administration may be formulated to be immediate and/or modified release. Modified release formulations include delayed-, sustained-, pulsed-, controlled-, targeted and programmed release. Thus the chemical compounds of the disclosure may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The pharmaceutical or recreational drug formulations of the present disclosure also may be administered topically to the skin or mucosa, i.e. dermally or transdermally. Example pharmaceutical and recreational drug formulations for this purpose include gels, hydrogels, lotions, solutions, creams, ointments, dusting powders, cosmetics, oils, eye drops, dressings, foams, films, skin patches, wafers, implants, sponges, fibres, bandages and microemulsions. Liposomes may also be used. Example carriers include alcohol, water, mineral oil, liquid petrolatum, white petrolatum, glycerin, polyethylene glycol and propylene glycol. Penetration enhancers may be incorporate (see: for example, Finnin, B. and Morgan, T. M., 1999 J. Pharm. Sci, 88 (10), 955-958).

Other means of topical administration include delivery by electroporation, iontophoresis, phonophoresis, sonophoresis and microneedle or needle-free (e.g., Powderject™, Bioject™, etc.) injection.

Pharmaceutical and recreational drug formulations for inhalation or insufflation include solutions and suspensions in pharmaceutically acceptable, aqueous or organic solvents, or mixtures thereof, and powders. The liquid or solid pharmaceutical compositions can contain suitable pharmaceutically acceptable excipients. In some embodiments, the pharmaceutical compositions are administered by the oral or nasal respiratory route for local or systemic effect. Pharmaceutical compositions in pharmaceutically acceptable solvents can be nebulized by use of inert gases. Nebulized solutions can be inhaled directly from the nebulizing device or the nebulizing device can be attached to a face mask tent, or intermittent positive pressure breathing machine. Solution, suspension, or powder pharmaceutical compositions can be administered, e.g., orally or nasally, from devices that deliver the formulation in an appropriate manner.

In further embodiments, in which the halogenated psilocybin compounds of present disclosure are used as a recreational drug, the compounds may be included in compositions such as food or food product, a beverage, a food seasoning, a personal care product, such as a cosmetic, perfume or bath oil, or oils (both for topical administration as massage oil, or to be burned or aerosolized). The chemical compounds of the present disclosure may also be included in a “vape” product, which may also include other drugs, such as nicotine, and flavorings.

The pharmaceutical formulations comprising the chemical compounds of the present disclosure may be used to treat a subject, and in particular to treat a psychiatric disorder in a subject. Accordingly, the present disclosure includes in a further embodiment, a method for treating a psychiatric disorder, the method comprising administering to a subject in need thereof a pharmaceutical formulation comprising a chemical compound or salts thereof having the formula (I):—

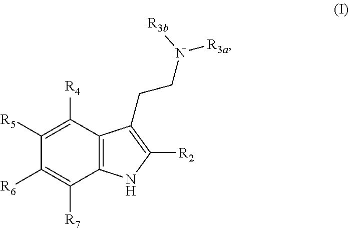

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein R_{3A} and R_{3B} are a hydrogen atom, or an alkyl group, an aryl group, or an acyl group, wherein the pharmaceutical formulation is administered in an effective amount to treat the psychiatric disorder in the subject.

Psychiatric disorders that may be treated include, for example, neurodevelopmental disorders such as intellectual disability, global development delay, communication disorders, autism spectrum disorder, and attention-deficit hyperactivity disorder (ADHD); bipolar and related disorders, such as mania, and depressive episodes; anxiety disorder, such as generalized anxiety disorder (GAD), agoraphobia, social anxiety disorder, specific phobias (natural events, medical, animal, situational, for example), panic disorder, and separation anxiety disorder; stress disorders, such as acute stress disorder, adjustment disorders, post-traumatic stress disorder (PTSD), and reactive attachment disorder; dissociative disorders, such as dissociative amnesia, dissociative identity disorder, and depersonalization/derealization disorder; somatoform disorders, such as somatic symptom disorders, illness anxiety disorder, conversion disorder, and factitious disorder; eating disorders, such as anorexia nervosa, bulimia nervosa, rumination disorder, pica, and binge-eating disorder; sleep disorders, such as narcolepsy, insomnia disorder, hypersomnolence, breathing-related sleep disorders, parasomnias, and restless legs syndrome; disruptive disorders, such as kleptomania, pyromania, intermittent explosive disorder, conduct disorder, and oppositional defiant disorder; depressive disorders, such as disruptive mood dysregulation disorder, major depressive disorder, persistent depressive disorder (dysthymia), premenstrual dysphoric disorder, substance/medication-induced depressive disorder, postpartum depression, and depressive disorder caused by another medical condition, for example, psychiatric and existential distress within life-threatening cancer situations (ACS Pharmacol Transl Sci 4: 553-562; J Psychiatr Res 137: 273); substance-related disorders, such as alcohol-related disorders, cannabis related disorders, inhalant-use related disorders, stimulant use disorders, and tobacco use disorders; neurocognitive disorders, such as delirium; schizophrenia; compulsive disorders, such as obsessive compulsive disorders (OCD), body dysmorphic disorder, hoarding disorder, trichotillomania disorder, excoriation disorder, substance/medication induced obsessive-compulsive disorder, and obsessive-compulsive disorder related to another medical condition; and personality disorders, such as antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, schizotypal personality disorder.

In an aspect, the compounds of the present disclosure may be used to be contacted with a 5-HT_{2A} receptor to thereby modulate the 5-HT_{2A} receptor. Such contacting includes bringing a compound of the present disclosure and 5-HT_{2A} receptor together under in vitro conditions, for example, by introducing the compounds in a sample containing a 5-HT_{2A} receptor, for example, a sample containing purified 5-HT_{2A} receptors, or a sample containing cells comprising 5-HT_{2A} receptors. In vitro conditions further include the conditions described in Example 3 hereof. Contacting further includes bringing a compound of the present disclosure and 5-HT_{2A} receptor together under in vivo conditions. Such in vivo conditions include the administration to an animal or human subject, for example, of a pharmaceutically effective amount of the compound of the present disclosure, when the compound is formulated together with a pharmaceutically active carrier, diluent or excipient, as hereinbefore described, to thereby treat the subject. Upon having contacted the 5-HT_{2A} receptor, the compound may activate the 5-HT_{2A} receptor or inhibit the 5-HT_{2A} receptor.

Thus, in a further aspect, the condition that may be treated in accordance herewith can be any 5-HT_{2A} receptor mediated disorder. Such disorders include, but are not limited to schizophrenia, psychotic disorder, attention deficit hyperactivity disorder, autism, and bipolar disorder.

The chemical compounds of the present disclosure may also be used as a feedstock material for other psilocybin derivatives. Thus in one embodiment, the chemical compounds of the present disclosure may be in used manufacture of a pharmaceutical or recreational drug formulation, wherein the manufacture may comprise derivatizing a chemical compound or salts thereof having the formula (I):

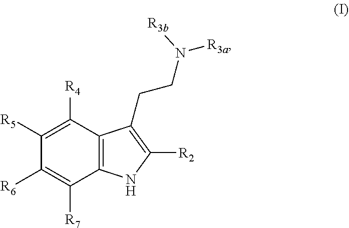

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein R_{3A} and R_{3B} are a hydrogen atom, an alkyl group, an aryl group, or an acyl group.

In order to use the compound having formula (I) as a feedstock, one or more halogen atoms may be replaced by any atoms or groups, for example hydrocarbon groups. Those of skill in the art will be generally familiar with methods that may be used to substitute halogen groups. In this respect, guidance may be found in Schnepel C. et al. (2017) Chem. Eur. J. 23:12064-12086; Durak L. J. et al. (2016) ACS Catal. 6: 1451; Runguphan W. et al. (2013) Org Lett 15: 2850; Corr M. J. et al. (2017) Chem. Sci. 8: 2039; and Roy A. D. et al. Chem. Comm. 4831.

Turning now to methods of making the halogenated psilocybin derivatives, it is noted that the psilocybin compounds of the present disclosure may be prepared in any suitable manner, including any organic chemical synthesis methods, biosynthetic methods, or a combination thereof. Synthesis generally may involve selecting a psilocybin precursor compound, and modifying the psilocybin precursor compound to form a halogenated psilocybin derivative. In this respect, it is noted that a non-halogenated psilocybin derivative may be selected and modified to form psilocybin, which subsequently may be halogenated, or, alternatively, a halogenated psilocybin derivative may be selected to subsequently form a halogenated psilocybin. Suitable psilocybin precursor compounds include compounds comprising an indole prototype structure (see: FIG. 2), including, for example tryptophan, tryptamine, 4-hydroxyindole, 4-hydroxytryptophan, 4-hydroxytryptamine, norbaeocystin and baeocystin, and halogenated forms thereof, notably, with respect to the indole prototype structure, 2-, 5-, 6-, 7-halogenated forms thereof. The halogenated psilocybin precursor compounds may be provided in a more or less chemically pure form, for example, in the form of a psilocybin precursor preparation having a purity of at least about 95%, at least about 96%, at least about 97%, at least about 98%, at least about 99%, or at least 99.9%. The psilocybin precursor compounds may be chemically synthesized, or obtained from a fine chemical manufacturer.

In one embodiment of the present disclosure the halogenated psilocybin derivatives may be formed biosynthetically. Accordingly, the present disclosure further includes in one embodiment, a method of making a halogenated psilocybin derivative the method comprising:
(a) contacting a halogenated psilocybin precursor compound with a host cell comprising a psilocybin biosynthetic enzyme complement; and
(b) growing the host cell to produce a halogenated psilocybin compound or salts thereof having the formula (I):

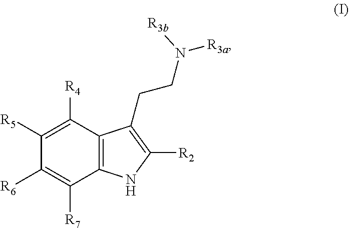

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, hydrogen atom or an alkyl group or O-alkyl group, and wherein R_{3A} and R_{3B} are a hydrogen atom, or an alkyl group, an aryl group, or an acyl group of the chemical compound.

Implementation of the foregoing example embodiment initially involves providing halogenated psilocybin precursor compounds and host cells having a psilocybin biosynthetic enzyme complement. Accordingly, next, exemplary halogenated psilocybin precursor compounds and example host cells that may be selected and used in accordance with the present disclosure will be described. Thereafter, example methodologies and techniques will be described to contact and use the halogenated psilocybin precursor compounds and cells to produce example halogenated psilocybin compounds.

A variety of halogenated psilocybin precursor compounds may be selected, prepared and used. In some embodiments, for example, the halogenated psilocybin precursor compound is a compound comprising a halogenated indole prototype structure. Examples of such compounds are halogenated tryptophan, halogenated tryptamine, halogenated 4-hydroxyindole, halogenated 4-hydroxytryptophan, halogenated 4-hydroxytryptamine, halogenated norbaeocystin, and halogenated baeocystin, and in particular the 2-halogenated, 5-halogenated, 6-halogenated and 7-halogenated forms (with respect to the indole prototype structure) of any of the foregoing.

Turning now to the host cells that can be used in accordance with the present disclosure, it is initially noted that a variety of host cells may be selected in accordance with the present disclosure, including microorganism host cells, plant host cells, and animal host cells.

In accordance herewith the host cell includes a psilocybin biosynthetic enzyme complement. Such cells can be obtained in at least two ways. First, in some embodiments, host cells may be selected in which a psilocybin biosynthetic enzyme complement is naturally present. Generally cells naturally producing psilocybin for example, cells of fungal species belonging to the genus psilocybe, are suitable in this respect. Second, in some embodiments, a host cell that not naturally produces psilocybin may be modulated to produce a psilocybin biosynthetic enzyme complement. Thus, for example, a nucleic acid sequence encoding a psilocybin biosynthetic enzyme complement may be introduced into a host cell, and upon cell growth the host cells can make the psilocybin biosynthetic enzyme complement.

Typically a nucleic acid sequence encoding one or more enzymes constituting a psilocybin biosynthetic enzyme complement further includes one or more additional nucleic acid sequences, for example, a nucleic acid sequences controlling expression of the one or more enzymes, and these one or more additional nucleic acid sequences together with the nucleic acid sequence encoding the one or more enzymes can be said to form a chimeric nucleic acid sequence.

A host cell which upon cultivation expresses the chimeric nucleic acid can be selected and used in accordance with the present disclosure. Suitable host cells in this respect include, for example, microbial cells, such as bacterial cells, yeast cells, for example, and algal cells or plant cells. A variety of techniques and methodologies to manipulate host cells to introduce nucleic acid sequences in cells and attain expression exists and are well known to the skilled artisan. These methods include, for example, cation based methods, for example, lithium ion or calcium ion based methods, electroporation, biolistics, and glass beads based methods. As will be known to those of skill in the art, depending on the host cell selected, the methodology to introduce nucleic acid material in the host cell may vary, and, furthermore, methodologies may be optimized for uptake of nucleic acid material by the host cell, for example, by comparing uptake of nucleic acid material using different conditions. Detailed guidance can be found, for example, in Sambrook et al., Molecular Cloning, a Laboratory Manual, Cold Spring Harbor Laboratory Press, 2012, Fourth Ed. It is noted that the chimeric nucleic acid is a non-naturally occurring chimeric nucleic acid sequence and can be said to be heterologous to the host cell.

In some embodiments, the one or more enzymes constituting a psilocybin enzyme complement can be selected from by a nucleic acid sequence selected from the nucleic acid sequences consisting of:
(a) SEQ.ID NO: 1, SEQ.ID NO: 3, SEQ.ID NO: 5, SEQ.ID NO: 7, SEQ.ID NO: 9, SEQ.ID NO 11, SEQ.ID NO: 36, SEQ.ID NO: 38, SEQ.ID NO: 40, SEQ. ID NO: 42, SEQ.ID NO: 44, SEQ.ID NO: 46, SEQ.ID NO: 48, SEQ.ID NO: 50, SEQ.ID NO: 52, SEQ.ID NO: 54, SEQ. ID NO: 56, SEQ.ID NO: 58, SEQ.ID NO: 60, SEQ.ID NO: 64, SEQ.ID NO: 66, SEQ.ID NO: 68, SEQ. ID NO: 70, and SEQ.ID NO: 72;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 2, SEQ.ID NO: 4, SEQ.ID NO: 6, SEQ.ID NO: 8, SEQ.ID NO: 10, SEQ.ID NO 12, SEQ.ID NO: 37, SEQ.ID NO: 39, SEQ.ID NO: 41, SEQ. ID NO: 43, SEQ.ID NO: 45, SEQ.ID NO: 47, SEQ.ID NO: 49, SEQ.ID NO: 51, SEQ.ID NO: 53, SEQ.ID NO: 55, SEQ. ID NO: 57, SEQ.ID NO: 59, SEQ.ID NO: 61, SEQ.ID NO: 63, SEQ.ID NO: 65, SEQ.ID NO: 67, SEQ.ID NO: 69, SEQ. ID NO: 71, and SEQ.ID NO: 73;
(f) a nucleic acid sequence that encodes a functional variant of any one of the amino acid sequences set forth in SEQ.ID NO: 2, SEQ.ID NO: 4, SEQ.ID NO: 6, SEQ.ID NO: 8, SEQ.ID NO: 10, SEQ.ID NO 12, SEQ.ID NO: 37, SEQ.ID NO: 39, SEQ.ID NO: 41, SEQ. ID NO: 43, SEQ.ID NO: 45, SEQ.ID NO: 47, SEQ.ID NO: 49, SEQ.ID NO: 51, SEQ.ID NO: 53, SEQ.ID NO: 55, SEQ. ID NO: 57, SEQ.ID NO: 59, SEQ.ID NO: 61, SEQ.ID NO: 63, SEQ.ID NO: 65, SEQ.ID NO: 67, SEQ.ID NO: 69, SEQ. ID NO: 71, and SEQ.ID NO: 73; and
(g) a nucleic acid sequence that hybridizes under stringent conditions to any one of the nucleic acid sequences set forth in (a), (b), (c), (d), (e) or (f).

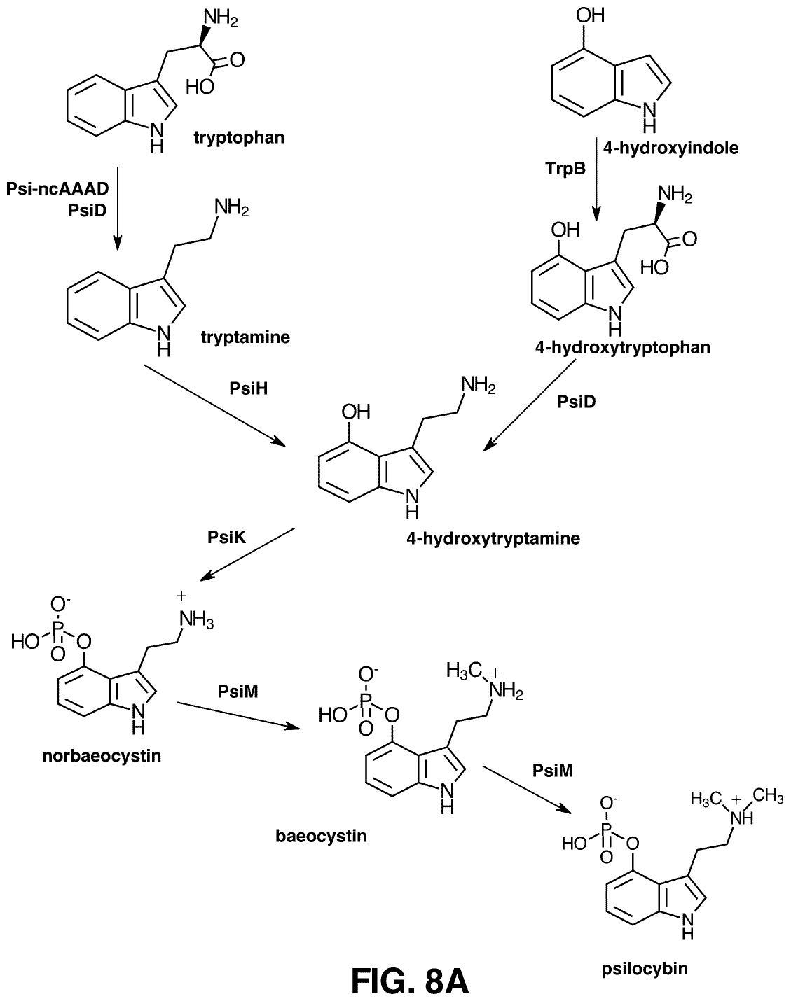

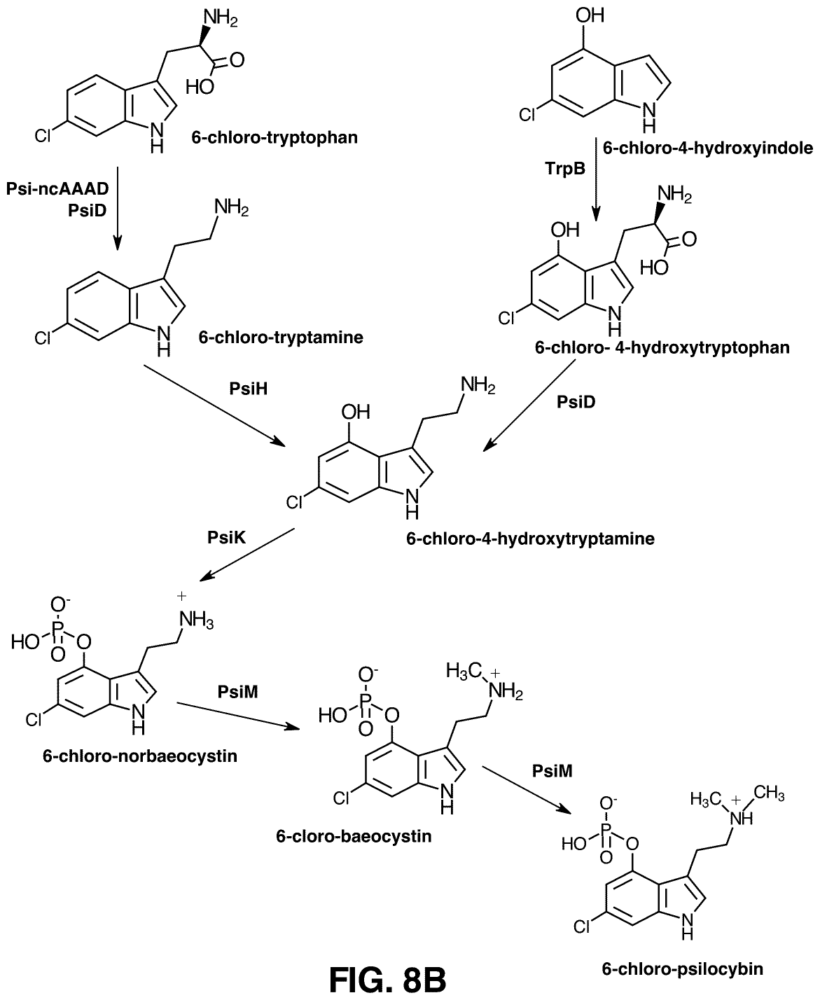

Thus any of the nucleic acid sequence set forth in (a), (b), (c), (d), (e), (f) or (g) may be selected and introduced into a host cell. In general, however the nucleic acid sequence is selected in conjunction with the selected psilocybin precursor compound, as hereinafter further discussed in reference to FIGS. 8A and 8B.

One example host cell that conveniently may be used is Escherichia coli. The preparation of the E. coli vectors may be accomplished using commonly known techniques such as restriction digestion, ligation, gel electrophoresis, DNA sequencing, the polymerase chain reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13 mp series of vectors, pBluescript etc. Suitable promoter sequences for use in E. coli include, for example, the T7 promoter, the T5 promoter, tryptophan (trp) promoter, lactose (lac) promoter, tryptophan/lactose (tac) promoter, lipoprotein (lpp) promoter, and λ phage PL promoter. Typically, cloning vectors contain a marker, for example, an antibiotic resistance marker, such as ampicillin or kanamycin resistance marker, allowing selection of transformed cells. Nucleic acid sequences may be introduced in these vectors, and the vectors may be introduced in E. coli by preparing competent cells, electroporation or using other well known methodologies to a person of skill in the art. E. coli may be grown in an appropriate medium, such as Luria-Broth medium and harvested. Recombinant expression vectors may readily be recovered from cells upon harvesting and lysing of the cells.

Another example host cell that may be conveniently used is a yeast cell. Example yeast host cells that can be used are yeast cells belonging to the genus Candida, Kiuyveronyces, Saccharomyces, Schizosaccharonyces, Pichia, Hansenula, and Yarrowia. In specific example embodiments, the yeast cell can be a Saccharomyces cerevisiae cell, a Yarrowia lipolytica cell, or Pichia pastoris cell.

A number of vectors exist for the expression of recombinant proteins in yeast host cells. Examples of vectors that may be used in yeast host cells include, for example, Yip type vectors, YEp type vectors, YRp type vectors, YCp type vectors, pGPD-2, pAO815, pGAPZ, pGAPZa, pHIL-D2, pHIL-S1, pPIC3L5K, pPIC9K, pPICZ, pPICZa, pPIC3K, pHWO10, pPUZZLE and 2 μm plasmids. Such vectors are known to the art and are, for example, described in Cregg et al, Mol Biotechnol (2000) 16(1): 23-52. Suitable promoter sequences for use in yeast host cells are also known and described, for example, in Mattanovich et al., Methods Mol. Biol., 2012, 824:329-58, and in Romanos et al, 1992, Yeast 8: 423-488. Examples of suitable promoters for use in yeast host cells include promoters of glycolytic enzymes, like triosephosphate isomerase (TPI), phosphoglycerate kinase (PGK), glyceraldehyde-3-phosphate dehydrogenase (GAPDH or GAP) and variants thereof; lactase (LAC) and galactosidase (GAL), P. pastoris glucose-6-phosphate isomerase promoter (PPGI), the 3-phosphoglycerate kinase promoter (PPGK), the glycerol aldehyde phosphate dehydrogenase promoter (PGAP), translation elongation factor promoter (PTEF), S. cerevisiae eno/ase (ENO-1), S. cerevisiae galactokinase (GAL1), S. cerevisiae alcohol dehydrogenase/glyceraldehyde-3-phosphate dehydrogenase (ADH1, ADH2/GAP), S. cerevisiae triose phosphate isomerase (TPI), S. cerevisiae metallothionein (CUP1), and S. cerevisiae 3-phosphoglycerate kinase (PGK), and the maltase gene promoter (MAL). Marker genes suitable for use in yeast host cells are also known to the art. Thus, antibiotic resistance markers, such as ampicillin resistance markers, can be used in yeast, as well as marker genes providing genetic functions for essential nutrients, for example, leucine (LEU2), tryptophan (TRP1 and TRP2), uracil (URA3, URA5, URA6), histidine (HIS3), and the like. Methods for introducing vectors into yeast host cells can, for example, be found in S. Kawai et al., 2010, Bioeng. Bugs 1(6): 395-403.

Further, guidance with respect to the preparation of expression vectors and introduction thereof into host cells, including in E. coli cells, yeast cells, and other host cells, may be found in, for example: Sambrook et al., Molecular Cloning, a Laboratory Manual, Cold Spring Harbor Laboratory Press, 2012, Fourth Ed.

Thus, to briefly recap, a host cell comprising a chimeric nucleic acid comprising (i) a nucleic acid sequence controlling expression in a host cell and (ii) a nucleic acid sequence encoding a psilocybin biosynthetic enzyme complement, can be prepared in accordance with the present disclosure.

In accordance herewith, host cells are grown to multiply and to express a chimeric nucleic acid. Expression of the chimeric nucleic acid results in the biosynthetic production in the host cell of a psilocybin biosynthetic enzyme complement. Growth media and growth conditions can vary depending on the host cell that is selected, as will be readily appreciated to those of ordinary skill in the art. Growth media typically contain a carbon source, one or several nitrogen sources, essential salts including salts of potassium, sodium, magnesium, phosphate and sulphate, trace metals, water soluble vitamins, and process aids including but not limited to antifoam agents, protease inhibitors, stabilizers, ligands and inducers. Example carbon sources are e.g. mono- or disaccharides. Example nitrogen sources are, e.g. ammonia, urea, amino acids, yeast extract, corn steep liquor and fully or partially hydrolyzed proteins. Example trace metals are e.g. Fe, Zn, Mn, Cu, Mo and H₃BO₃. Example water soluble vitamins are e.g. biotin, pantothenate, niacin, thiamine, p-aminobenzoic acid, choline, pyridoxine, folic acid, riboflavin and ascorbic acid. Further, specific example media include liquid culture media for the growth of yeast cells and bacterial cells including, Luria-Bertani (LB) broth for bacterial cell cultivation, and yeast extract peptone dextrose (YEPD or YPD), for yeast cell cultivation. Further media and growth conditions can be found in Sambrook et al., Molecular Cloning, a Laboratory Manual, Cold Spring Harbor Laboratory Press, 2012, Fourth Ed.

In order for the host cells to produce the halogenated psilocybin compound, the cells are provided with a psilocybin precursor compound. Thus in accordance herewith, host cells may be contacted with a psilocybin precursor compound. In some embodiments, a psilocybin precursor compound can be exogenously supplied, for example, by including a psilocybin precursor compound in the growth medium of the host cells, and growing the host cells in a medium including the psilocybin precursor compound.

Referring next to FIGS. 8A and 8B, shown therein is an example natural biosynthetic pathway showing the conversion of example psilocybin precursor compounds to form psilocybin (FIG. 8A). Thus, as can be appreciated from FIG. 8A, various psilocybin precursor compounds may be selected and prepared in halogenated form, in conjunction with a psilocybin biosynthetic enzyme complement.

Thus, by way of example, referring to FIG. 8B, a chlorinated psilocybin precursor compound, for example, 6-chloro-4-hydroxy indole may be selected and contacted with a host cell comprising a psilocybin biosynthetic enzyme complement comprising PsiD, (e.g. SEQ.ID NO: 2) PsiH (e.g. SEQ.ID NO: 4), PsiK (e.g. SEQ.ID NO; 6) and PsiM (e.g. SEQ.ID NO: 8), and upon growth of the cells 6-chloro-psilocybin can be formed. Or by way of another example, 6-chloro-tryptophan may be selected and contacted with a host cell comprising a psilocybin biosynthetic enzyme complement comprising TrpB (SEQ.ID NO: 12) PsiD, (e.g. SEQ.ID NO: 2), PsiK (e.g. SEQ.ID NO; 6) and PsiM (e.g. SEQ.ID NO: 8), and upon growth of the cells 6-chloro-psilocybin can be formed. It will be clear to those of skill in the art that by referring to FIGS. 8A and 8B, other halogenated psilocybin precursors may be selected (e.g. 2-chloro tryptophan, 4-chloro-tryptophan, 5-chloro-tryptophan, 7-chlorotryptophan; 6-bromo-tryptophan, 6-fluoro-tryptophan, 6-iodotryptophan; and 6-chlorotryptamine, 6-chloro-4-hydroxytryptophan, 6-hydroxy-4-chloro-tryptamine, 6-chloro-norbaeocystin or 6-chloro-baeocystin), and contacted with a host cell comprising an appropriate psilocybin biosynthetic enzyme complement to produce a halogenated psilocybin derivative.

Thus, in one further example embodiment, a halogenated indole, such as 6-chloro-4-hydroxy indole, for example (see: FIG. 8B), may be selected and contacted with a host cell comprising a psilocybin biosynthetic enzyme complement comprising (i) a nucleic acid encoding TrpB selected from SEQ.ID NO: 12, SEQ.ID NO: 65, SEQ.ID NO: 67, SEQ.ID NO: 69, SEQ.ID NO: 71, and SEQ.ID NO: 73, or a nucleic acid sequence encoding a polypeptide sequence substantially similar to any of the aforementioned sequences; (ii) a nucleic acid encoding PsiD selected from SEQ.ID NO: 2, SEQ.ID NO: 10, SEQ.ID NO: 37, SEQ.ID NO: 39, SEQ.ID NO: 41, SEQ.ID NO: 43, SEQ.ID NO: 45, SEQ.ID NO: 47, SEQ.ID NO: 49, SEQ.ID NO: 51, SEQ.ID NO: 53, SEQ.ID NO: 55, and SEQ.ID NO: 57, or a nucleic acid sequence encoding a polypeptide sequence substantially similar to any of the aforementioned sequences; (iii) a nucleic acid encoding PsiK encoded by SEQ.ID NO: 6, or a nucleic acid sequence encoding a polypeptide sequence substantially similar thereto; and (iv) a nucleic acid encoding PsiM selected from SEQ.ID NO: 8, SEQ.ID NO: 59 and SEQ.ID NO: 61 or a nucleic acid sequence encoding a polypeptide sequence substantially similar to any of the aforementioned sequences to produce a halogenated psilocybin derivative.

Thus, in one further example embodiment, a halogenated tryptophan, such as 6-chloro-4-hydroxy tryptophan, for example (see: FIG. 8B), may be selected and contacted with a host cell comprising a psilocybin biosynthetic enzyme complement comprising (i) a nucleic acid encoding PsiD selected from SEQ.ID NO: 2, SEQ.ID NO: 10, SEQ.ID NO: 37, SEQ.ID NO: 39, SEQ.ID NO: 41, SEQ.ID NO: 43, SEQ.ID NO: 45, SEQ.ID NO: 47, SEQ.ID NO: 49, SEQ.ID NO: 51, SEQ.ID NO: 53, SEQ.ID NO: 55, and SEQ.ID NO: 57, or a nucleic acid sequence encoding a polypeptide sequence substantially similar to any of the aforementioned sequences; (ii) a nucleic acid encoding PsiK encoded by SEQ.ID NO: 6, or a nucleic acid sequence encoding a polypeptide sequence substantially similar thereto; and (iii) a nucleic acid encoding PsiM selected from SEQ.ID NO: 8, SEQ.ID NO: 59 and SEQ.ID NO: 61 or a nucleic acid sequence encoding a polypeptide sequence substantially similar to any of the aforementioned sequences to produce a halogenated psilocybin derivative.

Thus, in one further example embodiment, a halogenated tryptamine, such as 6-chloro-4-hydroxy tryptamine, for example (see: FIG. 8B), may be selected and contacted with a host cell comprising a psilocybin biosynthetic enzyme complement comprising (i) a nucleic acid encoding PsiK encoded by SEQ.ID NO: 6, or a nucleic acid sequence encoding a polypeptide sequence substantially similar thereto; and (ii) and a nucleic acid encoding PsiM selected from SEQ.ID NO: 8, SEQ.ID NO: 59 and SEQ.ID NO: 61 or a nucleic acid sequence encoding a polypeptide sequence substantially similar to any of the aforementioned sequences to produce a halogenated psilocybin derivative.

Thus, in one further example embodiment, a halogenated norbaeocystin, such as 6-chloro-norbaeocystin, or a halogenated baeocystin may, such as 6-chlorobaeocystin, for example (see: FIG. 8B), may be selected and contacted with a host cell comprising a psilocybin biosynthetic enzyme complement comprising a nucleic acid encoding PsiM selected from SEQ.ID NO: 8, SEQ.ID NO: 59 and SEQ.ID NO: 61 or a nucleic acid sequence encoding a polypeptide sequence substantially similar to any of the aforementioned sequences to produce a halogenated psilocybin derivative.

Thus, in one further example embodiment, a halogenated tryptophan, such as 6-chloro-4-hydroxy tryptophan, for example (see: FIG. 8B), may be selected and contacted with a host cell comprising a psilocybin biosynthetic enzyme complement comprising (i) a nucleic acid encoding PsiD selected from SEQ.ID NO: 2, SEQ.ID NO: 10, SEQ.ID NO: 37, SEQ.ID NO: 39, SEQ.ID NO: 41, SEQ.ID NO: 43, SEQ.ID NO: 45, SEQ.ID NO: 47, SEQ.ID NO: 49, SEQ.ID NO: 51, SEQ.ID NO: 53, SEQ.ID NO: 55, and SEQ.ID NO: 57, or a nucleic acid sequence encoding a polypeptide sequence substantially similar to any of the aforementioned sequences; (ii) a nucleic acid encoding PsiH encoded by SEQ.ID NO: 4, or a nucleic acid sequence encoding a polypeptide sequence substantially similar thereto; (iii) a nucleic acid encoding PsiK encoded by SEQ.ID NO: 6, or a nucleic acid sequence encoding a polypeptide sequence substantially similar thereto; and (iv) and a nucleic acid encoding PsiM selected from SEQ.ID NO: 8, SEQ.ID NO: 59 and SEQ.ID NO: 61 or a nucleic acid sequence encoding a polypeptide sequence substantially similar to any of the aforementioned sequences to produce a halogenated psilocybin derivative.

Thus, in one further example embodiment, a halogenated tryptamine, such as 6-chloro-tryptamine, for example (see: FIG. 8B), may be selected and contacted with a host cell comprising a psilocybin biosynthetic enzyme complement comprising (i) a nucleic acid encoding PsiH encoded by SEQ.ID NO: 4, or a nucleic acid sequence encoding a polypeptide sequence substantially similar thereto; (ii) a nucleic acid encoding PsiK encoded by SEQ.ID NO: 6, or a nucleic acid sequence encoding a polypeptide sequence substantially similar thereto; and (iii) and a nucleic acid encoding PsiM selected from SEQ.ID NO: 8, SEQ.ID NO: 59 and SEQ.ID NO: 61 or a nucleic acid sequence encoding a polypeptide sequence substantially similar to any of the aforementioned sequences to produce a halogenated psilocybin derivative.

In some embodiments, the psilocybin can be a halogenated psilocybin precursor compound which is exogenously supplied to a host cell, for example by inclusion in the host cell's growth medium. Thus, for example, referring to FIG. 8, it will be understood that in accordance herewith, for example, 6-chloro-tryptophan or 6-chloro-4-hydroxy indole, may be included in the growth medium of a host cell comprising a psilocybin biosynthetic enzyme complement.

It is noted that in some embodiments, the host cells may in addition to the psilocybin biosynthetic enzyme complement include a halogenase. In such embodiments the cell may be contacted with a non-halogenated psilocybin precursor compound, and halogenation may occur in vivo in the host cells. Thus, referring again to FIG. 8, by way of example, non-halogenated tryptophan may be selected and contacted with a host cell comprising a psilocybin biosynthetic enzyme complement comprising PsiD, (e.g. SEQ.ID NO: 2) PsiH (e.g. SEQ.ID NO: 4). PsiK (e.g. SEQ.ID NO; 6) and PsiM (e.g. SEQ.ID NO: 8), the host cells further including a halogenase, and upon growth of the host cells in the presence of a halogen, for example in the form of a halogen salt, sodium chloride (NaCl) or sodium bromide (NaBr) halogenated psilocybin derivatives can be formed.

Suitable halogenases that may be used in accordance herewith include, for example, a PyrH halogenase of Streptomyces rugosporus (SEQ.ID NO: 13; SEQ.ID NO: 14), which may, for example, be contacted with non-halogenated tryptophan to form 5-halo-tryptophan; a KtZR halogenase of Kutzneria sp. 744 (SEQ.ID NO: 15; SEQ.ID NO: 16), which may be contacted with non-halogenated tryptophan to form 6-halo-tryptophan; a SstH halogenase of Streptomyces toxytrinici (SEQ.ID NO: 17; SEQ.ID NO: 18), which may be contacted with non-halogenated tryptophan to form 6-halo-tryptophan; a ThaI halogenase of Streptomyces albogriseolus (SEQ.ID NO: 19; SEQ.ID NO: 20), which may be contacted with non-halogenated tryptophan to form 6-halo-tryptophan; a BorH halogenase (see: SEQ.ID NO: 21; SEQ.ID NO: 22), which may be contacted with non-halogenated tryptophan to form 6-halo-tryptophan; a KtzQ halogenase of Kutzneria sp. 744 (SEQ.ID NO: 23; SEQ.ID NO: 24), which may be contacted with non-halogenated tryptophan to form 7-halo-tryptophan; a PrnA halogenase of Pseudomonas fluorescens (SEQ.ID NO: 25; SEQ.ID NO: 26), which may be contacted with non-halogenated tryptophan and other psilocybin precursor compounds to form 7-halo-tryptophan or 2-halo-tryptophan and other 7-halo or 2-halo psilocybin precursor compounds; a RebH halogenase of Lentzea aerocolonigenes (SEQ.ID NO: 27; SEQ.ID NO: 28), which may be contacted with non-halogenated tryptophan to form 7-halo-tryptophan; and a SatH halogenase of Streptomyces albus (SEQ.ID NO: 29), which may be contacted with non-halogenated tryptophan to form 6-halo-tryptophan.

It is noted that halogenases comprising amino acid sequences substantially similar to any of the aforementioned halogenase sequences may be prepared and used to modulate the substrate specificity and/or the enzyme's catalytic efficiency. Thus, for example, halogenases comprising amino acid sequences substantially similar to SEQ.ID NO: 14; SEQ.ID NO: 16; SEQ.ID NO: 18; SEQ.ID NO: 20; SEQ.ID NO: 22; SEQ.ID NO: 24; SEQ.ID NO: 26; SEQ.ID NO: 28, and SEQ.ID NO: 29, may be prepared and used to obtain 2-halo, 5-halo, 6-halo or 7-halo tryptophan, or halogenases comprising amino acid sequences substantially similar to SEQ.ID NO: 14; SEQ.ID NO: 16; SEQ.ID NO: 18; SEQ.ID NO: 20; SEQ.ID NO: 22; SEQ.ID NO: 24; SEQ.ID NO: 26; SEQ.ID NO: 28, and SEQ.ID NO: 29 may be prepared and used to halogenate other non-halogenated psilocybin precursor compounds, such as tryptamine, 4-hydroxyindole, 4-hydroxytryptophan, 4-hydroxytryptamine, norbaeocystin, and baeocystin, for example, to form halogenated forms thereof.

Thus, for example, the native polypeptide sequence of a RebH halogenase Lentzea aerocolonigenes (SEQ.ID NO: 27; SEQ.ID NO: 28) may be modulated to thereby modulate the substrate specificity. Thus, for example, a modulated Lentzea aerocolonigenes RebH halogenase, which may be referred to as a RebH-Y455W mutant, set forth in SEQ.ID NO: 30 prefers tryptamine over tryptophan as a substrate and when contacted with tryptamine forms 7-halo-tryptamine.

By way of a further example, a modulated Lentzea aerocolonigenes RebH halogenase, which may be referred to as a RebH-N470S mutant, set forth in SEQ.ID NO: 31 prefers tryptamine over tryptophan as a substrate and when contacted with tryptamine forms 7-halo-tryptamine.

By way of a further example, a modulated Lentzea aerocolonigenes RebH halogenase, which may be referred to as a RebH-8F mutant, set forth in SEQ.ID NO: 32 prefers tryptamine over tryptophan as a substrate and when contacted with tryptamine forms 6-halo-tryptamine.

By way of a further example, a modulated Lentzea aerocolonigenes RebH halogenase, which may be referred to as a RebH-10F mutant, set forth in SEQ.ID NO: 33 prefers tryptamine over tryptophan as a substrate and when contacted with tryptamine forms 5-halo-tryptamine.

By way of a further example, a modulated Lentzea aerocolonigenes RebH halogenase, which may be referred to as a RebH-3SS mutant, set forth in SEQ.ID NO: 34 has an altered substrate acceptance profile and converts tricyclic tryptoline derivatives.

By way of yet a further example, a modulated Lentzea aerocolonigenes RebH halogenase, which may be referred to as a RebH-3SS mutant set forth in SEQ.ID NO: 35 has an altered substrate acceptance profile and converts a broad range of large indoles and carbazoles.

The foregoing examples of halogenases exhibiting modulated substrate specificity and/or catalytic efficiency are known to the art and are documented in, for example, Andorfer M. C., et al. (2016) Chem. Sci. 7: 3720; Andorfer M. C. et al. (2017) Catalysis 7: 1897; Glenn W. S., et al. (2011) J. Am. Chem. Soc. 133: 19346; and Payne J. T. et al. (2015) Angew. Chem. Int. Ed. 54: 4226.

Accordingly, in one embodiment the halogenase can be encoded by a nucleic acid selected from:
(a) SEQ.ID NO: 13, SEQ.ID NO: 15, SEQ. ID NO: 17, SEQ.ID NO: 19, SEQ. ID NO: 21, SEQ.ID NO: 23, SEQ.ID NO: 25, and SEQID NO: 27;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 14, SEQ.ID NO: 16, SEQ. ID NO: 18, SEQ.ID NO: 20, SEQ. ID NO: 22, SEQ.ID NO: 24, SEQ.ID NO: 26, SEQ.ID NO: 28, SEQ.ID NO: 29, SEQ.ID NO: 30, SEQ.ID NO: 31, SEQ.ID NO: 32. SEQ.ID NO: 33, SEQ.ID NO: 34, and SEQ.ID NO: 35;
(f) a nucleic acid sequence that encodes a functional variant of any one of the amino acid sequences set forth in SEQ.ID NO: 14, SEQ.ID NO: 16, SEQ. ID NO: 18, SEQ.ID NO: 20, SEQ. ID NO: 22, SEQ.ID NO: 24, SEQ.ID NO: 26, SEQ.ID NO: 28, SEQ.ID NO: 29, SEQ.ID NO: 30, SEQ.ID NO: 31, SEQ.ID NO: 32. SEQ.ID NO: 33, SEQ.ID NO: 34, and SEQ.ID NO: 35; and
(g) a nucleic acid sequence that hybridizes under stringent conditions to any one of the nucleic acid sequences set forth in (a), (b), (c), (d), (e) or (f).

In one further embodiment, the host cell can comprise an acetyl transferase capable of acetylating R₃ₐ or R_{3b}, wherein the cell comprises a compound having chemical formula (I):

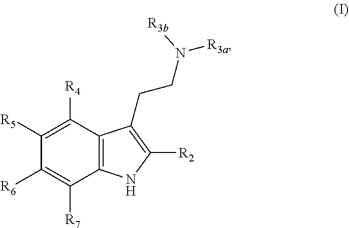

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein at least one of R_{3A} and R_{3B} is a hydrogen atom,
to form a compound having the chemical formula (XIX):

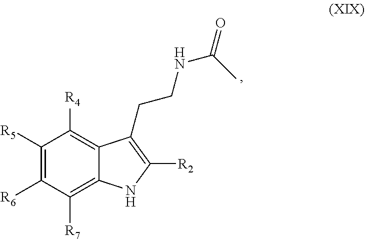

wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group.

In some embodiments, R₃ₐ and R_{3b} are each a hydrogen atom.

Suitable acyl transferases that may be used in accordance herewith include a PsmF acetyl transferase obtained from Streptomyces griseofuscus, including a PsmF N-acetyltransferase comprising polypeptide sequence SEQ.ID NO: 63 set forth herein, and further including a PsmF polypeptide encoded by a nucleic acid selected from:
(a) SEQ.ID NO: 62;
(b) a nucleic acid sequence that is substantially identical the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having an amino acid sequences set forth in SEQ.ID NO: 63;
(f) a nucleic acid sequence that encodes a functional variant of the amino acid sequence set forth in SEQ.ID NO: 63; and
(g) a nucleic acid sequence that hybridizes under stringent conditions to any one of the nucleic acid sequences set forth in (a), (b), (c), (d), (e) or (f).

It will be clear to those of skill in the art that a significant variety of different halogenated and non-halogenated psilocybin precursor compounds may be selected. FIGS. 8A and 8B in this respect provide guidance and allow a person of skill in the art to select appropriate psilocybin precursor compounds and a matching a psilocybin biosynthetic enzyme complement.

Upon production by the host cells of the halogenated psilocybin compounds in accordance with the methods of the present disclosure, the halogenated psilocybin compounds may be extracted from the host cell suspension, and separated from other constituents within the host cell suspension, such as media constituents and cellular debris. Separation techniques will be known to those of skill in the art and include, for example, solvent extraction (e.g. butane, chloroform, ethanol), column chromatography based techniques, high-performance liquid chromatography (HPLC), for example, and/or countercurrent separation (CCS) based systems. The recovered halogenated psilocybin compounds may be obtained in a more or less pure form, for example, a preparation of halogenated psilocybin compounds of at least about 60% (w/w), about 70% (w/w), about 80% (w/w), about 90% (w/w), about 95% (w/w), about 96% (w/w), about 97% (w/w), about 98% (w/w) or about 99% (w/w) purity may be obtained. Thus, in this manner, halogenated psilocybin derivatives in more or less pure form may be prepared.

Similarly, other methods of making the halogenated psilocybin compounds that may be used in accordance herewith may yield preparations of halogenated compounds of at least about 60% (w/w), about 70% (w/w), about 80% (w/w), about 90% (w/w), about 95% (w/w), about 96% (w/w), about 97% (w/w), about 98% (w/w), or about 99% (w/w) purity.

It will now be clear form the foregoing that novel halogenated psilocybin derivatives are disclosed herein. The halogenated psilocybin compounds may be formulated for use as a pharmaceutical drug or recreational drug. The halogenated psilocybin compounds may also be used as a feedstock to produce other psilocybin derivatives.

### Summary of Sequences

SEQ.ID NO: 1 sets forth a Psilocybe cubensis nucleic acid sequence encoding a PsiD polypeptide.

SEQ.ID NO: 2 sets forth a deduced amino acid sequence of a Psilocybe cubensis PsiD polypeptide.

SEQ.ID NO: 3 sets forth a Psilocybe cubensis nucleic acid sequence encoding a PsiH polypeptide.

SEQ.ID NO: 4 sets forth a deduced amino acid sequence of a Psilocybe cubensis PsiH polypeptide.

SEQ.ID NO: 5 sets forth a Psilocybe cubensis nucleic acid sequence encoding a PsiK polypeptide.

SEQ.ID NO: 6 sets forth a deduced amino acid sequence of a Psilocybe cubensis PsiK polypeptide.

SEQ.ID NO: 7 sets forth a Psilocybe cubensis nucleic acid sequence encoding a PsiM polypeptide.

SEQ.ID NO: 8 sets forth a deduced amino acid sequence of a Psilocybe cubensis PsiM polypeptide.

SEQ.ID NO: 9 sets forth a Psilocybe cubensis nucleic acid sequence encoding a Psi-ncAAAD polypeptide.

SEQ.ID NO: 10 sets forth a deduced amino acid sequence of a Psilocybe cubensis Psi-ncAAAD polypeptide.

SEQ.ID NO: 11 sets forth a Psilocybe cubensis nucleic acid sequence encoding a TrpB polypeptide.

SEQ.ID NO: 12 sets forth a deduced amino acid sequence of a Psilocybe cubensis TrpB polypeptide.

SEQ.ID NO: 13 sets forth a Streptomyces rugosporus nucleic acid sequence encoding a PyrH halogenase polypeptide.

SEQ.ID NO: 14 sets forth a deduced amino acid sequence of a Streptomyces rugosporus PyrH halogenase polypeptide.

SEQ.ID NO: 15 sets forth a Kutzneria sp. 744 nucleic acid sequence encoding a KtzR halogenase polypeptide.

SEQ.ID NO: 16 sets forth a deduced amino acid sequence of a Kutzneria sp. 744 KtzR halogenase polypeptide.

SEQ.ID NO: 17 sets forth a Streptomyces toxytrinici nucleic acid sequence encoding a SttH halogenase polypeptide.

SEQ.ID NO: 18 sets forth a deduced amino acid sequence of a Streptomyces toxytrinici SttH halogenase polypeptide.

SEQ.ID NO: 19 sets forth a Streptomyces albogriseolus nucleic acid sequence encoding a ThaI halogenase polypeptide.

SEQ.ID NO: 20 sets forth a deduced amino acid sequence of a Streptomyces albogriseolus ThaI halogenase polypeptide.

SEQ.ID NO: 21 sets forth a nucleic acid sequence encoding a BorH halogenase polypeptide of an unidentified soil bacterium.

SEQ.ID NO: 22 sets forth a deduced amino acid sequence of a BorH halogenase polypeptide of an unidentified soil bacterium.

SEQ.ID NO: 23 sets forth a Kutzneria sp. 744 nucleic acid sequence encoding a KtzQ halogenase polypeptide.

SEQ.ID NO: 24 sets forth a deduced amino acid sequence of a Kutzneria sp. 744 KtzQ halogenase polypeptide.

SEQ.ID NO: 25 sets forth a Pseudomonas fluorescens nucleic acid sequence encoding a PrnA halogenase polypeptide.

SEQ.ID NO: 26 sets forth a deduced amino acid sequence of a Pseudomonas fluorescens PrnA halogenase polypeptide.

SEQ.ID NO: 27 sets forth a nucleic acid sequence encoding a Lentzea areocolonigenes RebH halogenase polypeptide.

SEQ.ID NO: 28 sets forth a deduced amino acid sequence of a Lentzea aerocologinenes RebH halogenase polypeptide.

SEQ.ID NO: 29 sets forth a deduced amino acid sequence of a Streptomyces albus SatH halogenase polypeptide.

SEQ.ID NO: 30 sets forth a deduced amino acid sequence of a mutated Lentzea aerocologinenes RebH halogenase polypeptide.

SEQ.ID NO: 31 sets forth a deduced amino acid sequence of another mutated Lentzea aerocologinenes RebH halogenase polypeptide.

SEQ.ID NO: 32 sets forth a deduced amino acid sequence of another mutated Lentzea aerocologinenes RebH halogenase polypeptide.

SEQ.ID NO: 33 sets forth a deduced amino acid sequence of another mutated Lentzea aerocologinenes RebH halogenase polypeptide.

SEQ.ID NO: 34 sets forth a deduced amino acid sequence of another mutated Lentzea aerocologinenes RebH halogenase polypeptide.

SEQ.ID NO: 35 sets forth a deduced amino acid sequence of another mutated Lentzea aerocologinenes RebH halogenase polypeptide.

SEQ.ID NO: 36 sets forth a nucleic acid sequence encoding a Bacillus atrophaeus BaTDC tryptophan decarboxylase polypeptide.

SEQ.ID NO: 37 sets forth a deduced amino acid sequence of a Bacillus atrophaeus BaTDC tryptophan decarboxylase polypeptide.

SEQ.ID NO: 38 sets forth a nucleic acid sequence encoding a Ruminococcus gnayus RgnTDC tryptophan decarboxylase polypeptide.

SEQ.ID NO: 39 sets forth a deduced amino acid sequence of a Ruminococcus gnayus RgnTDC tryptophan decarboxylase polypeptide.

SEQ.ID NO: 40 sets forth a nucleic acid sequence encoding a Streptomyces griseofuscus SgTDC tryptophan decarboxylase polypeptide.

SEQ.ID NO: 41 sets forth a deduced amino acid sequence of a Streptomyces griseofuscus SgTDC tryptophan decarboxylase polypeptide.

SEQ.ID NO: 42 sets forth a nucleic acid sequence encoding a Ceriporiopsis subvermispora CsTDC tryptophan decarboxylase polypeptide.

SEQ.ID NO: 43 sets forth a deduced amino acid sequence of a Ceriporiopsis subvermispora CsTDC tryptophan decarboxylase polypeptide.

SEQ.ID NO: 44 sets forth a nucleic acid sequence encoding a Clostridium sporogenes ClostSporTDC tryptophan decarboxylase polypeptide.

SEQ.ID NO: 45 sets forth a deduced amino acid sequence of a Clostridium sporogenes ClostSporTDC tryptophan decarboxylase polypeptide.

SEQ.ID NO: 46 sets forth a nucleic acid sequence encoding a Homo sapiens HsDDC DOPA decarboxylase polypeptide.

SEQ.ID NO: 47 sets forth a deduced amino acid sequence of a Homo sapiens HsDDC DOPA decarboxylase polypeptide.

SEQ.ID NO: 48 sets forth a nucleic acid sequence encoding an Ophiorrhiza pumila OpTDC tryptophan decarboxylase polypeptide.

SEQ.ID NO: 49 sets forth a deduced amino acid sequence of an Ophiorrhiza pumila OpTDC tryptophan decarboxylase polypeptide.

SEQ.ID NO: 50 sets forth a nucleic acid sequence encoding an Oryza sativa OsTDC1 tryptophan decarboxylase polypeptide.

SEQ.ID NO: 51 sets forth a deduced amino acid sequence of an Oryza sativa OsTDC1 tryptophan decarboxylase polypeptide.

SEQ.ID NO: 52 sets forth a nucleic acid sequence encoding another Oryza sativa OsTDC2 tryptophan decarboxylase polypeptide.

SEQ.ID NO: 53 sets forth a deduced amino acid sequence of another Oryza sativa OsTDC2 tryptophan decarboxylase polypeptide.

SEQ.ID NO: 54 sets forth a nucleic acid sequence encoding a Xenorhabdus doucetiae XdTDC decarboxylase polypeptide.

SEQ.ID NO: 55 sets forth a deduced amino acid sequence of a Xenorhabdus doucetiae XdTDC decarboxylase polypeptide.

SEQ.ID NO: 56 sets forth a nucleic acid sequence encoding a Camptotheca acuminata CaTDC tryptophan decarboxylase polypeptide.

SEQ.ID NO: 57 sets forth a deduced amino acid sequence of a Camptotheca acuminata CaTDC tryptophan decarboxylase polypeptide.

SEQ.ID NO: 58 sets forth a nucleic acid sequence encoding an Oryctolagus cuniculus OcINMT indolethylamine N-methyltransferase polypeptide.

SEQ.ID NO: 59 sets forth a deduced amino acid sequence of an Oryctolagus cuniculus OcINMT indolethylamine N-methyltransferase polypeptide.

SEQ.ID NO: 60 sets forth a nucleic acid sequence encoding a Hordeum vulgare HvNMT N-methyltransferase polypeptide.

SEQ.ID NO: 61 sets forth a deduced amino acid sequence of a Hordeum vulgare HvNMT N-methyltransferase polypeptide.

SEQ.ID NO: 62 sets forth a nucleic acid sequence encoding a Streptomyces griseofuscus PsmF N-acetyltransferase polypeptide.

SEQ.ID NO: 63 sets forth a deduced amino acid sequence of a Streptomyces griseofuscus PsmF N-acetyltransferase polypeptide.

SEQ.ID NO: 64 sets forth a nucleic acid sequence encoding a mutated Thermotoga maritima TmTrpB-2F3 tryptophan synthase subunit B polypeptide.

SEQ.ID NO: 65 sets forth a deduced amino acid sequence of a mutated Thermotoga maritima TmTrpB-2F3 tryptophan synthase subunit B polypeptide.

SEQ.ID NO: 66 sets forth a nucleic acid sequence encoding a mutated Pyrococcus furiosus PfTrpB-0A9 tryptophan synthase subunit B polypeptide.

SEQ.ID NO: 67 sets forth a deduced amino acid sequence of a mutated Pyrococcus furiosus PfTrpB-0A9 tryptophan synthase subunit B polypeptide.

SEQ.ID NO: 68 sets forth a nucleic acid sequence encoding another mutated Pyrococcus furiosus PfTrpB-2A6 tryptophan synthase subunit B polypeptide.

SEQ.ID NO: 69 sets forth a deduced amino acid sequence of a mutated Pyrococcus furiosus PfTrpB-2A6 tryptophan synthase subunit B polypeptide.

SEQ.ID NO: 70 sets forth a nucleic acid sequence encoding another mutated Pyrococcus furiosus PfTrpB-7E6 tryptophan synthase subunit B polypeptide.

SEQ.ID NO: 71 sets forth a deduced amino acid sequence of a mutated Pyrococcus furiosus PfTrpB-7E6 tryptophan synthase subunit B polypeptide.

SEQ.ID NO: 72 sets forth a nucleic acid sequence encoding another mutated Pyrococcus furiosus PfTrpB-Quat tryptophan synthase subunit B polypeptide.

SEQ.ID NO: 73 sets forth a deduced amino acid sequence of a mutated Pyrococcus furiosus PfTrpB-Quat tryptophan synthase subunit B polypeptide.

SEQ.ID NO: 74 sets forth a nucleic acid sequence used as integration cassette XII-4::TADH1-PsiH-HA-PPGK1-PTDH3-CPR-c-myc-TCYC1 for homologous recombination-based genome editing of Saccharomyces cerevisiae strain Sc-1.

SEQ.ID NO: 75 sets forth a nucleic acid sequence used as integration cassette XII-5::TADH1-PsiK-V5-PPGK1-PTDH3-PsiM-FLAG-TCYC1 for homologous recombination-based genome editing of Saccharomyces cerevisiae strain Sc-1.

SEQ.ID NO: 76 sets forth a nucleic acid sequence of modified pMM1 vector pMM1-PTDH3-ClostSporTDC-His-TCYC1 for engineering of Saccharomyces cerevisiae strain Sc-1.

SEQ.ID NO: 77 sets forth a nucleic acid sequence for Saccharomyces cerevisiae promoter PGK1.

SEQ.ID NO: 78 sets forth a nucleic acid sequence for Saccharomyces cerevisiae promoter TDH3.

SEQ.ID NO: 79 sets forth a nucleic acid sequence for Saccharomyces cerevisiae promoter CLN1.

SEQ.ID NO: 80 sets forth a nucleic acid sequence for Saccharomyces cerevisiae promoter UGA1.

SEQ.ID NO: 81 sets forth a nucleic acid sequence encoding a synthetic HA epitope tag polypeptide

SEQ.ID NO: 82 sets forth deduced amino acid sequence of a synthetic HA epitope tag polypeptide

SEQ.ID NO: 83 sets forth a nucleic acid sequence encoding a synthetic c-MYC epitope tag polypeptide

SEQ.ID NO: 84 sets forth deduced amino acid sequence of a synthetic c-MYC epitope tag polypeptide

SEQ.ID NO: 85 sets forth a nucleic acid sequence encoding a synthetic FLAG epitope tag polypeptide

SEQ.ID NO: 86 sets forth deduced amino acid sequence of a synthetic FLAG epitope tag polypeptide

SEQ.ID NO: 87 sets forth a nucleic acid sequence encoding a synthetic V5 epitope tag polypeptide

SEQ.ID NO: 88 sets forth deduced amino acid sequence of a synthetic V5 epitope tag polypeptide

SEQ.ID NO: 89 sets forth a nucleic acid sequence encoding a synthetic HIS epitope tag polypeptide

SEQ.ID NO: 90 sets forth deduced amino acid sequence of a synthetic HIS epitope tag polypeptide

SEQ.ID NO: 91 sets forth a nucleic acid sequence of pMM1 vector

SEQ.ID NO: 92 sets forth a nucleic acid sequence of pCDM4 vector

SEQ.ID NO: 93 sets forth a nucleic acid sequence of pETM6 vector

SEQ.ID NO: 94 sets forth a nucleic acid sequence of pET28a(+) vector

SEQ.ID NO: 95 sets forth a nucleic acid sequence encoding a Psilocybe cubensis PcCPR cytochrome P450 reductase polypeptide.

SEQ.ID NO: 96 sets forth a deduced amino acid sequence of a Psilocybe cubensis PcCPR cytochrome P450 reductase polypeptide.

SEQ.ID NO: 97 sets forth a nucleic acid sequence of pETM6-H10 vector.

SEQ.ID NO: 98 sets forth a nucleic acid sequence of pRSM3 vector.

SEQUENCE LISTING
SEQ. ID NO: 1
ATGCAGGTGATACCCGCGTGCAACTCGGCAGCAATAAGATCACTATGT
CCTACTCCCGAGTCTTTTAGAAACATGGGATGGCTCTCTGTCAGCGAT
GCGGTCTACAGCGAGTTCATAGGAGAGTTGGCTACCCGCGCTTCCAAT
CGAAATTACTCCAACGAGTTCGGCCTCATGCAACCTATCCAGGAATTC
AAGGCTTTCATTGAAAGCGACCCGGTGGTGCACCAAGAATTTATTGAC
ATGTTCGAGGGCATTCAGGACTCTCCAAGGAATTATCAGGAACTATGT
AATATGTTCAACGATATCTTTCGCAAAGCTCCCGTCTACGGAGACCTT
GGCCCTCCCGTTTATATGATTATGGCCAAATTAATGAACACCCGAGCG
GGCTTCTCTGCATTCACGAGACAAAGGTTGAACCTTCACTTCAAAAAA
CTTTTCGATACCTGGGGATTGTTCCTGTCTTCGAAAGATTCTCGAAAT
GTTCTTGTGGCCGACCAGTTCGACGACAGACATTGCGGCTGGTTGAAC
GAGCGGGCCTTGTCTGCTATGGTTAAACATTACAATGGACGCGCATTT
GATGAAGTCTTCCTCTGCGATAAAAATGCCCCATACTACGGCTTCAAC
TCTTACGACGACTTCTTTAATCGCAGATTTCGAAACCGAGATATCGAC
CGACCTGTAGTCGGTGGAGTTAACAACACCACCCTCATTTCTGCTGCT
TGCGAATCACTTTCCTACAACGTCTCTTATGACGTCCAGTCTCTCGAC
ACTTTAGTTTTCAAAGGAGAGACTTATTCGCTTAAGCATTTGCTGAAT
AATGACCCTTTCACCCCACAATTCGAGCATGGGAGTATTCTACAAGGA
TTCTTGAACGTCACCGCTTACCACCGATGGCACGCACCCGTCAATGGG
ACAATCGTCAAAATCATCAACGTTCCAGGTACCTACTTTGCGCAAGCC
CCGAGCACGATTGGCGACCCTATCCCGGATAACGATTACGACCCACCT
CCTTACCTTAAGTCTCTTGTCTACTTCTCTAATATTGCCGCAAGGCAA
ATTATGTTTATTGAAGCCGACAACAAGGAAATTGGCCTCATTTTCCTT
GTGTTCATCGGCATGACCGAAATCTCGACATGTGAAGCCACGGTGTCC
GAAGGTCAACACGTCAATCGTGGCGATGACTTGGGAATGTTCCATTTC
GGTGGTTCTTCGTTCGCGCTTGGTCTGAGGAAGGATTGCAGGGCAGAG
ATCGTTGAAAAGTTCACCGAACCCGGAACAGTGATCAGAATCAACGAA
GTCGTCGCTGCTCTAAAGGCTTAG
SEQ. ID NO: 2
MQVIPACNSAAIRSLCPTPESFRNMGWLSVSDAVYSEFIGELATRASN
RNYSNEFGLMQPIQEFKAFIESDPVVHQEFIDMFEGIQDSPRNYQELC
NMFNDIFRKAPVYGDLGPPVYMIMAKLMNTRAGFSAFTRQRLNLHFKK
LFDTWGLFLSSKDSRNVLVADQFDDRHCGWLNERALSAMVKHYNGRAF
DEVFLCDKNAPYYGFNSYDDFFNRRFRNRDIDRPVVGGVNNTTLISAA
CESLSYNVSYDVQSLDTLVFKGETYSLKHLLNNDPFTPQFEHGSILQG
FLNVTAYHRWHAPVNGTIVKIINVPGTYFAQAPSTIGDPIPDNDYDPP
PYLKSLVYFSNIAARQIMFIEADNKEIGLIFLVFIGMTEISTCEATVS
EGQHVNRGDDLGMFHFGGSSFALGLRKDCRAEIVEKFTEPGTVIRINE
VVAALKA
SEQ. ID NO: 3
ATGATCGCTGTACTATTCTCCTTCGTCATTGCAGGATGCATATACTAC
ATCGTTTCTCGTAGAGTGAGGCGGTCGCGCTTGCCACCAGGGCCGCCT
GGCATTCCTATTCCCTTCATTGGGAACATGTTTGATATGCCTGAAGAA
TCTCCATGGTTAACATTTCTACAATGGGGACGGGATTACAGTCTGTCT
TGCCGCGTTGACTTCTAATATATGAACAGCTAATATATTGTCAGACAC
CGATATTCTCTACGTGGATGCTGGAGGGACAGAAATGGTTATTCTTAA
CACGTTGGAGACCATTACCGATCTATTAGAAAAGCGAGGGTCCATTTA
TTCTGGCCGGTGAGCTGATGTTGAGTTTTTTGCAATTGAATTTGTGGT
CACACGTTTCCAGACTTGAGAGTACAATGGTCAACGAACTTATGGGGT
GGGAGTTTGACTTAGGGTTCATCACATACGGCGACAGGTGGCGCGAAG
AAAGGCGCATGTTCGCCAAGGAGTTCAGTGAGAAGGGCATCAAGCAAT
TTCGCCATGCTCAAGTGAAAGCTGCCCATCAGCTTGTCCAACAGCTTA
CCAAAACGCCAGACCGCTGGGCACAACATATTCGCCAGTAAGTACTAC
TTGAGGAAAATAGCGTACGCTTCGCTGACCGGTCCGTACATCAAAGTC
AGATAGCGGCAATGTCACTGGATATTGGTTATGGAATTGATCTTGCAG
AAGACGACCCTTGGCTGGAAGCGACCCATTTGGCTAATGAAGGCCTCG
CCATAGCATCAGTGCCGGGCAAATTTTGGGTCGATTCGTTCCCTTCTC
GTGAGCATCCTTCTTCTATGTAGGAAGGGAAGGAGTCTAACAAGTGTT
AGTAAAATACCTTCCTGCTTGGTTCCCAGGTGCTGTCTTCAAGCGCAA
AGCGAAGGTCTGGCGAGAAGCCGCCGACCATATGGTTGACATGCCTTA
TGAAACTATGAGGAAATTAGCAGTTAGTCAAATGCGTTCTCCCCGTAT
TTTTTCAATACTCTAACTTCAGCTCACAGCCTCAAGGATTGACTCGTC
CGTCGTATGCTTCAGCTCGTCTGCAAGCCATGGATCTCAACGGTGACC
TTGAGCATCAAGAACACGTAATCAAGAACACAGCCGCAGAGGTTAATG
TCGGTAAGTCAAAAGCGTCCGTCGGCAATTCAAAATTCAGGCGCTAAA
GTGGGTCTTCTCACCAAGGTGGAGGCGATACTGTAAGGATTTCTCAAT
CGTTAGAGTATAAGTGTTCTAATGCAGTACATACTCCACCAACCAGAC
TGTCTCTGCTATGTCTGCGTTCATCTTGGCCATGGTGAAGTACCCTGA
GGTCCAGCGAAAGGTTCAAGCGGAGCTTGATGCTCTGACCAATAACGG
CCAAATTCCTGACTATGACGAAGAAGATGACTCCTTGCCATACCTCAC
CGCATGTATCAAGGAGCTTTTCCGGTGGAATCAAATCGCACCCCTCGC
TATACCGCACAAATTAATGAAGGACGACGTGTACCGCGGGTATCTGAT
TCCCAAGAACACTCTAGTCTTCGCAAACACCTGGTGAGGCTGTCCATT
CATTCCTAGTACATCCGTTGCCCCACTAATAGCATCTTGATAACAGGG
CAGTATTAAACGATCCAGAAGTCTATCCAGATCCCTCTGTGTTCCGCC
CAGAAAGATATCTTGGTCCTGACGGGAAGCCTGATAACACTGTACGCG
ACCCACGTAAAGCGGCATTTGGCTATGGACGACGAAATTGGTAAGTGC
GCTTTCAGAACCCCCCCTTCCGTTGACTAGTGCCATGCGCGCATACAA
TATCGCTATTGATCTGATATAACTTCCCTGCGGCATTTATTTTGGCAT
TCCTTTAGTCCCGGAATTCATCTAGCGCAGTCGACGGTTTGGATTGCA
GGGGCAACCCTCTTATCAGCGTTCAATATCGAGCGACCTGTCGATCAG
AATGGGAAGCCCATTGACATACCGGCTGATTTTACTACAGGATTCTTC
AGGTAGCTAATTTCCGTCTTTGTGTGCATAATACCCCTAACGACGCAC
GTTTACCTTTTTGTAAAGACACCCAGTGCCTTTCCAGTGCAGGTTTGT
TCCTCGAACAGAGCAAGTCTCACAGTCGGTATCCGGACCCTGA
SEQ. ID NO: 4
MIAVLFSFVIAGCIYYIVSRRVRRSRLPPGPPGIPIPFIGNMFDMPEE
SPWLTFLQWGRDYNTDILYVDAGGTEMVILNTLETITDLLEKRGSIYS
GRLESTMVNELMGWEFDLGFITYGDRWREERRMFAKEFSEKGIKQFRH
AQVKAAHQLVQQLTKTPDRWAQHIRHQIAAMSLDIGYGIDLAEDDPWL
EATHLANEGLAIASVPGKFWVDSFPSLKYLPAWFPGAVFKRKAKVWRE
AADHMVDMPYETMRKLAPQGLTRPSYASARLQAMDLNGDLEHQEHVIK
NTAAEVNVGGGDTTVSAMSAFILAMVKYPEVQRKVQAELDALTNNGQI
PDYDEEDDSLPYLTACIKELFRWNQIAPLAIPHKLMKDDVYRGYLIPK
NTLVFANTWAVLNDPEVYPDPSVFRPERYLGPDGKPDNTVRDPRKAAF
GYGRRNCPGIHLAQSTVWIAGATLLSAFNIERPVDQNGKPIDIPADFT
TGFFRHPVPFQCRFVPRTEQVSQSVSGP
SEQ. ID NO: 5
ATGGCGTTCGATCTCAAGACTGAAGACGGCCTCATCACATATCTCACT
AAACATCTTTCTTTGGACGTCGACACGAGCGGAGTGAAGCGCCTTAGC
GGAGGCTTTGTCAATGTAACCTGGCGCATTAAGCTCAATGCTCCTTAT
CAAGGTCATACGAGCATCATCCTGAAGCATGCTCAGCCGCACATGTCT
ACGGATGAGGATTTTAAGATAGGTGTAGAACGTTCGGTTTACGAATAC
CAGGCTATCAAGCTCATGATGGCCAATCGGGAGGTTCTGGGAGGCGTG
GATGGCATAGTTTCTGTGCCAGAAGGCCTGAACTACGACTTAGAGAAT
AATGCATTGATCATGCAAGATGTCGGGAAGATGAAGACCCTTTTAGAT
TATGTCACCGCCAAACCGCCACTTGCGACGGATATAGCCCGCCTTGTT
GGGACAGAAATTGGGGGGTTCGTTGCCAGACTCCATAACATAGGCCGC
GAGAGGCGAGACGATCCTGAGTTCAAATTCTTCTCTGGAAATATTGTC
GGAAGGACGACTTCAGACCAGCTGTATCAAACCATCATACCCAACGCA
GCGAAATATGGCGTCGATGACCCCTTGCTGCCTACTGTGGTTAAGGAC
CTTGTGGACGATGTCATGCACAGCGAAGAGACCCTTGTCATGGCGGAC
CTGTGGAGTGGAAATATTCTTCTCCAGTTGGAGGAGGGAAACCCATCG
AAGCTGCAGAAGATATATATCCTGGATTGGGAACTTTGCAAGTACGGC
CCAGCGTCGTTGGACCTGGGCTATTTCTTGGGTGACTGCTATTTGATA
TCCCGCTTTCAAGACGAGCAGGTCGGTACGACGATGCGGCAAGCCTAC
TTGCAAAGCTATGCGCGTACGAGCAAGCATTCGATCAACTACGCCAAA
GTCACTGCAGGTATTGCTGCTCATATTGTGATGTGGACCGACTTTATG
CAGTGGGGGAGCGAGGAAGAAAGGATAAATTTTGTGAAAAAGGGGGTA
GCTGCCTTTCACGACGCCAGGGGCAACAACGACAATGGGGAAATTACG
TCTACCTTACTGAAGGAATCATCCACTGCGTAA
SEQ. ID NO: 6
MHIRNPYRTPIDYQALSEAFPPLKPFVSVNADGTSSVDLTIPEAQRAF
TAALLHRDFGLTMTIPEDRLCPTVPNRLNYVLWIEDIFNYTNKTLGLS
DDRPIKGVDIGTGASAIYPMLACARFKAWSMVGTEVERKCIDTARLNV
VANNLQDRLSILETSIDGPILVPIFEATEEYEYEFTMCNPPFYDGAAD
MQTSDAAKGFGFGVGAPHSGTVIEMSTEGGESAFVAQMVRESLKLRTR
CRWYTSNLGKLKSLKEIVGLLKELEISNYAINEYVQGSTRRYAVAWSF
TDIQLPEELSRPSNPELSSLF
SEQ. ID NO: 7
ATGCATATCAGAAATCCTTACCGTACACCAATTGACTATCAAGCACTT
TCAGAGGCCTTCCCTCCCCTCAAGCCATTTGTGTCTGTCAATGCAGAT
GGTACCAGTTCTGTTGACCTCACTATCCCAGAAGCCCAGAGGGCGTTC
ACGGCCGCTCTTCTTCATCGTGACTTCGGGCTCACCATGACCATACCA
GAAGACCGTCTGTGCCCAACAGTCCCCAATAGGTTGAACTACGTTCTG
TGGATTGAAGATATTTTCAACTACACGAACAAAACCCTCGGCCTGTCG
GATGACCGTCCTATTAAAGGCGTTGATATTGGTACAGGAGCCTCCGCA
ATTTATCCTATGCTTGCCTGTGCTCGGTTCAAGGCATGGTCTATGGTT
GGAACAGAGGTCGAGAGGAAGTGCATTGACACGGCCCGCCTCAATGTC
GTCGCGAACAATCTCCAAGACCGTCTCTCGATATTAGAGACATCCATT
GATGGTCCTATTCTCGTCCCCATTTTCGAGGCGACTGAAGAATACGAA
TACGAGTTTACTATGTGTAACCCTCCATTCTACGACGGTGCTGCCGAT
ATGCAGACTTCGGATGCTGCCAAAGGATTTGGATTTGGCGTGGGCGCT
CCCCATTCTGGAACAGTCATCGAAATGTCGACTGAGGGAGGTGAATCG
GCTTTCGTCGCTCAGATGGTCCGTGAGAGCTTGAAGCTTCGAACACGA
TGCAGATGGTACACGAGTAACTTGGGAAAGCTGAAATCCTTGAAAGAA
ATAGTGGGGCTGCTGAAAGAACTTGAGATAAGCAACTATGCCATTAAC
GAATACGTTCAGGGGTCCACACGTCGTTATGCCGTTGCGTGGTCTTTC
ACTGATATTCAACTGCCTGAGGAGCTTTCTCGTCCCTCTAACCCCGAG
CTCAGCTCTCTTTTCTAG
SEQ. ID NO: 8
MHIRNPYRTPIDYQALSEAFPPLKPFVSVNADGTSSVDLTIPEAQRAF
TAALLHRDFGLTMTIPEDRLCPTVPNRLNYVLWIEDIFNYTNKTLGLS
DDRPIKGVDIGTGASAIYPMLACARFKAWSMVGTEVERKCIDTARLNV
VANNLQDRLSILETSIDGPILVPIFEATEEYEYEFTMCNPPFYDGAAD
MQTSDAAKGFGFGVGAPHSGTVIEMSTEGGESAFVAQMVRESLKLRTR
CRWYTSNLGKLKSLKEIVGLLKELEISNYAINEYVQGSTRRYAVAWSF
TDIQLPEELSRPSNPELSSLF
SEQ. ID NO: 9
ATGCCTTCCAGTCACCCTCACATTACTCATCGCTATCGGGTTCCTTCG
AGTGACGACCATGAACGTATATCTGCTCTGTTCTTGGGTCCCAAAGCA
GAAAATGCCGCATTTCTCCAGCAATGGTTGACCACGGTCGTCGCACAG
CAAAAGGCTGCCCGCGATGCATACTTCCCGGATGACAATGCTTTTATT
ACTACAGACATGCAAACTTCCCCCGCCTTTGCTCAGACTACTAAAGTA
ATCGCCTCCAATCTCACCGAATTATTGACTGCACTCGGTGAAAGGTCG
ATTCCTTTCTTCTCACCTCGGTACAGCGGCCATATGTCTGTGGACCAA
AGTCTACCTGCCATTCTCGGATTCTTATCGACCACATTTTATAATCCT
AACAATGTTGCCTTCGAGGCTAGTCCATTCACGACCCTCATCGAGGAA
GAAGTTGGCTTGCAACTCTCTGAAATGCTGGGTTATAATCGGCTAAAT
AACACCGAGAAACCTCTCGCCTGGGGACATATTGCATCAGGTGGAACT
GTTGCAAACTTGGAAGCGATGTGGGCGGCGCGAAACCTCAAGTTTTAC
CCTCTCTCACTCCGTGATGCTTCAGCCGAAGGCGCAGAGATGGAATTC
ATTCGTGACACATTCTCCGTCAAAACCTGTGTTGGTGACAAAAAATTA
TTAAAGGATTGCAGCCCATGGGAACTCCTCAATTTGCATGTTTCTACT
ATCTTAGACATGCCCGACCGTCTGCACGACGAGTACAATATTTCACCT
CAGTTCCTCGAAAAGGTTATGCGAAAGTATATCATCCAGTCTACCAAC
AAAGACACGTTGATGCAGCGTTGGGGACTTACCCAACAACCTGTCGTT
TTATCCCCGAGCACAAACCATTATTCCTGGCCAAAGGCTGCAGCTGTG
CTCGGTATTGGCTCAGACAACCTTCGCAACGTCCCAGTAGACATCCAA
GCCCACATGGACATAAACGAACTCGATCGTATGTTAAAAATTTGCTTG
GACGAGGAGACGCCAGTATATCAAGTAGTTGCTGTTATCGGTACCACC
GAAGAGGGCGGTGTCGATCGCATTACGGAGATCCTGAAGCTGCGCCAA
AAGTATGAAGCTTTGGGGCTGTCTTTTGCCATCCATGCAGATGCTGCT
TGGGGAGGCTATTTTGCAACCATGCTACCCAAAGATACATTGGGTCGA
AACCGGACTAGGCTTCCCAAAGAGGACACTACCTCGGGCTTTGTCCCT
CACGTCGGTCTGCGCGAGGAGAGCGCGTTACAACTCAGCCATATAAAG
TATGCCGATTCTATTACTATCGACCCGCACAAGGCAGGCTATGTTCCT
TACCCCGCTGGGGCACTCTGTTATCGCGACGGAAGAATGAGGTACCTG
CTTACATGGTCCGCGCCCTACCTTGCCCAAGGCAACGAGGGCCAAAGT
ATCGGAATATACGGGATCGAAGGAAGCAAACCTGGTGCAGCAGCATCC
GCGGTATTCATGGCGCACGAAACCATTGGCCTGACTCCTTCTGGATAC
GGGAACCTTCTTGGCCAGGCAATGTTTACATGTCGCCGATACGCTGCT
CACTGGTCTGCAATGTCAACGGATACTACCAGTTTCACTGTCACCCCG
TTCAATCCTATCCCTGCTGACATCGACCCCAACGCTGACCCCGCAAAG
GTCGAAGAGCAAAAACAGTTCATCAGAGATCGTATCTTGTTCAAATCG
AACGAGGAAATATACAACGATTCTGAGGCTATGGAACTCTTGCACCAA
CTTGGGTCCGATCTCAATATCAACGTTTTCGCATGCAACTTCCGCGAC
CGCGATAATAATCTCAACACCGACGTCGAGGAAGCCAACTGGCTCAAT
AACCGTATTTTCCAACGCTTTTCTGTTACAAGTGCTGAGGAGAACCCA
TTGGAAACGCCATTCTTCCTCAGCTCAACTACATTGAAACAATCCGAA
TACGGCGTCTGCGCAACCGAAGTAAAGAGACGCATGGGACTTGTTGGT
GACCAGGATGTTATAGTCCTGAGGAACGTCGTTATGTCTCCATTTACT
ACAACGAACGACTTTGTGGGAACTCTGGCAAACACCTTCCAAAAGATC
GTTGAGGAGGAGGTCGAGTATGCACGGATCCGCAACGATATGAAACCT
AGCATTCACACCTTCCTTCTTCATGGTTCAGGAGAGCAATACTATCTT
GTCCACACCCCAACGATCCATATGGCCAGCGGCCGTCGCCAAATCATC
CTTTCAGTAAATGTTGAAGGCCAAGTTCGGCAGGCGATACATGCCCAT
GAAAGAGTTGAAGCAGTGATTGTACATAACACTGTGCCCCTCCGCCTT
GACGAAATCGTTGACGGAGGATCATTTGACGGCATACTCACCATCGGA
AAGAGGAAAACTAGTTTCAAAGTGAAGATTTCAAACATTAAAGTAGTC
AAGAAGCGCTCTCTGATGACTGAGGACCTGGAATCTGCGTACCCATCG
TTGATGCCATTCTATTTCTACGGGACTCAAGGACACGCTCATCTCGAC
CATGTCATTACTGTCGTTCCTAACATCCATCTGAGTGCTGGCGAAATA
CAGTACAAATTCGACGACGAGGTGTCAAGCGAGGACCTCGCCAAGGGC
CTCATTGTTGTTGCTGAGAACGTACACGAGGCATCCATGCAGCCCTTC
CCGCTCATGAAAGATTTCAAGATCACCAACCAATTCTTCTTCAGCTCC
GGGCAAATACTCCGCGTCAAAGTGTACAGAGATCCATACCCGGCATCG
ACAATGGATCCCATCCCTCTCCACGACATCAAGAACCAGCCCGTCGTG
ACACAAGGCACCATCACGCTCGTCGGAAATATTTACGTCGATTCTGAT
GCGCTCAACGTCGCTTCCGAGCCTACTGCCGACGAAGACGCGGCGCAT
GTTCCTCACGCTCGCAACATGTACGGCGAGATGACCGCTGGAACGATC
AAAGGCTGGCAAAACGCTGTTCGTCATTTCCACAACAAATTGGAGACT
GTTGCTCCGACGAAGTAG
SEQ. ID NO: 10
MPSSHPHITHRYRVPSSDDHERISALFLGPKAENAAFLQQWLTTVVAQ
QKAARDAYFPDDNAFITTDMQTSPAFAQTTKVIASNLTELLTALGERS
IPFFSPRYSGHMSVDQSLPAILGFLSTTFYNPNNVAFEASPFTTLIEE
EVGLQLSEMLGYNRLNNTEKPLAWGHIASGGTVANLEAMWAARNLKFY
PLSLRDASAEGAEMEFIRDTFSVKTCVGDKKLLKDCSPWELLNLHVST
ILDMPDRLHDEYNISPQFLEKVMRKYIIQSTNKDTLMQRWGLTQQPVV
LSPSTNHYSWPKAAAVLGIGSDNLRNVPVDIQAHMDINELDRMLKICL
DEETPVYQVVAVIGTTEEGGVDRITEILKLRQKYEALGLSFAIHADAA
WGGYFATMLPKDTLGRNRTRLPKEDTTSGFVPHVGLREESALQLSHIK
YADSITIDPHKAGYVPYPAGALCYRDGRMRYLLTWSAPYLAQGNEGQS
IGIYGIEGSKPGAAASAVFMAHETIGLTPSGYGNLLGQAMFTCRRYAA
HWSAMSTDTTSFTVTPFNPIPADIDPNADPAKVEEQKQFIRDRILFKS
NEEIYNDSEAMELLHQLGSDLNINVFACNFRDRDNNLNTDVEEANWLN
NRIFQRFSVTSAEENPLETPFFLSSTTLKQSEYGVCATEVKRRMGLVG
DQDVIVLRNVVMSPFTTTNDFVGTLANTFQKIVEEEVEYARIRNDMKP
SIHTFLLHGSGEQYYLVHTPTIHMASGRRQIILSVNVEGQVRQAIHAH
ERVEAVIVHNTVPLRLDEIVDGGSFDGILTIGKRKTSFKVKISNIKVV
KKRSLMTEDLESAYPSLMPFYFYGTQGHAHLDHVITVVPNIHLSAGEI
QYKFDDEVSSEDLAKGLIVVAENVHEASMQPFPLMKDFKITNQFFFSS
GQILRVKVYRDPYPASTMDPIPLHDIKNQPVVTQGTITLVGNIYVDSD
ALNVASEPTADEDAAHVPHARNMYGEMTAGTIKGWQNAVRHFHNKLET
VAPTK
SEQ. ID NO: 11
ATGGAGGCTATCAAAAAGGTTTTTGAGAACAAAAAGGCGGAGGGCATT
CCTGTGTTGGTGACCTTTGTTACTGCAGGATATCCTCGTCCCGAAGAT
ACTGTTCCCATCTTGCTGGCCATGGAGGCCGGTGGTGCTGATATCATC
GAGCTTGGTATGCCATTTTCAGACCCAATTGCAGATGGTCCTGTCATC
CAGGAAACGAACACAATCGCCGTTGCAAACCAGGTAGATTATACCACT
GTTCTCGGACAACTTCGGGAAGCCCGCAAACAAGGGCTCAAGGCACCC
GTTCTTCTGATGGGATATTATAACCCCATATTGGCTTACGGAGAAGAC
AGATCTATTCAAGATGCGGCTGAAGCTGGAGCCAATGGGTTTATTATG
GTCGACCTTCCACCCGAGGAGGCTGTCGCTTTTCGAGAGAAATGTATC
AAATCCAACCTCTCATATGTTCCTCTAATTGCACCCTCAACGACTCTG
TCGCGTATAAAGTTCCTCTCAACAATTGCAGACACGTTCATCTATGTC
GTGTCTAAAATGGGAACCACCGGATCCTCAGAGAAGGTTGCCATGAAT
AACGCCCTTCCCACCATCATCGATCGTATTCGCGAGTACGCTGAAGTT
CCTTTAGCAGTCGGATTTGGAGTCGCCACTCGGGCTCACTTCAACTAC
GTCGCCGATTCCGGTGCTGATGGTGTCGTTATTGGCACCAAACTCGTT
AACGTTATTAAAGAGTCACCGCAAGGGGAAGCACCCAAAAATGTTGAG
GCATACTGCCGTGAGATGAGCCAAAAGGGAGAAACAAATCGCGTCAAA
TCTCCACCAACTGCCCGTGCTGCCAGCTCCGAATCAATTCCTGTTGTT
GTTCCTTCTGTTCTCCCCGCACGTTTCGGAGAATTCGGAGGACAATAC
GTTCCCGAAGCTCTTGTCGATTGTCTGGTTGAACTAGAAGAAGCTCAC
AAATCTGCCATGGCTGATCCTGAATTCCAGAAGGAACTACAATCGCAT
GCCGGATATGCAAATCGTCCTTCACAAATATACCTCGCCGAAAATCTC
ACCAAGGATGCTGGGGGTGCAAATATTTGGTTGAAACGTGAAGATTTG
AACCACACAGGTTCCCACAAAATCAATAACGCTTTGGGACAAATTCTG
CTTGCCCGGAGAATCGGAAAGACCAGAATTATCGCAGAAACAGGTGCC
GGCCAGCATGGTGTTGCAACAGCGACTGTTTGCGCTAAGTTTGGAATG
GAATGTGTTATCTACATGGGCGCAGAAGATGTGCGACGGCAAGCTCTA
AATGTATTCAGGATTGAGATGCTAGGAGCAAAAGTTGTTCCTGTTACT
TCAGGATCATGCACATTGAAGGACGCTGTAAACGAGGCCTTCCGTGAC
TGGGTGACAAACCTTTCTACGACGCATTATTTGGTTGGCTCTGTAATT
GGACCTCATCCCTTCCCCACCATTGTCCGAGATTTCCAAAAGGTCATT
GGTCAAGAGATCAAGGCTCAGATGTTGGCCGCCCGCGGCAAACTTCCT
GATGTCGTCGTCGCTTGTGTTGGTGGAGGAAGCAATGCTATCGGTACG
TTCTATGATTTTATTGGCGACAAGAGTGTACGTCTAGTTGGGGTGGAA
GCAGGAGGAGAAGGTATTGACGGAGACCGACATAGCGCCACACTTTCG
ATGGGGCAACCGGGAGTACTTCACGGTGTTAGAACATATATTCTACAA
GACAAGGCCGGTCAAATCATCGAGACGCACTCAATCAGCGCTGGATTG
GATTATCCCGGCGTTGGACCAGAACATGCTTGGCTAAAGGACTCTAAA
AGAGCAGAATATGTTGTCGCCACAGACGAAGAAGCACTTCGCGGTTTC
CGTATGCTAACACAAAGGGAGGGAATTATTCCTGCCCTTGAATCTTCC
CATGCGATCTGGGAGGCTGTCAGGATTGCCCGCACCATGTCGAAGGAC
CAGGATCTTGTTGTGTGTTTGTCTGGCCGAGGTGATAAAGACGTTGAG
CAAATTTCTCAACTTCTTCCCAAGTGGGCGGATATTCTAGACTGGCAT
GTTTCTTCCCATGCCGTTGGACACACAACAAAATTCTAA
SEQ. ID NO: 12
MEAIKKVFENKKAEGIPVLVTFVTAGYPRPEDTVPILLAMEAGGADII
ELGMPFSDPIADGPVIQETNTIAVANQVDYTTVLGQLREARKQGLKAP
VLLMGYYNPILAYGEDRSIQDAAEAGANGFIMVDLPPEEAVAFREKCI
KSNLSYVPLIAPSTTLSRIKFLSTIADTFIYVVSKMGTTGSSEKVAMN
NALPTIIDRIREYAEVPLAVGFGVATRAHFNYVADSGADGVVIGTKLV
NVIKESPQGEAPKNVEAYCREMSQKGETNRVKSPPTARAASSESIPVV
VPSVLPARFGEFGGQYVPEALVDCLVELEEAHKSAMADPEFQKELQSH
AGYANRPSQIYLAENLTKDAGGANIWLKREDLNHTGSHKINNALGQIL
LARRIGKTRIIAETGAGQHGVATATVCAKFGMECVIYMGAEDVRRQAL
NVFRIEMLGAKVVPVTSGSCTLKDAVNEAFRDWVTNLSTTHYLVGSVI
GPHPFPTIVRDFQKVIGQEIKAQMLAARGKLPDVVVACVGGGSNAIGT
FYDFIGDKSVRLVGVEAGGEGIDGDRHSATLSMGQPGVLHGVRTYILQ
DKAGQIIETHSISAGLDYPGVGPEHAWLKDSKRAEYVVATDEEALRGF
RMLTQREGIIPALESSHAIWEAVRIARTMSKDQDLVVCLSGRGDKDVE
QISQLLPKWADILDWHVSSHAVGHTTKF
SEQ. ID NO: 13
ATGGAAAGGCGGAAGCGTGAGCGTCTTGGCTCACTCGGCCGACCGACC
AAAAAGGAGCTCCGCATGATCCGATCTGTGGTGATCGTGGGTGGTGGC
ACGGCGGGCTGGATGACCGCCTCCTACCTCAAGGCCGCCTTCGACGAC
CGCATCGACGTAACGCTCGTGGAGTCAGGGAACGTCAGGCGGATCGGG
GTCGGCGAAGCGACCTTCAGCACGGTCCGCCACTTCTTCGACTACCTG
GGCCTCGACGAGCGCGAGTGGCTGCCCCGCTGCGCCGGCGGCTACAAG
CTCGGCATCCGCTTCGAGAACTGGAGCGAGCCGGGCGAGTACTTCTAC
CACCCGTTCGAGCGCCTGCGCGTCGTCGACGGCTTCAACATGGCCGAG
TGGTGGCTCGCGGTCGGCGACCGCAGGACGTCCTTCAGCGAGGCCTGC
TATCTCACGCACCGGCTGTGCGAGGCCAAGCGGGCGCCCCGCATGCTC
GACGGCTCGCTCTTCGCCTCCCAGGTGGACGAGTCGCTCGGCCGCTCG
ACCCTGGCCGAGCAGCGCGCCCAGTTCCCGTACGCCTACCACTTCGAC
GCCGACGAGGTCGCCCGCTACCTGTCGGAGTACGCCATCGCCCGCGGC
GTCCGCCACGTGGTCGACGACGTGCAGCACGTCGGCCAGGACGAGCGC
GGCTGGATCAGCGGCGTCCACACCAAGCAGCACGGCGAGATCAGCGGC
GACCTGTTCGTCGACTGCACCGGCTTCCGCGGCCTGCTCATCAACCAG
ACGCTGGGCGGCAGGTTCCAGTCCTTCTCCGACGTGCTGCCCAACAAC
CGGGCGGTCGCGCTGCGCGTCCCGCGGGAGAACGACGAGGACATGCGG
CCGTACACGACGGCGACCGCGATGAGCGCCGGCTGGATGTGGACGATC
CCGCTGTTCAAGCGCGACGGCAACGGCTACGTCTACTCCGACGAGTTC
ATCTCGCCGGAGGAGGCCGAGCGCGAGCTGCGGTCCACCGTCGCCCCC
GGCCGCGACGACCTGGAGGCCAACCACATCCAGATGCGGATCGGCAGG
AACGAGCGCACCTGGATCAACAACTGCGTCGCCGTCGGCCTGTCCGCC
GCCTTCGTCGAGCCGCTGGAGTCGACCGGCATCTTCTTCATCCAGCAC
GCCATCGAGCAGCTCGTGAAGCACTTCCCCGGCGAGCGCTGGGACCCG
GTGCTGATCAGCGCGTACAACGAGCGCATGGCGCACATGGTCGACGGG
GTCAAGGAGTTCCTCGTCCTCCACTACAAGGGCGCCCAGCGCGAGGAC
ACCCCCTACTGGAAGGCCGCCAAGACCAGGGCCATGCCCGACGGCCTC
GCCCGCAAGCTGGAGCTGTCCGCCTCCCACCTGCTGGACGAGCAGACG
ATCTACCCCTACTACCACGGCTTCGAGACCTATTCGTGGATCACCATG
AACCTCGGTCTCGGCATCGTGCCCGAGCGGCCGCGTCCCGCGCTCCTG
CACATGGACCCGGCGCCCGCGCTGGCCGAGTTCGAACGGCTCAGGCGC
GAGGGCGACGAGCTGATCGCCGCCCTGCCCAGCTGCTACGAGTACCTC
GCCAGCATCCAATGA
SEQ. ID NO: 14
MERRKRERLGSLGRPTKKELRMIRSVVIVGGGTAGWMTASYLKAAFDD
RIDVTLVESGNVRRIGVGEATFSTVRHFFDYLGLDEREWLPRCAGGYK
LGIRFENWSEPGEYFYHPFERLRVVDGFNMAEWWLAVGDRRTSFSEAC
YLTHRLCEAKRAPRMLDGSLFASQVDESLGRSTLAEQRAQFPYAYHFD
ADEVARYLSEYAIARGVRHVVDDVQHVGQDERGWISGVHTKQHGEISG
DLFVDCTGFRGLLINQTLGGRFQSFSDVLPNNRAVALRVPRENDEDMR
PYTTATAMSAGWMWTIPLFKRDGNGYVYSDEFISPEEAERELRSTVAP
GRDDLEANHIQMRIGRNERTWINNCVAVGLSAAFVEPLESTGIFFIQH
AIEQLVKHFPGERWDPVLISAYNERMAHMVDGVKEFLVLHYKGAQRED
TPYWKAAKTRAMPDGLARKLELSASHLLDEQTTYPYYHGFETYSWITM
NLGLGIVPERPRPALLHMDPAPALAEFERLRREGDELIAALPSCYEYL
ASIQ
SEQ. ID NO: 15
ATGACCGCCGCCTACCTGAAGACCGCGTTCGGCGACCGGCTTTCGATC
ACCGTCGTGGAATCGAGCAGAATCGGGACCATCGGCGTCGGTGAGGCG
ACCTTCAGCGACATCCAGCACTTCTTCCAGTTCCTCAACCTCCGGGAA
CAGGACTGGATGCCGGCCTGCAACGCCACCTACAAGCTGGGCATCCGC
TTCGAGAACTGGCGGCATGTCGGCCATCATTTCTACCAGCCGTTCGAG
CAGATCCGCCCGGTCTACGGCTTTCCGCTGACCGACTGGTGGCTGCAC
GACGCGCCGACCGACCGCTTCGACACCGACTGCTTCGTCATGCCCAAC
CTGTGCGAGGCCGGGCGCAGCCCGCGCCACCTCGACGGCACGCTGGCC
GACGAGGACTTCGTGGAGGAGGGCGACGAGCTCGCCAACCGCACCATG
TCCGAGCACCAGGGCAAGTCGCAGTTCCCGTACGCCTACCACTTCGAG
GCGGCGCTGCTGGCCAAGTTCCTCACCGGGTACGCGGTGGACCGCGGC
GTCGAGCACGTCGTCGACGACGTCCTGGACGTGCGGCTGGACCAGCGC
GGGTGGATCGAGCACGTGGTCACCGCCGAGCACGGCGAGATCCACGGC
GACCTGTTCGTCGACTGCACCGGTTTCCGGGGACTGCTGCTGAACAAG
GCGCTCGGTGTGCCGTTCGTGTCCTATCAGGACACTCTGCCCAACGAC
AGCGCGGTCGCCCTCCAGGTCCCGCTGGACATGCAGCGGCGCGGCATC
GTGCCGAACACCACGGCCACCGCGCGGGAGGCCGGCTGGATCTGGACC
ATCCCGCTGTTCGGTCGGGTCGGCACCGGGTACGTCTACGCCAAGGAC
TACCTCTCGCCCGAGGAGGCCGAGCGCACGCTGCGCGAGTTCGTCGGC
CCGGCGGCCGCGGACGTGGAGGCCAACCACATCCGCATGCGGATCGGC
CGCAGCCAGGAGTCCTGGCGGAACAACTGCGTGGCCATCGGCCTGTCC
AGCGGCTTCGTCGAGCCGCTGGAGTCGACCGGGATCTTCTTCATCCAC
CACGCCATCGAGCAGCTGGTGAAGCACTTCCCGGCGGCCGACTGGAAC
CCGAAGTCCCGGGACATGTACAACTCCGCGGTCGCGCACGTGATGGAC
GGCATCCGCGAGTTCCTGGTGATCCACTACCGGGGCGCGGCGCGGGCC
GACAACCAGTACTGGCGCGACACCAAGACCCGCCCGCTGCCGGACGGC
CTGGCCGAGCGCATCGAGTGCTGGCAGACGCAGCTGCCGGACACCGAG
ACGATCTACCCGTACTACCACGGGCTGCCGCCCTACTCCTACATGTGC
ATCCTGATGGGCGGCGGGGCCATCCGGACGCCGGCCTCGGCCGCGTTG
GCCCTGACCGACCAGGGGGCCGCGCAGAAGGAGTTCGCCGCCGTCCGG
GACCGGGCCGCGCAGCTGCGCGACACGCTGCCCAGCCACTACGAGTAC
CTCGCGCGGATGCGTGGTCTGGATGTCTGA
SEQ. ID NO: 16
MTAAYLKTAFGDRLSITVVESSRIGTIGVGEATFSDIQHFFQFLNLRE
QDWMPACNATYKLGIRFENWRHVGHHFYQPFEQIRPVYGFPLTDWWLH
DAPTDRFDTDCFVMPNLCEAGRSPRHLDGTLADEDFVEEGDELANRTM
SEHQGKSQFPYAYHFEAALLAKFLTGYAVDRGVEHVVDDVLDVRLDQR
GWIEHVVTAEHGEIHGDLFVDCTGFRGLLLNKALGVPFVSYQDTLPND
SAVALQVPLDMQRRGIVPNTTATAREAGWIWTIPLFGRVGTGYVYAKD
YLSPEEAERTLREFVGPAAADVEANHIRMRIGRSQESWRNNCVAIGLS
SGFVEPLESTGIFFIHHAIEQLVKHFPAADWNPKSRDMYNSAVAHVMD
GIREFLVIHYRGAARADNQYWRDTKTRPLQKEFAAVRDRAAQLRDTLP
SHYEYLARMRGLDV
SEQ. ID NO: 17
ATGAACACGAGAAATCCGGACAAGGTCGTCATCGTCGGCGGCGGCACG
GCGGGATGGATGACCGCCTCGTACCTGAAGAAGGCGTTCGGCGAGCGC
GTGTCCGTGACCCTCGTCGAATCGGGCACCATCGGCACCGTCGGCGTC
GGCGAGGCGACCTTCAGCGACATCCGGCACTTCTTCGAATTCCTCGAC
CTGCGGGAGGAGGAATGGATGCCGGCCTGCAACGCCACGTACAAGCTG
GCCGTCCGCTTCCAGGACTGGCAGCGCCCCGGCCACCACTTCTACCAC
CCGTTCGAGCAGATGCGCTCCGTAGACGGGTTCCCCCTGACCGACTGG
TGGCTGCAGAACGGGCCGACCGACCGGTTCGACAGGGACTGCTTCGTG
ATGGCGTCGCTGTGCGACGCGGGGCGCAGCCCGCGCTATCTCAACGGT
TCCCTGCTGCAGCAGGAGTTCGACGAGCGCGCGGAGGAGCCGGCGGGG
CTCACCATGTCCGAGCACCAGGGCAAGACGCAGTTCCCCTACGCCTAC
CACTTCGAGGCGGCCCTGCTCGCCGAATTCCTTTCTGGGTACTCCAAG
GACCGCGGCGTGAAGCACGTGGTCGACGAGGTGCTGGAGGTGAAGCTC
GACGACCGCGGATGGATCAGCCACGTTGTCACCAAGGAGCACGGCGAC
ATCGGCGGCGACCTGTTCGTCGACTGCACCGGTTTCCGCGGCGTACTG
CTGAACCAGGCGCTGGGTGTGCCCTTCGTCTCCTACCAGGACACCCTG
CCCAATGACAGCGCGGTCGCCCTCCAGGTGCCGCTCGACATGGAGGCG
CGGGGCATCCCGCCGTACACCCGGGCCACCGCCAAGGAGGCCGGGTGG
ATCTGGACGATCCCGCTGATCGGCCGGATCGGCACCGGGTACGTGTAC
GCCAAGGACTACTGCTCGCCGGAGGAGGCCGAGCGCACCCTGCGCGAG
TTCGTCGGTCCGGAGGCCGCCGACGTCGAGGCCAACCACATCCGCATG
CGGATCGGGCGCAGCGAGCAGTCCTGGAAGAACAACTGCGTCGCGATC
GGGCTCTCCAGTGGCTTCGTCGAACCCCTCGAGTCGACCGGCATCTTC
TTCATCCACCACGCCATCGAGCAGTTGGTGAAGCACTTCCCGGCGGGC
GACTGGCACCCGCAGCTGCGCGCCGGCTACAACTCGGCCGTCGCCAAC
GTCATGGACGGGGTCCGGGAGTTCCTCGTCCTGCACTACCTGGGCGCC
GCGCGCAACGACACCCGGTACTGGAAGGACACCAAGACCCGGGCGGTT
CCGGATGCCCTGGCGGAGCGCATCGAGCGCTGGAAGGTGCAGCTGCCC
GACTCCGAGAACGTCTTCCCCTACTACCACGGCCTGCCGCCGTACTCG
TACATGGCCATCCTGCTCGGGACCGGCGCGATCGGGCTGCGACCGTCG
CCGGCGCTCGCACTGGCCGACCCGGCGGCGGCGGAGAAGGAGTTCACC
GCGATCCGGGACAGGGCGCGGTTCCTGGTCGACACCCTGCCGAGCCAG
TACGAGTACTTCGCCGCGATGGGGCAGCGTGTCTGA
SEQ. ID NO: 18
MNTRNPDKVVIVGGGTAGWMTASYLKKAFGERVSVTLVESGTIGTVGV
GEATFSDIRHFFEFLDLREEEWMPACNATYKLAVRFQDWQRPGHHFYH
PFEQMRSVDGFPLTDWWLQNGPTDRFDRDCFVMASLCDAGRSPRYLNG
SLLQQEFDERAEEPAGLTMSEHQGKTQFPYAYHFEAALLAEFLSGYSK
DRGVKHVVDEVLEVKLDDRGWISHVVTKEHGDIGGDLFVDCTGFRGVL
LNQALGVPFVSYQDTLPNDSAVALQVPLDMEARGIPPYTRATAKEAGW
IWTIPLIGRIGTGYVYAKDYCSPEEAERTLREFVGPEAADVEANHIRM
RIGRSEQSWKNNCVAIGLSSGFVEPLESTGIFFIHHAIEQLVKHFPAG
DWHPQLRAGYNSAVANVMDGVREFLVLHYLGAARNDTRYWKDTKTRAV
PDALAERIERWKVQLPDSENVFPYYHGLPPYSYMAILLGTGAIGLRPS
PALALADPAAAEKEFTAIRDRARFLVDTLPSQYEYFAAMGQRV
SEQ. ID NO: 19
ATGGACAATCGAATCAAGACAGTCGTGATCCTGGGCGGCGGCACCGCC
GGCTGGATGACTGCGGCGTATCTCGGCAAGGCGCTGCAGAACACGGTG
AAGATCGTGGTCCTGGAGGCGCCCACCATCCCGCGGATCGGTGTGGGC
GAAGCCACCGTCCCCAACCTGCAGCGCGCGTTCTTCGACTACCTCGGC
ATCCCCGAGGAGGAGTGGATGCGCGAGTGCAACGCCAGTTACAAGATG
GCGGTGAAGTTCATCAACTGGCGCACACCGGGGGAGGGCAGCCCGGAT
CCCCGGACGCTCGACGACGGTCACACCGACACCTTCCACCACCCCTTC
GGACTGCTGCCGTCCGCCGACCAGATACCGCTCTCCCACTACTGGGCG
GCCAAGCGGCTCCAGGGCGAGACCGACGAGAACTTCGACGAGGCGTGT
TTCGCCGACACCGCGATCATGAACGCCAAGAAGGCGCCCCGCTTCCTC
GACATGCGGCGGGCGACCAACTACGCCTGGCACTTCGACGCCAGCAAG
GTCGCCGCGTTCCTGCGCAACTTCGCCGTCACCAAGCAGGCGGTCGAG
CACGTCGAGGACGAGATGACCGAGGTCCTCACCGACGAACGGGGCTTC
ATCACCGCCCTGCGCACCAAGTCGGGACGGATCCTGCAGGGTGACCTC
TTCGTCGACTGCTCCGGCTTCCGCGGGCTGCTGATCAACAAGGCCATG
GAGGAGCCGTTCATCGACATGAGCGACCACCTCCTGTGCAACAGCGCG
GTCGCCACGGCCGTACCGCACGACGACGAGAAGAACGGTGTCGAGCCG
TACACCTCCTCGATCGCCATGGAGGCCGGCTGGACCTGGAAGATTCCC
ATGCTGGGCCGGTTCGGCAGCGGTCACGTCTACTCCGACCACTTCGCC
ACCCAGGACGAGGCCACCCTGGCCTTCTCGAAGCTGTGGGGCCTCGAC
CCGGACAACACCGAGTTCAACCACGTCCGCTTCCGGGTCGGCCGCAAC
CGCAGGGCCTGGGTGCGCAACTGCGTCAGCGTCGGCCTCGCGTCGTGC
TTCGTGGAGCCGCTGGAGTCCAGCGGCATCTACTTCATCTACGCGGCC
ATCCACATGCTGGCCAAGCACTTCCCCGACAAGACCTTCGACAAGGTG
CTCGTCGACCGGTTCAACCGGGAGATCGAGGAGATGTTCGACGACACC
CGGGACTTCCTCCAGGCGCACTACTACTTCTCGCCGCGCGTCGACACG
CCGTTCTGGCGGGCGAACAAGGAACTGAAGCTGGCCGACTCCATCAAG
GACAAGGTCGAGACGTACCGGGCCGGGCTGCCGGTCAACCTGCCGGTC
ACCGACGAGGGGACCTACTACGGCAACTTCGAGGCCGAGTTCCGGAAC
TTCTGGACCAACGGCAGCTACTACTGCATCTTCGCCGGCCTCGGCCTG
ATGCCGCGCAACCCGCTGCCCGCGCTCGCCTACAAGCCGCAGTCGATC
GCGGAGGCCGAGCTGCTGTTCGCCGACGTCAAGCGCAAGGGGGACACC
CTGGTCGAGTCGCTGCCGTCGACCTATGACCTGCTGCGTCAGTTGCAC
GGTGCGTCGTGA
SEQ. ID NO: 20
MDNRIKTVVILGGGTAGWMTAAYLGKALQNTVKIVVLEAPTIPRIGVG
EATVPNLQRAFFDYLGIPEEEWMRECNASYKMAVKFINWRTPGEGSPD
PRTLDDGHTDTFHHPFGLLPSADQIPLSHYWAAKRLQGETDENFDEAC
FADTAIMNAKKAPRFLDMRRATNYAWHFDASKVAAFLRNFAVTKQAVE
HVEDEMTEVLTDERGFITALRTKSGRILQGDLFVDCSGFRGLLINKAM
EEPFIDMSDHLLCNSAVATAVPHDDEKNGVEPYTSSIAMEAGWTWKIP
MLGRFGSGHVYSDHFATQDEATLAFSKLWGLDPDNTEFNHVRFRVGRN
RRAWVRNCVSVGLASCFVEPLESSGIYFIYAAIHMLAKHFPDKTFDKV
LVDRFNREIEEMFDDTRDFLQAHYYFSPRVDTPFWRANKELKLADSIK
DKVETYRAGLPVNLPVTDEGTYYGNFEAEFRNFWTNGSYYCIFAGLGL
MPRNPLPALAYKPQSIAEAELLFADVKRKGDTLVESLPSTYDLLRQLH
GAS
SEQ. ID NO: 21
ATGGACAACCGCATCAACCGGATCGTCATCCTCGGCGGCGGGACCGCG
GGATGGATGACCGCCTCCTACCTGGCGAAGGCGCTCGGTGACACCGTC
ACGATCACCCTGCTGGAAGCACCGGCGATCGGCCGGATCGGGGTCGGC
GAGGCCACCGTGCCCAACCTGCAGCGCGTGTTCTTCGACTTCCTCGGC
CTCCGCGAGGAGGAGTGGATGCCGGAGTGCAACGCCGCGTTCAAGACG
GCCGTGAAGTTCATCAACTGGCGTACCCCCGGGCCCGGCGAGGCCAAG
GCCCGCACGATCGACGGCCGGCCGGACCACTTCTACCACCCGTTCGGG
CTGCTGCCCGAGCACGGCCAGGTGCCGCTGTCGCACTACTGGGCCTAC
AACCGGGCGGCCGGCACCACCGACGAGCCGTTCGACTACGCCTGTTTC
GCCGAGACCGCCGCCATGGACGCGGTCCGCGCGCCCAAGTGGCTCGAC
GGGCGGCCCGCCACCCGGTATGCCTGGCACTTCGACGCCCACCTGGTC
GCCGAGTTCCTGCGCCGGCACGCCACCGAGCGGCTCAACGTGGAGCAC
GTGCAGGGTGAGATGCAGCAGGTGCTGCGCGACGAGCGGGGCTTCATC
ACCGCCCTGCGCACGGTCGAGGGCCGGGACCTCGAGGGTGACCTGTTC
ATCGACTGCTCCGGCTTCCGCGGCCTGCTGATCAACAAGGCCATGGAG
GAGCCCTTCATCGACATGAACGACCAGCTGCTGTGCAACCGCGCGGTG
GCCACGGCGATCAAGCACGACGACGACGCGCACGGGGTCGAGCCGTAC
ACCTCGGCGATCGCCATGCGGTCCGGCTGGAGCTGGAAGATCCCGATG
CTGGGCCGCTTCGGGACCGGCTACGTCTACTCCAGCCGGTTCGCCGAG
AAGGACGAGGCCACCCTCGACTTCTGCCGGATGTGGGGCCTGGACCCG
GAGAACACCCCGCTCAACCAGGTGGCCTTCCGGGTCGGCCGCAACCGC
CGGGCCTGGGTGAAGAACTGCGTGAGCATCGGCCTGGCGTCGTGCTTC
CTGGAGCCACTGGAGTCCACCGGCATCTACTTCATCACCGCGGCCATC
TACCAGCTGACTCAGCACTTCCCGGACCGCACCTTCGCGCTCGCGTTG
AGCGACGCGTTCAACCACGAGATCGAAGCCATGTTCGACGACACCCGG
GACTTCATCCAGGCGCACTTCTACGTGTCGCCGCGCACCGACACGCCG
TTCTGGAAGGCGAACAAGGACCTGCACCTGCCCGAGCAGATGCGCGAG
AAGATCGCGATGTACAAGGCCGGCCTGCCGATCAACGCGCCGGTCACC
GACGAGTCGACCTACTACGGCAGGTTCGAGGCCGAGTTCCGCAACTTC
TGGACCAACGGCAGCTACTACTGCATCTTCGCGGGCCTCGGGCTGCGC
CCGGACAACCCGCTGCCGATGCTGCGACACCGTCCGGAGCAGGTCCGC
GAGGCGCAGGCGCTGTTCGCCGGGGTGAAGGACAAGCAGCGGGAGTTG
GTGGAGACGCTGCCGAGCAACCTGGAGTTCCTGCGCAGCCTGCACGGC
AAGTAG
SEQ. ID NO: 22
MDNRINRIVILGGGTAGWMTASYLAKALGDTVTITLLEAPAIGRIGVG
EATVPNLQRVFFDFLGLREEEWMPECNAAFKTAVKFINWRTPGPGEAK
ARTIDGRPDHFYHPFGLLPEHGQVPLSHYWAYNRAAGTTDEPFDYACF
AETAAMDAVRAPKWLDGRPATRYAWHFDAHLVAEFLRRHATERLNVEH
VQGEMQQVLRDERGFITALRTVEGRDLEGDLFIDCSGFRGLLINKAME
EPFIDMNDQLLCNRAVATAIKHDDDAHGVEPYTSAIAMRSGWSWKIPM
LGRFGTGYVYSSRFAEKDEATLDFCRMWGLDPENTPLNQVAFRVGRNR
RAWVKNCVSIGLASCFLEPLESTGIYFITAAIYQLTQHFPDRTFALAL
SDAFNHEIEAMFDDTRDFIQAHFYVSPRTDTPFWKANKDLHLPEQMRE
KIAMYKAGLPINAPVTDESTYYGRFEAEFRNFWINGSYYCIFAGLGLR
PDNPLPMLRHRPEQVREAQALFAGVKDKQRELVETLPSNLEFLRSLHG
K
SEQ. ID NO: 23
ATGGATGACAATCGAATTCGGAGCATCCTTGTCCTTGGCGGCGGCACG
GCCGGCTGGATGTCCGCCTGCTACCTGAGCAAGGCGCTCGGGCCCGGC
GTCGAGGTCACCGTGCTCGAGGCGCCCTCCATCTCGCGCATCCGGGTC
GGCGAGGCCACCATTCCCAACCTGCACAAGGTCTTCTTCGACTTCCTG
GGCATCGCCGAGGACGAGTGGATGCGGGAGTGCAACGCCAGCTACAAG
GCCGCGGTCCGGTTCGTCAACTGGCGGACGCCGGGCGACGGCCAGGCC
ACGCCGCGGCGGCGTCCGGACGGCCGCCCCGACCACTTCGACCACCTG
TTCGGCCAGCTGCCCGAGCACGAGAACCTGCCGCTGTCGCAGTACTGG
GCGCACCGGCGCCTCAACGGCCTGACCGACGAACCCTTCGACCGCTCC
TGCTACGTGCAGCCCGAGCTGCTGGACCGCAAGCTCTCGCCGAGGTTG
ATGGACGGCACGAAACTGGCCAGCTACGCCTGGCACTTCGACGCCGAC
CTGGTGGCCGACTTCCTCTGCCGGTTCGCCGTGCAGAAGCTGAACGTG
ACCCACGTCCAGGACGTGTTCACGCATGCCGACCTCGACCAGCGCGGC
CACATCACGGCCGTCAACACCGAGTCCGGCCGCACGCTGGCCGCCGAC
CTGTTCATCGACTGCAGCGGCTTCCGCAGCGTGCTCATGGGCAAGGTC
ATGCAGGAGCCGTTCCTGGACATGAGCAAGCACCTGCTCAACGACCGC
GCGGTGGCGCTGATGCTCCCGCACGACGACGAGAAGGTGGGCATCGAG
CCGTACACCTCGTCGCTGGCCATGCGGTCGGGCTGGTCGTGGAAGATC
CCGCTGCTCGGCCGGTTCGGCTCCGGCTACGTCTACTCCAGCCAGTTC
ACCTCCCAGGACGAGGCGGCCGAGGAGCTCTGCCGCATGTGGGACGTC
GACCCGGCGGAGCAGACGTTCAACAACGTCCGGTTCCGGGTCGGCCGC
AGCCGCCGGGCCTGGGTGCGCAACTGCGTCGCCATCGGCGTGTCCGCC
ATGTTCGTGGAGCCGCTGGAGTCGACCGGCCTGTACTTCAGTTACGCC
TCGCTCTACCAGCTGGTGAAGCACTTCCCGGACAAGCGGTTCCGGCCG
ATCCTGGCCGACCGGTTCAACCGCGAGGTGGCGACCATGTACGACGAC
ACCCGCGACTTCCTCCAGGCGCACTTCAGCCTGTCGCCGCGTGACGAC
TCCGAGTTCTGGCGGGCCTGCAAGGAGCTGCCGTTCGCGGACGGGTTC
GCCGAGAAGGTCGAGATGTACAGGGCCGGGCTGCCGGTCGAACTGCCG
GTCACCATCGACGACGGGCACTACTACGGCAATTTCGAGGCCGAGTTC
CGCAACTTCTGGACCAACTCGAACTACTACTGCATCTTCGCCGGGCTC
GGTTTCCTGCCCGAGCATCCGCTGCCGGTGCTCGAATTCCGCCCGGAG
GCCGTCGATCGCGCGGAGCCGGTGTTCGCCGCGGTGCGCCGGCGCACG
GAGGAGCTGGTCGCCACCGCCCCGACCATGCAGGCCTACCTGCGGCGC
CTGCACCAGGGCACGTAG
SEQ. ID NO: 24
MDDNRIRSILVLGGGTAGWMSACYLSKALGPGVEVTVLEAPSISRIRV
GEATIPNLHKVFFDFLGIAEDEWMRECNASYKAAVRFVNWRTPGDGQA
TPRRRPDGRPDHFDHLFGQLPEHENLPLSQYWAHRRLNGLTDEPFDRS
CYVQPELLDRKLSPRLMDGTKLASYAWHFDADLVADFLCRFAVQKLNV
THVQDVFTHADLDQRGHITAVNTESGRTLAADLFIDCSGFRSVLMGKV
MQEPFLDMSKHLLNDRAVALMLPHDDEKVGIEPYTSSLAMRSGWSWKI
PLLGRFGSGYVYSSQFTSQDEAAEELCRMWDVDPAEQTFNNVRFRVGR
SRRAWVRNCVAIGVSAMFVEPLESTGLYFSYASLYQLVKHFPDKRFRP
ILADRFNREVATMYDDTRDFLQAHFSLSPRDDSEFWRACKELPFADGF
AEKVEMYRAGLPVELPVTIDDGHYYGNFEAEFRNFWTNSNYYCIFAGL
GFLPEHPLPVLEFRPEAVDRAEPVFAAVRRRTEELVATAPTMQAYLRR
LHQGT
SEQ. ID NO: 25
ATGAACAAGCCGATCAAGAATATCGTCATCGTGGGCGGCGGTACTGCG
GGCTGGATGGCCGCCTCGTACCTCGTCCGGGCCCTCCAACAGCAGGCG
AACATTACGCTCATCGAATCTGCGGCGATCCCTCGGATCGGCGTGGGC
GAAGCGACCATCCCAAGTTTGCAGAAGGTGTTCTTCGATTTCCTCGGG
ATACCGGAGCGGGAATGGATGCCCCAAGTGAACGGCGCGTTCAAGGCC
GCGATCAAGTTCGTGAATTGGAGAAAGTCTCCCGACCCCTCGCGCGAC
GATCACTTCTACCATTTGTTCGGCAACGTGCCGAACTGCGACGGCGTG
CCGCTTACCCACTACTGGCTGCGCAAGCGCGAACAGGGCTTCCAGCAG
CCGATGGAGTACGCGTGCTACCCGCAGCCCGGGGCACTCGACGGCAAG
CTGGCACCGTGCCTGTCCGACGGCACCCGCCAGATGTCCCACGCGTGG
CACTTCGACGCGCACCTGGTGGCCGACTTCTTGAAGCGCTGGGCCGTC
GAGCGCGGGGTGAACCGCGTGGTCGATGAGGTGGTGGACGTTCGCCTG
AACAACCGCGGCTACATCTCCAACCTGCTCACCAAGGAGGGGCGGACG
CTGGAGGCGGACCTGTTCATCGACTGCTCCGGCATGCGGGGGCTCCTG
ATCAATCAGGCGCTGAAGGAACCCTTCATCGACATGTCCGACTACCTG
CTGTGCGACAGCGCGGTCGCCAGCGCCGTGCCCAACGACGACGCGCGC
GATGGGGTCGAGCCGTACACCTCCTCGATCGCCATGAACTCGGGATGG
ACCTGGAAGATTCCGATGCTGGGCCGGTTCGGCAGCGGCTACGTCTTC
TCGAGCCATTTCACCTCGCGCGACCAGGCCACCGCCGACTTCCTCAAA
CTCTGGGGCCTCTCGGACAATCAGCCGCTCAACCAGATCAAGTTCCGG
GTCGGGCGCAACAAGCGGGCGTGGGTCAACAACTGCGTCTCGATCGGG
CTGTCGTCGTGCTTTCTGGAGCCCCTGGAATCGACGGGGATCTACTTC
ATCTACGCGGCGCTTTACCAGCTCGTGAAGCACTTCCCCGACACCTCG
TTCGACCCGCGGCTGAGCGACGCTTTCAACGCCGAGATCGTCCACATG
TTCGACGACTGCCGGGATTTCGTCCAAGCGCACTATTTCACCACGTCG
CGCGATGACACGCCGTTCTGGCTCGCGAACCGGCACGACCTGCGGCTC
TCGGACGCCATCAAAGAGAAGGTTCAGCGCTACAAGGCGGGGCTGCCG
CTGACCACCACGTCGTTCGACGATTCCACGTACTACGAGACCTTCGAC
TACGAATTCAAGAATTTCTGGTTGAACGGCAACTACTACTGCATCTTT
GCCGGCTTGGGCATGCTGCCCGACCGGTCGCTGCCGCTGTTGCAGCAC
CGACCGGAGTCGATCGAGAAAGCCGAGGCGATGTTCGCCAGCATCCGG
CGCGAGGCCGAGCGTCTGCGCACCAGCCTGCCGACAAACTACGACTAC
CTGCGGTCGCTGCGTGACGGCGACGCGGGGCTGTCGCGCGGCCAGCGT
GGGCCGAAGCTCGCAGCGCAGGAAAGCCTGTAG
SEQ. ID NO: 26
MNKPIKNIVIVGGGTAGWMAASYLVRALQQQANITLIESAAIPRIGVG
EATIPSLQKVFFDFLGIPEREWMPQVNGAFKAAIKFVNWRKSPDPSRD
DHFYHLFGNVPNCDGVPLTHYWLRKREQGFQQPMEYACYPQPGALDGK
LAPCLSDGTRQMSHAWHFDAHLVADFLKRWAVERGVNRVVDEVVDVRL
NNRGYISNLLTKEGRTLEADLFIDCSGMRGLLINQALKEPFIDMSDYL
LCDSAVASAVPNDDARDGVEPYTSSIAMNSGWTWKIPMLGRFGSGYVF
SSHFTSRDQATADFLKLWGLSDNQPLNQIKFRVGRNKRAWVNNCVSIG
LSSCFLEPLESTGIYFIYAALYQLVKHFPDTSFDPRLSDAFNAEIVHM
FDDCRDFVQAHYFTTSRDDTPFWLANRHDLRLSDAIKEKVQRYKAGLP
LTTTSFDDSTYYETFDYEFKNFWLNGNYYCIFAGLGMLPDRSLPLLQH
RPESIEKAEAMFASIRREAERLRTSLPTNYDYLRSLRDGDAGLSRGQR
GPKLAAQESL
SEQ. ID NO: 27
ATGTCCGGCAAGATTGACAAGATCCTCATCGTCGGCGGCGGCACCGCC
GGATGGATGGCCGCGTCCTATCTCGGCAAGGCCCTGCAGGGCACCGCG
GACATCACACTGCTGCAGGCACCCGACATCCCGACGCTCGGGGTCGGC
GAGGCCACGATCCCCAATCTGCAGACGGCGTTCTTCGACTTCCTCGGA
ATCCCCGAGGACGAGTGGATGCGGGAGTGCAACGCGAGCTACAAGGTC
GCCATCAAGTTCATCAACTGGCGCACCGCGGGCGAGGGGACGTCCGAG
GCCCGCGAGCTCGACGGAGGGCCCGACCACTTCTACCACTCCTTCGGT
CTGCTCAAGTACCACGAGCAGATTCCGCTGTCGCACTACTGGTTCGAC
CGTTCGTACCGGGGGAAGACCGTCGAGCCGTTCGACTACGCCTGCTAC
AAGGAACCCGTCATCCTCGACGCCAACAGGTCACCGCGCAGGCTCGAC
GGTTCCAAGGTGACGAACTACGCGTGGCACTTCGACGCGCACCTCGTC
GCCGACTTCCTGCGCCGGTTCGCCACCGAGAAGCTCGGCGTGCGCCAC
GTCGAGGACCGCGTCGAGCACGTCCAGCGCGACGCCAACGGCAACATC
GAGTCGGTTCGCACGGCAACGGGGCGTGTCTTCGATGCCGACCTCTTC
GTCGACTGCTCGGGCTTCCGCGGGCTGCTGATCAACAAGGCGATGGAG
GAGCCCTTCCTCGACATGAGCGATCACCTGCTCAACGACAGCGCCGTC
GCCACCCAGGTGCCGCACGACGACGACGCGAACGGTGTGGAACCGTTC
ACCTCGGCGATCGCCATGAAGTCGGGCTGGACGTGGAAGATCCCGATG
CTCGGCAGGTTCGGCACCGGGTACGTCTACTCGAGCCGGTTCGCCACC
GAGGACGAGGCGGTGCGCGAGTTCTGCGAGATGTGGCACCTCGACCCG
GAGACCCAGCCCCTCAACAGGATCCGGTTCCGGGTCGGCCGCAACCGG
CGCGCGTGGGTCGGCAACTGCGTCAGCATCGGCACGTCGTCGTGCTTC
GTGGAACCACTGGAGTCGACGGGCATCTACTTCGTCTACGCCGCGCTG
TACCAGCTGGTGAAGCACTTCCCCGACAAGAGCCTCAACCCCGTGCTG
ACCGCCAGGTTCAACCGCGAGATCGAGACGATGTTCGACGACACGCGC
GACTTCATCCAGGCGCACTTCTACTTCTCGCCGCGCACGGACACCCCG
TTCTGGAGGGCCAACAAGGAGCTGCGCCTGGCGGACGGCATGCAGGAG
AAGATCGACATGTACCGCGCGGGCATGGCGATCAACGCGCCCGCGTCC
GACGACGCCCAGCTCTACTACGGCAACTTCGAGGAGGAGTTCCGCAAC
TTCTGGAACAACAGCAACTACTACTGCGTGCTGGCCGGCCTCGGTCTG
GTGCCCGACGCACCCTCACCACGCCIGGCGCACATGCCACAGGCGACG
GAGTCGGTGGACGAGGTCTTCGGCGCCGTCAAGGACCGGCAGCGGAAC
CTGCTCGAGACCCTGCCGAGCCTCCACGAGTTCCTGAGGCAACAGCAC
GGCCGCTGA
SEQ. ID NO: 28
MSGKIDKILIVGGGTAGWMAASYLGKALQGTADITLLQAPDIPTLGVG
EATIPNLQTAFFDFLGIPEDEWMRECNASYKVAIKFINWRTAGEGTSE
ARELDGGPDHFYHSFGLLKYHEQIPLSHYWFDRSYRGKTVEPFDYACY
KEPVILDANRSPRRLDGSKVTNYAWHFDAHLVAKAMEEPFLDMSDHLL
NDSAVATQVPHDDDANGVEPFTSAIAMKSGWTWKIPMLGRFGTGYVYS
SRFATEDEAVREFCEMWHLDPETQPLNRIRFRVGRNRRAWVGNCVSIG
TSSCFVEPLESTGIYFVYAALYQLVKHFPDKSLNPVLTARFNREIETM
FDDTRDFIQAHFYFSPRTDTPFWRANKELRLADGMQEKIDMYRAGMAI
NAPASDDAQLYYGNFEEEFRNFWNNSNYYCVLAGLGLVPDAPSPRLAH
MPQATESVDEVFGAVKDRQRNLLETLPSLHEFLRQQHGR
SEQ. ID NO: 29
MLNNVVIVGGGTAGWMTASYFKAAFGERINITLVESGNIGAVGVGEAT
FSDIRHFFDFLGLKEKDWMPACNATYKLAVRFENWREEGHYFYHPFEQ
MRSVNGFPLADWWLREKPTDRFDKDCFVMPSIIDAGLSPRHLDGSLID
QPFVEGAEEMQGLTMSEHQGKTQFPYAYQFEAALLAKYLTTYSVERGV
KHIVDDVQQVNLDERGWIRNVTTAEHGEIGGDLFIDCTGFRGLLLNKA
LEEPFISFQDTLPNDSAVALQVPMDMERRGILPCTTATAQDAGWIWTI
PLMGRVGTGYVYAKDYISPEEAERTLREFVGPAAADAEANHIKMRVGR
SENSWVKNCVAIGLSSGFVEPLESTGIFFIHHAIEQLAKNFPGEDWNP
VQRNLYNDSVAHVMDGVREFLVLHYVAAKRSDTQYWRDTKTRKIPDAL
AERIEKWKTQLPDTETVFPYYHGLPPYSYHCVLLGMGGIDVKPSPALA
LGDSAAAEREFAEIREKTRHLTSVLPKAYDYFSRMR
SEQ. ID NO: 30
MSGKIDKILIVGGGTAGWMAASYLGKALQGTADITLLQAPDIPTLGVG
EATIPNLQTAFFDFLGIPEDEWMRECNASYKVAIKFINWRTAGEGTSE
ARELDGGPDHFYHSFGLLKYHEQIPLSHYWFDRSYRGKTVEPFDYACY
KEPVILDANRSPRRLDGSKVTNYAWHFDAHLVAKAMEEPFLDMSDHLL
NDSAVATQVPHDDDANGVEPFTSAIAMKSGWTWKIPMLGRFGTGYVYS
SRFATEDEAVREFCEMWHLDPETQPLNRIRFRVGRNRRAWVGNCVSIG
TSSCFVEPLESTGIYFVYAALYQLVKHFPDKSLNPVLTARFNREIETM
FDDTRDFIQAHFYFSPRTDTPFWRANKELRLADGMQEKIDMYRAGMAI
NAPASDDAQLYWGNFEEEFRNFWNNSNYYCVLAGLGLVPDAPSPRLAH
MPQATESVDEVFGAVKDRQRNLLETLPSLHEFLRQQHGR
SEQ. ID NO: 31
MSGKIDKILIVGGGTAGWMAASYLGKALQGTADITLLQAPDIPTLGVG
EATIPNLQTAFFDFLGIPEDEWMRECNASYKVAIKFINWRTAGEGTSE
ARELDGGPDHFYHSFGLLKYHEQIPLSHYWFDRSYRGKTVEPFDYACY
KEPVILDANRSPRRLDGSKVTNYAWHFDAHLVADFLRRFATEKLGVRH
VEDRVEHVQRDANGNIESVRTATGRVFDADLFVDCSGFRGLLINKAME
EPFLDMSDHLLNDSAVATQVPHDDDANGVEPFTSAIAMKSGWTWKIPM
LGRFGTGYVYSSRFATEDEAVREFCEMWHLDPETQPLNRIRFRVGRNR
RAWVGNCVSIGTSSCFVEPLESTGIYFVYAALYQLVKHFPDKSLNPVL
TARFNREIETMFDDTRDFIQAHFYFSPRTDTPFWRANKELRLADGMQE
KIDMYRAGMAINAPASDDAQLYYGNFEEEFRNFWNNSSYYCVLAGLGL
VPDAPSPRLAHMPQATESVDEVFGAVKDRQRNLLETLPSLHEFLRQQH
GR
SEQ. ID NO: 32
MSGKIDKILIVGGGTAGWMAASYLGKALQGTADITLLQAPDIPTLGVG
EATMPNLQTAFFDFLGIPEDEWMRECNASYKVAIKFINWRTAGEGTSE
ARELDGGPDHFYHPFGLLKYHEQIPLSHYWFDRLYRGKTVEPFDYACY
KEPVILDANRSPRRLDGSKVTSYAWHFDAHLVAKAMEEPFLDMSDHLL
NDSAVATQVPHDDDANGVEPFTSAIAMKSGWTWKIPMLGRFGTGYVYS
SRFATEDEAVREFCEMWHLDPETQPLNRIRFRVGRNRRAWVGNCVSIG
TSSCFVEPLESTGIYFVYAALYQLVKHFPDKSFNPVLTARFNREIETM
FDDTRDFIQAHFYFSPRTDTPFWRANKELRLADGMQEKIDMYRAGMAI
NAPAPDDAQLYWGNFEEEFRNLWNNSSYYCVLAGLGLVPDAPSPRLAH
MPRATESVDEVFGAVKDQQRNLLETLPSLHEFLRQQHGR
SEQ. ID NO: 33
MSGKIDKILIVGGGTAGWMAASYLGKALQGTADITLLQAPDIPTLGVG
EATHPNLQTAFFDFLGIPEDEWMRECNASYKVAIKFINWRTAGEGTSE
ARELDGGPDHFYHSFGLLKYHEQIPLSHYWFDRSYRGKTVEPFDYACY
KEPVILDANRSPRRLDGSKVTNYAWHFDAHLVAKAMEEPFLDMSDHLL
NDSAVATQVPHDDDANGVEPFTSAIAMKSGWTWKIPMLGRFGTGYVYS
SRFATEDEAVREFCEMWHLDPETQPLNRIRFRVGRNRRAWVGNCVSIG
TSSCFVEPLESTGIYFVYAALYQLVKHFPDKSFNPVLTARFNREIETM
FDDTRDFIQAHFYFSPRTDTPFWRANKELRLADGMQEKIDMYRAGMAI
NAPASDDAQLYYGNFEEEFRNCWNNSSYYCVLAGLGLVPDAPSPRLAH
MPRATESVDEVFGAVKDQQRNLLETLPSLHEFLRQQHGR
SEQ. ID NO: 34
MSGKIDKILIVGGGTAGWMAASYLGKALQGTADITLLQAPDIPTLGVG
EATIPNLQTAFFDFLGIPEDEWMRECNASYKVAIKFINWRTAGEGTSE
ARELDGGPDHFYHSFSLLKYHEQIPLSHYWFDRSYRGKTVEPFDYACY
KEPVILDANRSPRRLDGSKVTNYAWHFDAHLVAKAMEEPFLDMSDHLL
NDSAVATQVPHDDDANGVEPFTSAIAMKSGWTWKIPMLGRFGTGYVYS
SRFATEDEAVREFCEMWHLDPETQPLNRIRFRVGRNRRAWVGNCVSIG
TSSCFVEPLESTGIYFVYAALYQLVKHFPDKSLNPVLTARFNREIETM
FDDTRDFIQAHFYFSPRTDTPFWRANKELRLADGMQEKIDMYRAGMAI
NAPASDDAQLYYGNFEEEFRNFWNNSSYYCVLAGLGLVPDAPSPRLAH
MPQATESVDEVFGAVKDRQRNLLETLPSLHEFLRQQHGR
SEQ. ID NO: 35
MSGKIDKILIVGGGTAGWMAASYLGKALQGTADITLLQAPDIPTLGVG
EATIPNLQTAFFDFLGIPEDEWMRECNASYKVAIKFINWRTAGEGTSE
ARELDGGPDHFYHSFGLLKYHEQIPLSHYWFDRSYRGKTVEPFDYACY
KEPVILDANRSPRRLDGSKVTNYAWHFDAHLVADFLRRFATEKLGVRH
VEDRVEHVQRDANGNIESVRTATGRVFDADLFVDCSGFRGLLINKAME
EPFLDMSDHLLNDSAVATQVPHDDDANGVEPFTSAIAMKSGWTWKIPM
LGRFGTGYVYSSRFATEDEAVREFCEMWHLDPETQPLNRIRFRVGRNR
RAWVGNCVSIGTSSCFVEPLESTGIYFVYAALYQLVKHFPDKSLNPVL
TARFNREIETMFDDTRDFIQAHFYFSPRTDTPFWRANKELRLADGMQE
KIDMYRAGMVINAPASDDAQLYYGNFEEEFRNFWNNSNYYCVLAGLGL
VPDAPSPRLAHMPQATESVDEVFGAVKDRQRNLLETLPSLHEFLRQQH
GR
SEQ. ID NO: 36
ATGATGTCTGAAAATTTGCAATTGTCAGCTGAAGAAATGAGACAATTG
GGTTACCAAGCAGTTGATTTGATCATCGATCACATGAACCATTTGAAG
TCTAAGCCAGTTTCAGAAACAATCGATTCTGATATCTTGAGAAATAAG
TTGACTGAATCTATCCCAGAAAATGGTTCAGATCCAAAGGAATTGTTG
CATTTCTTGAACAGAAACGTTTTTAATCAAATTACACATGTTGATCAT
CCACATTTCTTGGCTTTTGTTCCAGGTCCAAATAATTACGTTGGTGTT
GTTGCAGATTTCTTGGCTTCTGGTTTTAATGTTTTTCCAACTGCATGG
ATTGCTGGTGCAGGTGCTGAACAAATCGAATTGACTACAATTAATTGG
TTGAAATCTATGTTGGGTTTTCCAGATTCAGCTGAAGGTTTATTTGTT
TCTGGTGGTTCAATGGCAAATTTGACAGCTTTGACTGTTGCAAGACAG
GCTAAGTTGAACAACGATATCGAAAATGCTGTTGTTTACTTCTCTGAT
CAAACACATTTCTCAGTTGATAGAGCATTGAAGGTTTTAGGTTTTAAA
CATCATCAAATCTGTAGAATCGAAACAGATGAACATTTGAGAATCTCT
GTTTCAGCTTTGAAGAAACAAATTAAAGAAGATAGAACTAAGGGTAAA
AAGCCATTCTGTGTTATTGCAAATGCTGGTACTACAAATTGTGGTGCT
GTTGATTCTTTGAACGAATTAGCAGATTTGTGTAACGATGAAGATGTT
TGGTTGCATGCTGATGGTTCTTATGGTGCTCCAGCTATCTTGTCTGAA
AAGGGTTCAGCTATGTTGCAAGGTATTCATAGAGCAGATTCTTTGACT
TTAGATCCACATAAGTGGTTGTTCCAACCATACGATGTTGGTTGTGTT
TTGATCAGAAACTCTCAATATTTGTCAAAGACTTTTAGAATGATGCCA
GAATACATCAAGGATTCAGAAACTAACGTTGAAGGTGAAATTAATTTC
GGTGAATGTGGTATCGAATTGTCAAGAAGATTCAGAGCTTTGAAGGTT
TGGTTGTCTTTTAAAGTTTTCGGTGTTGCTGCTTTTAGACAAGCAATC
GATCATGGTATCATGTTAGCAGAACAAGTTGAAGCATTTTTGGGTAAA
GCAAAAGATTGGGAAGTTGTTACACCAGCTCAATTGGGTATCGTTACT
TTTAGATACATTCCATCTGAATTGGCATCAACAGATACTATTAATGAA
ATTAATAAGAAATTGGTTAAGGAAATCACACATAGAGGTTTCGCTATG
TTATCTACTACAGAATTGAAGGAAAAGGTTGTTATTAGATTGTGTTCA
ATTAATCCAAGAACTACAACTGAAGAAATGTTGCAAATCATGATGAAG
ATTAAAGCATTGGCTGAAGAAGTTTCTATTTCATACCCATGTGTTGCT
GAATAA
SEQ. ID NO: 37
MMSENLQLSAEEMRQLGYQAVDLIIDHMNHLKSKPVSETIDSDILRNK
LTESIPENGSDPKELLHFLNRNVFNQITHVDHPHFLAFVPGPNNYVGV
VADFLASGFNVFPTAWIAGAGAEQIELTTINWLKSMLGFPDSAEGLFV
SGGSMANLTALTVARQAKLNNDIENAVVYFSDQTHFSVDRALKVLGFK
HHQICRIETDEHLRISVSALKKQIKEDRTKGKKPFCVIANAGTTFQPY
DVGCVLIRNSQYLSKTFRMMPEYIKDSETNVEGEINFGECGIELSRRF
RALKVWLSFKVFGVAAFRQAIDHGIMLAEQVEAFLGKAKDWEVVTPAQ
LGIVTFRYIPSELASTDTINEINKKLVKEITHRGFAMLSTTELKEKVV
IRLCSINPRTTTEEMLQIMMKIKALAEEVSISYPCVAE
SEQ. ID NO: 38
ATGTCTCAAGTGATCAAAAAAAAGAGAAACACTTTCATGATCGGTACCG
AATATATATTGAACTCTACCCAATTAGAAGAGGCTATTAAATCTTTCG
TCCATGATTTCTGTGCCGAAAAGCACGAAATTCACGACCAACCAGTTG
TCGTTGAAGCTAAGGAACACCAAGAAGATAAGATTAAGCAAATCAAAA
TTCCAGAAAAGGGTCGTCCAGTCAATGAAGTTGTTTCTGAAATGATGA
ACGAAGTCTACAGATACAGAGGTGATGCCAACCATCCAAGATTCTTCT
CTTTCGTTCCAGGTCCAGCTTCCTCTGTCTCCTGGTTGGGTGACATCA
TGACTTCTGCTTACAACATCCACGCTGGTGGTTCTAAATTGGCCCCAA
TGGTTAACTGTATCGAACAAGAAGTCTTGAAATGGCTCGCAAAGCAAG
TTGGTTTCACCGAAAACCCTGGTGGTGTTTTCGTTTCCGGTGGTTCTA
TGGCTAACATTACTGCTTTGACAGCCGCTAGAGACAACAAGCTTACCG
ATATTAACTTGCACTTGGGTACTGCTTACATTTCCGACCAAACCCACT
CTTCAGTTGCTAAGGGTTTACGTATCATTGGTATCACTGACTCCAGAA
TCAGAAGAATTCCAACTAACTCCCACTTCCAAATGGACACCACTAAGT
TGGAAGAAGCTATCGAAACCGACAAGAAGTCCGGTTACATCCCATTTG
TTGTTATCGGTACTGCCGGTACCACCAACACTGGTTCCATTGACCCAT
TGACTGAAATTTCCGCTTTGTGTAAGAAGCATGACATGTGGTTCCACA
TTGATGGTGCCTACGGTGCTTCAGTTTTGTTGTCTCCAAAGTACAAGT
CTTTGTTAACAGGTACCGGTTTGGCTGACTCTATCTCTTGGGATGCTC
ACAAGTGGTTATTTCAAACTTACGGTTGTGCTATGGTTTTGGTCAAGG
ATATTAGAAACTTGTTTCATTCCTTCCACGTCAATCCAGAATACTTGA
AGGATCTGGAAAACGACATCGATAACGTTAACACCTGGGACATCGGCA
TGGAATTGACCAGACCAGCCAGAGGTTTGAAGTTGTGGTTGACTTTGC
AAGTTCTAGGTTCTGATCTTATTGGTTCTGCCATTGAACACGGTTTCC
AATTGGCTGTCTGGGCTGAAGAAGCCTTGAATCCAAAGAAGGACTGGG
AAATTGTCTCTCCTGCTCAAATGGCTATGATCAACTTCAGATATGCTC
CAAAGGACTTGACCAAGGAAGAACAAGATATCTTGAACGAAAAGATCT
CTCACCGTATTTTGGAATCCGGTTACGCTGCTATCTTCACTACCGTCC
TAAACGGTAAGACTGTCTTGAGAATCTGCGCTATTCACCCAGAAGCTA
CTCAAGAAGACATGCAACACACCATTGATTTGTTGGACCAATACGGTA
GAGAAATCTACACTGAAATGAAGAAGGCTTAG
SEQ. ID NO: 39
MSQVIKKKRNTFMIGTEYILNSTQLEEAIKSFVHDFCAEKHEIHDQPV
VVEAKEHQEDKIKQIKIPEKGRPVNEVVSEMMNEVYRYRGDANHPRFF
SFVPGPASSVSWLGDIMTSAYNIHAGGSKLAPMVNCIEQEVLKWLAKQ
VGFTENPGGVFVSGGSMANITALTAARDNKLTDINLHLGTAYISDQTH
SSVAKGLRIIGITDSRIRRIPTNSHFQMDTTKLEEAIETDKKSGYIPF
VVIGTAGTTNTGSIDPLTEISALCKKHDMWFHIDGAYGASVLLSPKYK
SLLTGTGLADSISWDAHKWLFQTYGCAMVLVKDIRNLFHSFHVNPEYL
KDLENDIDNVNTWDIGMELTRPARGLKLWLTLQVLGSDLIGSAIEHGF
QLAVWAEEALNPKKDWEIVSPAQMAMINFRYAPKDLTKEEQDILNEKI
SHRILESGYAAIFTTVLNGKTVLRICAIHPEATQEDMQHTIDLLDQYG
REIYTEMKKA
SEQ. ID NO: 40
ATGAAGCCAGCTGAAGCTAAACCACCTCACATGGACCATGATACTTTC
AGATCCTTGGGTCACCAAGCTATTGACTGGATTGCTGATTACTGGCAA
CACTTGGCTGACAGACCAGTTGCTCCATTGGTTGAACCAGGTACTGTT
AGAGCCCAATTGCCTACCACTCCACCAGAGTACGGTGAAGACTTCCCA
GCTTTGTTATCTGACTTGGAAAGAATTGTCTTGCCAGGCTTGTTGCAC
TGGCAACACCCAAGATTCTTCGGTTACTTCCCAGCTAACGCTTCCGGT
CCAGCCGTTTTAGCTGAATTATTATCTGCTGGTCTAGGTGTCCAAGGT
ATGAACTGGAACACTTCTCCAGCTTGTACCGAAGTTGAACAACAAATG
TTGGACTGGTTCGTTCATTTGTTAGGGTTGCCAGAACACTTCAGAGGT
GGTGGTGTTATCCAAGACACTGCCTCCTCCGCCGTTTTGGTCGCTCTA
TTGACTGCTTTGCACAGAGCTTCTGCTGGTAGAACCAGAGATCACGGT
ACTGGTGAATGTGGTTACAGGGTTTACCTAACCGCTGAAACTCACTCC
GCTGCTAGAAAGGCTGCCGTCATCACCGGTTTGGGTTTGCGTGCTGTT
TGTGAAGTCGCAACTGATGCTGACGGTGCTATGGATGCCGCTGACTTG
GCCGCTCGTATTCAAGCCGATTTGGCTGCCGGTTTGACCCCATTGATG
GTTGTTGCTACCCGTGGTACCACCTCTGATTTGTCCTTCGATCCATTG
GAAGACATCGGTCCAGTCTGTAGAAGACATGGTGTCTGGTTACACGTT
GACGCTGCTTATGCTGGTGTTGCTGCAGTGTGCGATGAATTGCATTGG
GTTAATGATGGTGTTAGATACGCTGATTCTTACTGTACTAACCCACAC
AAGTGGTTGTTGACCAACTTCGACTGTGATTTATTATGGGTTGCCGAC
CCAGAAGCTTTGGTCAACGCTTTGTCCGTTTTGCCTGCTTACTTGAGA
AACCCAGCTTCTGAATCAGGTAGAGTTACTGACTACAGACACTGGCAA
GTTCCATTGGGCAGAAGATTCAGAGCCTTGAAGTTGTGGTCTGTCTTA
AGATGGTACGGTGCCGAAGGTTTGAGATCTCACATCAGAACTGGTGTC
AGACACGCCAGATTGTTCGCTGACCTGGTCAGAGAAGATGACCGTTTC
ACCTTGTTGGCGCCAGCTACTTTGGGTTTGGTCACTTTCCGTCACGCT
GGTTCTGACACCGACAACAGAAACTTGTTGCACGCTATCAACGCCGAA
GGTACAACTTTTTTAACTCATAGTGAAAAGAACGGTACCTTTTTCTTG
AGATTTGCTACCGGTGGTACCCACACTGAAGACCATCACGTCCACGAA
GCTTGGAGAGCTGTCCAAAATGCCGTCCAAAACGCCGTCCCAAGAGCC
CAACATGCCACCGATGGTACTGCCGATGCTTTGAACGGTTAA
SEQ. ID NO: 41
MKPAEAKPPHMDHDTFRSLGHQAIDWIADYWQHLADRPVAPLVEPGTV
RAQLPTTPPEYGEDFPALLSDLERIVLPGLLHWQHPRFFGYFPANASG
PAVLAELLSAGLGVQGMNWNTSPACTEVEQQMLDWFVHLLGLPEHFRG
GGVIQDTASSAVLVALLTALHRASAGRTRDHGTGECGYRVYLTAETHS
AARKAAVITGLGLRAVCEVATDADGAMDAADLAARIQADLAAGLTPLM
VVATRGTTSDLSFDPLEDIGPVCRRHGVWLHVDAAYAGVAAVCDELHW
VNDGVRYADSYCTNPHKWLLTNFDCDLLWVADPEALVNALSVLPAYLR
NPASESGRVTDYRHWQVPLGRRFRALKLWSVLRWYGAEGLRSHIRTGV
RHARLFADLVREDDRFTLLAPATLGLVTFRHAGSDTDNRNLLHAINAE
GTTFLTHSEKNGTFFLRFATGGTHTEDHHVHEAWRAVQNAVQNAVPRA
QHATDGTADALNG
SEQ. ID NO: 42
ATGGATATTGAAGCTTTTAGAAAAGCAGGTTACCAAGCTATCGATAGA
ATTTGTGACTACTACTACTCTTTGCAAAATAGACCAGTTGTTCCATCA
GTTCAACCAGGTTATTTGTTAGATGCATTACCAGATTCTCCACCAGAA
CAAGGTGAAGATTTCACTGTTATTGCTGATGATTACCAAAAGTACATC
TTGCCAGGTTTGACACATTGGCAACATCCATCTTTCTTTGCATACTTT
CCAACAGCTTGTACTTTTGAAGGTATTTTGGGTGACTTGTATTCTACA
TCAACTGCAAATCCAGGTTTTAATTGGTTGGCTTCTCCAGCATGTACT
GAATTAGAAATGGTTGTTATGGATTGGTCTGCTAAGTTGTTAGGTTTG
TCAGAACATTTCTTGCATTCTTCAGGTAAAGGTGGTGGTGTTATTCAA
ACTACAGCATCAGAATTGGCTTTAGTTGTTGTTGTTGCTGCAAGAGAA
AGATACTTGAGAATTCATCCAGATGCTAAGGCAGATGAATTGGTTATA
TATACTACAACTCAAACACATTCTTTGGGTGTTAAAGCTGGTTTAGTT
TTTGGTATGGAATGTAGAGCATTGGAAGTTAAAGCTGAAGATGCTTAC
GCATTAAGAGGTGCAACTTTGAAGTCAGCTTTAGAAGAAGATGAAAAG
AGAGGTAAAAGACCTTTTATTTTGGTTGCAACAGTTGGTACAACTTCT
TCAGGTGCTATTGATAGATTGGATGAAATTGGTCAAGTTTCTGAAGAT
TATCCATCATTATGGATTCATGTTGATGCTGCATGGGCAGGTGTTACT
TTGGCTTGTCCAGAATACAGAGGTACAGCTCAATTAGAAAACATCAAC
GCTTATGCAACATCTTTCGGTACTAACTTCCATAAGTGGGGTTTGGTT
AACTTTGATGCTGCATTGTTGTGGGTTAAGGATAGAAAGGATTTGACA
GATGCTTTAGATGTTACTCCAGAATTCTTGAGAACAAAACAAGGTGAC
GCTGGTGCAGTTGTTGATTTTAGAAATTGGCATTTGGGTTTGGGTAGA
AGATTCAGATCTTTGAAGGTTTGGTTCGTTTTGAGATCATACGGTGTT
GAAGGTTTTAGAAACTACATCAGACAAGGTATTAAATTGAATGAACAT
TTTACTTCTTTAATTAGAGCTTCTTTGGATTTTTCATTAGTTACAGCA
CCATCATTCGCTTTGACAGTTTTTAGATTGACTCCAGCTGGTGCATCT
TTGACTGGTTCAGAATTGAACGAATTAAACAGAGCTTTCTACGCAAGA
TTATCTTCAAGACATGATATCATGTTGACACAAACTGTTTTGAACGGT
GTTTTCTGTATCAGATTTGCAGTTGGTGCTGCAAGAACTCAACAAGAA
CATATTGATACAGCTTGGGATTTGTTACAACAAGAAGGTGCTGTTGCA
GTTCAAGAATACATGCAAAAGATTTCTAAGGATGTTCAATAA
SEQ. ID NO: 43
MDIEAFRKAGYQAIDRICDYYYSLQNRPVVPSVQPGYLLDALPDSPPE
QGEDFTVIADDYQKYILPGLTHWQHPSFFAYFPTACTFEGILGDLYST
STANPGFNWLASPACTELEMVVMDWSAKLLGLSEHFLHSSGKGGGVIQ
TTASELALVVVVAARERYLRIHPDAKADELVIYTTTQTHSLGVKAGLV
FGMECRALEVKAEDAYALRGATLKSALEEDEKRGKRPFILVATVGTTS
SGAIDRLDEIGQVSEDYPSLWIHVDAAWAGVTLACPEYRGTAQLENIN
AYATSFGTNFHKWGLVNFDAALLWVKDRKDLTDALDVTPEFLRTKQGD
AGAVVDFRNWHLGLGRRFRSLKVWFVLRSYGVEGFRNYIRQGIKLNEH
FTSLIRASLDFSLVTAPSFALTVFRLTPAGASLTGSELNELNRAFYAR
LSSRHDIMLTQTVLNGVFCIRFAVGAARTQQEHIDTAWDLLQQEGAVA
VQEYMQKISKDVQ
SEQ. ID NO: 44
ATGAAGTTCTGGAGAAAGTACACACAACAAGAAATGGATGAAAAGATT
ACTGAATCTTTGGAAAAGACTTTGAACTACGATAACACTAAGACAATC
GGTATTCCAGGTACTAAGTTGGATGATACAGTTTTCTATGATGATCAT
TCTTTCGTTAAGCATTCACCATACTTGAGAACTTTTATTCAAAACCCA
AACCATATCGGTTGTCATACTTATGATAAGGCTGATATCTTGTTCGGT
GGTACATTCGATATCGAAAGAGAATTAATCCAATTGTTAGCAATCGAT
GTTTTGAACGGTAACGATGAAGAATTTGATGGTTACGTTACTCAAGGT
GGTACAGAAGCTAACATCCAAGCAATGTGGGTTTACAGAAACTACTTC
AAGAAAGAAAGAAAGGCTAAGCATGAAGAAATCGCTATCATCACTTCA
GCAGATACACATTACTCTGCATACAAAGGTTCAGATTTGTTGAACATC
GATATTATTAAGGTTCCAGTTGATTTTTATTCAAGAAAAATTCAAGAA
AATACATTGGATTCAATTGTTAAAGAAGCTAAAGAAATTGGTAAAAAG
TACTTCATCGTTATCTCTAACATGGGTACTACAATGTTTGGTTCAGTT
GATGATCCAGATTTGTACGCTAACATCTTCGATAAGTACAATTTGGAA
TACAAAATTCATGTTGATGGTGCATTTGGTGGTTTTATATATCCAATT
GATAATAAGGAATGTAAAACTGATTTCTCTAATAAGAACGTTTCTTCA
ATCACATTAGATGGTCATAAGATGTTGCAAGCTCCATACGGTACTGGT
ATCTTCGTTTCAAGAAAGAATTTGATCCATAACACTTTGACAAAGGAA
GCAACTTACATCGAAAATTTGGATGTTACATTGTCTGGTTCAAGATCT
GGTTCAAATGCTGTTGCAATTTGGATGGTTTTAGCTTCTTATGGTCCA
TACGGTTGGATGGAAAAGATTAATAAGTTGAGAAATAGAACTAAATGG
TTGTGTAAGCAATTGAACGATATGAGAATTAAATATTACAAAGAAGAT
TCAATGAATATTGTTACAATTGAAGAACAATATGTTAATAAGGAAATC
GCTGAAAAGTACTTTTTAGTTCCAGAAGTTCATAACCCAACTAACAAC
TGGTACAAGATCGTTGTTATGGAACATGTTGAATTGGATATCTTGAAC
TCTTTGGTTTACGATTTGAGAAAGTTTAATAAGGAACATTTGAAGGCA
ATGTAA
SEQ. ID NO: 45
MKFWRKYTQQEMDEKITESLEKTLNYDNTKTIGIPGTKLDDTVFYDDH
SFVKHSPYLRTFIQNPNHIGCHTYDKADILFGGTFDIERELIQLLAID
VLNGNDEEFDGYVTQGGTEANIQAMWVYRNYFKKERKAKHEEIAIITS
ADTHYSAYKGSDLLNIDIIKVPVDFYSRKIQENTLDSIVKEAKEIGKK
YFIVISNMGTTMFGSVDDPDLYANIFDKYNLEYKIHVDGAFGGFIYPI
DNKECKTDFSNKNVSSITLDGHKMLQAPYGTGIFVSRKNLIHNTLTKE
ATYIENLDVTLSGSRSGSNAVAIWMVLASYGPYGWMEKINKLRNRTKW
LCKQLNDMRIKYYKEDSMNIVTIEEQYVNKEIAEKYFLVPEVHNPTNN
WYKIVVMEHVELDILNSLVYDLRKFNKEHLKAM
SEQ. ID NO: 46
ATGAATGCATCTGAATTCAGAAGAAGAGGTAAAGAAATGGTTGATTAC
ATGGCTAACTACATGGAAGGTATTGAAGGTAGACAAGTTTATCCAGAT
GTTGAACCAGGTTACTTGAGACCATTAATTCCAGCTGCAGCTCCACAA
GAACCAGATACATTTGAAGATATTATTAACGATGTTGAAAAGATTATC
ATGCCAGGTGTTACACATTGGCATTCACCATATTTCTTTGCATACTTT
CCAACTGCTTCTTCATATCCAGCAATGTTGGCTGATATGTTATGTGGT
GCAATTGGTTGTATTGGTTTTTCTTGGGCAGCTTCACCAGCTTGTACA
GAATTGGAAACTGTTATGATGGATTGGTTAGGTAAAATGTTGGAATTA
CCAAAGGCATTTTTGAACGAAAAGGCTGGTGAAGGTGGTGGTGTTATT
CAAGGTTCTGCATCAGAAGCTACATTAGTTGCTTTGTTAGCAGCTAGA
ACTAAAGTTATTCATAGATTGCAAGCAGCTTCTCCAGAATTAACACAA
GCAGCTATCATGGAAAAATTGGTTGCATATTCTTCAGATCAAGCTCAT
TCTTCAGTTGAAAGAGCTGGTTTGATCGGTGGTGTTAAGTTGAAGGCA
ATCCCATCTGATGGTAATTTTGCTATGAGAGCATCAGCTTTGCAAGAA
GCATTAGAAAGAGATAAAGCAGCTGGTTTGATTCCATTTTTCATGGTT
GCTACTTTAGGTACTACAACTTGTTGTTCTTTCGATAATTTGTTAGAA
GTTGGTCCAATTTGTAATAAGGAAGATATTTGGTTGCATGTTGATGCA
GCTTACGCAGGTTCAGCTTTTATTTGTCCAGAATTCAGACATTTGTTG
AACGGTGTTGAATTCGCTGATTCTTTTAATTTCAATCCACATAAATGG
TTGTTAGTTAACTTCGATTGTTCAGCAATGTGGGTTAAGAAAAGAACA
GATTTGACTGGTGCTTTTAGATTAGATCCAACATATTTGAAGCATTCT
CATCAAGATTCAGGTTTGATCACTGATTACAGACATTGGCAAATTCCA
TTGGGTAGAAGATTCAGATCTTTGAAGATGTGGTTCGTTTTTAGAATG
TACGGTGTTAAGGGTTTGCAAGCTTACATCAGAAAGCATGTTCAATTG
TCTCATGAATTCGAATCATTAGTTAGACAAGATCCAAGATTCGAAATC
TGTGTTGAAGTTATTTTGGGTTTAGTTTGTTTCAGATTGAAGGGTTCT
AATAAGGTTAACGAAGCATTGTTGCAAAGAATTAATTCAGCTAAGAAA
ATTCATTTGGTTCCATGTCATTTGCGTGATAAGTTCGTTTTGAGATTC
GCTATTTGTTCAAGAACTGTTGAATCAGCACATGTTCAAAGAGCTTGG
GAACATATCAAGGAATTGGCAGCTGATGTTTTGAGAGCTGAAAGAGAA
TAA
SEQ. ID NO: 47
MNASEFRRRGKEMVDYMANYMEGIEGRQVYPDVEPGYLRPLIPAAAPQ
EPDTFEDIINDVEKIIMPGVTHWHSPYFFAYFPTASSYPAMLADMLCG
AIGCIGFSWAASPACTELETVMMDWLGKMLELPKAFLNEKAGEGGGVI
QGSASEATLVALLAARTKVIHRLQAASPELTQAAIMEKLVAYSSDQAH
SSVERAGLIGGVKLKAIPSDGNFAMRASALQEALERDKAAGLIPFFMV
ATLGTTTCCSFDNLLEVGPICNKEDIWLHVDAAYAGSAFICPEFRHLL
NGVEFADSFNFNPHKWLLVNFDCSAMWVKKRTDLTGAFRLDPTYLKHS
HQDSGLITDYRHWQIPLGRRFRSLKMWFVFRMYGVKGLQAYIRKHVQL
SHEFESLVRQDPRFEICVEVILGLVCFRLKGSNKVNEALLQRINSAKK
IHLVPCHLRDKFVLRFAICSRTVESAHVQRAWEHIKELAADVLRAERE
SEQ. ID NO: 48
ATGGGTTCTATCTCAGAAAACTGTGATGATTCTATTTCATTAGCTGCA
CCTTTTAGACCATTGGAACCAGAAGAATTCAGAAAGCAAGCTCATGTT
ATGGTTGATTTCATCGCAGATTACTACAAGAACATTGAAAATTATCCA
GTTTTGTCACAAGTTGAACCAGGTTATTTGAAAAATAGATTGCCAGAA
ACTGCTCCACATTTGCCAGAATCTTTCGAAACAATCTTGAAGGATATT
AAGAAAGATATCGTTCCTGGTATGACTAATTGGTTGTCACCAAATTTC
TTTGCTTATTTTCCAGCAACTGTTTCTTCAGCTGCATTTGTTGGTGAA
ATGTTGTGTACAGGTTTTAATTCTGTTGGTTTTAATTGGTTAGCTTCA
CCAGCATCTACAGAATTGGAAATGGTTGTTATTGATTGGTTAGCTAAC
ATGTTGAAGTTGCCAAAGTCTTTTATGTTTCATGGTACTGGTGGTGGT
GTTATTCAAGGTACTACATCTGAAGCTATCTTGTGTACATTGATTGCT
GCAAGAGATGGTGCTTTGGAAAAGATTGGTATGGAAAACGTTGGTAAA
TTAGTTGTTTACGGTTCAGATCAAACTCATTCTTTCTTTCAAAAGACT
TGTAAGGTTGCTGGTATCTTCCCATGTAACATCAAGTTGATCCCAACT
ACAAGAGAAGATAACTTCTCTATGTCACCAATCGCATTGAGAGAACAA
ATTGAAGCTGATGTTGCAGATGGTTTAGTTCCAATTTTCTTGTGTACT
ACAGTTGGTACTACATCAACTGCTGCAATTGATCCAGTTTCTGAAGTT
GCTAAGGTTGCAAACGATTTCAATATTTGGGTTCATGTTGATGCTGCT
TATGCTGGTTCTGCATGTATTTGTCCAGAATTCAGACAATACTTAGAT
GGTATCGAATTGGTTGATTCTTTTTCATTGTCTCCACATAAGTGGTTG
TTGTGTTTCTTGGATTGTTGTTGTTTGTGGTTGAAGAAACCACATTTG
ATGGTTAAGGCTTTGTCAACTAACCCAGAATATTTGAGAAATAAGAGA
TCTGAATTCGATGGTGTTGTTGATTTCAAGGATTGGCAAATCGGTACA
GGTAGAAGATTCAAAGCTTTGAGATTATGGTTGGTTATGAGATCATAC
GGTGTTGAAAATTTGAAGAGACATATCTTGTCTGATGTTCAAATGGCT
AAGATGTTCGAAGGTTTAGTTAAATCTGATCCAAGATTCGAAATCATC
GTTCCAAGAGCTTTCGCATTAGTTTGTTTCAGATTGAACCCTGGTAAA
GGTTATGATGATGAAATCGATAAGGAAATCTTGAATAAGGAATTGTTA
GATTTGATTAATTCAACAGGTAGAGCTTACATGACTCATACAAAAGCA
GGTGGTATCTATATGTTGAGATTTGCTGTTGGTACTACATTGACTGAA
GAACATCATGTTTACGCTGCATGGGAATTGATTAAAGAATGTACAGAT
GCATCTTTGACTAAAACAAATATTATTGAATAA
SEQ. ID NO: 49
MGSISENCDDSISLAAPFRPLEPEEFRKQAHVMVDFIADYYKNIENYP
VLSQVEPGYLKNRLPETAPHLPESFETILKDIKKDIVPGMTNWLSPNF
FAYFPATVSSAAFVGEMLCTGFNSVGFNWLASPASTELEMVVIDWLAN
MLKLPKSFMFHGTGGGVIQGTTSEAILCTLIAARDGALEKIGMENVGK
LVVYGSDQTHSFFQKTCKVAGIFPCNIKLIPTTREDNFSMSPIALREQ
IEADVADGLVPIFLCTTVGTTSTAAIDPVSEVAKVANDFNIWVHVDAA
YAGSACICPEFRQYLDGIELVDSFSLSPHKWLLCFLDCCCLWLKKPHL
MVKALSTNPEYLRNKRSEFDGVVDFKDWQIGTGRRFKALRLWLVMRSY
GVENLKRHILSDVQMAKMFEGLVKSDPRFEIIVPRAFALVCFRLNPGK
GYDDEIDKEILNKELLDLINSTGRAYMTHTKAGGIYMLRFAVGTTLTE
EHHVYAAWELIKECTDASLTKTNIIE
SEQ. ID NO: 50
ATGGGTTCATTAGATACAAATCCAACTGCTTTTTCTGCATTTCCAGCT
GGTGAAGGTGAAACTTTTCAACCATTGAACGCAGATGATGTTAGATCA
TATTTGCATAAGGCTGTTGATTTCATCTCTGATTACTACAAATCAGTT
GAATCTATGCCAGTTTTGCCAAACGTTAAGCCAGGTTATTTGCAAGAT
GAATTAAGAGCATCACCACCAACTTACTCTGCTCCATTCGATGTTACA
ATGAAGGAATTGAGATCTTCAGTTGTTCCTGGTATGACTCATTGGGCA
TCTCCAAATTTCTTTGCTTTCTTTCCATCAACAAATTCTGCTGCAGCT
ATTGCTGGTGACTTGATTGCATCAGCTATGAATACAGTTGGTTTTACT
TGGCAAGCTTCTCCAGCAGCTACTGAAATGGAAGTTTTGGCATTAGAT
TGGTTGGCTCAAATGTTGAATTTGCCAACTTCTTTTATGAACAGAACT
GGTGAAGGTAGAGGTACTGGTGGTGGTGTTATTTTGGGTACTACATCT
GAAGCTATGTTGGTTACATTAGTTGCAGCTAGAGATGCAGCTTTGAGA
AGATCTGGTTCAGATGGTGTTGCAGGTTTGCATAGATTAGCTGTTTAT
GCAGCTGATCAAACACATTCAACTTTCTTTAAGGCATGTAGATTGGCT
GGTTTTGATCCAGCAAATATTAGATCTATTCCAACAGGTGCTGAAACT
GATTATGGTTTAGATCCAGCAAGATTGTTAGAAGCTATGCAAGCAGAT
GCTGATGCAGGTTTAGTTCCAACATACGTTTGTGCAACTGTTGGTACT
ACATCTTCAAATGCTGTTGATCCAGTTGGTGCTGTTGCAGATGTTGCA
GCTAGATTTGCAGCTTGGGTTCATGTTGATGCAGCTTACGCAGGTTCA
GCTTGTATTTGTCCAGAATTCAGACATCATTTGGATGGTGTTGAAAGA
GTTGATTCTATCTCAATGTCTCCACATAAGTGGTTGATGACATGTTTA
GATTGTACTTGTTTGTACGTTAGAGATACACATAGATTGACTGGTTCT
TTAGAAACTAACCCAGAATACTTGAAAAATCATGCTTCAGATTCTGGT
GAAGTTACAGATTTGAAAGATATGCAAGTTGGTGTTGGTAGAAGATTC
AGAGGTTTGAAGTTGTGGATGGTTATGAGAACTTATGGTGTTGCTAAG
TTGCAAGAACATATCAGATCAGATGTTGCTATGGCAAAAGTTTTTGAA
GATTTGGTTAGAGGTGACGATAGATTCGAAGTTGTTGTTCCAAGAAAC
TTCGCTTTGGTTTGTTTCAGAATTAGAGCTGGTGCAGGTGCAGCTGCA
GCTACAGAAGAAGATGCAGATGAAGCTAACAGAGAATTGATGGAAAGA
TTGAATAAGACTGGTAAAGCATACGTTGCTCATACAGTTGTTGGTGGT
AGATTTGTTTTGAGATTTGCAGTTGGTTCTTCATTACAAGAAGAACAT
CATGTTAGATCTGCTTGGGAATTGATTAAGAAAACTACAACTGAAATG
ATGAACTAA
SEQ. ID NO: 51
MGSLDTNPTAFSAFPAGEGETFQPLNADDVRSYLHKAVDFISDYYKSV
ESMPVLPNVKPGYLQDELRASPPTYSAPFDVTMKELRSSVVPGMTHWA
SPNFFAFFPSTNSAAAIAGDLIASAMNTVGFTWQASPAATEMEVLALD
WLAQMLNLPTSFMNRTGEGRGTGGGVILGTTSEAMLVTLVAARDAALR
RSGSDGVAGLHRLAVYAADQTHSTFFKACRLAGFDPANIRSIPTGAET
DYGLDPARLLEAMQADADAGLVPTYVCATVGTTSSNAVDPVGAVADVA
ARFAAWVHVDAAYAGSACICPEFRHHLDGVERVDSISMSPHKWLMTCL
DCTCLYVRDTHRLTGSLETNPEYLKNHASDSGEVTDLKDMQVGVGRRF
RGLKLWMVMRTYGVAKLQEHIRSDVAMAKVFEDLVRGDDRFEVVVPRN
FALVCFRIRAGAGAAAATEEDADEANRELMERLNKTGKAYVAHTVVGG
RFVLRFAVGSSLQEEHHVRSAWELIKKTTTEMMN
SEQ. ID NO: 52
ATGGAAGGTGTTGGTGGTGGTGGTGGTGGTGAAGAATGGTTGAGACCA
ATGGATGCAGAACAATTAAGAGAATGTGGTCATAGAATGGTTGATTTC
GTTGCAGATTACTACAAGTCAATCGAAGCTTTTCCAGTTTTGTCTCAA
GTTCAACCAGGTTATTTGAAAGAAGTTTTGCCAGATTCAGCTCCAAGA
CAACCAGATACTTTAGATTCTTTGTTCGATGATATCCAACAAAAGATT
ATCCCAGGTGTTACACATTGGCAATCACCAAACTACTTCGCATACTAC
CCATCTAATTCTTCTACTGCTGGTTTCTTGGGTGAAATGTTGTCTGCT
GCTTTTAATATCGTTGGTTTTTCATGGATCACTTCTCCAGCTGCAACA
GAATTGGAAGTTATTGTTTTAGATTGGTTCGCAAAGATGTTGCAATTG
CCATCACAATTCTTATCTACTGCTTTGGGTGGTGGTGTTATTCAAGGT
ACAGCTTCAGAAGCAGTTTTGGTTGCATTGTTAGCTGCAAGAGATAGA
GCTTTGAAGAAACATGGTAAACATTCTTTAGAAAAATTGGTTGTTTAC
GCATCAGATCAAACTCATTCTGCTTTACAAAAAGCATGTCAAATCGCT
GGTATTTTCTCTGAAAACGTTAGAGTTGTTATTGCTGATTGTAATAAG
AACTACGCTGTTGCACCAGAAGCAGTTTCAGAAGCTTTGTCTATCGAT
TTGTCTTCAGGTTTGATCCCATTTTTCATTTGTGCAACAGTTGGTACT
ACATCTTCATCTGCTGTTGATCCATTACCAGAATTGGGTCAAATCGCT
AAGTCAAACGATATGTGGTTCCATATCGATGCTGCTTATGCTGGTTCT
GCATGTATTTGTCCAGAATACAGACATCATTTGAATGGTGTTGAAGAA
GCAGATTCTTTTAATATGAATGCTCATAAGTGGTTCTTGACTAACTTC
GATTGTTCATTGTTGTGGGTTAAGGATAGATCATTTTTGATCCAATCA
TTATCTACAAATCCAGAATTCTTGAAAAATAAGGCTTCACAAGCAAAC
TCTGTTGTTGATTTCAAGGATTGGCAAATCCCATTGGGTAGAAGATTC
AGATCATTGAAGTTGTGGATGGTTTTGAGATTGTACGGTGTTGATAAT
TTGCAATCTTACATCAGAAAGCATATCCATTTGGCTGAACATTTCGAA
CAATTGTTGTTGTCAGATTCAAGATTCGAAGTTGTTACTCCAAGAACA
TTTTCATTGGTTTGTTTCAGATTAGTTCCACCAACTTCTGATCATGAA
AACGGTAGAAAGTTGAACTACGATATGATGGATGGTGTTAATTCATCT
GGTAAAATTTTCTTGTCACATACAGTTTTGTCTGGTAAATTCGTTTTG
AGATTTGCTGTTGGTGCACCATTAACTGAAGAAAGACATGTTGATGCT
GCATGGAAGTTGTTGAGAGATGAAGCTACAAAGGTTTTAGGTAAAATG
GTTTAA
SEQ. ID NO: 53
MEGVGGGGGGEEWLRPMDAEQLRECGHRMVDFVADYYKSIEAFPVLSQ
VQPGYLKEVLPDSAPRQPDTLDSLFDDIQQKIIPGVTHWQSPNYFAYY
PSNSSTAGFLGEMLSAAFNIVGFSWITSPAATELEVIVLDWFAKMLQL
PSQFLSTALGGGVIQGTASEAVLVALLAARDRALKKHGKHSLEKLVVY
ASDQTHSALQKACQIAGIFSENVRVVIADCNKNYAVAPEAVSEALSID
LSSGLIPFFICATVGTTSSSAVDPLPELGQIAKSNDMWFHIDAAYAGS
ACICPEYRHHLNGVEEADSFNMNAHKWFLTNFDCSLLWVKDRSFLIQS
LSTNPEFLKNKASQANSVVDFKDWQIPLGRRFRSLKLWMVLRLYGVDN
LQSYIRKHIHLAEHFEQLLLSDSRFEVVTPRTFSLVCFRLVPPTSDHE
NGRKLNYDMMDGVNSSGKIFLSHTVLSGKFVLRFAVGAPLTEERHVDA
AWKLLRDEATKVLGKMV
SEQ. ID NO: 54
ATGACAAAGCCATTCCATCCAAATTTGAACGTTGATGCATTGTTTTTA
GGTCCAAAGTCTGAAAACGCTGTTTTCTTTAGAGAAATGATGGATTAT
GCAGTTTCTGAACACATGCATTGGAGATCAGGTTTTCATCCAGAAGAT
TCTACATTGGTTACTTCAGTTGATAGATACGAACATAACTACAGAGAA
ACATTGTACAGAACTGAAGGTATTTTGAATCAATTATCTGCTAAGTTG
AAAAATTCTTCAATCCCATTTTTCTCTCCAAGATACTTAGGTCATATT
TCTTCAGATACTTTGATGGTTTCTAATTTGGCTTATGTTATGGCTATG
ATGTACAACCCAAACAACTGTTCTTATGAAGCTTCACCAACTACAACT
GAATTGGAATTAGAATCTGGTTTGGATTTGTGTAGAATGTTCGGTTAC
AATCCACAACAATCTTGGGGTCATATTACATCAGGTGGTACTGTTGCA
AATTACGAAGGTTTGTGGGTTGCTAGAAATTTGAAGACATTGTCTTTC
GCAATCTCACAACATCCAAAGGCTAAGGATTTGATCGAACATAAGACT
CCAAAGCAATTGAAAAATATGCCAACATCTGAATTGTTAGATTTGATC
ACTGAATTACAAAAGAGAGGTTTGTTCGAAGAAATCAGAGATATGACA
TGTAGAGGTACTGGTGTTAAGGCTCAAATTTTAGGTAAATTGTTAGTT
CCACAATCTAAGCATTATTCATGGATGAAAGCTGCAGATATCTTCGGT
ATCGGTCAAGAAAACATCATCCCATTGCCAGTTAATGATCATTACCAA
ACAGATGTTGCTCAAATGAGAAAGATCACTTTCGGTTTGATTGAACAA
GGTGAACCAATTTTAGCAATGATTGCTGTTGTTGGTACAACTGAAGTT
GGTGCAATCGATAGAATCGATGAAGTTATTGCTTTAAGAAAAGAATGT
GAAGAAAGATACGGTGAATCTTTCTACATCCATGTTGATGCTGCTTAT
GCTGGTTACGCTTGTTCAATGTTGTTGAACGAACAGGGTGAATTCGTT
GAATACGATGAATTGGCAAACTACCATAGATCTGCTGGTATTATGCCA
GAAAATATTTCATGGCCAAAACCAGAAGTTTACCAATCTTTTAAAGCT
TTGAAAGATGTTGATTCAATTACAGTTGATCCACATAAGGTTGGTTTT
ATTCAATATGCTGCAGGTGCAATTTGTATGAAGGATAAGAGAATTTTG
GAATTAATTTCTTCACATGCTGCATACGTTTTTGAAGGTCATGCTGAT
AAGACATCTACTAAAAATAGAGGTATTTTGGGTTCTTCAATTATGGAA
GGTTCTAAATCAGGTGCAACAGCTGCAGCTTTGTGGGCAGCTCATAGA
TTGTTACCATTGAACATCAACGGTTACGGTAAAGTTATTGCAGCTGGT
ATCGTTACTGCACAATCTTTGTTGAATAAGTTGGCTAATTTGCCACCA
ATCGAAATCGGTAAACATAGATTCGAAGTTCATATTTTTCCATCACCA
GATTTCCACATGATCAACTTCACTTTTAAAGAAGTTGGTAACGAATCT
TTGTCAAACCATAACGCTTTGAATAAGAGATTGTATGAATTATGTTCT
TACTCAGCAGGTAGAGCTTACACAAATGATTTCTTGACATCTTCAACT
ATTTTAGATTACAAGGAATACGGTGACACTCCTTGGCATCATGCTCAA
AAATGTGGTTTTTCAAGATCAGAATGGGAAAAAGTTAGAAACATCTAT
GTTTTGAGAGCAGCTGTTATGACATACTGTTTGAGAGATGAAGAACAT
TTCGAAGAATTCTGGGAACAATTGCAAGCTATCTTCGTTAAGAAATTG
ACTCAAATCGTTGATGAAGAAGATAAGAAAGCAAGATTGAGATTAGAA
CCAGATGTTCCTTTTATTGGTTAA
SEQ. ID NO: 55
MTKPFHPNLNVDALFLGPKSENAVFFREMMDYAVSEHMHWRSGFHPED
STLVTSVDRYEHNYRETLYRTEGILNQLSAKLKNSSIPFFSPRYLGHI
SSDTLMVSNLAYVMAMMYNPNNCSYEASPTTTELELESGLDLCRMFGY
NPQQSWGHITSGGTVANYEGLWVARNLKTLSFAISQHPKAKDLIEHKT
PKQLKNMPTSELLDLITELQKRGLFEEIRDMTCRGTGVKAQILGKLLV
PQSKHYSWMKAADIFGIGQENIIPLPVNDHYQTDVAQMRKITFGLIEQ
GEPILAMIAVVGTTEVGAIDRIDEVIALRKECEERYGESFYIHVDAAY
AGYACSMLLNEQGEFVEYDELANYHRSAGIMPENISWPKPEVYQSFKA
LKDVDSITVDPHKVGFIQYAAGAICMKDKRILELISSHAAYVFEGHAD
KTSTKNRGILGSSIMEGSKSGATAAALWAAHRLLPLNINGYGKVIAAG
IVTAQSLLNKLANLPPIEIGKHRFEVHIFPSPDFHMINFTFKEVGNES
LSNHNALNKRLYELCSYSAGRAYTNDFLTSSTILDYKEYGDTPWHHAQ
KCGFSRSEWEKVRNIYVLRAAVMTYCLRDEEHFEEFWEQLQAIFVKKL
TQIVDEEDKKARLRLEPDVPFIG
SEQ. ID NO: 56
ATGGGTTCTTTGGATTCAAACTACGATACTGAATCTCCAGCTTCAGTT
GGTCAATTCAATCCATTAGATCCAGAAGAATTCAGAAAGCAAGCTCAT
TGTATCGTTGATTTCATCGCAGATTACTACAAGAACATTGAATCTTAT
CCAGTTTTGTCACAAGTTGATCCAGGTTACAGACATTCAAGATTGGGT
AAAAATGCTCCATACAGATCTGAACCATTCGAATCAATCTTGAAGGAT
GTTCAAAAGGATATCATCCCTGGTATGACTCATTGGATGTCTCCAAAT
TTCTTTGCTCATTTTCCAGCAACTGTTTCTTCAGCTGCATTTGTTGGT
GAAATGTTGTGTACATGTTTCAACTCTGTTGGTTTTAATTGGTTAGCA
TCACCAGCTGCAACAGAATTGGAAATGGTTGTTATTGATTGGTTAGCT
AACATGTTGAAGTTGCCAAAGTCTTTTATGTTTTCAGGTACTGGTGGT
GGTGTTTTGCAAGGTACTACATCTGAAGCTATCTTGTGTACATTGATT
GCTGCATCACCAATGCATTTCGAAATCGTTGGTGTTAAGACTTCTACA
TCATTCGTTGTTTATGGTTCTGATCAAACTCATTCAACATACGCTAAA
GCATGTAAATTAGCAGGTATTTTGCCATGTAACATCAGATCTATTCCA
ACTACAGCTGATTCAAACTTCTCTGTTTCACCATTGTTGTTGAGAAGA
GCTATTGAAGCAGATAAAGCTGCAGGTATGGTTCCATTGTATATTTGT
GCTACAGTTGGTACTACATCTACTACAGCAATTGATCCATTATCTTCA
TTGGCTGATGTTGCAAATGATTATGGTGTTTGGTTTCATGTTGATGCT
GCATACGCTGGTTCTGCATGTATTTGTCCAGAATTCAGACATTATTTG
GATGGTATCGAAAGAGCAGATTCTTTGTCATTGTCTCCACATAAGTGG
TTGTTGTCTTACTTAGATTGTTGTTGTTTGTGGGTTAAGTCACCATCT
TTATTGGTTAAGGCTTTATCAACTGATCCAGAATATTTGAAAAATCAA
CCATCAGAATCTAAGTCAGTTGTTGATTACAAGGATTGGCAAGTTGGT
ACAGGTAGAAGATTCAAAGCTTTGAGATTGTGGTTCGTTATGAGATCT
TATGGTGTTGCAAATTTGCAATCACATATCAGAACTGATGTTCAAATG
GCTAAGATGTTCGAAGGTTTCGTTAAGTCTGATCCAAGATTCGAAATT
TTGGTTCCAAGAGTTTTCTCTTTGGTTTGTTTCAGATTGAACCCAATC
TCTGGTTCAGATCCAACTGGTACAGAAGCTTTGAACAGAAAGTTATTG
GATTGGGTTAATTCTACAGGTAGAGTTTATATGACTCATACAAAAGTT
GGTGGTATCTATATGTTGAGATTTGCTGTTGGTGCAACTTTAACAGAA
AAGAGACATGTTTCTTCAGCTTGGAAGTTGATTAAAGAAGGTGCAGAT
GTTTTGTTGAAGGAAGATTAA
SEQ. ID NO: 57
MGSLDSNYDTESPASVGQFNPLDPEEFRKQAHCIVDFIADYYKNIESY
PVLSQVDPGYRHSRLGKNAPYRSEPFESILKDVQKDIIPGMTHWMSPN
FFAHFPATVSSAAFVGEMLCTCFNSVGFNWLASPAATELEMVVIDWLA
NMLKLPKSFMFSGTGGGVLQGTTSEAILCTLIAASPMHFEIVGVKTST
SFVVYGSDQTHSTYAKACKLAGILPCNIRSIPTTADSNFSVSPLLLRR
AIEADKAAGMVPLYICATVGTTSTTAIDPLSSLADVANDYGVWFHVDA
AYAGSACICPEFRHYLDGIERADSLSLSPHKWLLSYLDCCCLWVKSPS
LLVKALSTDPEYLKNQPSESKSVVDYKDWQVGTGRRFKALRLWFVMRS
YGVANLQSHIRTDVQMAKMFEGFVKSDPRFEILVPRVFSLVCFRLNPI
SGSDPTGTEALNRKLLDWVNSTGRVYMTHTKVGGIYMLRFAVGATLTE
KRHVSSAWKLIKEGADVLLKED
SEQ. ID NO: 58
ATGGAAGGTGGTTTTACTGGTGGTGACGAATACCAAAAGCATTTCTTG
CCAAGAGATTACTTAAACACATACTACTCATTCCAATCTGGTCCATCA
CCAGAAGCTGAAATGTTGAAGTTTAATTTGGAATGTTTGCATAAGACT
TTCGGTCCAGGTGGTTTGCAAGGTGACACTTTAATTGATATTGGTTCT
GGTCCAACAATCTATCAAGTTTTGGCTGCATGTGAATCTTTTAAAGAT
ATCACTTTGTCAGATTTTACAGATAGAAATAGAGAAGAATTGGCTAAA
TGGTTGAAGAAAGAACCAGGTGCATACGATTGGACTCCAGCTTTGAAG
TTCGCATGTGAATTAGAAGGCAACTCTGGTAGATGGCAAGAAAAAGCT
GAAAAGTTGAGAGCAACAGTTAAGAGAGTTTTGAAGTGTGATGCTAAT
TTGTCAAATCCATTAACTCCAGTTGTTTTGCCACCAGCAGATTGTGTT
TTGACATTGTTAGCTATGGAATGTGCATGTTGTTCTTTGGATGCTTAT
AGAGCTGCATTGAGAAATTTGGCATCATTGTTAAAACCAGGTGGTCAT
TTGGTTACTACAGTTACTTTGCAATTATCTTCATACATGGTTGGTGAA
AGAGAATTTTCTTGTGTTGCTTTGGAAAAAGAAGAAGTTGAACAAGCT
GTTTTAGATGCAGGTTTCGATATCGAACAATTGTTGTACTCTCCACAA
TCTTACTCAGCTTCTACAGCACCAAATAGAGGTGTTTGTTTCTTGGTT
GCTAGAAAGAAACCAGGTTCATAA
SEQ. ID NO: 59
MEGGFTGGDEYQKHFLPRDYLNTYYSFQSGPSPEAEMLKFNLECLHKT
FGPGGLQGDTLIDIGSGPTIYQVLAACESFKDITLSDFTDRNREELAK
WLKKEPGAYDWTPALKFACELEGNSGRWQEKAEKLRATVKRVLKCDAN
LSNPLTPVVLPPADCVLTLLAMECACCSLDAYRAALRNLASLLKPGGH
LVTTVTLQLSSYMVGEREFSCVALEKEEVEQAVLDAGFDIEQLLYSPQ
SYSASTAPNRGVCFLVARKKPGS
SEQ. ID NO: 60
ATGGATAAGATCTCTGCACCATTTTTCTCTGGTACTTCACCAGCTGCA
GCTTCAGTTGCTGGTGTTGATGAAGATGATAGATTGTGTTTCCAAGCT
CAAGAATTGATGTTCGCATACAACATCTCTATGGTTTTGAGAGCAGCT
ATTCAATTAGGTTTGTTAGATGCTTTGTCAGCAGCTGGTGGTAAAGCA
TTGACTCCAAACGAATTAGTTGAAAACGTTGAAACATCTTCAAATAAG
GCTGAAGCAGCTGCAGCTGTTGATAGAATTTTGAGATATTTGTCTTGT
TTCAACGTTGTTACTTGTTCTTCAGAAGCAGCTGGTCCAGATGGTACA
TTAGTTAGAAGATACACTACAGGTCCATTGTGTAGATGGTTAACTAAA
GATAGAGGTGACGGTACATTATCTCCATTTGCTGTTTTTGTTGTTGAT
CCAGATCATTTGTTTCCTTGGCATCATATTGCAGAAGCTGTTACTGCT
GGTGGTCCATCAGCATTCGAAAGAACACAAAAGTGGCCATACTACGAA
TACATGGGTAAAAATCAAAGATTGGGTACTTTGTTCGATAACGCAATG
GCTCAACATTCTGTTATTTTGGTTACAAAGATGTTAGAAAGATTCAAA
GGTTTTGATGGTGTTCAAAGATTAGTTGATGTTGGTGGTGGTACTGGT
TCTACATTGGGTATGATCACTTCAAAGTACAAGCACATGACAGGTATT
AATTACGATTTGCCACATGTTATTGCTCAAGGTTTGCCATTACCAGGT
GTTGAACATGTTGCAGGTGACATGTACGAATCTATTCCAACTGGTGAC
GCTGTTTTGTTACAATGGATTACATTGATGTTGAACGATGATGAATTC
GTTAAGATCTTGTCAAATTGTCATAATGCTTTGCCAAAAGATGGTAAA
GTTATTGTTGTTGATGGTATTTTACCAGAAAACCCAGATTCTTCATTG
ACTGCAAGAGATGCTTTTACATTAGATATCATCATGTTCGTTTTGTTT
AAAGGTGCAAAGCAAAGAACTGAAAAGGAATTCGCAAGATTAGCTAAG
CAAGCAGGTTTTACTGGTGGTATTAAGAAAACTTATATTTTCTTTAAT
TTTTACGCTTTGGAATTCACTAAATAA
SEQ. ID NO: 61
MDKISAPFFSGTSPAAASVAGVDEDDRLCFQAQELMFAYNISMVLRAA
IQLGLLDALSAAGGKALTPNELVENVETSSNKAEAAAAVDRILRYLSC
FNVVTCSSEAAGPDGTLVRRYTTGPLCRWLTKDRGDGTLSPFAVFVVD
PDHLFPWHHIAEAVTAGGPSAFERTQKWPYYEYMGKNQRLGTLFDNAM
AQHSVILVTKMLERFKGFDGVQRLVDVGGGTGSTLGMITSKYKHMTGI
NYDLPHVIAQGLPLPGVEHVAGDMYESIPTGDAVLLQWITLMLNDDEF
VKILSNCHNALPKDGKVIVVDGILPENPDSSLTARDAFTLDIIMFVLF
KGAKQRTEKEFARLAKQAGFTGGIKKTYIFFNFYALEFTK
SEQ. ID NO: 62
ATGAACACCTTCAGAACAGCCACTGCCAGAGACATACCTGATGTAGCA
GCAACTCTTACGGAAGCCTTCGCAACTGATCCACCCACGCAGTGGGTG
TTCCCCGACGGTACTGCCGCCGTCAGCAGGTTCTTTACACATGTTGCA
GATAGGGTTCACACGGCCGGTGGTATTGTTGAGCTACTACCAGACAGA
GCCGCCATGATTGCATTGCCACCACACGTGAGGCTGCCAGGAGAAGCT
GCCGACGGAAGGCAGGCGGAAATTCAGAGAAGGCTGGCAGACAGGCAC
CCGCTGACACCTCACTACTACCTGCTGTTTTACGGAGTTAGAACGGCA
CACCAGGGTTCGGGATTGGGCGGAAGAATGCTGGCCAGATTAACTAGC
AGAGCTGATAGGGACAGGGTGGGTACATATACTGAGGCATCCACCTGG
CGTGGCGCTAGACTGATGCTGAGACATGGATTCCATGCTACAAGGCCA
CTAAGATTGCCAGATGGACCCAGCATGTTTCCACTTTGGAGAGATCCA
ATCCATGATCATTCTGATTAG
SEQ. ID NO: 63
MNTFRTATARDIPDVAATLTEAFATDPPTQWVFPDGTAAVSRFFTHVA
DRVHTAGGIVELLPDRAAMIALPPHVRLPGEAADGRQAEIQRRLADRH
PLTPHYYLLFYGVRTAHQGSGLGGRMLARLTSRADRDRVGTYTEASTW
RGARLMLRHGFHATRPLRLPDGPSMFPLWRDPIHDHSD
SEQ. ID NO: 64
ATGAAAGGATATTTCGGACCATACGGTGGCCAGTACGTACCAGAAATA
TTAATGGGTGCCTTAGAGGAGTTAGAGGCAGCATACGAGGAGATTATG
AAGGATGAGAGCTTCTGGAAGGAGTTCAACGATCTACTGAGGGATTAC
GCAGGCAGACCAACGCCATTGTACTTTGCCAGGAGATTGTCTGAGAAG
TACGGCGCCCGTGTTTACTTGAAGCGTGAGGATCTGCTGCACACTGGA
GCACACAAGATAAATAACGCTATCGGACAGGTTTTATTGGCCAAATTA
ATGGGGAAGACACGTATCATAGCCGAGACGGGAGCTGGGCAGCATGGA
GTCGCTACTGCTACCGCTGCTGCCCTGTTCGGAATGGAATGTGTGATC
TACATGGGTGAAGAGGACACAATCAGACAGAAGTTGAACGTGGAGCGT
ATGAAATTATTAGGGGCTAAAGTTGTCCCTGTTAAGTCTGGCAGTAGG
ACCTTGAAGGATGCGATAGACGAGGCTTTGAGAGACTGGATTACTAAT
TTACAGACAACATATTATGTTATCGGATCTGTTGTTGGTCCCCACCCT
TACCCAATTATCGTAAGGAATTTCCAGAAGGTTATCGGTGAGGAGACC
AAGAAGCAAATACCAGAAAAGGAAGGTCGTTTGCCAGACTATATAGTT
GCCTGCGTAGGCGGCGGTAGCAATGCCGCAGGTATATTTTACCCATTC
ATAGACTCTGGAGTAAAGCTGATAGGTGTTGAGGCAGGTGGCGAGGGA
TTGGAGACAGGTAAACACGCAGCCTCGTTATTAAAGGGTAAAATTGGC
TATTTACATGGATCGAAGACCTTTGTTCTACAAGATGACTGGGGTCAA
GTCCAAGTGAGCCATTCGGTGTCAGCTGGTCTTGACTATTCAGGAGTA
GGACCTGAGCATGCTTATTGGAGAGAGACAGGGAAGGTTCTGTACGAC
GCAGTGACTGACGAAGAGGCTTTGGACGCATTTATAGAGTTATCAAGA
CTAGAGGGCATTATACCCGCTTTAGAGTCATCGCATGCTCTAGCATAT
TTGAAGAAGATAAATATAAAAGGTAAGGTTGTGGTGGTCAACCTATCA
GGGAGAGGGGATAAAGACCTGGAGTCAGTCTTAAACCATCCATACGTG
AGAGAAAGAATTAGATGA
SEQ. ID NO: 65
MKGYFGPYGGQYVPEILMGALEELEAAYEEIMKDESFWKEFNDLLRDY
AGRPTPLYFARRLSEKYGARVYLKREDLLHTGAHKINNAIGQVLLAKL
MGKTRIIAETGAGQHGVATATAAALFGMECVIYMGEEDTIRQKLNVER
MKLLGAKVVPVKSGSRTLKDAIDEALRDWITNLQTTYYVIGSVVGPHP
YPIIVRNFQKVIGEETKKQIPEKEGRLPDYIVACVGGGSNAAGIFYPF
IDSGVKLIGVEAGGEGLETGKHAASLLKGKIGYLHGSKTFVLQDDWGQ
VQVSHSVSAGLDYSGVGPEHAYWRETGKVLYDAVTDEEALDAFIELSR
LEGIIPALESSHALAYLKKINIKGKVVVVNLSGRGDKDLESVLNHPYV
RERIR
SEQ. ID NO: 66
ATGTGGTTCGGTGAGTTTGGTGGACAATATGTGCCAGAGACTTTAGTG
GGTCCTCTTAAGGAATTGGAAAAGGCATATAAAAGGTTCAAGGACGAT
GAGGAGTTCAACAGGCAACTAAACTATTATTTGAAGACATGGGCCGGT
AGACCAACGCCCTTGTATTATGCTAAGAGGTTAACTGAAAAGATTGGC
GGCGCGAAAGTGTATCTGAAAAGAGAAGACCTAGTTCATGGTGGAGCA
CACAAGACAAATAATGCCATTGGACAAGCACTATTGGCAAAGCTAATG
GGTAAAACTAGATTGATAGCTGAGACAGGAGCGGGTCAACATGGGGTC
GCGACAGCGATGGCTGGTGCACTACTGGGGATGAAGGTAGATATTTAC
ATGGGTGCTGAGGACGTTGAGCGTCAGAAACTAAATGTCTTCAGGATG
AAGCTATTAGGTGCCAATGTTATACCTGTAAATTCTGGCTCAAGAACA
CTAAAGGACGCCTTCGACGAGGCTCTTAGAGACTGGGTTGCCACTTTC
GAGTATACTCATTACTTGATCGGTTCAGTGGTTGGACCACATCCATAC
CCAACCATCGTTAGGGACTTTCAGAGCGTGATTGGTAGAGAGGCTAAG
GCACAGATCTTAGAAGCAGAGGGACAGCTACCTGACGTCATAGTTGCC
TGCGTCGGCGGTGGCTCTAACGCAATGGGTATATTCTATCCATTCGTT
AATGACAAGAAGGTTAAATTAGTAGGAGTCGAAGCTGGCGGAAAGGGG
TTAGAGTCGGGTAAACACTCAGCAAGCTTAAATGCAGGACAGGTAGGG
GTGTCCCACGGCATGTTGTCGTATTTCTTGCAAGACGAGGAAGGTCAG
ATAAAGCCAAGTCATTCAATTGCTCCAGGCCTTGACCACCCCGGTGTT
GGTCCAGAGCACGCTTACTTAAAGAAGATTCAAAGGGCCGAGTACGTC
GCTGTAACAGACGAAGAGGCATTGAAAGCTTTCCATGAGCTATCCAGA
ACTGAGGGGATTATACCCGCCCTTGAGTCTGCCCATGCTGTGGCGTAC
GCCATGAAGTTAGCTAAAGAGATGTCCCGTGACGAAATCATCATTGTA
AATCTATCAGGGAGAGGAGACAAGGATTTGGACATTGTATTGAAGGCA
AGCGGAAATGTTTGA
SEQ. ID NO: 67
MWFGEFGGQYVPETLVGPLKELEKAYKRFKDDEEFNRQLNYYLKTWAG
RPTPLYYAKRLTEKIGGAKVYLKREDLVHGGAHKTNNAIGQALLAKLM
GKTRLIAETGAGQHGVATAMAGALLGMKVDIYMGAEDVERQKLNVFRM
KLLGANVIPVNSGSRTLKDAFDEALRDWVATFEYTHYLIGSVVGPHPY
PTIVRDFQSVIGREAKAQILEAEGQLPDVIVACVGGGSNAMGIFYPFV
NDKKVKLVGVEAGGKGLESGKHSASLNAGQVGVSHGMLSYFLQDEEGQ
IKPSHSIAPGLDHPGVGPEHAYLKKIQRAEYVAVTDEEALKAFHELSR
TEGIIPALESAHAVAYAMKLAKEMSRDEIIIVNLSGRGDKDLDIVLKA
SGNV
SEQ. ID NO: 68
ATGTGGTTTGGGGAATTCGGTGGTCAATACGTGCCAGAAACCTTAGTT
GGGCCTTTAAAGGAGCTTGAGAAAGCTTACAAAAGATTTAAGGACGAC
GAGGAGTTTAATAGGCAATTAAACTATTACTTGAAGACTTGGGCAGGT
AGACCAACACCACTTTATTACGCGAAGAGACTTACAGAGAAGATCGGT
GGCGCTAAGGTTTATTTGAAGCGTGAGGATTTGGTTCATGGTGGAGCA
CATAAGACAAACAATGCCATCGGGCAAGCCTTGTTGGCCAAATTGATG
GGTAAGACTAGGTTGATAGCTGGCACAGGTGCCGGCCAGCATGGCGTT
GCAACAGCAATGGCTGGTGCTCTTTTAGGGATGAAGGTGGATATCTAT
ATGGGCGCCGAGGACGTTGAAAGACAAAAGCTTAACGTATTCAGAATG
AAGTTATTAGGTGCGAATGTTATTCCAGTGAATTCAGGTTCCAGGACG
CTGAAGGACGCAATAGACGAAGCCTTGAGAGACTGGGTTGCTACTTTT
GAGTACACACATTATTTGATTGGTTCCGTGGTTGGACCCCACCCGTAC
CCTACAATAGTTAGAGATTTTCAATCTGTTATCGGTAGAGAGGCGAAG
GCTCAAATTCCAGAGGCCGAGGGACAGCTACCCGATGTTATAGTCGCC
TGCGTTGGAGGAGGATCAAATGCGATGGGAATCTTCTACCCTTTTGTA
AATGATAAGAAAGTTAAGTTAGTCGGAGTTGAGGCCGGTGGTAAAGGG
CTAGAAAGCGGCAAACACTCGGCATCTCTTAATGCCGGTCAAGTAGGA
GTAAGTCACGGGATGTTATCTTACTTCTTGCAGGATGAGGAAGGTCAA
ATTAAGCCATCTCATTCTATAGCCCCAGGATTAGACTATCCCGGTGTT
GGGCCTGAGCACGCTTATCTGAAGAAGATACAGCGTGCTGAGTACGTT
GCAGTAACTGATGAAGAGGCTTTGAAGGCTTTTCACGAGTTATCAAGA
ACTGAGGGAATAATTCCTGCCTTGGAATCGGCACATGCGGTTGCCTAC
GCCATGAAGCTTGCTAAGGAAATGTCAAGAGACGAAATCATTATAGTG
AATTTATCGGGAAGGGGCGATAAGGACTTGGACATTGTTTTAAAAGCA
AGTGGCAATGTATAA
SEQ. ID NO: 69
MWFGEFGGQYVPETLVGPLKELEKAYKRFKDDEEFNRQLNYYLKTWAG
RPTPLYYAKRLTEKIGGAKVYLKREDLVHGGAHKTNNAIGQALLAKLM
GKTRLIAGTGAGQHGVATAMAGALLGMKVDIYMGAEDVERQKLNVFRM
KLLGANVIPVNSGSRTLKDAIDEALRDWVATFEYTHYLIGSVVGPHPY
PTIVRDFQSVIGREAKAQIPEAEGQLPDVIVACVGGGSNAMGIFYPFV
NDKKVKLVGVEAGGKGLESGKHSASLNAGQVGVSHGMLSYFLQDEEGQ
IKPSHSIAPGLDYPGVGPEHAYLKKIQRAEYVAVTDEEALKAFHELSR
TEGIIPALESAHAVAYAMKLAKEMSRDEIIIVNLSGRGDKDLDIVLKA
SGNV
SEQ. ID NO: 70
ATGTGGTTTGGTGAGTTTGGTGGTCAGTATGTGCCTGAGACGCTAGTT
GGGCCACTGAAGGAGTTAGAGAAAGCGTACAAGAGATTCAAAGATGAC
GAGGAGTTTAACCGTCAGTTGAATTATTATCTTAAGACGTGGGCTGGT
AGGCCGACCCCTCTATACTACGCTAAGCGTTTAACCGAAAAGATAGGT
GGCGCCAAGATCTATCTGAAGAGAGAGGACCTAGTCCACGGCGGGGCT
CACAAAACTAACAACGCAATTGGTCAGGCACCATTAGCAAAGTTAATG
GGCAAAACACGTCTAATTGCCGAAACTGGTGCTGGTCAACATGGAGTT
GCTACGGCAATGGCGGGCGCACTGTTAGGTATGAAAGTGGATATCTAT
ATGGGAGCAGAAGACGTGGAGAGACAGAAGATGAACGTGTTCCGTATG
AAACTATTGGGAGCTAATGTTATCCCGGTTAACTCTGGCTCAAGAACT
GCAAAGGACGCCATAAACGAAGCTTTGAGGGATTGGGAAGCCACATTC
GAGTACACACACTACCTTATAGGCTCAGTCGTAGGCCCGCATCCCTAC
CCCACCATAGTTCGTGACTTCCAGAGTGTTATTGGCCGTGAGGCCAAG
GCACAGATCTTGGAAGCTGAAGGTCAATTGCCTGACGTAATAGTTGCA
TGTGTGGGTGGCGGTTCTAACGCTATGGGTATATTCTACCCCTTCGTG
AACGATAAGAAAGTAAAGCTAGTTGGAGTGGAAGCAGGTGGCAAAGGG
TTGGAGTCGGGCAAGCACTCAGCCTCATTAAACGCAGGTCAGGTAGGC
GTTCTGCACGGTATGTTATCCTATTTCTTACAGGACGAGGAAGGCCAA
ATCAAACCATCGCACTCAATCGCACCTGGTTTAGATTACCCAGGAGTC
GGTCCCGAGCACGCATATTTAAAGAAAATCCAGAGAGCAGAGTACGTC
ACGGTGACGGATGAGGAAGCCCTTAAAGCGTTTCACGAGCTGTCGAGG
ACCGAGGGCATAATACCTGCTCTTGAAAGTGCCCACGCAGTTGCCTAT
GCTATGAAGTTGGCCAAGGAGATGAGTAGAGACGAGATTATCATAGTA
AATCTGTCAGGCAGAGGTGACAAGGATCTTGACATAGTCCTTAAGGCG
AGCGGTAATGTTTTGGAGTAA
SEQ. ID NO: 71
MWFGEFGGQYVPETLVGPLKELEKAYKRFKDDEEFNRQLNYYLKTWAG
RPTPLYYAKRLTEKIGGAKIYLKREDLVHGGAHKTNNAIGQAPLAKLM
GKTRLIAETGAGQHGVATAMAGALLGMKVDIYMGAEDVERQKMNVFRM
KLLGANVIPVNSGSRTAKDAINEALRDWEATFEYTHYLIGSVVGPHPY
PTIVRDFQSVIGREAKAQILEAEGQLPDVIVACVGGGSNAMGIFYPFV
NDKKVKLVGVEAGGKGLESGKHSASLNAGQVGVLHGMLSYFLQDEEGQ
IKPSHSIAPGLDYPGVGPEHAYLKKIQRAEYVTVTDEEALKAFHELSR
TEGIIPALESAHAVAYAMKLAKEMSRDEIIIVNLSGRGDKDLDIVLKA
SGNVLE
SEQ. ID NO: 72
ATGTGGTTCGGAGAGTTCGGCGGGCAGTACGTCCCAGAAACGCTAGTC
GGCCCACTGAAGGAGCTGGAGAAAGCATACAAAAGATTCAAGGATGAC
GAGGAGTTCAACAGGCAATTAAACTATTACCTAAAGACCTGGGCTGGT
AGGCCCACCCCTTTATACTATGCAAAACGTTTAACGGAGAAAATCGGT
GGCGCAAAAGTGTACTTGAAGAGAGAAGACTTAGTCCACGGTGGTGCT
CATAAAACTAACAACGCTATAGGGCAGGCACTTTTAGCTAAGTTGATG
GGTAAAACGAGGTTGATTGCGGGTACCGGTGCCGGTCAACATGGCGTT
GCTACGGCAATGGCGGGAGCCCTATTGGGCATGAAGGTTGACATTTAT
ATGGGCGCTGAGGACGTGGAACGTCAGAAACTGAACGTCTATCGTATG
AAATTGCTTGGTGCCAACGTTATACCAGTCAATTCCGGCTCGCGTACG
GTTAAAGACGCTTTCGATGAGGCACTATGTGATAGAGTTGCCACTTTC
GAATACACCCACTATCTTATTGGTACTGTCTGGGGTCCACATCCGTAT
CCAACTATTGTAAGGGACTTCCAGACTGTTATCGGAAGAGAGGCAAAG
GCCCAAATATTAGAGGCTGAGGGAAGGTTACCCGACGCCATTGTTGCA
TGCGTAGGCGGTGGTAGTAACGCAATGGGTATTTTCTATCCCTTCGTT
AACGATAAGAAGGTTAAGTTAGTGGGTGTCGAGGCCGGTGGTAAGGGG
CTTGAGTCCGGTAAACATTCCGCATCTTTGAATGCTGGGCAAGTAGGT
GTCAGTCATGGAATGTTATCATACTTTCTACAGGACGAGGAAGGACAG
ATAAAACCTTCTCATTCTATAGCGCCAGGTCTTGACCACCCTGGCGTT
GGACCAGAGCATGCTTATTTGAAGAAGATCCAGAGAGCTGAATATGTA
GCTGTTACGGATGAGGAAGCTCTAAAGGCGTTCCACGAGTTAAGTAGG
ACTGAGGGAATTATACCGGCATTAGAGTCTGCCCACGCTGTAGCTTAC
GCAATGAAGCTGGCCAAAGAGATGAGCCGTGACGAGATCATCATCGTA
AACTTGTCTGGCAGAGGCGATAAGGACCTAGACATCGTATTAAAGGCC
TCGGGGAACGTCCTAGAGTAG
SEQ. ID NO: 73
MWFGEFGGQYVPETLVGPLKELEKAYKRFKDDEEFNRQLNYYLKTWAG
RPTPLYYAKRLTEKIGGAKVYLKREDLVHGGAHKTNNAIGQALLAKLM
GKTRLIAGTGAGQHGVATAMAGALLGMKVDIYMGAEDVERQKLNVYRM
KLLGANVIPVNSGSRTVKDAFDEALCDRVATFEYTHYLIGTVWGPHPY
PTIVRDFQTVIGREAKAQILEAEGRLPDAIVACVGGGSNAMGIFYPFV
NDKKVKLVGVEAGGKGLESGKHSASLNAGQVGVSHGMLSYFLQDEEGQ
IKPSHSIAPGLDHPGVGPEHAYLKKIQRAEYVAVTDEEALKAFHELSR
TEGIIPALESAHAVAYAMKLAKEMSRDEIIIVNLSGRGDKDLDIVLKA
SGNVLE
SEQ. ID NO: 74
GTATCCGGCTGTTCCTTCATAGCCCTTTCAATGAACGTTGCAGCCCTT
TGAAGATTGGCCATTTTGTCAGGACTCGAGCCTGACAGTTGGACCAAC
GCAACTTTAATTTTTTGTGAAAGAATCTTCGAAGCACTCATACTGGCG
ATCTTCACGCCCTCCTGCTATTACAAAAGCTGTGTTTTTACAAGAATC
AAATTAAGTTAGCAAGATATTATACAACATTATTGATAATTTCAATAT
CGTGTTCGTACCTGATGACGTATCTGTGCATTGATAAGGCCCGCATGG
TTTCAGAAAGCAGAGCGGAACGATTCCAAATTAGTGGCCTTGTGCTTT
GCATGTCAATTGTGTTACCTTCAGCTCGTGGATTTGTTTTATCAATAC
ACAGTCTACAGTCAAGAATTTTTTTTATCAAATTTTGCGTTCGAGCGT
ATAAAATAGCCGCTGTAGCTACTTAAGTTCCTGTTCAGCGATAGTTTT
TTTCCATCACACGTACTATGGCAATTAAGTCCTCAGCGAGCTCGCATG
GAATGCGTGCGATGAGCGACCTCATGCTATACCTGAGAAAGCAACCTG
ACCTACAGGAAAGAGTTACTCAAGAATAAGAATTTTCGTTTTAAAACC
TAAGAGTCACTTTAAAATTTGTATACACTTATTTTTTTTATAACTTAT
TTAATAATAAAAATCATAAATCATAAGAAATTCGCTTATTTAGAAGTG
TCAACAACGTATCTACCAACGGAATGCGTGCGATTCAGGCGTAGTCTG
GAACGTCGTATGGGTATGGACCAGAGACGGATTGAGAAACTTGTTCGG
TTCTTGGGACGAATCTACATTGGAATGGAACTGGGTGCCTGAAGAAAC
CAGTGGTGAAATCAGCTGGGATGTCAATTGGCTTACCGTTTTGGTCAA
CTGGTCTTTCAATGTTGAAAGCAGACAACAAAGTAGCACCGGCAATCC
AAACAGTAGATTGAGCTAGGTGAATACCTGGGCAGTTTCTTCTACCGT
AACCGAAAGCAGCCTTTCTTGGGTCTCTAACAGTGTTGTCTGGTTTAC
CATCAGGACCCAAGTATCTTTCTGGACGGAAAACGGATGGATCTGGAT
AGACTTCTGGGTCATTCAAAACTGCCCAGGTGTTAGCAAAAACCAATG
TGTTCTTTGGAATCAAATAACCTCTGTAAACATCATCCTTCATCAATT
TATGAGGGATGGCTAATGGAGCAATTTGGTTCCATCTGAATAATTCCT
TGATACAAGCGGTCAAATATGGGAGTGAGTCGTCTTCCTCATCGTAGT
CAGGGATTTGACCGTTGTTGGTCAAAGCATCCAATTCAGCTTGAACCT
TTCTTTGCACTTCTGGGTATTTAACCATGGCCAATATGAAAGCGGACA
TAGCAGAGACGGTAGTGTCGCCACCACCAACGTTAACTTCAGCAGCAG
TGTTCTTGATAACATGTTCTTGGTGTTCTAAATCACCGTTCAAGTCCA
TAGCTTGTAATCTAGCAGAAGCGTAAGATGGTCTGGTTAGACCTTGTG
GAGCCAACTTTCTCATAGTTTCGTATGGCATGTCAACCATGTGGTCAG
CGGCTTCTCTCCAGACCTTGGCCTTACGCTTGAAGACGGCACCTGGGA
ACCAAGCTGGCAAGTACTTCAAAGATGGGAAGGAGTCAACCCAGAACT
TACCTGGAACACTAGCAATGGCCAAACCTTCGTTGGCTAAGTGGGTAG
CTTCTAACCATGGGTCATCTTCGGCTAGATCGATACCGTAACCAATGT
CCAAAGACATAGCAGCGATTTGATGTCTGATGTGTTGAGCCCACCGGT
CCGGAGTCTTGGTCAATTGTTGAACCAATTGGTGAGCAGCCTTGACTT
GAGCGTGTCTGAATTGCTTGATACCCTTTTCAGAGAATTCCTTAGCAA
ACATTCTTCTTTCTTCTCTCCAACGGTCACCGTAAGTGATAAAACCCA
GATCAAATTCCCAACCCATCAATTCATTGACCATAGTGGATTCCAATC
TACCGGAGTAGATGGAACCACGTTTTTCCAAGAGATCCGTGATAGTTT
CCAAGGTGTTTAAAATGACCATTTCGGTACCACCAGCATCTACGTACA
AGATATCAGTGTTGTAGTCACGACCCCATTGCAAGAAAGTCAACCATG
GAGATTCTTCTGGCATGTCGAACATGTTACCAATAAATGGGATTGGAA
TTCCTGGTGGACCAGGTGGCAATCTGGATCTACGAACTCTTCTGGAGA
CGATGTAGTAAATACAACCAGCAATGACGAAAGAGAACAAGACAGCAA
TCATTGTTTTATATTTGTTGTAAAAAGTAGATAATTACTTCCTTGATG
ATCTGTAAAAAAGAGAAAAAGAAAGCATCTAAGAACTTGAAAAACTAC
GAATTAGAAAAGACCAAATATGTATTTCTTGCATTGACCAATTTATGC
AAGTTTATATATATGTAAATGTAAGTTTCACGAGGTTCTACTAAACTA
AACCACCCCCTTGGTTAGAAGAAAAGAGTGTGTGAGAACAGGCTGTTG
TTGTCACACGATTCGGACAATTCTGTTTGAAAGAGAGAGAGTAACAGT
ACGATCGAACGAACTTTGCTCTGGAGATCACAGTGGGCATCATAGCAT
GTGGTACTAAACCCTTTCCCGCCATTCCAGAACCTTCGATTGCTTGTT
ACAAAACCTGTGAGCCGTCGCTAGGACCTTGTTGTGTGACGAAATTGG
AAGCTGCAATCAATAGGAAGACAGGAAGTCGAGCGTGTCTGGGTTTTT
TCAGTTTTGTTCTTTTTGCAAACAACAGTTTATTCCTGGCATCCACTA
AATATAATGGAGCCCGCTTTTTAAGCTGGCATCCAGAAAAAAAAAGAA
TCCCAGCACCAAAATATTGTTTTCTTCACCAACCATCAGTTCATAGGT
CCATTCTCTTAGCGCAACTACAGAGAACAGGGGCACAAACAGGCAAAA
AACGGGCACAACCTCAATGGAGTGATGCAACCTGCCTGGAGTAAATGA
TGACACAAGGCAATTGACCCACGCATGTATCTATCTCATTTTCTTACA
CCTTCTATTACCTTCTGCTCTCTCTGATTTGGAAAAAGCTGAAAAAAA
AGGTTGAAACCAGTTCCCTGAAATTATTCCCCTACTTGACTAATAAGT
ATATAAAGACGGTAGGTATTGATTGTAATTCTGTAAATCTATTTCTTA
AACTTCTTAAATTCTACTTTTATAGTTAGTCTTTTTTTTAGTTTTAAA
ACACCAAGAACTTAGTTTCGAATAAACACACATAAACAAACAAAATGG
CTTCTAGTTCTTCCGATGTCTTCGTTTTGGGTCTAGGTGTTGTTTTGG
CTGCCTTGTATATCTTCAGAGACCAATTATTCGCTGCTTCTAAGCCAA
AGGTGGCTCCAGTTTCCACTACGAAGCCTGCCAACGGTTCCGCTAACC
CAAGAGACTTCATCGCCAAGATGAAACAAGGTAAGAAGAGAATCGTAA
TCTTCTACGGTTCTCAAACTGGTACCGCTGAAGAATATGCTATTCGTT
TGGCTAAGGAAGCTAAGCAAAAGTTCGGTCTAGCCTCCTTGGTTTGTG
ATCCAGAAGAATACGATTTTGAAAAGTTGGACCAATTGCCAGAAGATT
CTATTGCTTTCTTCGTCGTTGCTACCTATGGTGAAGGTGAACCTACAG
ACAACGCTGTCCAATTGTTGCAAAACTTGCAAGATGAAAGCTTCGAAT
TCTCCTCTGGTGAGAGAAAGTTGTCAGGTTTGAAGTACGTTGTTTTTG
GTCTGGGTAACAAGACCTACGAACATTACAACCTCATTGGGAGAACTG
TTGACGCTCAATTGGCCAAGATGGGTGCTATCAGAATCGGTGAAAGAG
GTGAAGGTGATGATGACAAGTCCATGGAAGAAGACTACTTGGAATGGA
AGGATGGTATGTGGGAAGCGTTTGCCACTGCTATGGGTGTTGAAGAAG
GTCAAGGTGGTGACTCCGCTGATTTCGTCGTTTCCGAATTGGAATCTC
ACCCACCAGAAAAGGTTTACCAAGGTGAATTTTCTGCTAGAGCTTTAA
CCAAAACCAAGGGTATTCACGACGCTAAGAATCCTTTTGCTGCTCCAA
TTGCGGTTGCTAGAGAATTGTTCCAATCTGTTGTCGATAGAAACTGTG
TCCACGTCGAATTCAACATTGAAGGCTCTGGTATCACCTATCAACACG
GTGACCACGTTGGTTTGTGGCCATTGAATCCAGATGTTGAAGTCGAAC
GGTTGTTGTGTGTTTTAGGTTTAGCTGAAAAGAGAGATGCTGTCATCT
CCATTGAATCCTTAGACCCGGCTTTGGCTAAGGTTCCATTCCCAGTCC
CAACTACTTACGGTGCTGTGTTGAGACACTACATTGACATCTCTGCTG
TCGCCGGTAGACAAATCTTGGGTACTTTGTCCAAATTCGCTCCAACCC
CAGAAGCTGAAGCTTTCTTGAGAAACTTGAACACTAACAAGGAAGAAT
ACCACAACGTCGTCGCTAACGGTTGTTTGAAATTGGGTGAAATTTTGC
AAATCGCTACCGGTAACGACATTACTGTCCCACCAACTACTGCCAACA
CCACCAAATGGCCAATTCCATTCGACATCATTGTTTCTGCCATCCCAA
GATTGCAACCAAGATACTACTCTATCTCTTCTTCCCCAAAAATTCATC
CAAACACCATCCACGCTACCGTTGTTGTGCTCAAATACGAAAACGTTC
CAACCGAACCAATCCCAAGAAAGTGGGTTTACGGTGTCGGTAGTAACT
TCTTGTTGAATTTAAAGTACGCTGTTAACAAGGAACCAGTTCCATACA
TCACTCAAAATGGCGAACAAAGAGTCGGTGTCCCGGAATACTTGATTG
CTGGTCCACGTGGTTCTTACAAGACTGAATCTTTCTACAAGGCTCCAA
TCCATGTTAGACGTTCTACTTTCCGTTTGCCAACCAACCCAAAGTCTC
CAGTCATCATGATTGGTCCAGGTACTGGTGTCGCCCCATTCAGAGGCT
TCGTTCAAGAAAGAGTTGCCTTGGCCAGAAGATCCATCGAAAAGAACG
GTCCTGACTCTTTGGCTGACTGGGGTCGTATTTCCTTGTTCTACGGTT
GTAGAAGATCCGACGAAGACTTCTTGTACAAGGACGAATGGCCACAAT
ACGAAGCTGAGTTGAAGGGTAAGTTCAAGTTGCACTGTGCTTTCTCCA
GACAAAACTACAAGCCAGACGGTTCTAAGATTTACGTCCAAGATTTGA
TCTGGGAAGACAGAGAACACATTGCCGATGCCATCTTAAACGGTAAGG
GTTACGTCTACATCTGCGGTGAAGCTAAGTCCATGTCTAAACAAGTTG
AAGAAGTTCTAGCCAAGATCTTGGGCGAAGCCAAAGGTGGTTCCGGTC
CAGTTGAAGGTGTTGCTGAAGTCAAGTTACTGAAGGAACGGTCCAGAT
TGATGTTGGATGTCTGGTCTGAACAAAAGTTAATTTCTGAAGAAGATT
TGGAATGAATCGCGTGCATTCATCCGCTCTAACCGAAAAGGAAGGAGT
TAGACAACCTGAAGTCTAGGTCCCTATTTATTTTTTTATAGTTATGTT
AGTATTAAGAACGTTATTTATATTTCAAATTTTTCTTTTTTTTCTGTA
CAGACGCGTGTACGCATGTAACATTATACTGAAAACCTTGCTTGAGAA
GGTTTTGGGACGCTCGAAGATCGCGTCCCAATTCGCCCTATAGTGAGT
CGTATTACGCGCGCTCACTGGCCGTCGTTTTACAACGTCGTGACTGGG
AAAACCCTGGCGTTACCCCTGCAGGACTAGTGCTGAGGCATTAATAGT
TTACTCAATTCTTGAAGCCAATTTGTACAATTCCCCATTAGAGTCAAA
TAAAAGGATGCCTCACGGAGGTATGTTACCCGCGCTATTTCACATGGC
TCATTGAATTAGAGGTGGAATTTGGTGTACCCTCCCCTCCTCATCTGA
TGAAGTAGTGATCCGACAATTCTTAAAAGTTGTAGACATTACTTTTAC
CACCAACTAAGTTGTATTTATATTGCTACCCTTATCCTTTTATATCTA
ACTAGCGCTCATAAGGTTGGGGCAATACTAAAACTGTGTTCTTATTCA
ACTCATTAAATACGTGGCAGTACGTACCCTATTAGAAACAATAGGAAA
CAGCAGAGTCGGAAGAAGCCAAATGCCAGATTTGAAGTCCAAAACCTT
GTCAAGCCAATCTTTGGGAGCGGCTATTCCTCCAGAAATTGTGTACCA
AATACTTACATACCAGTTTAGGGATTTGTTAAGAAATGACCATCCAGG
TACGGCAGAAA
SEQ. ID NO: 75
CAATCTGGCGGCTTGAGTTCTCAACATGTTTTATTTTTTACTTATATT
GCTGGTAGGGTAAAAAAATATAACTCCTAGGAATAGGTTGTCTATATG
TTTTTGTCTTGCTTCTATAATTGTAACAAACAAGGAAAGGGAAAATAC
TGGGTGTAAAAGCCATTGAGTCAAGTTAGGTCATCCCTTTTATACAAA
ATTTTTCAATTTTTTTTCCAAGATTCTTGTACGATTAATTATTTTTTT
TTTGCGTCCTACAGCGTGATGAAAATTTCGCCTGCTGCAAGATGAGCG
GGAACGGGCGAAATGTGCACGCGCACAACTTACGAAACGCGGATGAGT
CACTGACAGCCACCGCAGAGGTTCTGACTCCTACTGAGCTCTATTGGA
GGTGGCAGAACCGGTACCGGAGGAGGCCGCTATAACCGGTTTGAATTT
ATTGTCACAGTGTCACATCAGCATTAAGTCCTCAGCGAGCTCGCATGG
AATGCGTGCGATGAGCGACCTCATGCTATACCTGAGAAAGCAACCTGA
CCTACAGGAAAGAGTTACTCAAGAATAAGAATTTTCGTTTTAAAACCT
AAGAGTCACTTTAAAATTTGTATACACTTATTTTTTTTATAACTTATT
TAATAATAAAAATCATAAATCATAAGAAATTCGCTTATTTAGAAGTGT
CAACAACGTATCTACCAACGGAATGCGTGCGATTCAGGTGGAGTCCAA
ACCCAACAAAGGATTTGGAATTGGCTTACCGGCAGTGGAGGATTCCTT
TAGCAACGTGGAAGTGATTTCACCGTTGTCGTTGTTACCTCTAGCATC
GTGGAAAGCAGCAACACCCTTCTTAACAAAGTTGATTCTTTCTTCTTC
AGAACCCCATTGCATGAAGTCAGTCCACATAACAATGTGAGCGGCGAT
ACCAGCGGTAACCTTGGCGTAGTTGATGGAATGCTTGGAAGTACGGGC
GTAAGATTGCAAGTAAGCTTGTCTCATGGTTGTACCAACTTGTTCGTC
TTGGAATCTGCTAATCAAGTAACAGTCACCCAAGAAGTAACCCAAATC
CAATGAAGCTGGACCGTACTTACACAATTCCCAGTCTAAGATGTAGAT
CTTTTGCAACTTAGATGGGTTACCTTCTTCAAGTTGCAACAAGATGTT
CCCAGACCACAAGTCAGCCATGACCAAAGTTTCTTCGGAGTGCATAAC
ATCGTCAACTAGATCCTTGACAACAGTTGGCAACAATGGATCATCGAC
GCCGTATTTAGCGGCGTTTGGGATAATAGTTTGGTACAATTGGTCAGA
GGTGGTTCTACCGACAATGTTACCAGAGAAGAACTTGAATTCTGGGTC
GTCTCTTCTTTCTCTACCTATGTTGTGCAATCTGGCGACGAAACCACC
AATCTCGGTACCAACCAATCTAGCAATATCGGTAGCCAAAGGTGGCTT
AGCAGTAACGTAGTCTAATAAGGTCTTCATTTTACCGACATCTTGCAT
AATCAAAGCATTGTTTTCCAAGTCATAGTTGAGACCTTCTGGAACAGA
GACAATACCATCAACACCACCCAAAACTTCTCTGTTAGCCATCATCAA
CTTGATAGCTTGGTATTCGTAGACAGAACGTTCAACACCGATTTTGAA
ATCTTCATCAGTAGACATGTGTGGTTGAGCGTGCTTCAAAATGATAGA
AGTGTGACCTTGGTATGGAGCGTTCAATTTAATACGCCAGGTGACGTT
AACGAAACCACCGGATAATCTCTTGACACCAGAAGTGTCAACATCTAA
AGACAAATGCTTGGTCAAATAGGTGATTAAACCGTCTTCAGTCTTCAA
GTCAAAGGCCATTGTTTTATATTTGTTGTAAAAAGTAGATAATTACTT
CCTTGATGATCTGTAAAAAAGAGAAAAAGAAAGCATCTAAGAACTTGA
AAAACTACGAATTAGAAAAGACCAAATATGTATTTCTTGCATTGACCA
ATTTATGCAAGTTTATATATATGTAAATGTAAGTTTCACGAGGTTCTA
CTAAACTAAACCACCCCCTTGGTTAGAAGAAAAGAGTGTGTGAGAACA
GGCTGTTGTTGTCACACGATTCGGACAATTCTGTTTGAAAGAGAGAGA
GTAACAGTACGATCGAACGAACTTTGCTCTGGAGATCACAGTGGGCAT
CATAGCATGTGGTACTAAACCCTTTCCCGCCATTCCAGAACCTTCGAT
TGCTTGTTACAAAACCTGTGAGCCGTCGCTAGGACCTTGTTGTGTGAC
GAAATTGGAAGCTGCAATCAATAGGAAGACAGGAAGTCGAGCGTGTCT
GGGTTTTTTCAGTTTTGTTCTTTTTGCAAACAACAGTTTATTCCTGGC
ATCCACTAAATATAATGGAGCCCGCTTTTTAAGCTGGCATCCAGAAAA
AAAAAGAATCCCAGCACCAAAATATTGTTTTCTTCACCAACCATCAGT
TCATAGGTCCATTCTCTTAGCGCAACTACAGAGAACAGGGGCACAAAC
AGGCAAAAAACGGGCACAACCTCAATGGAGTGATGCAACCTGCCTGGA
GTAAATGATGACACAAGGCAATTGACCCACGCATGTATCTATCTCATT
TTCTTACACCTTCTATTACCTTCTGCTCTCTCTGATTTGGAAAAAGCT
GAAAAAAAAGGTTGAAACCAGTTCCCTGAAATTATTCCCCTACTTGAC
TAATAAGTATATAAAGACGGTAGGTATTGATTGTAATTCTGTAAATCT
ATTTCTTAAACTTCTTAAATTCTACTTTTATAGTTAGTCTTTTTTTTA
GTTTTAAAACACCAAGAACTTAGTTTCGAATAAACACACATAAACAAA
CAAAATGCATATCAGAAACCCATATAGAACTCCAATTGACTACCAAGC
TTTGTCTGAAGCTTTCCCACCATTGAAGCCATTTGTTTCCGTTAACGC
TGATGGTACCTCCTCAGTTGACTTGACCATTCCAGAAGCCCAAAGAGC
TTTTACCGCTGCCCTTTTGCACAGAGACTTCGGCTTGACTATGACTAT
CCCAGAAGATCGTTTGTGTCCAACCGTTCCAAACAGATTGAACTACGT
TTTGTGGATTGAAGACATTTTCAACTACACCAACAAGACTTTGGGTTT
ATCTGACGACCGTCCAATCAAGGGTGTTGATATCGGTACCGGTGCTTC
TGCCATTTACCCAATGTTGGCTTGCGCCAGATTCAAGGCTTGGTCCAT
GGTTGGTACTGAAGTTGAAAGAAAGTGTATCGACACTGCTAGATTAAA
CGTTGTTGCTAACAACTTGCAAGATCGTCTATCCATCTTGGAAACCTC
TATTGACGGTCCAATTTTAGTCCCAATTTTCGAAGCTACCGAAGAATA
CGAATACGAATTCACCATGTGTAACCCACCTTTCTACGATGGTGCCGC
TGACATGCAAACTAGCGATGCTGCAAAGGGTTTTGGTTTCGGTGTCGG
TGCTCCACACTCTGGTACAGTCATCGAAATGTCTACTGAAGGTGGTGA
ATCCGCTTTCGTGGCTCAAATGGTTAGAGAATCTCTTAAGTTGAGAAC
CAGATGTAGATGGTACACTTCTAACTTAGGTAAGTTGAAATCTTTGAA
GGAAATTGTCGGTTTGTTGAAGGAATTAGAAATCTCTAATTACGCCAT
CAACGAATATGTCCAAGGTTCCACTAGAAGATACGCTGTCGCTTGGAG
TTTCACTGATATCCAATTGCCAGAAGAATTGTCCAGACCATCTAATCC
TGAATTGTCCTCTTTGTTCGACTACAAGGATGACGATGACAAATGAAT
CGCGTGCATTCATCCGCTCTAACCGAAAAGGAAGGAGTTAGACAACCT
GAAGTCTAGGTCCCTATTTATTTTTTTATAGTTATGTTAGTATTAAGA
ACGTTATTTATATTTCAAATTTTTCTTTTTTTTCTGTACAGACGCGTG
TACGCATGTAACATTATACTGAAAACCTTGCTTGAGAAGGTTTTGGGA
CGCTCGAAGATCGCGTACCCAATTCGCCCTATAGTGAGTCGTATTACG
CGCGCTCACTGGCCGTCGTTTTACAACGTCGTGACTGGGAAAACCCTG
GCGTTACCCCTGCAGGACTAGTGCTGAGGCATTAATGCAACTCAGAAG
TTTGACAGCAAGCAAGTTCATCATTCGAACTAGCCTTATTGTTTTAGT
TCAGTGACAGCGAACTGCCGTACTCGATGCTTTATTTCTCACGGTAGA
GCGGAAGAACAGATAGGGGCAGCGTGAGAAGAGTTAGAAAGTAAATTT
TTATCACGTCTGAAGTATTCTTATTCATAGGAAATTTTGCAAGGTTTT
TTAGCTCAATAACGGGCTAAGTTATATAAGGTGTTCACGCGATTTTCT
TGTTATGTATACCTCTTCTCTGAGGAATGGTACTACTGTCCTGATGTA
GGCTCCTTAAATTGGTGGGCAAGAATAACTTATCGATATTTTGTATAT
TGGTCTTGGAGTTCACCACGTAATGCCTGTTTAAGACCATCAGTTAAC
TCTAGTATTATTTGGTCTTGGCTACTGGCCGTTTGCTATTATTCAAGT
CTTTTGTGCCTTCCCGTCGGGTAAGGGAGTTATTTAGGGATACAGAAT
CTAACGAAAACTAAATCTCAATGATTAACTCTATTTAATCCTTTTTTG
AAAGGCAAAAGAGGTCCCTTGTTCACTTACAACGTTCTTAGCCAAATT
CGCTTATCACTTACTACTTCACGATATACAGAAGTAAAAACATATAAA
AAGATGTCTGTTTGTTTAGCCATCACAAAAGGTATCGCAG
SEQ. ID NO: 76
TGTTTTATATTTGTTGTAAAAAGTAGATAATTACTTCCTTGATGATCT
GTAAAAAAGAGAAAAAGAAAGCATCTAAGAACTTGAAAAACTACGAAT
TAGAAAAGACCAAATATGTATTTCTTGCATTGACCAATTTATGCAAGT
TTATATATATGTAAATGTAAGTTTCACGAGGTTCTACTAAACTAAACC
ACCCCCTTGGTTAGAAGAAAAGAGTGTGTGAGAACAGGCTGTTGTTGT
CACACGATTCGGACAATTCTGTTTGAAAGAGAGAGAGTAACAGTACGA
TCGAACGAACTTTGCTCTGGAGATCACAGTGGGCATCATAGCATGTGG
TACTAAACCCTTTCCCGCCATTCCAGAACCTTCGATTGCTTGTTACAA
AACCTGTGAGCCGTCGCTAGGACCTTGTTGTGTGACGAAATTGGAAGC
TGCAATCAATAGGAAGACAGGAAGTCGAGCGTGTCTGGGTTTTTTCAG
TTTTGTTCTTTTTGCAAACAACAGTTTATTCCTGGCATCCACTAAATA
TAATGGAGCCCGCTTTTTAAGCTGGCATCCAGAAAAAAAAAGAATCCC
AGCACCAAAATATTGTTTTCTTCACCAACCATCAGTTCATAGGTCCAT
TCTCTTAGCGCAACTACAGAGAACAGGGGCACAAACAGGCAAAAAACG
GGCACAACCTCAATGGAGTGATGCAACCTGCCTGGAGTAAATGATGAC
ACAAGGCAATTGACCCACGCATGTATCTATCTCATTTTCTTACACCTT
CTATTACCTTCTGCTCTCTCTGATTTGGAAAAAGCTGAAAAAAAAGGT
TGAAACCAGTTCCCTGAAATTATTCCCCTACTTGACTAATAAGTATAT
AAAGACGGTAGGTATTGATTGTAATTCTGTAAATCTATTTCTTAAACT
TCTTAAATTCTACTTTTATAGTTAGTCTTTTTTTTAGTTTTAAAACAC
CAAGAACTTAGTTTCGAATAAACACACATAAACAAACAAAGGATCCAT
GATGAAGTTCTGGAGAAAGTACACACAACAAGAAATGGATGAAAAGAT
TACTGAATCTTTGGAAAAGACTTTGAACTACGATAACACTAAGACAAT
CGGTATTCCAGGTACTAAGTTGGATGATACAGTTTTCTATGATGATCA
TTCTTTCGTTAAGCATTCACCATACTTGAGAACTTTTATTCAAAACCC
AAACCATATCGGTTGTCATACTTATGATAAGGCTGATATCTTGTTCGG
TGGTACATTCGATATCGAAAGAGAATTAATCCAATTGTTAGCAATCGA
TGTTTTGAACGGTAACGATGAAGAATTTGATGGTTACGTTACTCAAGG
TGGTACAGAAGCTAACATCCAAGCAATGTGGGTTTACAGAAACTACTT
CAAGAAAGAAAGAAAGGCTAAGCATGAAGAAATCGCTATCATCACTTC
AGCAGATACACATTACTCTGCATACAAAGGTTCAGATTTGTTGAACAT
CGATATTATTAAGGTTCCAGTTGATTTTTATTCAAGAAAAATTCAAGA
AAATACATTGGATTCAATTGTTAAAGAAGCTAAAGAAATTGGTAAAAA
GTACTTCATCGTTATCTCTAACATGGGTACTACAATGTTTGGTTCAGT
TGATGATCCAGATTTGTACGCTAACATCTTCGATAAGTACAATTTGGA
ATACAAAATTCATGTTGATGGTGCATTTGGTGGTTTTATATATCCAAT
TGATAATAAGGAATGTAAAACTGATTTCTCTAATAAGAACGTTTCTTC
AATCACATTAGATGGTCATAAGATGTTGCAAGCTCCATACGGTACTGG
TATCTTCGTTTCAAGAAAGAATTTGATCCATAACACTTTGACAAAGGA
AGCAACTTACATCGAAAATTTGGATGTTACATTGTCTGGTTCAAGATC
TGGTTCAAATGCTGTTGCAATTTGGATGGTTTTAGCTTCTTATGGTCC
ATACGGTTGGATGGAAAAGATTAATAAGTTGAGAAATAGAACTAAATG
GTTGTGTAAGCAATTGAACGATATGAGAATTAAATATTACAAAGAAGA
TTCAATGAATATTGTTACAATTGAAGAACAATATGTTAATAAGGAAAT
CGCTGAAAAGTACTTTTTAGTTCCAGAAGTTCATAACCCAACTAACAA
CTGGTACAAGATCGTTGTTATGGAACATGTTGAATTGGATATCTTGAA
CTCTTTGGTTTACGATTTGAGAAAGTTTAATAAGGAACATTTGAAGGC
AATGCATCATCATCATCATCATTAACCGCGGCTAGCTAAGATCCGCTC
TAACCGAAAAGGAAGGAGTTAGACAACCTGAAGTCTAGGTCCCTATTT
ATTTTTTTATAGTTATGTTAGTATTAAGAACGTTATTTATATTTCAAA
TTTTTCTTTTTTTTCTGTACAGACGCGTGTACGCATGTAACATTATAC
TGAAAACCTTGCTTGAGAAGGTTTTGGGACGCTCGAAGATCCAGCTGC
ATTAATGAATCGGCCAACGCGCGGGGAGAGGCGGTTTGCGTATTGGGC
GCTCTTCCGCTTCCTCGCTCACTGACTCGCTGCGCTCGGTCGTTCGGC
TGCGGCGAGCGGTATCAGCTCACTCAAAGGCGGTAATACGGTTATCCA
CAGAATCAGGGGATAACGCAGGAAAGAACATGTGAGCAAAAGGCCAGC
AAAAGGCCAGGAACCGTAAAAAGGCCGCGTTGCTGGCGTTTTTCCATA
GGCTCCGCCCCCCTGACGAGCATCACAAAAATCGACGCTCAAGTCAGA
GGTGGCGAAACCCGACAGGACTATAAAGATACCAGGCGTTTCCCCCTG
GAAGCTCCCTCGTGCGCTCTCCTGTTCCGACCCTGCCGCTTACCGGAT
ACCTGTCCGCCTTTCTCCCTTCGGGAAGCGTGGCGCTTTCTCATAGCT
CACGCTGTAGGTATCTCAGTTCGGTGTAGGTCGTTCGCTCCAAGCTGG
GCTGTGTGCACGAACCCCCCGTTCAGCCCGACCGCTGCGCCTTATCCG
GTAACTATCGTCTTGAGTCCAACCCGGTAAGACACGACTTATCGCCAC
TGGCAGCAGCCACTGGTAACAGGATTAGCAGAGCGAGGTATGTAGGCG
GTGCTACAGAGTTCTTGAAGTGGTGGCCTAACTACGGCTACACTAGAA
GAACAGTATTTGGTATCTGCGCTCTGCTGAAGCCAGTTACCTTCGGAA
AAAGAGTTGGTAGCTCTTGATCCGGCAAACAAACCACCGCTGGTAGCG
GTGGTTTTTTTGTTTGCAAGCAGCAGATTACGCGCAGAAAAAAAGGAT
CTCAAGAAGATCCTTTGATCTTTTCTACGGGGTCTGACGCTCAGTGGA
ACGAAAACTCACGTTAAGGGATTTTGGTCATGAGATTATCAAAAAGGA
TCTTCACCTAGATCCTTTTAAATTAAAAATGAAGTTTTAAATCAATCT
AAAGTATATATGAGTAAACTTGGTCTGACAGTTACCAATGCTTAATCA
GTGAGGCACCTATCTCAGCGATCTGTCTATTTCGTTCATCCATAGTTG
CCTGACTCCCCGTCGTGTAGATAACTACGATACGGGAGGGCTTACCAT
CTGGCCCCAGTGCTGCAATGATACCGCGAGACCCACGCTCACCGGCTC
CAGATTTATCAGCAATAAACCAGCCAGCCGGAAGGGCCGAGCGCAGAA
GTGGTCCTGCAACTTTATCCGCCTCCATCCAGTCTATTAATTGTTGCC
GGGAAGCTAGAGTAAGTAGTTCGCCAGTTAATAGTTTGCGCAACGTTG
TTGCCATTGCTACAGGCATCGTGGTGTCACGCTCGTCGTTTGGTATGG
CTTCATTCAGCTCCGGTTCCCAACGATCAAGGCGAGTTACATGATCCC
CCATGTTGTGCAAAAAAGCGGTTAGCTCCTTCGGTCCTCCGATCGTTG
TCAGAAGTAAGTTGGCCGCAGTGTTATCACTCATGGTTATGGCAGCAC
TGCATAATTCTCTTACTGTCATGCCATCCGTAAGATGCTTTTCTGTGA
CTGGTGAGTACTCAACCAAGTCATTCTGAGAATAGTGTATGCGGCGAC
CGAGTTGCTCTTGCCCGGCGTCAATACGGGATAATACCGCGCCACATA
GCAGAACTTTAAAAGTGCTCATCATTGGAAAACGTTCTTCGGGGCGAA
AACTCTCAAGGATCTTACCGCTGTTGAGATCCAGTTCGATGTAACCCA
CTCGTGCACCCAACTGATCTTCAGCATCTTTTACTTTCACCAGCGTTT
CTGGGTGAGCAAAAACAGGAAGGCAAAATGCCGCAAAAAAGGGAATAA
GGGCGACACGGAAATGTTGAATACTCATACTCTTCCTTTTTCAATATT
ATTGAAGCATTTATCAGGGTTATTGTCTCATGAGCGGATACATATTTG
AATGTATTTAGAAAAATAAACAAATAGGGGTTCCGCGCACATTTCCCC
GAAAAGTGCCACCTGAACGAAGCATCTGTGCTTCATTTTGTAGAACAA
AAATGCAACGCGAGAGCGCTAATTTTTCAAACAAAGAATCTGAGCTGC
ATTTTTACAGAACAGAAATGCAACGCGAAAGCGCTATTTTACCAACGA
AGAATCTGTGCTTCATTTTTGTAAAACAAAAATGCAACGCGAGAGCGC
TAATTTTTCAAACAAAGAATCTGAGCTGCATTTTTACAGAACAGAAAT
GCAACGCGAGAGCGCTATTTTACCAACAAAGAATCTATACTTCTTTTT
TGTTCTACAAAAATGCATCCCGAGAGCGCTATTTTTCTAACAAAGCAT
CTTAGATTACTTTTTTTCTCCTTTGTGCGCTCTATAATGCAGTCTCTT
GATAACTTTTTGCACTGTAGGTCCGTTAAGGTTAGAAGAAGGCTACTT
TGGTGTCTATTTTCTCTTCCATAAAAAAAGCCTGACTCCACTTCCCGC
GTTTACTGATTACTAGCGAAGCTGCGGGTGCATTTTTTCAAGATAAAG
GCATCCCCGATTATATTCTATACCGATGTGGATTGCGCATACTTTGTG
AACAGAAAGTGATAGCGTTGATGATTCTTCATTGGTCAGAAAATTATG
AACGGTTTCTTCTATTTTGTCTCTATATACTACGTATAGGAAATGTTT
ACATTTTCGTATTGTTTTCGATTCACTCTATGAATAGTTCTTACTACA
ATTTTTTTGTCTAAAGAGTAATACTAGAGATAAACATAAAAAATGTAG
AGGTCGAGTTTAGATGCAAGTTCAAGGAGCGAAAGGTGGATGGGTAGG
TTATATAGGGATATAGCACAGAGATATATAGCAAAGAGATACTTTTGA
GCAATGTTTGTGGAAGCGGTATTCGCAATATTTTAGTAGCTCGTTACA
GTCCGGTGCGTTTTTGGTTTTTTGAAAGTGCGTCTTCAGAGCGCTTTT
GGTTTTCAAAAGCGCTCTGAAGTTCCTATACTTTCTAGAGAATAGGAA
CTTCGGAATAGGAACTTCAAAGCGTTTCCGAAAACGAGCGCTTCCGAA
AATGCAACGCGAGCTGCGCACATACAGCTCACTGTTCACGTCGCACCT
ATATCTGCGTGTTGCCTGTATATATATATACATGAGAAGAACGGCATA
GTGCGTGTTTATGCTTAAATGCGTACTTATATGCGTCTATTTATGTAG
GATGAAAGGTAGTCTAGTACCTCCTGTGATATTATCCCATTCCATGCG
GGGTATCGTATGCTTCCTTCAGCACTACCCTTTAGCTGTTCTATATGC
TGCCACTCCTCAATTGGATTAGTCTCATCCTTCAATGCTATCATTTCC
TTTGATATTGGATCATACTAAGAAACCATTATTATCATGACATTAACC
TATAAAAATAGGCGTATCACGAGGCCCTTTCGTCTCGCGCGTTTCGGT
GATGACGGTGAAAACCTCTGACACATGCAGCTCCCGGAGACGGTCACA
GCTTGTCTGTAAGCGGATGCCGGGAGCAGACAAGCCCGTCAGGGCGCG
TCAGCGGGTGTTGGCGGGTGTCGGGGCTGGCTTAACTATGCGGCATCA
GAGCAGATTGTACTGAGAGTGCACCATATTTCACACCGCATAGATCCG
TCGAGTTCAAGAGAAAAAAAAAGAAAAAGCAAAAAGAAAAAAGGAAAG
CGCGCCTCGTTCAGAATGACACGTATAGAATGATGCATTACCTTGTCA
TCTTCAGTATCATACTGTTCGTATACATACTTACTGACATTCATAGGT
ATACATATATACACATGTATATATATCGTATGCTGCAGCTTTAAATAA
TCGGTGTCACTACATAAGAACACCTTTGGTGGAGGGAACATCGTTGGT
ACCATTGGGCGAGGTGGCTTCTCTTATGGCAACCGCAAGAGCCTTGAA
CGCACTCTCACTACGGTGATGATCATTCTTGCCTCGCAGACAATCAAC
GTGGAGGGTAATTCTGCTAGCCTCTGCAAAGCTTTCAAGAAAATGCGG
GATCATCTCGCAAGAGAGATCTCCTACTTTCTCCCTTTGCAAACCAAG
TTCGACAACTGCGTACGGCCTGTTCGAAAGATCTACCACCGCTCTGGA
AAGTGCCTCATCCAAAGGCGCAAATCCTGATCCAAACCTTTTTACTCC
ACGCACGGCCCCTAGGGCCTCTTTAAAAGCTTGACCGAGAGCAATCCC
GCAGTCTTCAGTGGTGTGATGGTCGTCTATGTGTAAGTCACCAATGCA
CTCAACGATTAGCGACCAGCCGGAATGCTTGGCCAGAGCATGTATCAT
ATGGTCCAGAAACCCTATACCTGTGTGGACGTTAATCACTTGCGATTG
TGTGGCCTGTTCTGCTACTGCTTCTGCCTCTTTTTCTGGGAAGATCGA
GTGCTCTATCGCTAGGGGACCACCCTTTAAAGAGATCGCAATCTGAAT
CTTGGTTTCATTTGTAATACGCTTTACTAGGGCTTTCTGCTCTGTCAT
CTTTGCCTTCGTTTATCTTGCCTGCTCATTTTTTAGTATATTCTTCGA
AGAAATCACATTACTTTATATAATGTATAATTCATTATGTGATAATGC
CAATCGCTAAGAAAAAAAAAGAGTCATCCGCTAGGTGGAAAAAAAAAA
ATGAAAATCATTACCGAGGCATAAAAAAATATAGAGTGTACTAGAGGA
GGCCAAGAGTAATAGAAAAAGAAAATTGCGGGAAAGGACTGTGTTATG
ACTTCCCTGACTAATGCCGTGTTCAAACGATACCTGGCAGTGACTCCT
AGCGCTCACCAAGCTCTTAAAACGGGAATTTATGGTGCACTCTCAGTA
CACGCGCCAGATCTGTTTAGCTTGCCTCGTCCCCGCCGGGTCACCCGG
CCAGCGACATGGAGGCCCAGAATACCCTCCTTGACAGTCTTGACGTGC
GCAGCTCAGGGGCATGATGTGACTGTCGCCCGTACATTTAGCCCATAC
ATCCCCATGTATAATCATTTGCATCCATACATTTTGATGGCCGCACGG
CGCGAAGCAAAAATTACGGCTCCTCGCTGCAGACCTGCGAGCAGGGAA
ACGCTCCCCTCACAGACGCGTTGAATTGTCCCCACGCCGCGCCCCTGT
AGAGAAATATAAAAGGTTAGGATTTGCCACTGAGGTTCTTCTTTCATA
TACTTCCTTTTAAAATCTTGCTAGGATACAGTTCTCACATCACATCCG
AACATAAACAACCATGGGTAAGGAAAAGACTCACGTTTCGAGGCCGCG
ATTAAATTCCAACATGGATGCTGATTTATATGGGTATAAATGGGCTCG
CGATAATGTCGGGCAATCAGGTGCGACAATCTATCGATTGTATGGGAA
GCCCGATGCGCCAGAGTTGTTTCTGAAACATGGCAAAGGTAGCGTTGC
CAATGATGTTACAGATGAGATGGTCAGACTAAACTGGCTGACGGAATT
TATGCCTCTTCCGACCATCAAGCATTTTATCCGTACTCCTGATGATGC
ATGGTTACTCACCACTGCGATCCCCGGCAAAACAGCATTCCAGGTATT
AGAAGAATATCCTGATTCAGGTGAAAATATTGTTGATGCGCTGGCAGT
GTTCCTGCGCCGGTTGCATTCGATTCCTGTTTGTAATTGTCCTTTTAA
CAGCGATCGCGTATTTCGTCTCGCTCAGGCGCAATCACGAATGAATAA
CGGTTTGGTTGATGCGAGTGATTTTGATGACGAGCGTAATGGCTGGCC
TGTTGAACAAGTCTGGAAAGAAATGCATAAGCTTTTGCCATTCTCACC
GGATTCAGTCGTCACTCATGGTGATTTCTCACTTGATAACCTTATTTT
TGACGAGGGGAAATTAATAGGTTGTATTGATGTTGGACGAGTCGGAAT
CGCAGACCGATACCAGGATCTTGCCATCCTATGGAACTGCCTCGGTGA
GTTTTCTCCTTCATTACAGAAACGGCTTTTTCAAAAATATGGTATTGA
TAATCCTGATATGAATAAATTGCAGTTTCATTTGATGCTCGATGAGTT
TTTCTAATCAGTACTGACAATAAAAAGATTCTTGTTTTCAAGAACTTG
TCATTTGTATAGTTTTTTTATATTGTAGTTGTTCTATTTTAATCAAAT
GTTAGCGTGATTTATATTTTTTTTCGCCTCGACATCATCTGCCCAGAT
GCGAAGTTAAGTGCGCAGAAAGTAATATCATGCGTCAATCGTATGTGA
ATGCTGGTCGCTATACTGCTGTCGATTCGATACTAACGCCGCCATCCA
GTGTCGAATTCGCCATTCAGGCTGCGCAACTGTTGGGAAGGGCGATCG
GTGCGGGCCTCTTCGCTATTACGCCAGCTGAATTGGAGCGACCTCATG
CTATACCTGAGAAAGCAACCTGACCTACAGGAAAGAGTTACTCAAGAA
TAAGAATTTTCGTTTTAAAACCTAAGAGTCACTTTAAAATTTGTATAC
ACTTATTTTTTTTATAACTTATTTAATAATAAAAATCATAAATCATAA
GAAATTCGCTTATTTAGAAGTGTCAACAACGTATCTACCAACGATTTG
ACCCTTTTCCATCTTTTCGTAAATTTCTGGCAAGGTAGACAAGCCGAC
AACCTTGATTGGAGACTTGACCAAACCTCTGGCGAAGAATTGTTAATT
AAGAGCTCAGATCTTTTGCGGCCGC
SEQ. ID NO: 77
GTTTGCAAAAAGAACAAAACTGAAAAAACCCAGACACGCTCGACTTCC
TGTCTTCCTATTGATTGCAGCTTCCAATTTCGTCACACAACAAGGTCC
TAGCGACGGCTCACAGGTTTTGTAACAAGCAATCGAAGGTTCTGGAAT
GGCGGGAAAGGGTTTAGTACCACATGCTATGATGCCCACTGTGATCTC
CAGAGCAAAGTTCGTTCGATCGTACTGTTACTCTCTCTCTTTCAAACA
GAATTGTCCGAATCGTGTGACAACAACAGCCTGTTCTCACACACTCTT
TTCTTCTAACCAAGGGGGTGGTTTAGTTTAGTAGAACCTCGTGAAACT
TACATTTACATATATATAAACTTGCATAAATTGGTCAATGCAAGAAAT
ACATATTTGGTCTTTTCTAATTCGTAGTTTTTCAAGTTCTTAGATGCT
TTCTTTTTCTCTTTTTTACAGATCATCAAGGAAGTAATTATCTACTTT
TTACAACAAATATAAAACA
SEQ. ID NO: 78
ACAGTTTATTCCTGGCATCCACTAAATATAATGGAGCCCGCTTTTTAA
GCTGGCATCCAGAAAAAAAAAGAATCCCAGCACCAAAATATTGTTTTC
TTCACCAACCATCAGTTCATAGGTCCATTCTCTTAGCGCAACTACAGA
GAACAGGGGCACAAACAGGCAAAAAACGGGCACAACCTCAATGGAGTG
ATGCAACCTGCCTGGAGTAAATGATGACACAAGGCAATTGACCCACGC
ATGTATCTATCTCATTTTCTTACACCTTCTATTACCTTCTGCTCTCTC
TGATTTGGAAAAAGCTGAAAAAAAAGGTTGAAACCAGTTCCCTGAAAT
TATTCCCCTACTTGACTAATAAGTATATAAAGACGGTAGGTATTGATT
GTAATTCTGTAAATCTATTTCTTAAACTTCTTAAATTCTACTTTTATA
GTTAGTCTTTTTTTTAGTTTTAAAACACCAAGAACTTAGTTTCGAATA
AACACACATAAACAAACAAA
SEQ. ID NO: 79
GCTGGATTGAGCTGAATGGTGCCAGGTCGAGGCTGGGAGGGAGACTAA
CTCGAAAGTGACGAAGACTCGAAAATTAAAAAAAAAGATACTGCAGAA
GGCAAGATTGAGAATGGAGTAAAGGCAGCGTGGGTCCCCTGTGGAAAC
CGCAGTTTTCCTGCGCCAAGTGGTACCGGTGCGAGTGCAGCAATTAAT
CTCTCGATATTTTCTTAGTATCTCTTTTTATATAAGAATATATTTTGG
AATTGGTAATGCTTATCTTCAATAGTTTCTTAGTTGAATGCACACTTA
AGAGCAAATTGGCCAAGGAGTTCTTCGTTCGCTTTAATTTATTTCCTG
GTTATTGTCAATTTATTCATCCCATCTCCCCAGGATAGAAGAAATTAG
TGTAATTTTGCTGACAATACATTTTAACGACGATAACAATAATAGCAA
TTAAATAAAATAGCACTACCACCACTCCACTGCTCGTTAGCTATTTCT
GTAAAATAAATAAAAAGATC
SEQ. ID NO: 80
ATTCGCGCTATCTCGATTTCTACCTATATAGTTAATCTCTGTACAAAA
ACAATCTTTCCAACTATCCATTAATCATAGTATATTATCAGCGTCGGC
GATTTTACCACGCTTGACAAAAGCCGCGGGCGGGATTCCTGTGGGTAG
TGGCACCGGCAGTTAATCTAATCAAAGGCGCTTGAAGGAAGAGATAGA
TAATAGAACAAAGCAATCGCCGCTTTGGACGGCAAATATGTTTATCCA
TTGGTGCGGTGATTGGATATGATTTGTCTCCAGTAGTATAAGCAAGCG
CCAGATCTGTTTACTGTAAAATTAAGTGAGTAATCTCGCGGGATGTAA
TGATTTAAGGGAATCTGGTTCAGGTTTTCACATATATTTGTATATAAG
GCCATTTGTAATTTCAATAGTTTTAGGATTTTTCCTTCTCCCAAAATA
CTCACTTACTGTGTTACATTACAGAAAGAACAGACAAGAAACCGTCAA
TAAGAAATATAACTAAGAACA
SEQ. ID NO: 81
TACCCATACGACGTTCCAGACTACGCC
SEQ. ID NO: 82
YPYDVPDYA
SEQ. ID NO: 83
GAACAAAAGTTAATTTCTGAAGAAGATTTGGAA
SEQ. ID NO: 84
EQKLISEEDL
SEQ. ID NO: 85
GACTACAAGGATGACGATGACAAA
SEQ. ID NO: 86
DYKDDDDK
SEQ. ID NO: 87
GGTAAGCCAATTCCAAATCCTTTGTTGGGTTTGGACTCCACC
SEQ. ID NO: 88
GKPIPNPLLGLDST
SEQ. ID NO: 89
CATCATCATCATCATCAT
SEQ. ID NO: 90
HHHHHH
SEQ. ID NO: 91
CAGGTGGCACTTTTCGGGGAAATGTGCGCGGAACCCCTATTTGTTTAT
TTTTCTAAATACATTCAAATATGTATCCGCTCATGAGACAATAACCCT
GATAAATGCTTCAATAATATTGAAAAAGGAAGAGTATGAGTATTCAAC
ATTTCCGTGTCGCCCTTATTCCCTTTTTTGCGGCATTTTGCCTTCCTG
TTTTTGCTCACCCAGAAACGCTGGTGAAAGTAAAAGATGCTGAAGATC
AGTTGGGTGCACGAGTGGGTTACATCGAACTGGATCTCAACAGCGGTA
AGATCCTTGAGAGTTTTCGCCCCGAAGAACGTTTTCCAATGATGAGCA
CTTTTAAAGTTCTGCTATGTGGCGCGGTATTATCCCGTATTGACGCCG
GGCAAGAGCAACTCGGTCGCCGCATACACTATTCTCAGAATGACTTGG
TTGAGTACTCACCAGTCACAGAAAAGCATCTTACGGATGGCATGACAG
TAAGAGAATTATGCAGTGCTGCCATAACCATGAGTGATAACACTGCGG
CCAACTTACTTCTGACAACGATCGGAGGACCGAAGGAGCTAACCGCTT
TTTTGCACAACATGGGGGATCATGTAACTCGCCTTGATCGTTGGGAAC
CGGAGCTGAATGAAGCCATACCAAACGACGAGCGTGACACCACGATGC
CTGTAGCAATGGCAACAACGTTGCGCAAACTATTAACTGGCGAACTAC
TTACTCTAGCTTCCCGGCAACAATTAATAGACTGGATGGAGGCGGATA
AAGTTGCAGGACCACTTCTGCGCTCGGCCCTTCCGGCTGGCTGGTTTA
TTGCTGATAAATCTGGAGCCGGTGAGCGTGGGTCTCGCGGTATCATTG
CAGCACTGGGGCCAGATGGTAAGCCCTCCCGTATCGTAGTTATCTACA
CGACGGGGAGTCAGGCAACTATGGATGAACGAAATAGACAGATCGCTG
AGATAGGTGCCTCACTGATTAAGCATTGGTAACTGTCAGACCAAGTTT
ACTCATATATACTTTAGATTGATTTAAAACTTCATTTTTAATTTAAAA
GGATCTAGGTGAAGATCCTTTTTGATAATCTCATGACCAAAATCCCTT
AACGTGAGTTTTCGTTCCACTGAGCGTCAGACCCCGTAGAAAAGATCA
AAGGATCTTCTTGAGATCCTTTTTTTCTGCGCGTAATCTGCTGCTTGC
AAACAAAAAAACCACCGCTACCAGCGGTGGTTTGTTTGCCGGATCAAG
AGCTACCAACTCTTTTTCCGAAGGTAACTGGCTTCAGCAGAGCGCAGA
TACCAAATACTGTTCTTCTAGTGTAGCCGTAGTTAGGCCACCACTTCA
AGAACTCTGTAGCACCGCCTACATACCTCGCTCTGCTAATCCTGTTAC
CAGTGGCTGCTGCCAGTGGCGATAAGTCGTGTCTTACCGGGTTGGACT
CAAGACGATAGTTACCGGATAAGGCGCAGCGGTCGGGCTGAACGGGGG
GTTCGTGCACACAGCCCAGCTTGGAGCGAACGACCTACACCGAACTGA
GATACCTACAGCGTGAGCTATGAGAAAGCGCCACGCTTCCCGAAGGGA
GAAAGGCGGACAGGTATCCGGTAAGCGGCAGGGTCGGAACAGGAGAGC
GCACGAGGGAGCTTCCAGGGGGAAACGCCTGGTATCTTTATAGTCCTG
TCGGGTTTCGCCACCTCTGACTTGAGCGTCGATTTTTGTGATGCTCGT
CAGGGGGGCGGAGCCTATGGAAAAACGCCAGCAACGCGGCCTTTTTAC
GGTTCCTGGCCTTTTGCTGGCCTTTTGCTCACATGTTCTTTCCTGCGT
TATCCCCTGATTCTGTGGATAACCGTATTACCGCCTTTGAGTGAGCTG
ATACCGCTCGCCGCAGCCGAACGACCGAGCGCAGCGAGTCAGTGAGCG
AGGAAGCGGAAGAGCGCCCAATACGCAAACCGCCTCTCCCCGCGCGTT
GGCCGATTCATTAATGCAGCTGGATCTTCGAGCGTCCCAAAACCTTCT
CAAGCAAGGTTTTCAGTATAATGTTACATGCGTACACGCGTCTGTACA
GAAAAAAAAGAAAAATTTGAAATATAAATAACGTTCTTAATACTAACA
TAACTATAAAAAAATAAATAGGGACCTAGACTTCAGGTTGTCTAACTC
CTTCCTTTTCGGTTAGAGCGGATCTTAGCTAGCCGCGGTACCAAGCTG
GTGGATCCTTTGTTTGTTTATGTGTGTTTATTCGAAACTAAGTTCTTG
GTGTTTTAAAACTAAAAAAAAGACTAACTATAAAAGTAGAATTTAAGA
AGTTTAAGAAATAGATTTACAGAATTACAATCAATACCTACCGTCTTT
ATATACTTATTAGTCAAGTAGGGGAATAATTTCAGGGAACTGGTTTCA
ACCTTTTTTTTCAGCTTTTTCCAAATCAGAGAGAGCAGAAGGTAATAG
AAGGTGTAAGAAAATGAGATAGATACATGCGTGGGTCAATTGCCTTGT
GTCATCATTTACTCCAGGCAGGTTGCATCACTCCATTGAGGTTGTGCC
CGTTTTTTGCCTGTTTGTGCCCCTGTTCTCTGTAGTTGCGCTAAGAGA
ATGGACCTATGAACTGATGGTTGGTGAAGAAAACAATATTTTGGTGCT
GGGATTCTTTTTTTTTCTGGATGCCAGCTTAAAAAGCGGGCTCCATTA
TATTTAGTGGATGCCAGGAATAAACTGTTGTTTGCAAAAAGAACAAAA
CTGAAAAAACCCAGACACGCTCGACTTCCTGTCTTCCTATTGATTGCA
GCTTCCAATTTCGTCACACAACAAGGTCCTAGCGACGGCTCACAGGTT
TTGTAACAAGCAATCGAAGGTTCTGGAATGGCGGGAAAGGGTTTAGTA
CCACATGCTATGATGCCCACTGTGATCTCCAGAGCAAAGTTCGTTCGA
TCGTACTGTTACTCTCTCTCTTTCAAACAGAATTGTCCGAATCGTGTG
ACAACAACAGCCTGTTCTCACACACTCTTTTCTTCTAACCAAGGGGGT
GGTTTAGTTTAGTAGAACCTCGTGAAACTTACATTTACATATATATAA
ACTTGCATAAATTGGTCAATGCAAGAAATACATATTTGGTCTTTTCTA
ATTCGTAGTTTTTCAAGTTCTTAGATGCTTTCTTTTTCTCTTTTTTAC
AGATCATCAAGGAAGTAATTATCTACTTTTTACAACAAATATAAAACA
GCGGCCGCAAAAGATCTGAGCTCTTAATTAACAATTCTTCGCCAGAGG
TTTGGTCAAGTCTCCAATCAAGGTTGTCGGCTTGTCTACCTTGCCAGA
AATTTACGAAAAGATGGAAAAGGGTCAAATCGTTGGTAGATACGTTGT
TGACACTTCTAAATAAGCGAATTTCTTATGATTTATGATTTTTATTAT
TAAATAAGTTATAAAAAAAATAAGTGTATACAAATTTTAAAGTGACTC
TTAGGTTTTAAAACGAAAATTCTTATTCTTGAGTAACTCTTTCCTGTA
GGTCAGGTTGCTTTCTCAGGTATAGCATGAGGTCGCTCCAATTCAGCT
GGCGTAATAGCGAAGAGGCCCGCACCGATCGCCCTTCCCAACAGTTGC
GCAGCCTGAATGGCGAATTCGACACTGGATGGCGGCGTTAGTATCGAA
TCGACAGCAGTATAGCGACCAGCATTCACATACGATTGACGCATGATA
TTACTTTCTGCGCACTTAACTTCGCATCTGGGCAGATGATGTCGAGGC
GAAAAAAAATATAAATCACGCTAACATTTGATTAAAATAGAACAACTA
CAATATAAAAAAACTATACAAATGACAAGTTCTTGAAAACAAGAATCT
TTTTATTGTCAGTACTGATTAGAAAAACTCATCGAGCATCAAATGAAA
CTGCAATTTATTCATATCAGGATTATCAATACCATATTTTTGAAAAAG
CCGTTTCTGTAATGAAGGAGAAAACTCACCGAGGCAGTTCCATAGGAT
GGCAAGATCCTGGTATCGGTCTGCGATTCCGACTCGTCCAACATCAAT
ACAACCTATTAATTTCCCCTCGTCAAAAATAAGGTTATCAAGTGAGAA
ATCACCATGAGTGACGACTGAATCCGGTGAGAATGGCAAAAGCTTATG
CATTTCTTTCCAGACTTGTTCAACAGGCCAGCCATTACGCTCGTCATC
AAAATCACTCGCATCAACCAAACCGTTATTCATTCGTGATTGCGCCTG
AGCGAGACGAAATACGCGATCGCTGTTAAAAGGACAATTACAAACAGG
AATCGAATGCAACCGGCGCAGGAACACTGCCAGCGCATCAACAATATT
TTCACCTGAATCAGGATATTCTTCTAATACCTGGAATGCTGTTTTGCC
GGGGATCGCAGTGGTGAGTAACCATGCATCATCAGGAGTACGGATAAA
ATGCTTGATGGTCGGAAGAGGCATAAATTCCGTCAGCCAGTTTAGTCT
GACCATCTCATCTGTAACATCATTGGCAACGCTACCTTTGCCATGTTT
CAGAAACAACTCTGGCGCATCGGGCTTCCCATACAATCGATAGATTGT
CGCACCTGATTGCCCGACATTATCGCGAGCCCATTTATACCCATATAA
ATCAGCATCCATGTTGGAATTTAATCGCGGCCTCGAAACGTGAGTCTT
TTCCTTACCCATGGTTGTTTATGTTCGGATGTGATGTGAGAACTGTAT
CCTAGCAAGATTTTAAAAGGAAGTATATGAAAGAAGAACCTCAGTGGC
AAATCCTAACCTTTTATATTTCTCTACAGGGGCGCGGCGTGGGGACAA
TTCAACGCGTCTGTGAGGGGAGCGTTTCCCTGCTCGCAGGTCTGCAGC
GAGGAGCCGTAATTTTTGCTTCGCGCCGTGCGGCCATCAAAATGTATG
GATGCAAATGATTATACATGGGGATGTATGGGCTAAATGTACGGGCGA
CAGTCACATCATGCCCCTGAGCTGCGCACGTCAAGACTGTCAAGGAGG
GTATTCTGGGCCTCCATGTCGCTGGCCGGGTGACCCGGCGGGGACGAG
GCAAGCTAAACAGATCTGGCGCGTGTACTGAGAGTGCACCATAAATTC
CCGTTTTAAGAGCTTGGTGAGCGCTAGGAGTCACTGCCAGGTATCGTT
TGAACACGGCATTAGTCAGGGAAGTCATAACACAGTCCTTTCCCGCAA
TTTTCTTTTTCTATTACTCTTGGCCTCCTCTAGTACACTCTATATTTT
TTTATGCCTCGGTAATGATTTTCATTTTTTTTTTTCCACCTAGCGGAT
GACTCTTTTTTTTTCTTAGCGATTGGCATTATCACATAATGAATTATA
CATTATATAAAGTAATGTGATTTCTTCGAAGAATATACTAAAAAATGA
GCAGGCAAGATAAACGAAGGCAAAGATGACAGAGCAGAAAGCCCTAGT
AAAGCGTATTACAAATGAAACCAAGATTCAGATTGCGATCTCTTTAAA
GGGTGGTCCCCTAGCGATAGAGCACTCGATCTTCCCAGAAAAAGAGGC
AGAAGCAGTAGCAGAACAGGCCACACAATCGCAAGTGATTAACGTCCA
CACAGGTATAGGGTTTCTGGACCATATGATACATGCTCTGGCCAAGCA
TTCCGGCTGGTCGCTAATCGTTGAGTGCATTGGTGACTTACACATAGA
CGACCATCACACCACTGAAGACTGCGGGATTGCTCTCGGTCAAGCTTT
TAAAGAGGCCCTAGGGGCCGTGCGTGGAGTAAAAAGGTTTGGATCAGG
ATTTGCGCCTTTGGATGAGGCACTTTCCAGAGCGGTGGTAGATCTTTC
GAACAGGCCGTACGCAGTTGTCGAACTTGGTTTGCAAAGGGAGAAAGT
AGGAGATCTCTCTTGCGAGATGATCCCGCATTTTCTTGAAAGCTTTGC
AGAGGCTAGCAGAATTACCCTCCACGTTGATTGTCTGCGAGGCAAGAA
TGATCATCACCGTAGTGAGAGTGCGTTCAAGGCTCTTGCGGTTGCCAT
AAGAGAAGCCACCTCGCCCAATGGTACCAACGATGTTCCCTCCACCAA
AGGTGTTCTTATGTAGTGACACCGATTATTTAAAGCTGCAGCATACGA
TATATATACATGTGTATATATGTATACCTATGAATGTCAGTAAGTATG
TATACGAACAGTATGATACTGAAGATGACAAGGTAATGCATCATTCTA
TACGTGTCATTCTGAACGAGGCGCGCTTTCCTTTTTTCTTTTTGCTTT
TTCTTTTTTTTTCTCTTGAACTCGACGGATCTATGCGGTGTGAAATAT
GGTGCACTCTCAGTACAATCTGCTCTGATGCCGCATAGTTAAGCCAGC
CCCGACACCCGCCAACACCCGCTGACGCGCCCTGACGGGCTTGTCTGC
TCCCGGCATCCGCTTACAGACAAGCTGTGACCGTCTCCGGGAGCTGCA
TGTGTCAGAGGTTTTCACCGTCATCACCGAAACGCGCGAGACGAAAGG
GCCTCGTGATACGCCTATTTTTATAGGTTAATGTCATGATAATAATGG
TTTCTTAGTATGATCCAATATCAAAGGAAATGATAGCATTGAAGGATG
AGACTAATCCAATTGAGGAGTGGCAGCATATAGAACAGCTAAAGGGTA
GTGCTGAAGGAAGCATACGATACCCCGCATGGAATGGGATAATATCAC
AGGAGGTACTAGACTACCTTTCATCCTACATAAATAGACGCATATAAG
TACGCATTTAAGCATAAACACGCACTATGCCGTTCTTCTCATGTATAT
ATATATACAGGCAACACGCAGATATAGGTGCGACGTGAACAGTGAGCT
GTATGTGCGCAGCTCGCGTTGCATTTTCGGAAGCGCTCGTTTTCGGAA
ACGCTTTGAAGTTCCTATTCCGAAGTTCCTATTCTCTAGAAAGTATAG
GAACTTCAGAGCGCTTTTGAAAACCAAAAGCGCTCTGAAGACGCACTT
TCAAAAAACCAAAAACGCACCGGACTGTAACGAGCTACTAAAATATTG
CGAATACCGCTTCCACAAACATTGCTCAAAAGTATCTCTTTGCTATAT
ATCTCTGTGCTATATCCCTATATAACCTACCCATCCACCTTTCGCTCC
TTGAACTTGCATCTAAACTCGACCTCTACATTTTTTATGTTTATCTCT
AGTATTACTCTTTAGACAAAAAAATTGTAGTAAGAACTATTCATAGAG
TGAATCGAAAACAATACGAAAATGTAAACATTTCCTATACGTAGTATA
TAGAGACAAAATAGAAGAAACCGTTCATAATTTTCTGACCAATGAAGA
ATCATCAACGCTATCACTTTCTGTTCACAAAGTATGCGCAATCCACAT
CGGTATAGAATATAATCGGGGATGCCTTTATCTTGAAAAAATGCACCC
GCAGCTTCGCTAGTAATCAGTAAACGCGGGAAGTGGAGTCAGGCTTTT
TTTATGGAAGAGAAAATAGACACCAAAGTAGCCTTCTTCTAACCTTAA
CGGACCTACAGTGCAAAAAGTTATCAAGAGACTGCATTATAGAGCGCA
CAAAGGAGAAAAAAAGTAATCTAAGATGCTTTGTTAGAAAAATAGCGC
TCTCGGGATGCATTTTTGTAGAACAAAAAAGAAGTATAGATTCTTTGT
TGGTAAAATAGCGCTCTCGCGTTGCATTTCTGTTCTGTAAAAATGCAG
CTCAGATTCTTTGTTTGAAAAATTAGCGCTCTCGCGTTGCATTTTTGT
TTTACAAAAATGAAGCACAGATTCTTCGTTGGTAAAATAGCGCTTTCG
CGTTGCATTTCTGTTCTGTAAAAATGCAGCTCAGATTCTTTGTTTGAA
AAATTAGCGCTCTCGCGTTGCATTTTTGTTCTACAAAATGAAGCACAG
ATGCTTCGTT
SEQ. ID NO: 92
GCTAACAGCGCGATTTGCTGGTGACCCAATGCGACCAGATGCTCCACG
CCCAGTCGCGTACCGTCTTCATGGGAGAAAATAATACTGTTGATGGGT
GTCTGGTCAGAGACATCAAGAAATAACGCCGGAACATTAGTGCAGGCA
GCTTCCACAGCAATGGCATCCTGGTCATCCAGCGGATAGTTAATGATC
AGCCCACTGACGCGTTGCGCGAGAAGATTGTGCACCGCCGCTTTACAG
GCTTCGACGCCGCTTCGTTCTACCATCGACACCACCACGCTGGCACCC
AGTTGATCGGCGCGAGATTTAATCGCCGCGACAATTTGCGACGGCGCG
TGCAGGGCCAGACTGGAGGTGGCAACGCCAATCAGCAACGACTGTTTG
CCCGCCAGTTGTTGTGCCACGCGGTTGGGAATGTAATTCAGCTCCGCC
ATCGCCGCTTCCACTTTTTCCCGCGTTTTCGCAGAAACGTGGCTGGCC
TGGTTCACCACGCGGGAAACGGTCTGATAAGAGACACCGGCATACTCT
GCGACATCGTATAACGTTACTGGTTTCACATTCACCACCCTGAATTGA
CTCTCTTCCGGGCGCTATCATGCCATACCGCGAAAGGTTTTGCGCCAT
TCGATGGTGTCCGGGATCTCGACGCTCTCCCTTATGCGACTCCTGCAT
TAGGAAGCAGCCCAGTAGTAGGTTGAGGCCGTTGAGCACCGCCGCCGC
AAGGAATGGTGCATGCAAGGAGATGGCGCCCAACAGTCCCCCGGCCAC
GGGGCCTGCCACCATACCCACGCCGAAACAAGCGCTCATGAGCCCGAA
GTGGCGAGCCCGATCTTCCCCATCGGTGATGTCGGCGATATAGGCGCC
AGCAACCGCACCTGTGGCGCCGGTGATGCCGGCCACGATGCGTCCGGC
GTAGCCTAGGATCGAGATCGATCTCGATCCCGCGAAATTAATACGACT
CACTATAGGGGAATTGTGAGCGGATAACAATTCCCCTCTAGAAATAAT
TTTGTTTAACTTTAAGAAGGAGATATACATATGGCAGATCTCAATTGG
ATATCGGCCGGCCACGCGATCGCTGACGTCGGTACCCTCGAGTCTGGT
AAAGAAACCGCTGCTGCGAAATTTGAACGCCAGCACATGGACTCGTCT
ACTAGTCGCAGCTTAATTAACCTAAACTGCTGCCACCGCTGAGCAATA
ACTAGCATAACCCCTTGGGGCCTCTAAACGGGTCTTGAGGGGTTTTTT
GCTAGCGAAAGGAGGAGTCGACACTGCTTCCGGTAGTCAATAAACCGG
TAAACCAGCAATAGACATAAGCGGCTATTTAACGACCCTGCCCTGAAC
CGACGACCGGGTCATCGTGGCCGGATCTTGCGGCCCCTCGGCTTGAAC
GAATTGTTAGACATTATTTGCCGACTACCTTGGTGATCTCGCCTTTCA
CGTAGTGGACAAATTCTTCCAACTGATCTGCGCGCGAGGCCAAGCGAT
CTTCTTCTTGTCCAAGATAAGCCTGTCTAGCTTCAAGTATGACGGGCT
GATACTGGGCCGGCAGGCGCTCCATTGCCCAGTCGGCAGCGACATCCT
TCGGCGCGATTTTGCCGGTTACTGCGCTGTACCAAATGCGGGACAACG
TAAGCACTACATTTCGCTCATCGCCAGCCCAGTCGGGCGGCGAGTTCC
ATAGCGTTAAGGTTTCATTTAGCGCCTCAAATAGATCCTGTTCAGGAA
CCGGATCAAAGAGTTCCTCCGCCGCTGGACCTACCAAGGCAACGCTAT
GTTCTCTTGCTTTTGTCAGCAAGATAGCCAGATCAATGTCGATCGTGG
CTGGCTCGAAGATACCTGCAAGAATGTCATTGCGCTGCCATTCTCCAA
ATTGCAGTTCGCGCTTAGCTGGATAACGCCACGGAATGATGTCGTCGT
GCACAACAATGGTGACTTCTACAGCGCGGAGAATCTCGCTCTCTCCAG
GGGAAGCCGAAGTTTCCAAAAGGTCGTTGATCAAAGCTCGCCGCGTTG
TTTCATCAAGCCTTACGGTCACCGTAACCAGCAAATCAATATCACTGT
GTGGCTTCAGGCCGCCATCCACTGCGGAGCCGTACAAATGTACGGCCA
GCAACGTCGGTTCGAGATGGCGCTCGATGACGCCAACTACCTCTGATA
GTTGAGTCGATACTTCGGCGATCACCGCTTCCCTCATACTCTTCCTTT
TTCAATATTATTGAAGCATTTATCAGGGTTATTGTCTCATGAGCGGAT
ACATATTTGAATGTATTTAGAAAAATAAACAAATAGCCAGCTCACTCG
GTCGCTACGCTCCGGGCGTGAGACTGCGGCGGGCGCTGCGGACACATA
CAAAGTTACCCACAGATTCCGTGGATAAGCAGGGGACTAACATGTGAG
GCAAAACAGCAGGGCCGCGCCGGTGGCGTTTTTCCATAGGCTCCGCCC
TCCTGCCAGAGTTCACATAAACAGACGCTTTTCCGGTGCATCTGTGGG
AGCCGTGAGGCTCAACCATGAATCTGACAGTACGGGCGAAACCCGACA
GGACTTAAAGATCCCCACCGTTTCCGGCGGGTCGCTCCCTCTTGCGCT
CTCCTGTTCCGACCCTGCCGTTTACCGGATACCTGTTCCGCCTTTCTC
CCTTACGGGAAGTGTGGCGCTTTCTCATAGCTCACACACTGGTATCTC
GGCTCGGTGTAGGTCGTTCGCTCCAAGCTGGGCTGTAAGCAAGAACTC
CCCGTTCAGCCCGACTGCTGCGCCTTATCCGGTAACTGTTCACTTGAG
TCCAACCCGGAAAAGCACGGTAAAACGCCACTGGCAGCAGCCATTGGT
AACTGGGAGTTCGCAGAGGATTTGTTTAGCTAAACACGCGGTTGCTCT
TGAAGTGTGCGCCAAAGTCCGGCTACACTGGAAGGACAGATTTGGTTG
CTGTGCTCTGCGAAAGCCAGTTACCACGGTTAAGCAGTTCCCCAACTG
ACTTAACCTTCGATCAAACCACCTCCCCAGGTGGTTTTTTCGTTTACA
GGGCAAAAGATTACGCGCAGAAAAAAAGGATCTCAAGAAGATCCTTTG
ATCTTTTCTACTGAACCGCTCTAGATTTCAGTGCAATTTATCTCTTCA
AATGTAGCACCTGAAGTCAGCCCCATACGATATAAGTTGTAATTCTCA
TGTTAGTCATGCCCCGCGCCCACCGGAAGGAGCTGACTGGGTTGAAGG
CTCTCAAGGGCATCGGTCGAGATCCCGGTGCCTAATGAGTGAGCTAAC
TTACATTAATTGCGTTGCGCTCACTGCCCGCTTTCCAGTCGGGAAACC
TGTCGTGCCAGCTGCATTAATGAATCGGCCAACGCGCGGGGAGAGGCG
GTTTGCGTATTGGGCGCCAGGGTGGTTTTTCTTTTCACCAGTGAGACG
GGCAACAGCTGATTGCCCTTCACCGCCTGGCCCTGAGAGAGTTGCAGC
AAGCGGTCCACGCTGGTTTGCCCCAGCAGGCGAAAATCCTGTTTGATG
GTGGTTAACGGCGGGATATAACATGAGCTGTCTTCGGTATCGTCGTAT
CCCACTACCGAGATGTCCGCACCAACGCGCAGCCCGGACTCGGTAATG
GCGCGCATTGCGCCCAGCGCCATCTGATCGTTGGCAACCAGCATCGCA
GTGGGAACGATGCCCTCATTCAGCATTTGCATGGTTTGTTGAAAACCG
GACATGGCACTCCAGTCGCCTTCCCGTTCCGCTATCGGCTGAATTTGA
TTGCGAGTGAGATATTTATGCCAGCCAGCCAGACGCAGACGCGCCGAG
ACAGAACTTAATGGGCCC
SEQ. ID NO: 93
GGGGAATTGTGAGCGGATAACAATTCCCCTCTAGAAATAATTTTGTTT
AACTTTAAGAAGGAGATATACATATGGCAGATCTCAATTGGATATCGG
CCGGCCACGCGATCGCTGACGTCGGTACCCTCGAGTCTGGTAAAGAAA
CCGCTGCTGCGAAATTTGAACGCCAGCACATGGACTCGTCTACTAGTC
GCAGCTTAATTAACCTAAACTGCTGCCACCGCTGAGCAATAACTAGCA
TAACCCCTTGGGGCCTCTAAACGGGTCTTGAGGGGTTTTTTGCTAGCG
AAAGGAGGAGTCGACTATATCCGGATTGGCGAATGGGACGCGCCCTGT
AGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCT
TCCTTTCTCGCCACGTTCGCCGGCTTTCCCCGTCAAGCTCTAAATCGG
GGGCTCCCTTTAGGGTTCCGATTTAGTGCTTTACGGCACCTCGACCCC
AAAAAACTTGATTAGGGTGATGGTTCACGTAGTGGGCCATCGCCCTGA
TAGACGGTTTTTCGCCCTTTGACGTTGGAGTCCACGTTCTTTAATAGT
GGACTCTTGTTCCAAACTGGAACAACACTCAACCCTATCTCGGTCTAT
TCTTTTGATTTATAAGGGATTTTGCCGATTTCGGCCTATTGGTTAAAA
AATGAGCTGATTTAACAAAAATTTAACGCGAATTTTAACAAAATATTA
ACGTTTACAATTTCTGGCGGCACGATGGCATGAGATTATCAAAAAGGA
TCTTCACCTAGATCCTTTTAAATTAAAAATGAAGTTTTAAATCAATCT
AAAGTATATATGAGTAAACTTGGTCTGACAGTTACCAATGCTTAATCA
GTGAGGCACCTATCTCAGCGATCTGTCTATTTCGTTCATCCATAGTTG
CCTGACTCCCCGTCGTGTAGATAACTACGATACGGGAGGGCTTACCAT
CTGGCCCCAGTGCTGCAATGATACCGCGAGACCCACGCTCACCGGCTC
CAGATTTATCAGCAATAAACCAGCCAGCCGGAAGGGCCGAGCGCAGAA
GTGGTCCTGCAACTTTATCCGCCTCCATCCAGTCTATTAATTGTTGCC
GGGAAGCTAGAGTAAGTAGTTCGCCAGTTAATAGTTTGCGCAACGTTG
TTGCCATTGCTACAGGCATCGTGGTGTCACGCTCGTCGTTTGGTATGG
CTTCATTCAGCTCCGGTTCCCAACGATCAAGGCGAGTTACATGATCCC
CCATGTTGTGCAAAAAAGCGGTTAGCTCCTTCGGTCCTCCGATCGTTG
TCAGAAGTAAGTTGGCCGCAGTGTTATCACTCATGGTTATGGCAGCAC
TGCATAATTCTCTTACTGTCATGCCATCCGTAAGATGCTTTTCTGTGA
CTGGTGAGTACTCAACCAAGTCATTCTGAGAATAGTGTATGCGGCGAC
CGAGTTGCTCTTGCCCGGCGTCAATACGGGATAATACCGCGCCACATA
GCAGAACTTTAAAAGTGCTCATCATTGGAAAACGTTCTTCGGGGCGAA
AACTCTCAAGGATCTTACCGCTGTTGAGATCCAGTTCGATGTAACCCA
CTCGTGCACCCAACTGATCTTCAGCATCTTTTACTTTCACCAGCGTTT
CTGGGTGAGCAAAAACAGGAAGGCAAAATGCCGCAAAAAAGGGAATAA
GGGCGACACGGAAATGTTGAATACTCATACTCTTCCTTTTTCAATCAT
GATTGAAGCATTTATCAGGGTTATTGTCTCATGAGCGGATACATATTT
GAATGTATTTAGAAAAATAAACAAATAGGTCATGACCAAAATCCCTTA
ACGTGAGTTTTCGTTCCACTGAGCGTCAGACCCCGTAGAAAAGATCAA
AGGATCTTCTTGAGATCCTTTTTTTCTGCGCGTAATCTGCTGCTTGCA
AACAAAAAAACCACCGCTACCAGCGGTGGTTTGTTTGCCGGATCAAGA
GCTACCAACTCTTTTTCCGAAGGTAACTGGCTTCAGCAGAGCGCAGAT
ACCAAATACTGTCCTTCTAGTGTAGCCGTAGTTAGGCCACCACTTCAA
GAACTCTGTAGCACCGCCTACATACCTCGCTCTGCTAATCCTGTTACC
AGTGGCTGCTGCCAGTGGCGATAAGTCGTGTCTTACCGGGTTGGACTC
AAGACGATAGTTACCGGATAAGGCGCAGCGGTCGGGCTGAACGGGGGG
TTCGTGCACACAGCCCAGCTTGGAGCGAACGACCTACACCGAACTGAG
ATACCTACAGCGTGAGCTATGAGAAAGCGCCACGCTTCCCGAAGGGAG
AAAGGCGGACAGGTATCCGGTAAGCGGCAGGGTCGGAACAGGAGAGCG
CACGAGGGAGCTTCCAGGGGGAAACGCCTGGTATCTTTATAGTCCTGT
CGGGTTTCGCCACCTCTGACTTGAGCGTCGATTTTTGTGATGCTCGTC
AGGGGGGCGGAGCCTATGGAAAAACGCCAGCAACGCGGCCTTTTTACG
GTTCCTGGCCTTTTGCTGGCCTTTTGCTCACATGTTCTTTCCTGCGTT
ATCCCCTGATTCTGTGGATAACCGTATTACCGCCTTTGAGTGAGCTGA
TACCGCTCGCCGCAGCCGAACGACCGAGCGCAGCGAGTCAGTGAGCGA
GGAAGCGGAAGAGCGCCTGATGCGGTATTTTCTCCTTACGCATCTGTG
CGGTATTTCACACCGCATATATGGTGCACTCTCAGTACAATCTGCTCT
GATGCCGCATAGTTAAGCCAGTATACACTCCGCTATCGCTACGTGACT
GGGTCATGGCTGCGCCCCGACACCCGCCAACACCCGCTGACGCGCCCT
GACGGGCTTGTCTGCTCCCGGCATCCGCTTACAGACAAGCTGTGACCG
TCTCCGGGAGCTGCATGTGTCAGAGGTTTTCACCGTCATCACCGAAAC
GCGCGAGGCAGCTGCGGTAAAGCTCATCAGCGTGGTCGTGAAGCGATT
CACAGATGTCTGCCTGTTCATCCGCGTCCAGCTCGTTGAGTTTCTCCA
GAAGCGTTAATGTCTGGCTTCTGATAAAGCGGGCCATGTTAAGGGCGG
TTTTTTCCTGTTTGGTCACTGATGCCTCCGTGTAAGGGGGATTTCTGT
TCATGGGGGTAATGATACCGATGAAACGAGAGAGGATGCTCACGATAC
GGGTTACTGATGATGAACATGCCCGGTTACTGGAACGTTGTGAGGGTA
AACAACTGGCGGTATGGATGCGGCGGGACCAGAGAAAAATCACTCAGG
GTCAATGCCAGCGCTTCGTTAATACAGATGTAGGTGTTCCACAGGGTA
GCCAGCAGCATCCTGCGATGCAGATCCGGAACATAATGGTGCAGGGCG
CTGACTTCCGCGTTTCCAGACTTTACGAAACACGGAAACCGAAGACCA
TTCATGTTGTTGCTCAGGTCGCAGACGTTTTGCAGCAGCAGTCGCTTC
ACGTTCGCTCGCGTATCGGTGATTCATTCTGCTAACCAGTAAGGCAAC
CCCGCCAGCCTAGCCGGGTCCTCAACGACAGGAGCACGATCATGCTAG
TCATGCCCCGCGCCCACCGGAAGGAGCTGACTGGGTTGAAGGCTCTCA
AGGGCATCGGTCGAGATCCCGGTGCCTAATGAGTGAGCTAACTTACAT
TAATTGCGTTGCGCTCACTGCCCGCTTTCCAGTCGGGAAACCTGTCGT
GCCAGCTGCATTAATGAATCGGCCAACGCGCGGGGAGAGGCGGTTTGC
GTATTGGGCGCCAGGGTGGTTTTTCTTTTCACCAGTGAGACGGGCAAC
AGCTGATTGCCCTTCACCGCCTGGCCCTGAGAGAGTTGCAGCAAGCGG
TCCACGCTGGTTTGCCCCAGCAGGCGAAAATCCTGTTTGATGGTGGTT
AACGGCGGGATATAACATGAGCTGTCTTCGGTATCGTCGTATCCCACT
ACCGAGATGTCCGCACCAACGCGCAGCCCGGACTCGGTAATGGCGCGC
ATTGCGCCCAGCGCCATCTGATCGTTGGCAACCAGCATCGCAGTGGGA
ACGATGCCCTCATTCAGCATTTGCATGGTTTGTTGAAAACCGGACATG
GCACTCCAGTCGCCTTCCCGTTCCGCTATCGGCTGAATTTGATTGCGA
GTGAGATATTTATGCCAGCCAGCCAGACGCAGACGCGCCGAGACAGAA
CTTAATGGGCCCGCTAACAGCGCGATTTGCTGGTGACCCAATGCGACC
AGATGCTCCACGCCCAGTCGCGTACCGTCTTCATGGGAGAAAATAATA
CTGTTGATGGGTGTCTGGTCAGAGACATCAAGAAATAACGCCGGAACA
TTAGTGCAGGCAGCTTCCACAGCAATGGCATCCTGGTCATCCAGCGGA
TAGTTAATGATCAGCCCACTGACGCGTTGCGCGAGAAGATTGTGCACC
GCCGCTTTACAGGCTTCGACGCCGCTTCGTTCTACCATCGACACCACC
ACGCTGGCACCCAGTTGATCGGCGCGAGATTTAATCGCCGCGACAATT
TGCGACGGCGCGTGCAGGGCCAGACTGGAGGTGGCAACGCCAATCAGC
AACGACTGTTTGCCCGCCAGTTGTTGTGCCACGCGGTTGGGAATGTAA
TTCAGCTCCGCCATCGCCGCTTCCACTTTTTCCCGCGTTTTCGCAGAA
ACGTGGCTGGCCTGGTTCACCACGCGGGAAACGGTCTGATAAGAGACA
CCGGCATACTCTGCGACATCGTATAACGTTACTGGTTTCACATTCACC
ACCCTGAATTGACTCTCTTCCGGGCGCTATCATGCCATACCGCGAAAG
GTTTTGCGCCATTCGATGGTGTCCGGGATCTCGACGCTCTCCCTTATG
CGACTCCTGCATTAGGAAGCAGCCCAGTAGTAGGTTGAGGCCGTTGAG
CACCGCCGCCGCAAGGAATGGTGCATGCAAGGAGATGGCGCCCAACAG
TCCCCCGGCCACGGGGCCTGCCACCATACCCACGCCGAAACAAGCGCT
CATGAGCCCGAAGTGGCGAGCCCGATCTTCCCCATCGGTGATGTCGGC
GATATAGGCGCCAGCAACCGCACCTGTGGCGCCGGTGATGCCGGCCAC
GATGCGTCCGGCGTAGCCTAGGATCGAGATCGATCTCGATCCCGCGAA
ATTAATACGACTCACTATA
SEQ. ID NO: 94
ATCCGGATATAGTTCCTCCTTTCAGCAAAAAACCCCTCAAGACCCGTT
TAGAGGCCCCAAGGGGTTATGCTAGTTATTGCTCAGCGGTGGCAGCAG
CCAACTCAGCTTCCTTTCGGGCTTTGTTAGCAGCCGGATCTCAGTGGT
GGTGGTGGTGGTGCTCGAGTGCGGCCGCAAGCTTGTCGACGGAGCTCG
AATTCGGATCCGCGACCCATTTGCTGTCCACCAGTCATGCTAGCCATA
TGGCTGCCGCGCGGCACCAGGCCGCTGCTGTGATGATGATGATGATGG
CTGCTGCCCATGGTATATCTCCTTCTTAAAGTTAAACAAAATTATTTC
TAGAGGGGAATTGTTATCCGCTCACAATTCCCCTATAGTGAGTCGTAT
TAATTTCGCGGGATCGAGATCTCGATCCTCTACGCCGGACGCATCGTG
GCCGGCATCACCGGCGCCACAGGTGCGGTTGCTGGCGCCTATATCGCC
GACATCACCGATGGGGAAGATCGGGCTCGCCACTTCGGGCTCATGAGC
GCTTGTTTCGGCGTGGGTATGGTGGCAGGCCCCGTGGCCGGGGGACTG
TTGGGCGCCATCTCCTTGCATGCACCATTCCTTGCGGCGGCGGTGCTC
AACGGCCTCAACCTACTACTGGGCTGCTTCCTAATGCAGGAGTCGCAT
AAGGGAGAGCGTCGAGATCCCGGACACCATCGAATGGCGCAAAACCTT
TCGCGGTATGGCATGATAGCGCCCGGAAGAGAGTCAATTCAGGGTGGT
GAATGTGAAACCAGTAACGTTATACGATGTCGCAGAGTATGCCGGTGT
CTCTTATCAGACCGTTTCCCGCGTGGTGAACCAGGCCAGCCACGTTTC
TGCGAAAACGCGGGAAAAAGTGGAAGCGGCGATGGCGGAGCTGAATTA
CATTCCCAACCGCGTGGCACAACAACTGGCGGGCAAACAGTCGTTGCT
GATTGGCGTTGCCACCTCCAGTCTGGCCCTGCACGCGCCGTCGCAAAT
TGTCGCGGCGATTAAATCTCGCGCCGATCAACTGGGTGCCAGCGTGGT
GGTGTCGATGGTAGAACGAAGCGGCGTCGAAGCCTGTAAAGCGGCGGT
GCACAATCTTCTCGCGCAACGCGTCAGTGGGCTGATCATTAACTATCC
GCTGGATGACCAGGATGCCATTGCTGTGGAAGCTGCCTGCACTAATGT
TCCGGCGTTATTTCTTGATGTCTCTGACCAGACACCCATCAACAGTAT
TATTTTCTCCCATGAAGACGGTACGCGACTGGGCGTGGAGCATCTGGT
CGCATTGGGTCACCAGCAAATCGCGCTGTTAGCGGGCCCATTAAGTTC
TGTCTCGGCGCGTCTGCGTCTGGCTGGCTGGCATAAATATCTCACTCG
CAATCAAATTCAGCCGATAGCGGAACGGGAAGGCGACTGGAGTGCCAT
GTCCGGTTTTCAACAAACCATGCAAATGCTGAATGAGGGCATCGTTCC
CACTGCGATGCTGGTTGCCAACGATCAGATGGCGCTGGGCGCAATGCG
CGCCATTACCGAGTCCGGGCTGCGCGTTGGTGCGGATATCTCGGTAGT
GGGATACGACGATACCGAAGACAGCTCATGTTATATCCCGCCGTTAAC
CACCATCAAACAGGATTTTCGCCTGCTGGGGCAAACCAGCGTGGACCG
CTTGCTGCAACTCTCTCAGGGCCAGGCGGTGAAGGGCAATCAGCTGTT
GCCCGTCTCACTGGTGAAAAGAAAAACCACCCTGGCGCCCAATACGCA
AACCGCCTCTCCCCGCGCGTTGGCCGATTCATTAATGCAGCTGGCACG
ACAGGTTTCCCGACTGGAAAGCGGGCAGTGAGCGCAACGCAATTAATG
TAAGTTAGCTCACTCATTAGGCACCGGGATCTCGACCGATGCCCTTGA
GAGCCTTCAACCCAGTCAGCTCCTTCCGGTGGGCGCGGGGCATGACTA
TCGTCGCCGCACTTATGACTGTCTTCTTTATCATGCAACTCGTAGGAC
AGGTGCCGGCAGCGCTCTGGGTCATTTTCGGCGAGGACCGCTTTCGCT
GGAGCGCGACGATGATCGGCCTGTCGCTTGCGGTATTCGGAATCTTGC
ACGCCCTCGCTCAAGCCTTCGTCACTGGTCCCGCCACCAAACGTTTCG
GCGAGAAGCAGGCCATTATCGCCGGCATGGCGGCCCCACGGGTGCGCA
TGATCGTGCTCCTGTCGTTGAGGACCCGGCTAGGCTGGCGGGGTTGCC
TTACTGGTTAGCAGAATGAATCACCGATACGCGAGCGAACGTGAAGCG
ACTGCTGCTGCAAAACGTCTGCGACCTGAGCAACAACATGAATGGTCT
TCGGTTTCCGTGTTTCGTAAAGTCTGGAAACGCGGAAGTCAGCGCCCT
GCACCATTATGTTCCGGATCTGCATCGCAGGATGCTGCTGGCTACCCT
GTGGAACACCTACATCTGTATTAACGAAGCTTACCCTCACAACGTTCC
AGTAACCGGGCATGTTCATCATCAGTAACCCGTATCGTGAGCATCCTC
TCTCGTTTCATCGGTATCATTACCCCCATGAACAGAAATCCCCCTTAC
ACGGAGGCATCAGTGACCAAACAGGAAAAAACCGCCCTTAACATGGCC
CGCTTTATCAGAAGCCAGACATTAACGCTTCTGGAGAAACTCAACGAG
CTGGACGCGGATGAACAGGCAGACATCTGTGAATCGCTTCACGACCAC
GCTGATGAGCTTTACCGCAGCTGCCTCGCGCGTTTCGGTGATGACGGT
GAAAACCTCTGACACATGCAGCTCCCGGAGACGGTCACAGCTTGTCTG
TAAGCGGATGCCGGGAGCAGACAAGCCCGTCAGGGCGCGTCAGCGGGT
GTTGGGGGGTGTCGGGGCGCAGCCATGACCCAGTCACGTAGCGATAGC
GGAGTGTATACTGGCTTAACTATGCGGCATCAGAGCAGATTGTACTGA
GAGTGCACCATATATGCGGTGTGAAATACCGCACAGATGCGTAAGGAG
AAAATACCGCATCAGGCGCTCTTCCGCTTCCTCGCTCACTGACTCGCT
GCGCTCGGTCGTTCGGCTGCGGCGAGCGGTATCAGCTCACTCAAAGGC
GGTAATACGGTTATCCACAGAATCAGGGGATAACGCAGGAAAGAACAT
GTGAGCAAAAGGCCAGCAAAAGGCCAGGAACCGTAAAAAGGCCGCGTT
GCTGGCGTTTTTCCATAGGCTCCGCCCCCCTGACGAGCATCACAAAAA
TCGACGCTCAAGTCAGAGGTGGCGAAACCCGACAGGACTATAAAGATA
CCAGGCGTTTCCCCCTGGAAGCTCCCTCGTGCGCTCTCCTGTTCCGAC
CCTGCCGCTTACCGGATACCTGTCCGCCTTTCTCCCTTCGGGAAGCGT
GGCGCTTTCTCATAGCTCACGCTGTAGGTATCTCAGTTCGGTGTAGGT
CGTTCGCTCCAAGCTGGGCTGTGTGCACGAACCCCCCGTTCAGCCCGA
CCGCTGCGCCTTATCCGGTAACTATCGTCTTGAGTCCAACCCGGTAAG
ACACGACTTATCGCCACTGGCAGCAGCCACTGGTAACAGGATTAGCAG
AGCGAGGTATGTAGGCGGTGCTACAGAGTTCTTGAAGTGGTGGCCTAA
CTACGGCTACACTAGAAGGACAGTATTTGGTATCTGCGCTCTGCTGAA
GCCAGTTACCTTCGGAAAAAGAGTTGGTAGCTCTTGATCCGGCAAACA
AACCACCGCTGGTAGCGGTGGTTTTTTTGTTTGCAAGCAGCAGATTAC
GCGCAGAAAAAAAGGATCTCAAGAAGATCCTTTGATCTTTTCTACGGG
GTCTGACGCTCAGTGGAACGAAAACTCACGTTAAGGGATTTTGGTCAT
GAACAATAAAACTGTCTGCTTACATAAACAGTAATACAAGGGGTGTTA
TGAGCCATATTCAACGGGAAACGTCTTGCTCTAGGCCGCGATTAAATT
CCAACATGGATGCTGATTTATATGGGTATAAATGGGCTCGCGATAATG
TCGGGCAATCAGGTGCGACAATCTATCGATTGTATGGGAAGCCCGATG
CGCCAGAGTTGTTTCTGAAACATGGCAAAGGTAGCGTTGCCAATGATG
TTACAGATGAGATGGTCAGACTAAACTGGCTGACGGAATTTATGCCTC
TTCCGACCATCAAGCATTTTATCCGTACTCCTGATGATGCATGGTTAC
TCACCACTGCGATCCCCGGGAAAACAGCATTCCAGGTATTAGAAGAAT
ATCCTGATTCAGGTGAAAATATTGTTGATGCGCTGGCAGTGTTCCTGC
GCCGGTTGCATTCGATTCCTGTTTGTAATTGTCCTTTTAACAGCGATC
GCGTATTTCGTCTCGCTCAGGCGCAATCACGAATGAATAACGGTTTGG
TTGATGCGAGTGATTTTGATGACGAGCGTAATGGCTGGCCTGTTGAAC
AAGTCTGGAAAGAAATGCATAAACTTTTGCCATTCTCACCGGATTCAG
TCGTCACTCATGGTGATTTCTCACTTGATAACCTTATTTTTGACGAGG
GGAAATTAATAGGTTGTATTGATGTTGGACGAGTCGGAATCGCAGACC
GATACCAGGATCTTGCCATCCTATGGAACTGCCTCGGTGAGTTTTCTC
CTTCATTACAGAAACGGCTTTTTCAAAAATATGGTATTGATAATCCTG
ATATGAATAAATTGCAGTTTCATTTGATGCTCGATGAGTTTTTCTAAG
AATTAATTCATGAGCGGATACATATTTGAATGTATTTAGAAAAATAAA
CAAATAGGGGTTCCGCGCACATTTCCCCGAAAAGTGCCACCTGAAATT
GTAAACGTTAATATTTTGTTAAAATTCGCGTTAAATTTTTGTTAAATC
AGCTCATTTTTTAACCAATAGGCCGAAATCGGCAAAATCCCTTATAAA
TCAAAAGAATAGACCGAGATAGGGTTGAGTGTTGTTCCAGTTTGGAAC
AAGAGTCCACTATTAAAGAACGTGGACTCCAACGTCAAAGGGCGAAAA
ACCGTCTATCAGGGCGATGGCCCACTACGTGAACCATCACCCTAATCA
AGTTTTTTGGGGTCGAGGTGCCGTAAAGCACTAAATCGGAACCCTAAA
GGGAGCCCCCGATTTAGAGCTTGACGGGGAAAGCCGGCGAACGTGGCG
AGAAAGGAAGGGAAGAAAGCGAAAGGAGCGGGCGCTAGGGCGCTGGCA
AGTGTAGCGGTCACGCTGCGCGTAACCACCACACCCGCCGCGCTTAAT
GCGCCGCTACAGGGCGCGTCCCATTCGCCA
SEQ. ID NO: 95
ATGGCTTCTAGTTCTTCCGATGTCTTCGTTTTGGGTCTAGGTGTTGTT
TTGGCTGCCTTGTATATCTTCAGAGACCAATTATTCGCTGCTTCTAAG
CCAAAGGTGGCTCCAGTTTCCACTACGAAGCCTGCCAACGGTTCCGCT
AACCCAAGAGACTTCATCGCCAAGATGAAACAAGGTAAGAAGAGAATC
GTAATCTTCTACGGTTCTCAAACTGGTACCGCTGAAGAATATGCTATT
CGTTTGGCTAAGGAAGCTAAGCAAAAGTTCGGTCTAGCCTCCTTGGTT
TGTGATCCAGAAGAATACGATTTTGAAAAGTTGGACCAATTGCCAGAA
GATTCTATTGCTTTCTTCGTCGTTGCTACCTATGGTGAAGGTGAACCT
ACAGACAACGCTGTCCAATTGTTGCAAAACTTGCAAGATGAAAGCTTC
GAATTCTCCTCTGGTGAGAGAAAGTTGTCAGGTTTGAAGTACGTTGTT
TTTGGTCTGGGTAACAAGACCTACGAACATTACAACCTCATTGGGAGA
ACTGTTGACGCTCAATTGGCCAAGATGGGTGCTATCAGAATCGGTGAA
AGAGGTGAAGGTGATGATGACAAGTCCATGGAAGAAGACTACTTGGAA
TGGAAGGATGGTATGTGGGAAGCGTTTGCCACTGCTATGGGTGTTGAA
GAAGGTCAAGGTGGTGACTCCGCTGATTTCGTCGTTTCCGAATTGGAA
TCTCACCCACCAGAAAAGGTTTACCAAGGTGAATTTTCTGCTAGAGCT
TTAACCAAAACCAAGGGTATTCACGACGCTAAGAATCCTTTTGCTGCT
CCAATTGCGGTTGCTAGAGAATTGTTCCAATCTGTTGTCGATAGAAAC
TGTGTCCACGTCGAATTCAACATTGAAGGCTCTGGTATCACCTATCAA
CACGGTGACCACGTTGGTTTGTGGCCATTGAATCCAGATGTTGAAGTC
GAACGGTTGTTGTGTGTTTTAGGTTTAGCTGAAAAGAGAGATGCTGTC
ATCTCCATTGAATCCTTAGACCCGGCTTTGGCTAAGGTTCCATTCCCA
GTCCCAACTACTTACGGTGCTGTGTTGAGACACTACATTGACATCTCT
GCTGTCGCCGGTAGACAAATCTTGGGTACTTTGTCCAAATTCGCTCCA
ACCCCAGAAGCTGAAGCTTTCTTGAGAAACTTGAACACTAACAAGGAA
GAATACCACAACGTCGTCGCTAACGGTTGTTTGAAATTGGGTGAAATT
TTGCAAATCGCTACCGGTAACGACATTACTGTCCCACCAACTACTGCC
AACACCACCAAATGGCCAATTCCATTCGACATCATTGTTTCTGCCATC
CCAAGATTGCAACCAAGATACTACTCTATCTCTTCTTCCCCAAAAATT
CATCCAAACACCATCCACGCTACCGTTGTTGTGCTCAAATACGAAAAC
GTTCCAACCGAACCAATCCCAAGAAAGTGGGTTTACGGTGTCGGTAGT
AACTTCTTGTTGAATTTAAAGTACGCTGTTAACAAGGAACCAGTTCCA
TACATCACTCAAAATGGCGAACAAAGAGTCGGTGTCCCGGAATACTTG
ATTGCTGGTCCACGTGGTTCTTACAAGACTGAATCTTTCTACAAGGCT
CCAATCCATGTTAGACGTTCTACTTTCCGTTTGCCAACCAACCCAAAG
TCTCCAGTCATCATGATTGGTCCAGGTACTGGTGTCGCCCCATTCAGA
GGCTTCGTTCAAGAAAGAGTTGCCTTGGCCAGAAGATCCATCGAAAAG
AACGGTCCTGACTCTTTGGCTGACTGGGGTCGTATTTCCTTGTTCTAC
GGTTGTAGAAGATCCGACGAAGACTTCTTGTACAAGGACGAATGGCCA
CAATACGAAGCTGAGTTGAAGGGTAAGTTCAAGTTGCACTGTGCTTTC
TCCAGACAAAACTACAAGCCAGACGGTTCTAAGATTTACGTCCAAGAT
TTGATCTGGGAAGACAGAGAACACATTGCCGATGCCATCTTAAACGGT
AAGGGTTACGTCTACATCTGCGGTGAAGCTAAGTCCATGTCTAAACAA
GTTGAAGAAGTTCTAGCCAAGATCTTGGGCGAAGCCAAAGGTGGTTCC
GGTCCAGTTGAAGGTGTTGCTGAAGTCAAGTTACTGAAGGAACGGTCC
AGATTGATGTTGGATGTCTGGTCTTGA
SEQ. ID NO: 96
MASSSSDVFVLGLGVVLAALYIFRDQLFAASKPKVAPVSTTKPANGSA
NPRDFIAKMKQGKKRIVIFYGSQTGTAEEYAIRLAKEAKQKFGLASLV
CDPEEYDFEKLDQLPEDSIAFFVVATYGEGEPTDNAVQLLQNLQDESF
EFSSGERKLSGLKYVVFGLGNKTYEHYNLIGRTVDAQLAKMGAIRIGE
RGEGDDDKSMEEDYLEWKDGMWEAFATAMGVEEGQGGDSADFVVSELE
SHPPEKVYQGEFSARALTKTKGIHDAKNPFAAPIAVARELFQSVVDRN
CVHVEFNIEGSGITYQHGDHVGLWPLNPDVEVERLLCVLGLAEKRDAV
ISIESLDPALAKVPFPVPTTYGAVLRHYIDISAVAGRQILGTLSKFAP
TPEAEAFLRNLNTNKEEYHNVVANGCLKLGEILQIATGNDITVPPTTA
NTTKWPIPFDIIVSAIPRLQPRYYSISSSPKIHPNTIHATVVVLKYEN
VPTEPIPRKWVYGVGSNFLLNLKYAVNKEPVPYITQNGEQRVGVPEYL
IAGPRGSYKTESFYKAPIHVRRSTFRLPTNPKSPVIMIGPGTGVAPFR
GFVQERVALARRSIEKNGPDSLADWGRISLFYGCRRSDEDFLYKDEWP
QYEAELKGKFKLHCAFSRQNYKPDGSKIYVQDLIWEDREHIADAILNG
KGYVYICGEAKSMSKQVEEVLAKILGEAKGGSGPVEGVAEVKLLKERS
RLMLDVWS
SEQ. ID NO: 97
GAAGAATTGTGAGCGGATAACAATTCCCCTCTAGAAATAATTTTGTTT
AACTTTAAGAAGGAGATATACATATGGCAGATCTCAATTGGATATCGG
CCGGCCACGCGATCGCTGACGTCGGTACCCTCGAGTCTGGTAAAGAAA
CCGCTGCTGCGAAATTTGAACGCCAGCACATGGACTCGTCTACTAGTC
GCAGCTTAATTAACCTAAACTGCTGCCACCGCTGAGCAATAACTAGCA
TAACCCCTTGGGGCCTCTAAACGGGTCTTGAGGGGTTTTTTGCTAGCG
AAAGGAGGAGTCGACTATATCCGGATTGGCGAATGGGACGCGCCCTGT
AGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCT
TCCTTTCTCGCCACGTTCGCCGGCTTTCCCCGTCAAGCTCTAAATCGG
GGGCTCCCTTTAGGGTTCCGATTTAGTGCTTTACGGCACCTCGACCCC
AAAAAACTTGATTAGGGTGATGGTTCACGTAGTGGGCCATCGCCCTGA
TAGACGGTTTTTCGCCCTTTGACGTTGGAGTCCACGTTCTTTAATAGT
GGACTCTTGTTCCAAACTGGAACAACACTCAACCCTATCTCGGTCTAT
TCTTTTGATTTATAAGGGATTTTGCCGATTTCGGCCTATTGGTTAAAA
AATGAGCTGATTTAACAAAAATTTAACGCGAATTTTAACAAAATATTA
ACGTTTACAATTTCTGGCGGCACGATGGCATGAGATTATCAAAAAGGA
TCTTCACCTAGATCCTTTTAAATTAAAAATGAAGTTTTAAATCAATCT
AAAGTATATATGAGTAAACTTGGTCTGACAGTTACCAATGCTTAATCA
GTGAGGCACCTATCTCAGCGATCTGTCTATTTCGTTCATCCATAGTTG
CCTGACTCCCCGTCGTGTAGATAACTACGATACGGGAGGGCTTACCAT
CTGGCCCCAGTGCTGCAATGATACCGCGAGACCCACGCTCACCGGCTC
CAGATTTATCAGCAATAAACCAGCCAGCCGGAAGGGCCGAGCGCAGAA
GTGGTCCTGCAACTTTATCCGCCTCCATCCAGTCTATTAATTGTTGCC
GGGAAGCTAGAGTAAGTAGTTCGCCAGTTAATAGTTTGCGCAACGTTG
TTGCCATTGCTACAGGCATCGTGGTGTCACGCTCGTCGTTTGGTATGG
CTTCATTCAGCTCCGGTTCCCAACGATCAAGGCGAGTTACATGATCCC
CCATGTTGTGCAAAAAAGCGGTTAGCTCCTTCGGTCCTCCGATCGTTG
TCAGAAGTAAGTTGGCCGCAGTGTTATCACTCATGGTTATGGCAGCAC
TGCATAATTCTCTTACTGTCATGCCATCCGTAAGATGCTTTTCTGTGA
CTGGTGAGTACTCAACCAAGTCATTCTGAGAATAGTGTATGCGGCGAC
CGAGTTGCTCTTGCCCGGCGTCAATACGGGATAATACCGCGCCACATA
GCAGAACTTTAAAAGTGCTCATCATTGGAAAACGTTCTTCGGGGCGAA
AACTCTCAAGGATCTTACCGCTGTTGAGATCCAGTTCGATGTAACCCA
CTCGTGCACCCAACTGATCTTCAGCATCTTTTACTTTCACCAGCGTTT
CTGGGTGAGCAAAAACAGGAAGGCAAAATGCCGCAAAAAAGGGAATAA
GGGCGACACGGAAATGTTGAATACTCATACTCTTCCTTTTTCAATCAT
GATTGAAGCATTTATCAGGGTTATTGTCTCATGAGCGGATACATATTT
GAATGTATTTAGAAAAATAAACAAATAGGTCATGACCAAAATCCCTTA
ACGTGAGTTTTCGTTCCACTGAGCGTCAGACCCCGTAGAAAAGATCAA
AGGATCTTCTTGAGATCCTTTTTTTCTGCGCGTAATCTGCTGCTTGCA
AACAAAAAAACCACCGCTACCAGCGGTGGTTTGTTTGCCGGATCAAGA
GCTACCAACTCTTTTTCCGAAGGTAACTGGCTTCAGCAGAGCGCAGAT
ACCAAATACTGTCCTTCTAGTGTAGCCGTAGTTAGGCCACCACTTCAA
GAACTCTGTAGCACCGCCTACATACCTCGCTCTGCTAATCCTGTTACC
AGTGGCTGCTGCCAGTGGCGATAAGTCGTGTCTTACCGGGTTGGACTC
AAGACGATAGTTACCGGATAAGGCGCAGCGGTCGGGCTGAACGGGGGG
TTCGTGCACACAGCCCAGCTTGGAGCGAACGACCTACACCGAACTGAG
ATACCTACAGCGTGAGCTATGAGAAAGCGCCACGCTTCCCGAAGGGAG
AAAGGCGGACAGGTATCCGGTAAGCGGCAGGGTCGGAACAGGAGAGCG
CACGAGGGAGCTTCCAGGGGGAAACGCCTGGTATCTTTATAGTCCTGT
CGGGTTTCGCCACCTCTGACTTGAGCGTCGATTTTTGTGATGCTCGTC
AGGGGGGCGGAGCCTATGGAAAAACGCCAGCAACGCGGCCTTTTTACG
GTTCCTGGCCTTTTGCTGGCCTTTTGCTCACATGTTCTTTCCTGCGTT
ATCCCCTGATTCTGTGGATAACCGTATTACCGCCTTTGAGTGAGCTGA
TACCGCTCGCCGCAGCCGAACGACCGAGCGCAGCGAGTCAGTGAGCGA
GGAAGCGGAAGAGCGCCTGATGCGGTATTTTCTCCTTACGCATCTGTG
CGGTATTTCACACCGCATATATGGTGCACTCTCAGTACAATCTGCTCT
GATGCCGCATAGTTAAGCCAGTATACACTCCGCTATCGCTACGTGACT
GGGTCATGGCTGCGCCCCGACACCCGCCAACACCCGCTGACGCGCCCT
GACGGGCTTGTCTGCTCCCGGCATCCGCTTACAGACAAGCTGTGACCG
TCTCCGGGAGCTGCATGTGTCAGAGGTTTTCACCGTCATCACCGAAAC
GCGCGAGGCAGCTGCGGTAAAGCTCATCAGCGTGGTCGTGAAGCGATT
CACAGATGTCTGCCTGTTCATCCGCGTCCAGCTCGTTGAGTTTCTCCA
GAAGCGTTAATGTCTGGCTTCTGATAAAGCGGGCCATGTTAAGGGCGG
TTTTTTCCTGTTTGGTCACTGATGCCTCCGTGTAAGGGGGATTTCTGT
TCATGGGGGTAATGATACCGATGAAACGAGAGAGGATGCTCACGATAC
GGGTTACTGATGATGAACATGCCCGGTTACTGGAACGTTGTGAGGGTA
AACAACTGGCGGTATGGATGCGGCGGGACCAGAGAAAAATCACTCAGG
GTCAATGCCAGCGCTTCGTTAATACAGATGTAGGTGTTCCACAGGGTA
GCCAGCAGCATCCTGCGATGCAGATCCGGAACATAATGGTGCAGGGCG
CTGACTTCCGCGTTTCCAGACTTTACGAAACACGGAAACCGAAGACCA
TTCATGTTGTTGCTCAGGTCGCAGACGTTTTGCAGCAGCAGTCGCTTC
ACGTTCGCTCGCGTATCGGTGATTCATTCTGCTAACCAGTAAGGCAAC
CCCGCCAGCCTAGCCGGGTCCTCAACGACAGGAGCACGATCATGCTAG
TCATGCCCCGCGCCCACCGGAAGGAGCTGACTGGGTTGAAGGCTCTCA
AGGGCATCGGTCGAGATCCCGGTGCCTAATGAGTGAGCTAACTTACAT
TAATTGCGTTGCGCTCACTGCCCGCTTTCCAGTCGGGAAACCTGTCGT
GCCAGCTGCATTAATGAATCGGCCAACGCGCGGGGAGAGGCGGTTTGC
GTATTGGGCGCCAGGGTGGTTTTTCTTTTCACCAGTGAGACGGGCAAC
AGCTGATTGCCCTTCACCGCCTGGCCCTGAGAGAGTTGCAGCAAGCGG
TCCACGCTGGTTTGCCCCAGCAGGCGAAAATCCTGTTTGATGGTGGTT
AACGGCGGGATATAACATGAGCTGTCTTCGGTATCGTCGTATCCCACT
ACCGAGATGTCCGCACCAACGCGCAGCCCGGACTCGGTAATGGCGCGC
ATTGCGCCCAGCGCCATCTGATCGTTGGCAACCAGCATCGCAGTGGGA
ACGATGCCCTCATTCAGCATTTGCATGGTTTGTTGAAAACCGGACATG
GCACTCCAGTCGCCTTCCCGTTCCGCTATCGGCTGAATTTGATTGCGA
GTGAGATATTTATGCCAGCCAGCCAGACGCAGACGCGCCGAGACAGAA
CTTAATGGGCCCGCTAACAGCGCGATTTGCTGGTGACCCAATGCGACC
AGATGCTCCACGCCCAGTCGCGTACCGTCTTCATGGGAGAAAATAATA
CTGTTGATGGGTGTCTGGTCAGAGACATCAAGAAATAACGCCGGAACA
TTAGTGCAGGCAGCTTCCACAGCAATGGCATCCTGGTCATCCAGCGGA
TAGTTAATGATCAGCCCACTGACGCGTTGCGCGAGAAGATTGTGCACC
GCCGCTTTACAGGCTTCGACGCCGCTTCGTTCTACCATCGACACCACC
ACGCTGGCACCCAGTTGATCGGCGCGAGATTTAATCGCCGCGACAATT
TGCGACGGCGCGTGCAGGGCCAGACTGGAGGTGGCAACGCCAATCAGC
AACGACTGTTTGCCCGCCAGTTGTTGTGCCACGCGGTTGGGAATGTAA
TTCAGCTCCGCCATCGCCGCTTCCACTTTTTCCCGCGTTTTCGCAGAA
ACGTGGCTGGCCTGGTTCACCACGCGGGAAACGGTCTGATAAGAGACA
CCGGCATACTCTGCGACATCGTATAACGTTACTGGTTTCACATTCACC
ACCCTGAATTGACTCTCTTCCGGGCGCTATCATGCCATACCGCGAAAG
GTTTTGCGCCATTCGATGGTGTCCGGGATCTCGACGCTCTCCCTTATG
CGACTCCTGCATTAGGAAGCAGCCCAGTAGTAGGTTGAGGCCGTTGAG
CACCGCCGCCGCAAGGAATGGTGCATGCAAGGAGATGGCGCCCAACAG
TCCCCCGGCCACGGGGCCTGCCACCATACCCACGCCGAAACAAGCGCT
CATGAGCCCGAAGTGGCGAGCCCGATCTTCCCCATCGGTGATGTCGGC
GATATAGGCGCCAGCAACCGCACCTGTGGCGCCGGTGATGCCGGCCAC
GATGCGTCCGGCGTAGCCTAGGATCGAGATCGATCTCGATCCCGCGAA
ATTAATACGACTCACTACG
SEQ. ID NO: 98
CGCTAACAGCGCGATTTGCTGGTGACCCAATGCGACCAGATGCTCCAC
GCCCAGTCGCGTACCGTCTTCATGGGAGAAAATAATACTGTTGATGGG
TGTCTGGTCAGAGACATCAAGAAATAACGCCGGAACATTAGTGCAGGC
AGCTTCCACAGCAATGGCATCCTGGTCATCCAGCGGATAGTTAATGAT
CAGCCCACTGACGCGTTGCGCGAGAAGATTGTGCACCGCCGCTTTACA
GGCTTCGACGCCGCTTCGTTCTACCATCGACACCACCACGCTGGCACC
CAGTTGATCGGCGCGAGATTTAATCGCCGCGACAATTTGCGACGGCGC
GTGCAGGGCCAGACTGGAGGTGGCAACGCCAATCAGCAACGACTGTTT
GCCCGCCAGTTGTTGTGCCACGCGGTTGGGAATGTAATTCAGCTCCGC
CATCGCCGCTTCCACTTTTTCCCGCGTTTTCGCAGAAACGTGGCTGGC
CTGGTTCACCACGCGGGAAACGGTCTGATAAGAGACACCGGCATACTC
TGCGACATCGTATAACGTTACTGGTTTCACATTCACCACCCTGAATTG
ACTCTCTTCCGGGCGCTATCATGCCATACCGCGAAAGGTTTTGCGCCA
TTCGATGGTGTCCGGGATCTCGACGCTCTCCCTTATGCGACTCCTGCA
TTAGGAAGCAGCCCAGTAGTAGGTTGAGGCCGTTGAGCACCGCCGCCG
CAAGGAATGGTGCATGCAAGGAGATGGCGCCCAACAGTCCCCCGGCCA
CGGGGCCTGCCACCATACCCACGCCGAAACAAGCGCTCATGAGCCCGA
AGTGGCGAGCCCGATCTTCCCCATCGGTGATGTCGGCGATATAGGCGC
CAGCAACCGCACCTGTGGCGCCGGTGATGCCGGCCACGATGCGTCCGG
CGTAGCCTAGGATCGAGATCGATCTCGATCCCGCGAAATTAATACGAC
TCACTATAGGGGAATTGTGAGCGGATAACAATTCCCCTCTAGAAATAA
TTTTGTTTAACTTTAAGAAGGAGATATACATATGGCAGATCTCAATTG
GATATCGGCCGGCCACGCGATCGCTGACGTCGGTACCCTCGAGTCTGG
TAAAGAAACCGCTGCTGCGAAATTTGAACGCCAGCACATGGACTCGTC
TACTAGTCGCAGCTTAATTAACCTAAACTGCTGCCACCGCTGAGCAAT
AACTAGCATAACCCCTTGGGGCCTCTAAACGGGTCTTGAGGGGTTTTT
TGCTAGCGAAAGGAGGAGTCGACAAGCTGACGACCGGGTCTCCGCAAG
TGGCACTTTTCGGGGAAATGTGCGCGGAACCCCTATTTGTTTATTTTT
CTAAATACATTCAAATATGTATCCGCTCATGAATTAATTCTTAGAAAA
ACTCATCGAGCATCAAATGAAACTGCAATTTATTCATATCAGGATTAT
CAATACCATATTTTTGAAAAAGCCGTTTCTGTAATGAAGGAGAAAACT
CACCGAGGCAGTTCCATAGGATGGCAAGATCCTGGTATCGGTCTGCGA
TTCCGACTCGTCCAACATCAATACAACCTATTAATTTCCCCTCGTCAA
AAATAAGGTTATCAAGTGAGAAATCACCATGAGTGACGACTGAATCCG
GTGAGAATGGCAAAAGTTTATGCATTTCTTTCCAGACTTGTTCAACAG
GCCAGCCATTACGCTCGTCATCAAAATCACTCGCATCAACCAAACCGT
TATTCATTCGTGATTGCGCCTGAGCGAGACGAAATACGCGGTCGCTGT
TAAAAGGACAATTACAAACAGGAATCGAATGCAACCGGCGCAGGAACA
CTGCCAGCGCATCAACAATATTTTCACCTGAATCAGGATATTCTTCTA
ATACCTGGAATGCTGTTTTCCCGGGGATCGCAGTGGTGAGTAACCATG
CATCATCAGGAGTACGGATAAAATGCTTGATGGTCGGAAGAGGCATAA
ATTCCGTCAGCCAGTTTAGTCTGACCATCTCATCTGTAACATCATTGG
CAACGCTACCTTTGCCATGTTTCAGAAACAACTCTGGCGCATCGGGCT
TCCCATACAATCGATAGATTGTCGCACCTGATTGCCCGACATTATCGC
GAGCCCATTTATACCCATATAAATCAGCATCCATGTTGGAATTTAATC
GCGGCCTAGAGCAAGACGTTTCCCGTTGAATATGGCTCATACTCTTCC
TTTTTCAATATTATTGAAGCATTTATCAGGGTTATTGTCTCATGAGCG
GATACATATTTGAATGTATTTAGAAAAATAAACAAATAGGCATGCAGC
GCTCTTCCGCTTCCTCGCTCACTGACTCGCTACGCTCGGTCGTTCGAC
TGCGGCGAGCGGTGTCAGCTCACTCAAAAGCGGTAATACGGTTATCCA
CAGAATCAGGGGATAAAGCCGGAAAGAACATGTGAGCAAAAAGCAAAG
CACCGGAAGAAGCCAACGCCGCAGGCGTTTTTCCATAGGCTCCGCCCC
CCTGACGAGCATCACAAAAATCGACGCTCAAGCCAGAGGTGGCGAAAC
CCGACAGGACTATAAAGATACCAGGCGTTTCCCCCTGGAAGCTCCCTC
GTGCGCTCTCCTGTTCCGACCCTGCCGCTTACCGGATACCTGTCCGCC
TTTCTCCCTTCGGGAAGCGTGGCGCTTTCTCATAGCTCACGCTGTTGG
TATCTCAGTTCGGTGTAGGTCGTTCGCTCCAAGCTGGGCTGTGTGCAC
GAACCCCCCGTTCAGCCCGACCGCTGCGCCTTATCCGGTAACTATCGT
CTTGAGTCCAACCCGGTAAGACACGACTTATCGCCACTGGCAGCAGCC
ATTGGTAACTGATTTAGAGGACTTTGTCTTGAAGTTATGCACCTGTTA
AGGCTAAACTGAAAGAACAGATTTTGGTGAGTGCGGTCCTCCAACCCA
CTTACCTTGGTTCAAAGAGTTGGTAGCTCAGCGAACCTTGAGAAAACC
ACCGTTGGTAGCGGTGGTTTTTCTTTATTTATGAGATGATGAATCAAT
CGGTCTATCAAGTCAACGAACAGCTATTCCGTTACTCTAGATTTCAGT
GCAATTTATCTCTTCAAATGTAGCACCTGAAGTCAGCCCCATACGATA
TAAGTTGTAATTCTCATGTTAGTCATGCCCCGCGCCCACCGGAAGGAG
CTGACTGGGTTGAAGGCTCTCAAGGGCATCGGTCGAGATCCCGGTGCC
TAATGAGTGAGCTAACTTACATTAATTGCGTTGCGCTCACTGCCCGCT
TTCCAGTCGGGAAACCTGTCGTGCCAGCTGCATTAATGAATCGGCCAA
CGCGCGGGGAGAGGCGGTTTGCGTATTGGGCGCCAGGGTGGTTTTTCT
TTTCACCAGTGAGACGGGCAACAGCTGATTGCCCTTCACCGCCTGGCC
CTGAGAGAGTTGCAGCAAGCGGTCCACGCTGGTTTGCCCCAGCAGGCG
AAAATCCTGTTTGATGGTGGTTAACGGCGGGATATAACATGAGCTGTC
TTCGGTATCGTCGTATCCCACTACCGAGATGTCCGCACCAACGCGCAG
CCCGGACTCGGTAATGGCGCGCATTGCGCCCAGCGCCATCTGATCGTT
GGCAACCAGCATCGCAGTGGGAACGATGCCCTCATTCAGCATTTGCAT
GGTTTGTTGAAAACCGGACATGGCACTCCAGTCGCCTTCCCGTTCCGC
TATCGGCTGAATTTGATTGCGAGTGAGATATTTATGCCAGCCAGCCAG
ACGCAGACGCGCCGAGACAGAACTTAATGGGCC

Hereinafter are provided examples of specific implementations for performing the methods of the present disclosure, as well as implementations representing the compositions of the present disclosure. The examples are provided for illustrative purposes only, and are not intended to limit the scope of the present disclosure in any way.

### EXAMPLES

### Example 1—Process for Making a First Halogenated Psilocybin Derivative from Halogenated Tryptophan Feedstock

Yeast (Saccharomyces cerevisiae) was genetically engineered to enable bioconversion of commercially obtained, halogenated, simple indole, tryptophan, or tryptamine feedstock to generate final product. The parent yeast (Saccharomyces cerevisiae) strain was CEN.PK with genotype Matα; ura3-52; trp1-289; leu2-3,112; his3Δ1; MAL2-8C; SUC2. The parent strain was engineered to include three enzymatic steps through chromosomal homologous recombination of integration cassettes as described previously (Dastmalchi et al., 2019, Nat. Chem Biol. 15: 384-390; Chen et al., 2018, Nat. Chem Biol. 14: 738-743). The three steps were comprised of PsiH (SEQ.ID NO: 4), PsiK (SEQ.ID NO: 6) and PsiM (SEQ.ID NO: 8), encoded by SEQ.ID NO: 3, SEQ.ID NO: 5 and SEQ.ID NO: 7, respectively, with addition of in-frame, C-terminal HA (SEQ.ID NO: 81, SEQ.ID NO: 82), V5 (SEQ.ID NO: 87, SEQ.ID NO: 88), and FLAG (SEQ.ID NO: 85, SEQ.ID NO: 86) epitope tags, respectively. DNA (SEQ.ID NO: 95) encoding a fifth enzyme, PcCPR (SEQ.ID NO: 96) was also integrated following PCR-based, C-terminal, in-frame addition of epitope tag c-MYC (SEQ.ID NO: 83, SEQ.ID NO: 84) to support functionality of PsiH. Integration cassettes were built using yeast promoter sequences amplified from S. cerevisiae genomic DNA as described (Dastmalchi et al. 2019; Chen et al. 2018) enabling constitutive gene expression. Amplified promoters included PGK1 (SEQ.ID NO: 77), TDH3 (SEQ.ID NO: 78), CLN1 (SEQ.ID NO: 79), and UGA1 (SEQ.ID NO: 80). Two integration cassettes were assembled: the first (SEQ.ID NO: 74) harbored tagged PsiH and PcCPR, and the second (SEQ.ID NO: 75) harbored tagged PsiK and PsiM. Successive genomic integration of these cassettes was performed as described previously (Chen et al. 2018). Following stable integration of these two cassettes, the strain was further manipulated by transformation with a yeast episomal vector encoding a promiscuous decarboxylase, ClostSporTDC (pMM1-pTDH3-ClostSporTDC-His-tCYC1) (SEQ.ID NO: 76) enabling efficient decarboxylation of halogenated tryptophan intermediates and/or feedstocks. For this Example, heterologous expression of a non-native or engineered TrpB gene was not necessary, as endogenous tryptophan synthase activity proved sufficient. The final engineered strain was called Sc-1. For scaled-up production of halogenated product, culturing was performed as follows. Seed cultures were inoculated in SD-drop-out medium overnight. The overnight culture was then divided into two flasks containing 500 ml each of SD-drop-out medium containing 2% (w/v) glucose, 0.3% (w/v) KH₂PO₄, 0.05% (w/v) MgSO₄·7H₂O, 0.5% (w/v) (NH₄)₂SO₄ plus 500 μM 6-fluorotryptophan (ThermoFisher Scientific) for conversion by Sc-1. Yeast cultures were grown for 48 h. Cultures were then centrifuged (10,000 g×5 minutes) to remove cellular content, and culture broth containing secreted halogenated product was stored at −80° C. until further processing. To determine successful biosynthesis of 6-fluoro-psilocybin (compound with formula (XI)), culture broth was analyzed using high-resolution LC-HESI-LTQ-Orbitrap-XL MS (Thermo Fisher Scientific), employing a modified version of a method described previously (Chang et al., 2015, Plant Physiol. 169: 1127-1140), with the exception that liquid chromatography was carried out using an UltiMate 3000 HPLC (Thermo Fisher Scientific) equipped with a Poroshell 120 SB-C18 column (Agilent Technologies) instead of an Accela HPLC system (Thermo Fisher Scientific) equipped with a Zorbax C18 column (Agilent Technologies). Briefly, 10 microliters of sample was injected at a flow rate of 0.5 mL/min and a gradient of solvent A (water with 0.1% of formic Acid) and solvent B (ACN with 0.1% formic Acid) as follows: 100% to 0% (v/v) solvent A over 5 min; isocratic at 0% (v/v) for 1 min; 0% to 100% (v/v) over 0.1 min; and isocratic at 100% (v/v) for 1.9 min. Total run time was 8 minutes. Heated ESI source and interface conditions were operated in positive ion mode as follows: vaporizer temperature, 400° C.; source voltage, 3 kV; sheath gas, 60 au, auxiliary gas, 20 au; capillary temperature, 380° C.; capillary voltage, 6 V; tube lens, 45 V. Instrumentation was performed as a single, HR scan event using Orbitrap detection of m/z in the range of 100-500 m/z. Ion injection time was 300 ms with scan time of 1 s. External and internal calibration procedures ensured <2 ppm error to facilitate elemental formulae predictions. Singly protonated product with exact m/z and expected elemental formula matching 6-fluoro-psilocybin having chemical formula (XI):

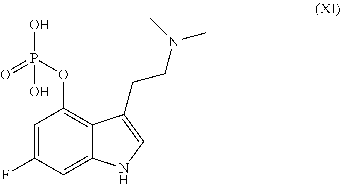 [SMILES: CN(C)CCc1c[nH]c2cc(F)cc(OP(=O)(O)O)c12]

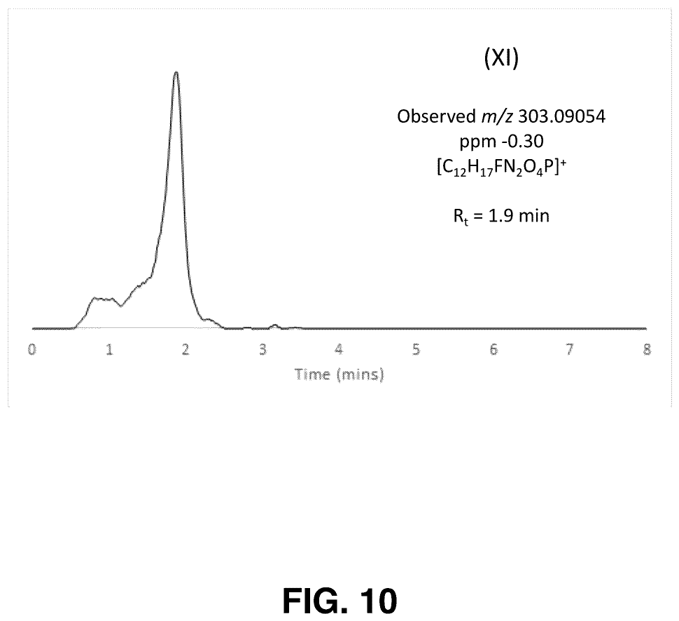

eluted at 1.9 minutes (EIC, see: FIG. 10).

### Example 2—Process for Making a Second Halogenated Psilocybin Derivative from Halogenated Tryptophan Feedstock

The same yeast strain (Sc-1) and procedures described in Example 1 were used to biosynthesize a halogenated psilocybin derivative with chemical formula (XIV), with the following exception: in place of 6-fluorotryptophan, 500 μM 7-bromotryptophan (Combi-Blocks, www.combi-blocks.com) was supplied as feedstock for bioconversion. Singly protonated product with exact m/z and expected elemental formula matching 7-bromo-psilocybin having chemical formula (XIV):

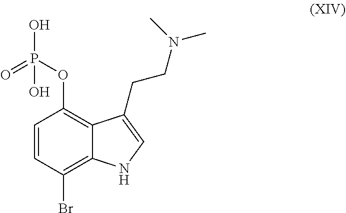 [SMILES: CN(C)CCc1c[nH]c2c(Br)ccc(OP(=O)(O)O)c12]

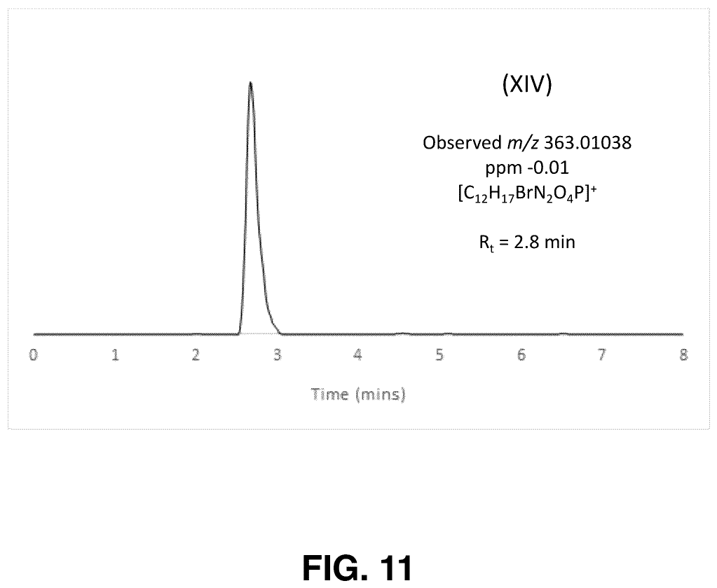

eluted at 2.8 minutes (EIC, see: FIG. 11).

### Example 3—Process for Making a Third Halogenated Psilocybin Derivative from Halogenated Indole Feedstock

The same yeast strain (Sc-1) and procedures described in Example 1 were used to biosynthesize a halogenated psilocybin derivative with chemical formula (XVI), with the following exception: in place of 6-fluorotryptophan, 500 μM 7-fluoroindole (Combi-Blocks, www.combi-blocks.com) was supplied as feedstock for bioconversion. Singly protonated product with exact m/z and expected elemental formula matching 7-fluoro-psilocybin compound having chemical formula (XVI):

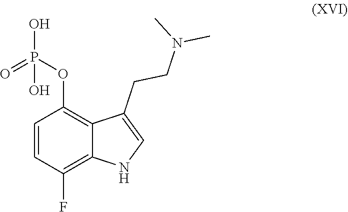 [SMILES: CN(C)CCc1c[nH]c2c(F)ccc(OP(=O)(O)O)c12]

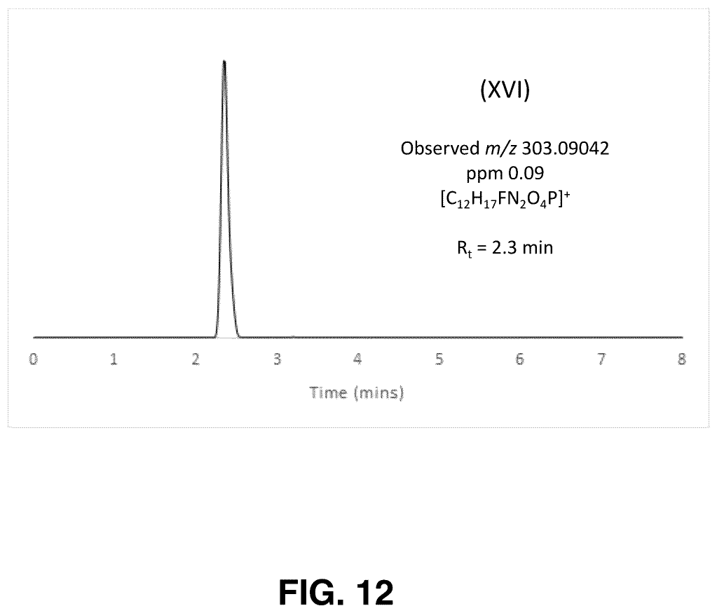

eluted at 2.3 minutes (EIC, see: FIG. 12).

### Example 4—Process for Making a Fourth Halogenated Psilocybin Derivative from Halogenated Indole Feedstock

The same yeast strain (Sc-1) and procedures described in Example 1 were used to biosynthesize a halogenated psilocybin derivative with formula (XVII), with the following exception: in place of 6-fluorotryptophan, 500 μM 7-chloroindole (Combi-Blocks, www.combi-blocks.com) was supplied as feedstock for bioconversion. Singly protonated product with exact m/z and expected elemental formula matching 7-chloro-psilocybin having formula (XVII):

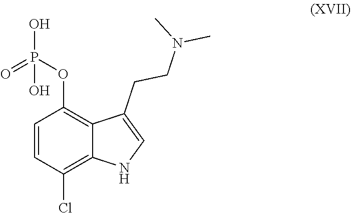 [SMILES: CN(C)CCc1c[nH]c2c(Cl)ccc(OP(=O)(O)O)c12]

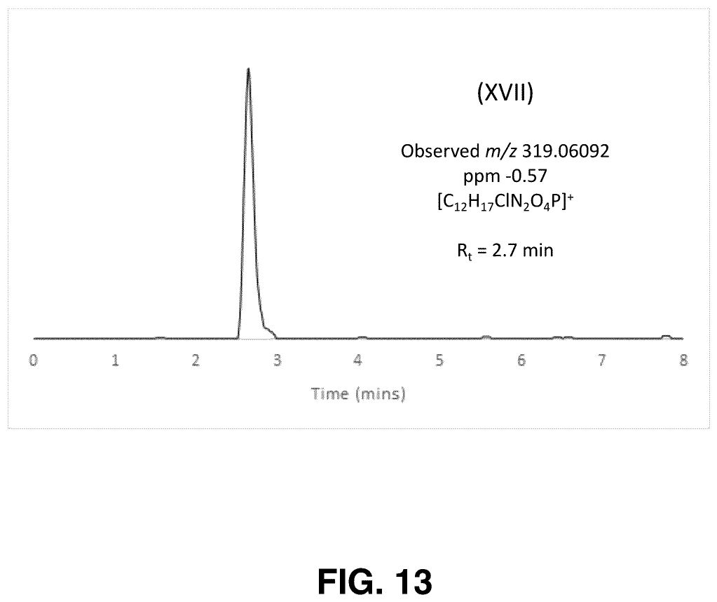

eluted at 2.7 minutes (EIC, see: FIG. 13).

### Example 5—Process for Making a Fifth Halogenated Psilocybin Derivative from Halogenated Indole Feedstock

E. coli strain Ec-1 was constructed as follows. For plasmid cloning, Top10 or XL1-blue strains were used depending on antibiotic markers. Standard LB media was used for culturing. For gene expression and feeding experiments, the parent host strain employed was BL21 (DE3). For this Example, heterologous expression of a non-native or engineered TrpB gene was not necessary, as endogenous tryptophan synthase activity proved sufficient. An in frame, C-terminally His-tagged (SEQ.ID NO: 89, SEQ.ID NO: 90) BaTDC (SEQ.ID NO: 36, SEQ.ID NO: 37) was generated by PCR, followed by cloning into the NdeI and XhoI sites of the plasmid pCDM4 (SEQ.ID NO: 92) where the expression of the gene was driven by a T7 promoter. In a second plasmid, an in-frame, C-terminally His-tagged (SEQ.ID NO: 89, SEQ.ID NO: 90) PsmF (SEQ.ID NO: 62, SEQ.ID NO: 63) was cloned into the NdeI and XhoI sites of pET28a(+) (SEQ.ID NO: 94) where the expression of the gene was also driven by a T7 promoter. The E. coli host strain BL21 (DE3) was transformed with both plasmids and selected for using streptomycin and kanamycin antibiotics. This strain (Ec-1, F-ompT hsdSB (rB-, mB-) gal dcm (DE3), pCDM4-BaTDC-His, pET28a(+)-PsmF-His) enabled production of N-acetylated products from modified indole feedstock. Further, the use of promiscuous BaTDC (SEQ.ID NO: 36, SEQ.ID NO: 37) allowed decarboxylation of a wide variety of halogenated tryptophan intermediates. Scaled-up culturing of engineered E. coli was conducted as follows: seed cultures were inoculated in AMM (Jones et al., 2015, Sci. Rep. 5: 11301) medium overnight. The overnight culture was then divided into two flasks containing 500 mL each of AMM medium additionally containing 0.5% (w/v) serine, 1M IPTG, 50 ug/L kanamycin and streptomycin, and 100 mg/L 7-fluoroindole (Combi-Blocks, www.combi-blocks.com) for conversion by the E. coli strain. Cultures were grown for 24 h. Cultures were then centrifuged (10,000 g×5 minutes) to remove cellular content, and culture broth containing secreted derivative was stored at −80 C until further processing. The N-(2-(7-difluoro-1H-indol-3-yl)ethyl)acetamide product having chemical formula (XV):

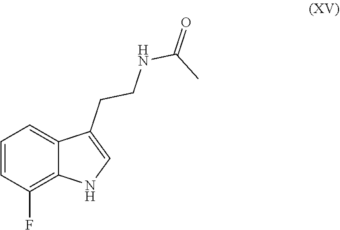 [SMILES: CC(=O)NCCc1c[nH]c2c(F)cccc12]

contained in 1 L of E. coli culture was extracted by ethyl acetate (3×300 ml). The organic layer was dried over Na₂SO₄, followed by concentration under reduced pressure. The residue was purified by column chromatography on silica gel using ethyl acetate-hexane (50→80%) as eluent to give the compound as a light yellow solid (22 mg). Following purification, high-resolution MS (HRMS), ¹H NMR, and selective ¹³C NMR were performed to assess purity, estimate total quantity, and confirm molecular structure. 1H NMR (400 MHz, CDCl₃): δ=1.96 (s, 3H), 2.97 (t, J=6.8 Hz, 2H,), 3.59 (dt, J=6.7, 6.1 Hz, 2H,), 5.7 (br, s, 1H), 6.91 (dd, J=11.2, 7.8 Hz, 1H), 7.03 (m, 2H), 7.35 (d, J=7.9 Hz, 1H), 8.6 (br, s, 1H). ¹³C NMR (100 MHz, CDCl₃): δ=23.3, 25.3, 39.8, 106.9 (d, J_{C, F}=16.1 Hz), 113.7, 114.4 (d, J_{C, F}=3.4 Hz), 119.6 (d, J_{C, F}=6.1 Hz), 122.8, 124.7, 131.1 (d, J_{C, F}=5.3 Hz), 149.7 (d, J_{C, F}=244.2 Hz), 170.3. HRMS (ESI) m/z: calcd. for C₁₂H₁₃FN₂O [M+H]⁺ 221.1090, found 221.1087. The compound having chemical formula (XV) was 95% (w/w) pure. It is noted that the acetyl group of the compound having chemical formula (XV) is included therein by virtue of the acetyl transferase having SEQ.ID NO: 63 in Escherichia coli strain Ec-1.

Cell lines for pharmacology assays. CHO-K1/Galpha15 (GenScript, M00257) (−5-HT_{2A}) and CHO-K1/5-HT_{2A} (GenScript, M00250) (+5-HT_{2A}) cells lines were used in both toxicology/growth inhibition (MTT) and calcium release assays. Briefly, CHO-K1/Galpha15 is a control cell line that constitutively expresses Galpha15 which is a promiscuous Gq protein. It is engineered as a host cell, allowing transfected receptor(s) to signal through the Gq signal transduction pathway and mobilize intracellular calcium from the endoplasmic reticulum (ER). These control cells lack any transgene encoding 5-HT_{2A} receptors, thus preventing calcium mobilization in response to 5-HT_{2A} activation. Conversely, CHO-K1/5-HT_{2A} cells stably express 5-HT_{2A} receptor in the CHO-K1 host background. This design enables Gq-11 expressed in CHO-K1 cells to mobilize intracellular calcium changes when 5-HT_{2A} receptors are activated by ligands.

Cell lines were maintained in Ham's F12 media plus 10% FBS in the presence of 100 ug/ml hygromycin for CHO-K1/Ga15 or 400 ug/ml G418 for CHO-K1/5-HT_{2A} unless indicated otherwise for specific assays. Cell maintenance was carried out as recommended by the cell supplier. Briefly, vials with cells were removed from the liquid nitrogen and thawed quickly in 37° C. water bath. Just before cells were completely thawed, vial exteriors were decontaminated with 70% ethanol spray. Cell suspension was then retrieved from the vial and added to warm (37° C.), ‘complete’ (non-dropout) growth media, and centrifuged at 1,000 rpm for 5 minutes. The supernatant was discarded and the cell pellet was then resuspended in another 10 ml of complete growth media, and added to a 10 cm cell culture dish (Greiner Bio-One #664160). The media was changed every third day until the cells reached ˜90% confluence. The ˜90% confluent cells were then split 10:1, and used either for maintenance or pharmacological study.

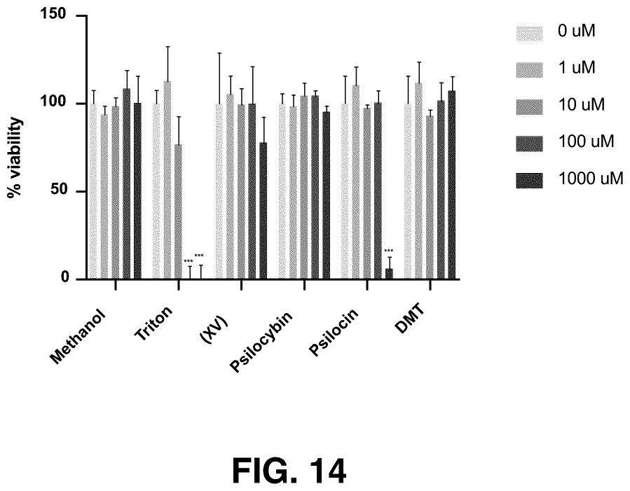

Assessment of cell viability upon treatment of glycosylated psilocybin derivatives. To establish suitable ligand concentrations for the calcium release assays, MTT (3-(4,5-dimethylthiazol-2-yl)-2,5-diphenyltetrazolium) assays were first performed. Results of these assays were conducted using both control ligands (e.g. psilocybin, psilocin, DMT) and novel derivatives, in part as a pre-screen for any remarkable toxic effects on cell cultures up to concentrations of 1 mM. A known cellular toxin (Triton X-100, Pyrgiotakis G. et al., 2009, Ann. Biomed. Eng. 37: 1464-1473) was included as a general marker of toxicity. Modified Chinese Hamster Ovary cells (CHO-K1/Ga15) were cultured using standard procedures using the manufacture's protocols (Genscript, M00257). Briefly, cells were cultured in Ham's F12 medium supplemented with 10% fetal bovine serum and 100 mg/ml Hygromycin B, and grown at 37° C. in the presence of 5% CO₂. To test the various compounds with the cell line, cells were seeded in a clear 96-well culture plate at 10,000 cells per well. After allowing cells to attach and grow for 24 hours, assay compounds were added at 1 μM, 10 μM, 100 μM, and 1 mM final concentrations. Methanol concentrations used are 0.001, 0.01, 0.1, and 1%. Triton concentrations used are 0.0001, 0.001, 0.01 and 0.1%. Cells were incubated with compounds for 48 hours before accessing cell viability with the MTT assay following the manufacture's protocol (MTT Cell Growth Assay Kit; Millipore Sigma, CTO2). MTT reagent was added to cells and allowed to incubate for 4 hours before solubilization with isopropanol plus 0.04 N HCl. Absorbance readings were performed at 570 nm with the reference at 630 nm on a SpectraMax iD3 plate reader. Non-treated cells were assigned 100% viability. Results of the cell viability assays are shown in FIG. 14. Bar graphs show the mean+/−SD (n=3). Significance (P<0.0001), as indicated by (***) was determined using 2-way ANOVA with Dunnett's multiple comparisons test. The results using compound with formula (XV) are indicated as “(XV)” on the x-axis.

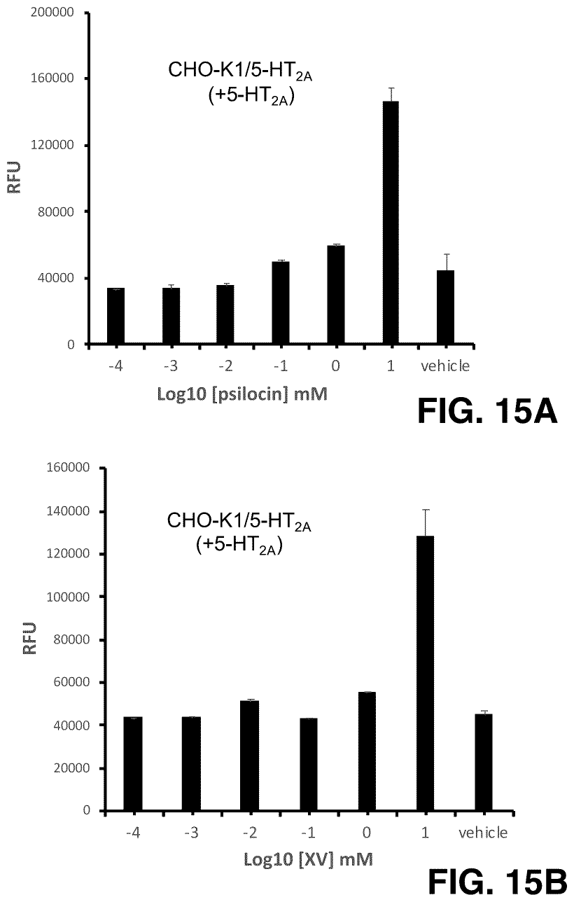

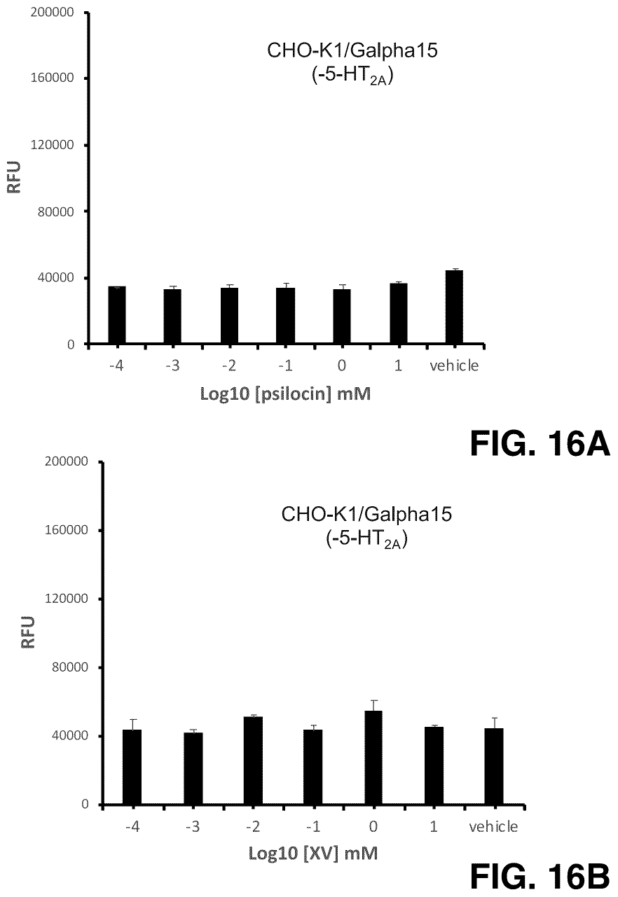

Increase in cytosolic calcium concentration by 5-HT_{2A} activation. Changes in intracellular calcium concentration due to the treatment with assay compounds was measured using Fluo-8 dye (Abcam, #ab112129) according to the manufacturer's instructions. Briefly, CHO-K1 cells stably expressing 5-HT_{2A} (Genscript #M00250) (+5-HT_{2A}) or lacking 5-HT_{2A} (Genscript, M00257) (−5-HT_{2A}) were seeded on black walled clear bottom 96-well plates (Thermo Scientific #NUNC165305), allowing 70,000 cells/well in 100 ul media (HAM's F12, GIBCO #11765-047) with 1% FBS (Thermo Scientific #12483020). Cultures were maintained in a humidified incubator at 37° C. and 5% CO₂. Fluo-8 dye was loaded into the cultures for 30 min at 37° C., followed by 30 min additional incubation at room temperature. Next, different dilutions of novel molecules and controls were prepared in serum-free culture media and added to the cells. Fluorescence (ex 490 nm/em 525 nm) obtained after the addition of molecules was expressed relative to values obtained before addition of the molecules (relative Fluo-8 fluorescence=Fmax/F0, where Fmax=maximum fluorescence and F0=baseline fluorescence). Fluorescence intensities were measured using a Spectramax ID3 plate reader (www.moleculardevices.com). Relative fluorescence (RFU) at increasing concentrations of compound was determined, illustrating concentration-dependent calcium flux. Psilocin, a known agonist with binding activity at 5-HT_{2A} (Rickli A. et al., 2016, Europ. Neuropsychopharmacol., 26: 1326-1337) was used as a positive control to establish assay functionality. The Example compound (XV) was then evaluated. Results are shown in FIGS. 15A-15B and 16A-16B. The results using different concentrations of psilocin and compound with formula (XV) (indicated as “(XV)”) in +5-HT_{2A} cell cultures are shown in FIG. 15A and FIG. 15B, respectively. The results using different concentrations of psilocin and compound with formula (XV) (indicated as “(XV)”) in −5-HT_{2A} cell cultures are shown in FIG. 16A and FIG. 16B, respectively. Results for negative control treatment with vehicle containing neither psilocin nor derivative are shown in FIGS. 15A-15B and 16A-16B. Error bars show+/−SD about the mean (n=3).

### Example 6—Process for Making a Sixth Halogenated Psilocybin Derivative from Halogenated Indole Feedstock

Escherichia coli strain Ec-1 was used to biosynthesize halogenated tryptamine derivative having chemical formula (II) from halogenated indole feedstock. The construction of Ec-1 is described in Example 5. Scaled-up culturing and material storage of engineered E. coli was conducted as described in Example 5, except that 4-chloroindole (Combi-Blocks, www.combi-blocks.com) was used in place of 7-fluoroindole. To assess product, high-resolution LC-HESI-LTQ-Orbitrap-XL MS analysis was conducted as described in Example 1. Singly protonated product with exact m/z and expected elemental formula matching 4-chloro-N-acetyltryptamine having chemical formula (II):

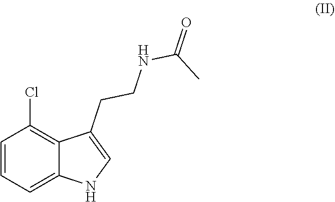 [SMILES: CC(=O)NCCc1c[nH]c2cccc(Cl)c12]

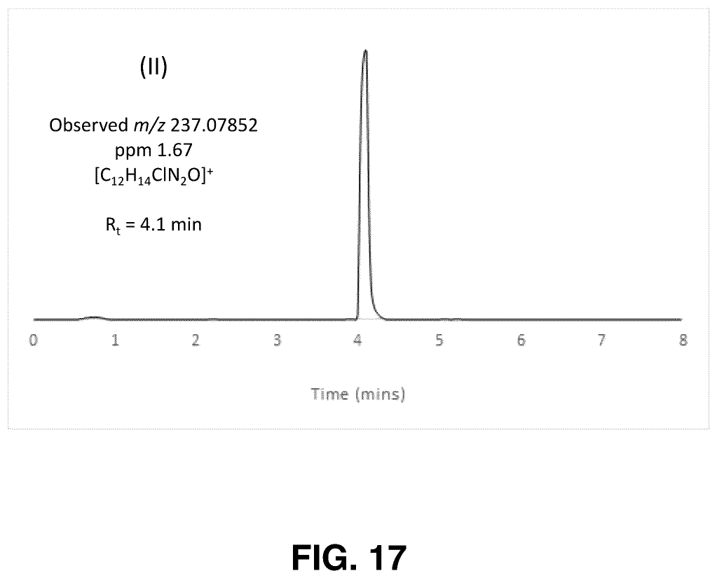

Eluted at 4.1 minutes (EIC, see: FIG. 17). It is noted that the acetyl group of the compound having chemical formula (II) is included therein by virtue of the acetyl transferase having SEQ.ID NO: 63 in Escherichia coli strain Ec-1.

### Example 7—Process for Making a Seventh Halogenated Psilocybin Derivative from Halogenated Indole Feedstock

Escherichia coli strain Ec-1 was used to biosynthesize halogenated tryptamine derivative having chemical formula (IX) from halogenated indole feedstock. The construction of Ec-1 is described in Example 5. Scaled-up culturing and material storage of engineered E. coli was conducted as described in Example 5, except that 5,6-difluoroindole (Combi-Blocks, www.combi-blocks.com) was used in place of 7-fluoroindole. N-(2-(5,6-difluoro-1H-indol-3-yl)ethyl)acetamide product having Chemical formula (IX):

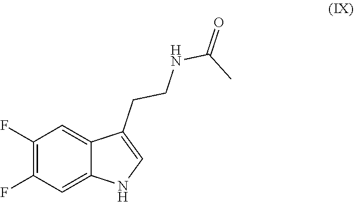 [SMILES: CC(=O)NCCc1c[nH]c2cc(F)c(F)cc12]

contained in 0.55 L of E. coli culture was extracted by ethyl acetate (3×300 ml). The organic layer was dried over Na₂SO₄, followed by concentration under reduced pressure. The residue was purified by column chromatography on silica gel using ethyl acetate-hexane (50→80%) as eluent to give the compound as a light yellow solid (11 mg). Following purification, high-resolution MS (HRMS), ¹H NMR, and selective ¹³C NMR were performed to assess purity, estimate total quantity, and confirm molecular structure. ¹H NMR (400 MHz, CDCl₃): δ=1.70-1.79 (m, 1H), 1.99-2.06 (m, 1H), 2.29 (s, 6H), 2.33-2.38 (m, 1H), 2.40-2.48 (m, 1H), 3.22 (t, 1H, J=8.0 Hz), 3.28-3.36 (m, 1H), 3.68 (t, 1H, J=8.4 Hz), 3.94 (br, 1H), 6.65 (d, 1H, J=8.4 Hz), 6.74 (t, 1H, J=7.6 Hz), 7.05 (t, 1H, J=7.6 Hz), 7.11 (d, 1H, J=7.6 Hz). ¹³C NMR (100 MHz, CDCl₃): δ=23.4, 25.2, 39.7, 99.1 (d, J_{C, F}=21.7 Hz), 105.4 ((d, J_{C, F}=20.0 Hz), 123.3 (d, J_{C, F}=3.7 Hz), 150.6 (dd, J_{C, F}=237.4, 17.2 Hz), 148.9 (dd, J_{C, F}=238.3, 18.1 Hz), 170.1. HRMS (ESI) calcd. for C₁₂H₁₂F₂N₂O 239.0996 [M+H]⁺, found 239.0989. The compound having chemical formula (IX) was 95% (w/w) pure. It is noted that the acetyl group of the compound having chemical formula (IX) is included therein by virtue of the acetyl transferase having SEQ.ID NO: 63 in Escherichia coli strain Ec-1.

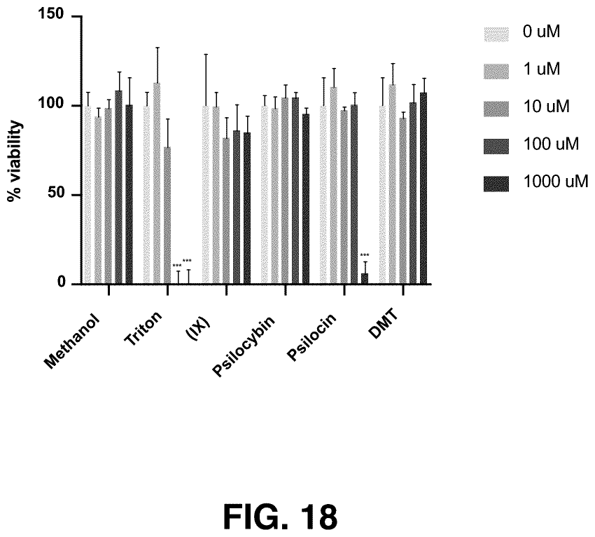

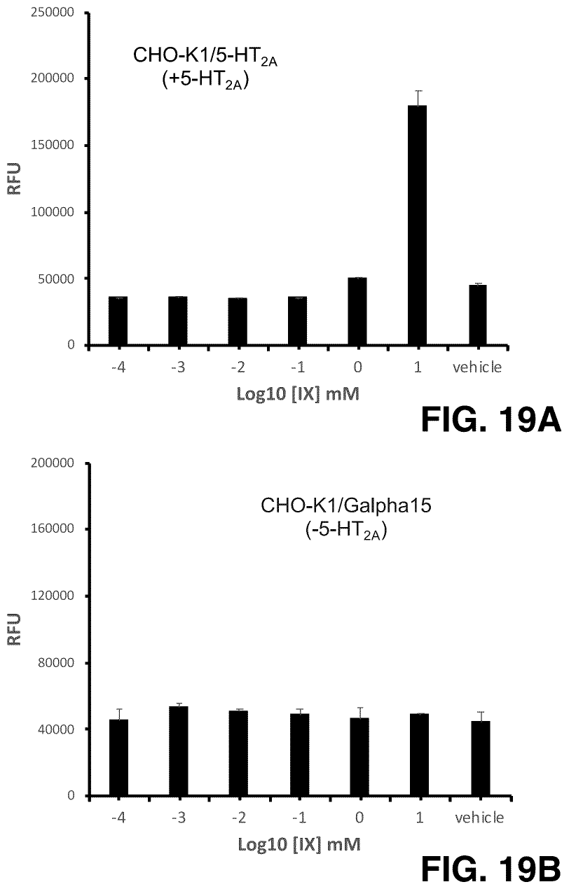

Efficacy testing was carried out as described in Example 5. MTT assay results are shown in FIG. 18, and calcium mobility assay results are shown in FIG. 19A (+5-HT_{2A} cells) and FIG. 19B (−5-HT_{2A} cells). In both cases, the example compound having the chemical formula (IX) is shown as “(IX)”.

### Example 8—Process for Making an Eighth Halogenated Psilocybin Derivative from Halogenated Indole Feedstock

Escherichia coli strain Ec-1 was used to biosynthesize halogenated tryptamine derivative having chemical formula (VI) from halogenated indole feedstock. The construction of Ec-1 is described in Example 5. Scaled-up culturing and material storage of engineered E. coli was conducted as described in Example 5, except that 4,6-difluoroindole (Combi-Blocks, www.combi-blocks.com) was used in place of 7-fluoroindole. To assess product, high-resolution LC-HESI-LTQ-Orbitrap-XL MS analysis was conducted as described in Example 1. Singly protonated product with exact m/z and expected elemental formula matching 4,6-difluoro-N-acetyltryptamine having chemical formula (VI):

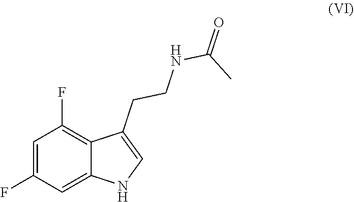 [SMILES: CC(=O)NCCc1c[nH]c2cc(F)cc(F)c12]

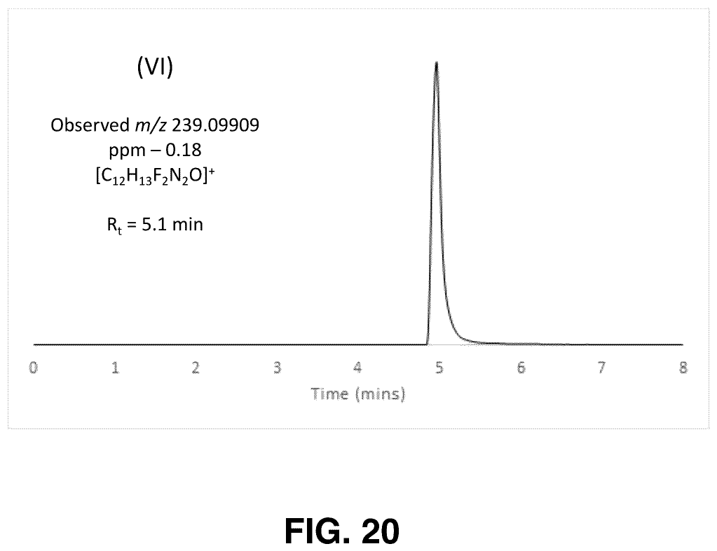

eluted at 5.1 minutes (EIC, see: FIG. 20).

### Example 9—Process for Making a Ninth Halogenated Psilocybin Derivative from Halogenated Indole Feedstock

Escherichia coli strain Ec-1 was used to biosynthesize halogenated tryptamine derivative having chemical formula (V) from halogenated indole feedstock. The construction of Ec-1 is described in Example 5. Scaled-up culturing and material storage of engineered E. coli was conducted as described in Example 5, except that 4,5-difluoroindole (Combi-Blocks, www.combi-blocks.com) was used in place of 7-fluoroindole. N-(2-(4,5-difluoro-1H-indol-3-yl)ethyl)acetamide product having chemical formula (V):

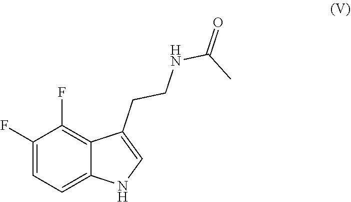 [SMILES: CC(=O)NCCc1c[nH]c2ccc(F)c(F)c12]

contained in 1 L of E. coli culture was extracted by ethyl acetate (3×600 ml). The organic layer was dried over Na₂SO₄, followed by concentration under reduced pressure. The residue was purified by flash chromatography on silica gel using ethyl acetate-hexane (50→80%) as eluent, followed by crystallization from EtOAc/Hexane to give the compound as a white solid (20 mg). Following purification, high-resolution MS (HRMS), ¹H NMR, and selective ¹³C NMR were performed to assess purity, estimate total quantity, and confirm molecular structure. ¹H NMR (400 MHz, CDCl₃): δ=1.96 (s, 3H), 3.04 (t, J=6.9 Hz, 2H,), 3.60 (dt, J=6.6, 6.4 Hz, 2H,), 5.64 (br, s, 1H), 7.01 (m, 3H), 8.41 (br, s, 1H). ¹³C NMR (100 MHz, CDCl₃): δ=23.4, 26.3, 40.5 (d, J_{C, F}=2.0 Hz), 106.6 (dd, J_{C, F}=7.7, 4.2 Hz), 111.7 (d, J_{C, F}=21.7 Hz), 124.0, 143.9 (dd, J_{C, F}=246.4, 14.5 Hz), 143.8 (dd, J_{C, F}=246.4, 14.5 Hz), 170.2. HRMS (ESI) m/z: calcd. for C₁₂H₁₂F₂N₂O [M+H]⁺ 239.0996, found 239.0989. The compound having chemical formula (V) was 95% (w/w) pure. It is noted that the acetyl group of the compound having chemical formula (II) is included therein by virtue of the acetyl transferase having SEQ.ID NO: 63 in Escherichia coli strain Ec-1.

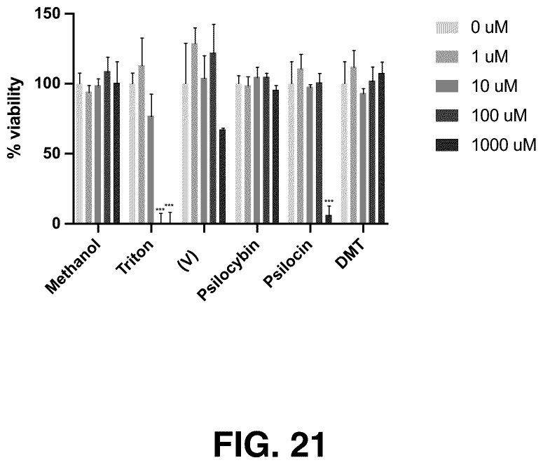

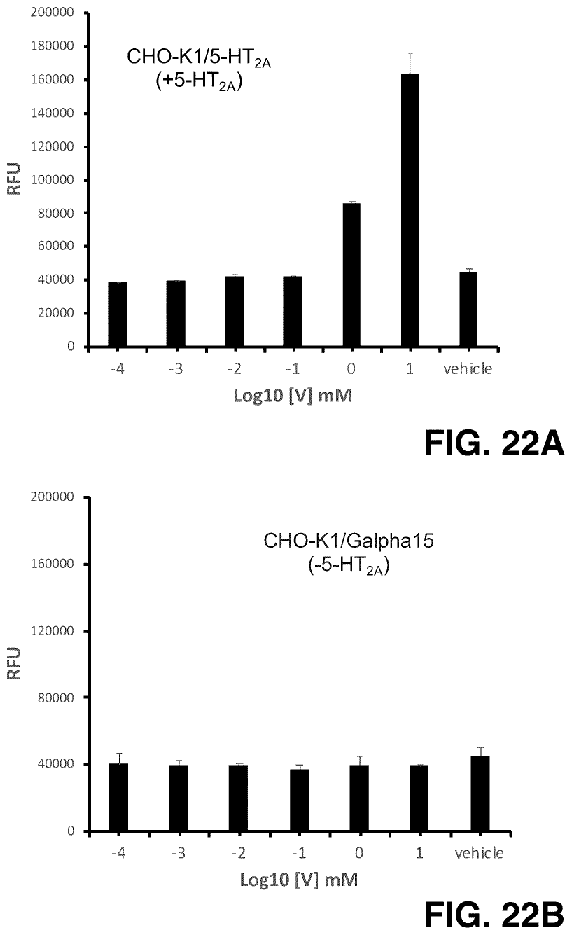

Efficacy testing was carried out as described in Example 5. MTT assay results are shown in FIG. 21, and calcium mobility assay results are shown in FIGS. 22A and 22B. In both cases, the example compound having the chemical formula (V) is shown as “(V)”.

### Example 10—Process for Making a Tenth Halogenated Psilocybin Derivative from Halogenated Indole Feedstock

E. coli strain Ec-2 was constructed as follows. For plasmid cloning, Top10 or XL1-blue strains were used depending on antibiotic markers. Standard LB media was used for culturing. For gene expression and feeding experiments, the parent host strain employed was BL21 (DE3). For this Example, heterologous expression of a non-native or engineered TrpB gene was not necessary, as endogenous tryptophan synthase activity proved sufficient. From plasmid pCDM4 (SEQ.ID NO: 92), the plasmid pCDM4-PsiD-FLAG was created by inserting an in-frame, C-terminally FLAG-tagged (SEQ.ID NO: 85) PsiD gene (SEQ.ID NO: 1) into the NdeI/XhoI site of pCDM4. The plasm id pETM6-H10-PsiK-V5-PsiM-FLAG was created by first cloning the in-frame, C-terminally V5-tagged (SEQ.ID NO: 87) PsiK (SEQ.ID NO: 5) into the NdeI/XhoI site of pETM6-H10 (SEQ.ID NO: 97) to create pETM6-H10-PsiK-V5. This intermediate plasm id was digested with SpeI and SalI, and in-frame, C-terminally FLAG tagged (SEQ.ID NO: 85) PsiM (SEQ.ID NO: 7) was cloned into the site with XbaI and SalI, nullifying the SpeI restriction site. In this setup, the T7 polymerase was able to drive the expression of the polycistronic DNA containing both PsiK and PsiM. The two target plasmids pCDM4-PsiD-FLAG and pETM6-H10-PsiK-V5-PsiM-FLAG were transformed into BL21 (DE3) cells, and antibiotics ampicillin plus streptomycin were used to select for the correct clones containing both plasmids. Scaled-up culturing of engineered E. coli was conducted as follows: seed cultures were inoculated in AMM (Jones et al. 2015, Sci Rep. 5: 11301) medium overnight. The overnight culture was then divided into two flasks containing 500 mL each of AMM medium additionally containing 0.5% (w/v) serine, 1M IPTG, 50 ug/L ampicillin and streptomyces, and 100 mg/L 4-hydroxy-6-bromoindole (BLDPharm, www.bldpharm.com) for conversion by Ec-2. Cultures were grown for 24 h. Cultures were then centrifuged (10,000 g×5 minutes) to remove cellular content, and culture broth containing secreted derivative was stored at −80 C until further processing. To assess product, high-resolution LC-HESI-LTQ-Orbitrap-XL MS analysis was conducted as described in Example 1. Singly protonated product with exact m/z and expected elemental formula matching 6-bromo-psilocybin having chemical formula (XIII):

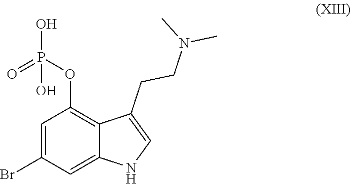 [SMILES: CN(C)CCc1c[nH]c2cc(Br)cc(OP(=O)(O)O)c12]

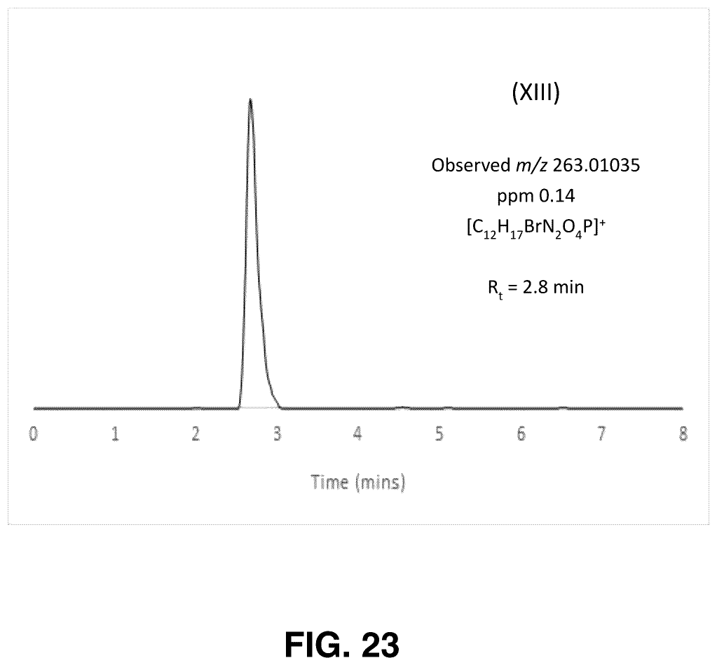

eluted at 2.8 minutes (IC, see: FIG. 23).

### Example 11—Process for Making an Eleventh Halogenated Psilocybin Derivative from Halogenated Indole Feedstock

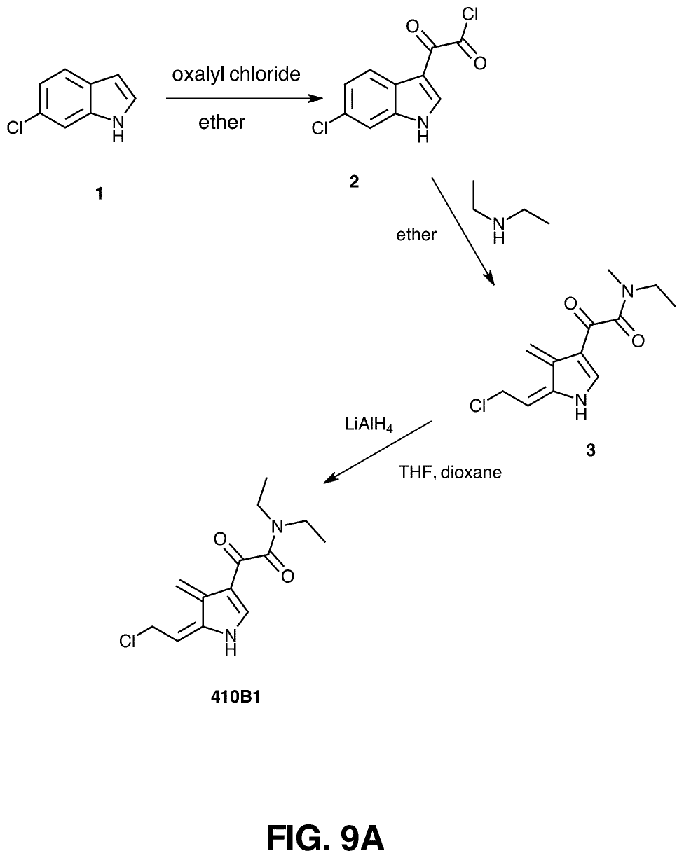

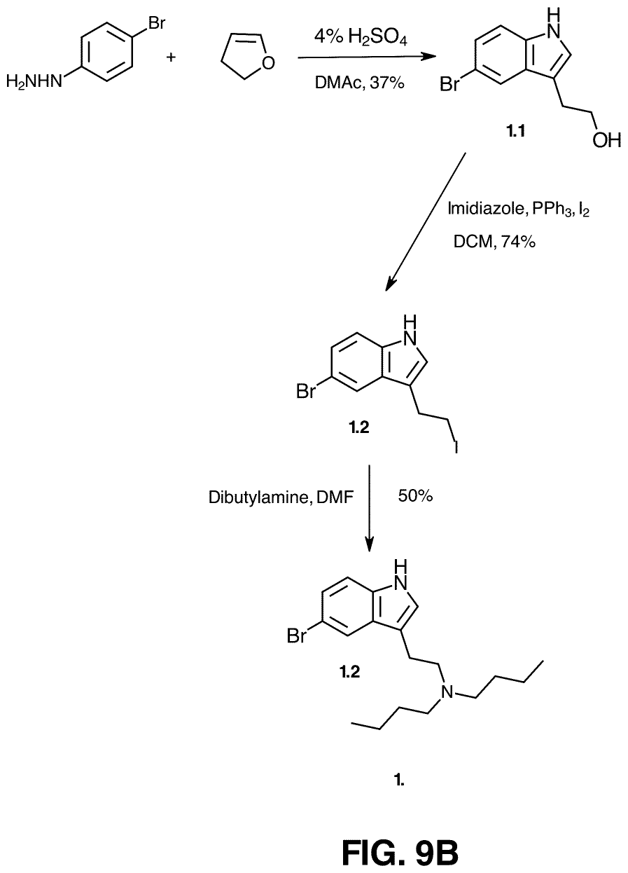

In a flame-dried round bottom flask under argon, 6-chloroindole (compound 1, see: FIG. 9A) (210 mg, 1.39 mmol, 1.00 eq) was dissolved in diethyl ether (4.0 mL) and cooled to 0° C. To this solution, oxalyl chloride (369 mg, 2.91 mmol, 2.10 eq) in diethyl ether (2.0 mL) was added dropwise over a period of 10 minutes. The reaction mixture was allowed to stir at room temperature for 4 hours. Additional ether (5.0 mL) was added to the reaction mixture which was then cooled to 0° C. and subsequently vacuum-filtered to yield a bright yellow solid (compound 2) (211 mg, 0.872 mmol, 63%). Compound 2 (see: FIG. 9A) was washed with cold ether (10 mL) and allowed to briefly dry before being resuspended in diethyl ether (3.0 mL) and cooled to 0° C. Diethylamine (510 mg, 6.97 mmol, 8.00 eq) was diluted with diethyl ether (3.0 mL) and added dropwise to the reaction flask, immediately precipitating out a white solid. This mixture was allowed to stir for 10 minutes at 0° C., warmed to room temperature, and stirred for an additional 1.5 hours. The visible precipitate was vacuum filtered to yield a solid which was washed with cold diethyl ether (10 mL). This solid was suspended in deionized water (50 mL), stirred for 10 minutes, vacuum-filtered, and allowed to dry overnight, yielding compound 3 (see: FIG. 9A) as a white powder (124 mg, 0.445 mmol, 51%). No further purification was required. ¹H NMR (400 MHz, CDCl₃): δ (ppm)=9.56 (s, 1H), 8.24 (d, J=8.5 Hz, 1H), 7.74 (d, J=3.0 Hz, 1H), 7.36 (dd, J=1.8, 0.6 Hz, 1H), 7.28 (d, J=1.8 Hz, 1H), 3.54 (q, J=7.1 Hz, 2H), 3.36 (q, J=7.1 Hz, 2H), 1.27 (t, J=7.2 Hz, 3H), 1.19 (t, J=7.1 Hz, 3H). A THF solution of lithium aluminum hydride (2.20 mL, 2.22 mmol, 5.00 eq, 1.0 M) was added to a 3-neck, flame-dried, round-bottom flask under argon. Anhydrous dioxane (3.0 mL) was added to the reaction mixture. In a separate vial, compound 3 (124 mg, 0.445 mmol) was dissolved in a THF (2.0 mL)/dioxane (1.0 mL) solution and added dropwise to the reaction vessel. This mixture was then allowed to reflux at 95° C. for 1.5 hours. The reaction mixture was cooled to 0° C. and quenched through dropwise addition of a water (1.0 mL)/THF (2.0 mL) solution, yielding a flocculent precipitate. This precipitate was removed through vacuum-filtration and the filter-cake was washed with diethyl ether (20 mL). The filtrate was collected, dried with MgSO₄, and solvent removed to yield a pale-yellow oil. Purification through column chromatography on silica gel (2% MeOH/DCM+1% NEt₃) yielded compound 410B01 (see: FIG. 9A), corresponding to compound with formula (X):

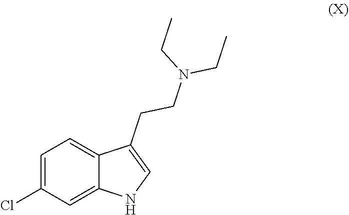 [SMILES: CCN(CC)CCc1c[nH]c2cc(Cl)ccc12]

as a clear, colourless oil (34 mg, 0.136 mmol, 30%). ¹H NMR (400 MHz, CDCl₃): δ (ppm)=8.37 (s, 1H), 7.49 (d, J=8.5z, 1H), 7.30 (d, J=1.8 Hz, 1H), 7.07 (dd, J=8.5, 1.8 Hz, 1H), 6.98 (dd, J=2.5, 1.1 Hz, 1H), 2.90 (high order m, 2H), 2.79 (high order m, 2H), 2.74-2.62 (q, J=7.1 Hz, 4H), 1.15-1.04 (t, J=7.1 Hz, 6H). 25 ¹³C NMR (100 MHz, CDCl₃): δ (ppm)=136.6, 127.8, 126.2, 122.2, 119.9, 119.7, 114.8, 111.0, 70.5, 53.5, 46.9, 22.8, 11.7. The compound having chemical formula (IX) was 95% (w/w) pure.

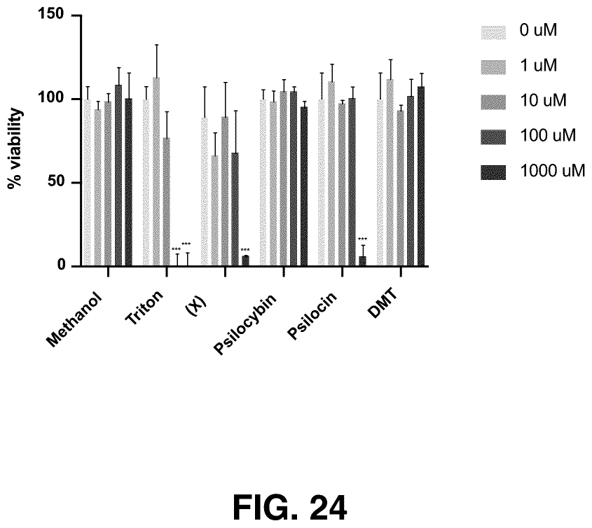

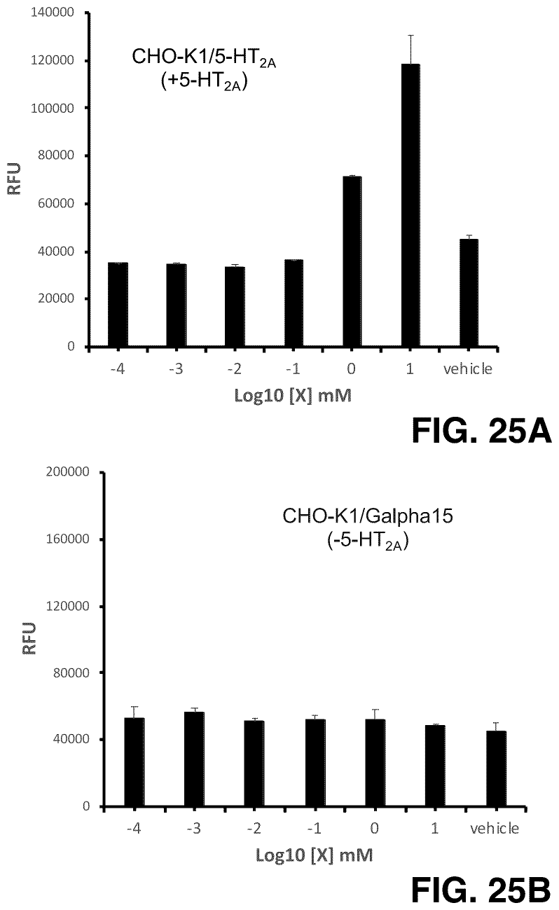

Efficacy testing was carried out as described in Example 5. MTT assay results are shown in FIG. 24, and calcium mobility assay results are shown in FIGS. 25A and 25B. In both cases, the example compound having chemical formula (X) is shown as “(X)”.

### Example 12—Process for Making a Twelfth Halogenated Psilocybin Derivative from Halogenated Indole Feedstock

A similar process as illustrated in FIG. 9A (Example 11) was followed. In a flame-dried round bottom flask under argon, 4-fluoroindole (compound 1) (200 mg, 1.48 mmol, 1.00 eq) was dissolved in diethyl ether (4.0 mL) and cooled to 0° C. To this solution, oxalyl chloride (394 mg, 3.11 mmol, 2.10 eq) in diethyl ether (2.0 mL) was added dropwise over a period of 10 minutes. The reaction mixture was allowed to stir at room temperature for 4 hours. Additional ether (5.0 mL) was added to the reaction mixture which was then cooled to 0° C. and subsequently vacuum-filtered to yield a bright yellow solid (compound 2) (264 mg, 1.17 mmol, 79%). Compound 2 was washed with cold ether (10 mL) and allowed to briefly dry before being resuspended in diethyl ether (3.0 mL) and cooled to 0° C. N-Methylethylamine (574 mg, 9.72 mmol, 8.00 eq) was diluted with diethyl ether (3.0 mL) and added dropwise to the reaction flask. This mixture was allowed to stir for 10 minutes at 0° C., warmed to room temperature, and stirred for an additional 1.5 hours, yielding a suspension of light-pink gooey solid. The diethyl ether was decanted, and the solid dissolved in dichloromethane (10 mL) and washed with water (3×10 mL). The organic phase was dried with MgSO₄ and solvent removed to yield a pale-pink glassy solid (128 mg, 0.516 mmol, 43%). ¹H NMR (400 MHz, CDCl₃): δ (ppm)=10.72 (s, 1H), 7.72 (d, J=3.8 Hz, 1H), 7.10-7.00 (m, 2H), 6.84 (ddd, J=11.1, 7.2, 1.6 Hz, 1H), 3.55 (q, J=7.2 Hz, 1H), 3.35 (q, J=7.1 Hz, 1H), 3.05 (s, 2H), 2.98 (s, 1H), 1.23 (t, J=7.2 Hz, 1H), 1.15 (t, J=7.1 Hz, 2H). A THF solution of lithium aluminum hydride (2.58 mL, 2.58 mmol, 5.00 eq, 1.0 M) was added to a 3-neck, flame-dried, round-bottom flask under argon. Anhydrous dioxane (3.0 mL) was added to the reaction mixture. In a separate vial, compound 3 (128 mg, 0.516 mmol) was dissolved in an anhydrous THF (2.0 mL)/dioxane (1.0 mL) solution and added dropwise to the reaction vessel. This mixture was then allowed to reflux at 95° C. for 2.5 hours. The reaction mixture was cooled to 0° C. and quenched through dropwise addition of a water (1 mL)/THF (2 mL) solution, yielding a flocculent precipitate. This precipitate was removed through vacuum-filtration and the filter-cake was washed with diethyl ether (20 mL). The filtrate was collected, dried with MgSO₄, and solvent removed to yield a pale-orange oil. Purification through column chromatography on silica gel (1% MeOH/CH₂Cl₂+1% NEt₃) yielded compound 456B01, corresponding to compound having chemical formula (IV):

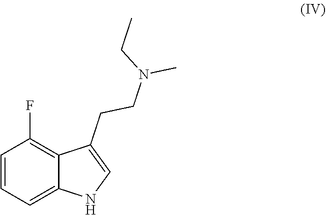 [SMILES: CCN(C)CCc1c[nH]c2cccc(F)c12]

as clear, colourless oily crystal (86 mg, 0.390 mmol, 76%). ¹H NMR (400 MHz, CDCl₃): δ (ppm)=8.68 (s, 1H), 7.12-6.99 (m, 2H), 6.91 (d, J=2.3 Hz, 1H), 6.73 (ddd, J=11.2, 6.9, 1.6 Hz, 1H), 3.04 (high order m, 2H), 2.75 (high order m, 2H), 2.57 (q, J=7.2 Hz, 2H), 2.38 (s, 3H), 1.12 (t, J=7.2 Hz, 3H). ¹³C NMR (100 MHz, CDCl₃): δ (ppm)=157.2 (d, J_{C-F}=245.6 Hz), 139.2 (d, J_{C-F}=11.8 Hz), 122.2 (d, J_{C-F}=8.2 Hz), 121.9, 107.2 (d, J_{C-F}=3.6 Hz), 104.2 (d, J_{C-F}=19.7 Hz), 58.5, 51.2, 41.5, 24.1, 12.1. The compound having chemical formula (IV) was 95% (w/w) pure.

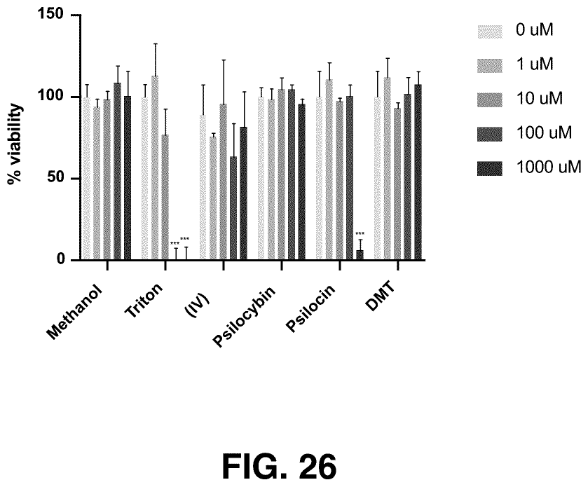

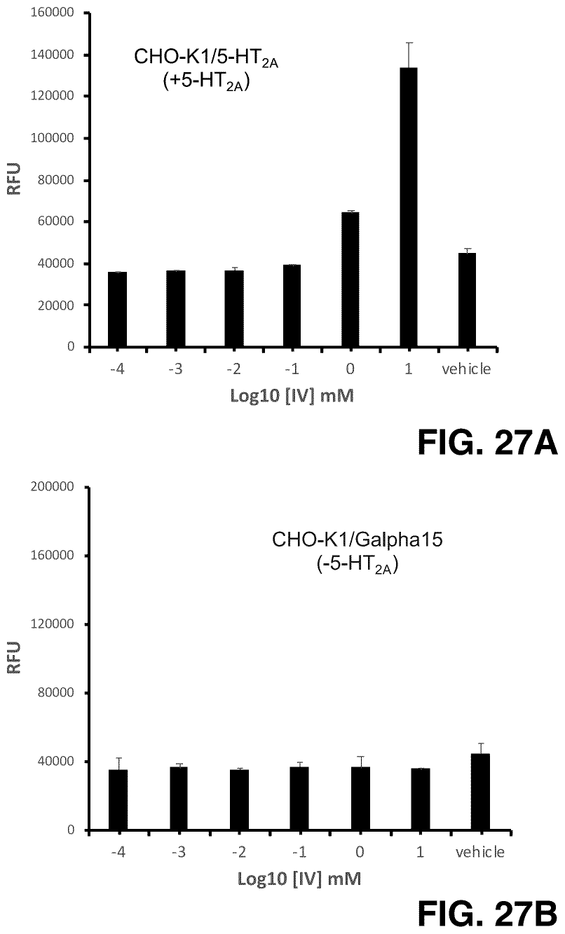

Efficacy testing was carried out as described in Example 5. MTT assay results are shown in FIG. 26, and calcium mobility assay results are shown in FIG. 27A and FIG. 27B. In both cases, the example compound having the chemical formula (IV) is shown as “(IV)”.

### Example 13—Process for Making a Thirteenth Halogenated Psilocybin Derivative from Halogenated Indole Feedstock

A thirteenth halogenated psilocybin derivative compound was synthesized according to the method of Example 11, except using 5-fluoroindole and N-methylethylamine as starting materials. Analysis indicated the isolation of a compound having chemical formula (VII):

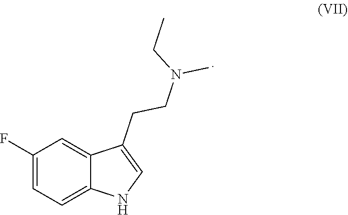 [SMILES: CCN(C)CCc1c[nH]c2ccc(F)cc12]

¹H NMR (400 MHz, CDCl₃): δ (ppm)=8.09 (s, 1H), 7.29-7.20 (m, 2H), 7.06 (d, J=2.4 Hz, 1H), 6.96-6.87 (m, 1H), 2.93-2.86 (m, 2H), 2.72-2.64 (m, 2H), 2.53 (q, J=7.2 Hz, 2H), 2.35 (s, 3H), 1.11 (t, J=7.2 Hz, 3H).

### Example 14—Process for Making a Fourteenth Halogenated Psilocybin Derivative from Halogenated Indole Feedstock

A fourteenth halogenated psilocybin derivative was synthesized according to the method of Example 11, except using 6-fluoroindole and N-methylethylamine as starting materials. Analysis indicated the isolation of a compound having chemical formula (XII):

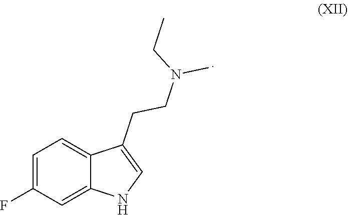 [SMILES: CCN(C)CCc1c[nH]c2cc(F)ccc12]

¹H NMR (400 MHz, CDCl₃): δ (ppm)=8.05 (s, 1H), 7.50 (ddt, J=8.7, 5.3, 0.6 Hz, 1H), 7.02 (ddd, J=9.7, 2.3, 0.5 Hz, 1H), 7.00-6.97 (m, 1H), 6.88 (ddd, J=9.6, 8.6, 2.3 Hz, 1H), 2.96-2.89 (m, 2H), 2.74-2.66 (m, 2H), 2.53 (q, J=7.2 Hz, 2H), 2.35 (s, 3H), 1.11 (t, J=7.2 Hz, 3H).

### Example 15—Process for Making a Fifteenth Halogenated Psilocybin Derivative from Halogenated Indole Feedstock

A fifteenth halogenated psilocybin derivative was synthesized according to the method of Example 11, except using 4-chloroindole as starting material. Analysis indicated the isolation of a compound having chemical formula (III):

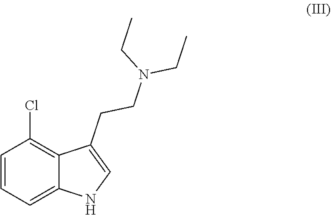 [SMILES: CCN(CC)CCc1c[nH]c2cccc(Cl)c12]

¹H NMR (400 MHz, CDCl₃): δ (ppm)=8.28 (s, 1H), 7.26-7.19 (m, 1H), 7.08-7.01 (m, 3H), 3.18-3.10 (m, 2H), 2.86-2.77 (m, 2H), 2.67 (q, J=7.1 Hz, 4H), 1.09 (t, J=7.1 Hz, 6H).

### Example 16—Process for Making a Sixteenth Halogenated Psilocybin Derivative from Halogenated Indole Feedstock

The initial reaction to generate compound 1.1 (see: FIG. 9B) followed a previously reported procedure with modifications (Campos et al. 2004, Org. Lett 6: 79-82). To a solution of 4-bromophenylhydrazine hydrochloride (300 mg, 1.34 mmol) in a mixture of N,N-dimethylacetamide (1.9 mL) and 4% H₂SO₄ (1.9 mL), was added 2,3-dihydrofuran (0.10 mL, 1.33 mmol). The reaction mixture was heated at 100° C. for 16 hours. Once the mixture was cooled to room temperature, ethyl acetate (15 mL) was added, and the organic solution was washed with water (2×6 mL), and dried over anhydrous MgSO₄. The organic solvent was removed in vacuo. The product (compound 1.1, (see: FIG. 9B)) was purified by flash chromatography on silica gel (eluted with a gradient of hexanes-EtOAc, 100:0 to 0:100) to afford an orange oil. Yield: 37%. The characterization of this first product agreed with previously reported procedure (Go et al. 2010, J. Med. Chem. 53: 6838-6850). ¹H NMR (400 MHz, CDCl₃) δ 8.13 (bs, 1H), 7.74 (d, J=1.7 Hz, 1H), 7.28 (dd, J=8.6, 1.9 Hz, 1H), 7.23 (d, J=8.6 Hz, 1H), 7.08 (d, J=2.3 Hz, 1H), 3.89 (t, J=6.3 Hz, 2H), 2.98 (t, J=6.4 Hz, 2H). The second reaction (see: FIG. 9B) followed a previously reported procedure (Lombardo et al. 2013, Angew. Chem. Int. Ed. 52: 12910-12914). Imidazole (73 mg, 1.1 mmol), triphenyl phosphine (140 mg, 0.54 mmol), and iodine (140 mg, 0.54 mmol) were added to a solution of compound 1.1 (see: FIG. 9B) (119 mg, 0.49 mmol) in anhydrous dichloromethane (5.0 mL) cooled in an ice-water bath. The reaction was stirred at room temperature for 6 hours. The mixture was quenched with saturated sodium thiosulfate (10 mL), extracted in dichloromethane (3×10 mL), and the combined organic solution was dried over anhydrous MgSO₄, and concentrated under reduced pressure. The crude product was purified by flash chromatography on silica gel (eluted with a gradient of hexanes-EtOAc, 100:0 to 50:50) to afford an orange oil. Yield of compound 1.2 (see: FIG. 9B): 74%. ¹H NMR (400 MHz, CDCl₃) δ 8.07 (s, 1H), 7.71-7.69 (m, 1H), 7.29 (dd, J=8.6, 1.7 Hz, 1H), 7.25 (d, J=8.6 Hz, 1H), 7.10 (d, J=2.4 Hz, 1H), 3.44-3.39 (m, 2H), 3.30 (t, J=7.0 Hz, 2H). To a solution of compound 1.2 (129 mg, 0.37 mmol) in anhydrous DMF (1.0 mL) was added di-n-butylamine (0.25 mL, 1.48 mmol). The reaction mixture was heated at 60° C. for 16 hours. The reaction was quenched with saturated sodium bicarbonate solution, extracted in ethyl acetate (3×20 mL), and the combined organic solution was washed with water (2×40 mL), brine (2×40 mL), dried over anhydrous MgSO₄ and concentrated under reduced pressure. The crude mixture was purified by flash column chromatography on silica gel (eluted with a gradient of hexanes-EtOAc, 100:0 to 0:100) to afford an orange oil. Yield of compound 1, with structure as indicated by compound (VIII):

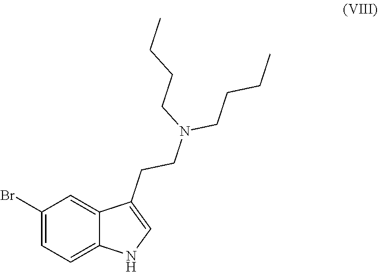 [SMILES: CCCCN(CCCC)CCc1c[nH]c2ccc(Br)cc12]

50%. ¹H NMR (400 MHz, CDCl₃) δ 8.16 (s, 1H), 7.72 (s, 1H), 7.28-7.22 (m, 1H), 7.22-7.18 (m, 1H), 7.03-6.99 (m, 1H), 2.89-2.82 (m, 2H), 2.81-2.73 (m, 2H), 2.54 (t, J=7.6 Hz, 4H), 1.52-1.43 (m, 5H), 1.33 (h, J=7.0 Hz, 4H), 0.93 (t, J=7.4 Hz, 6H) (Figure X, NMR). ¹³C NMR (100 MHz, CDCl₃) b 134.95, 129.58, 124.81, 122.83, 121.65, 114.83, 112.62, 112.57, 54.75, 54.01, 29.42, 22.87, 20.95, 14.27. HRMS (ESI, positive) m/z for C₁₈H₂₈BrN₂ [M+H]⁺ calcd. 351.1430, found 351.1427. The compound having chemical formula (VIII) was 95% (w/w) pure.

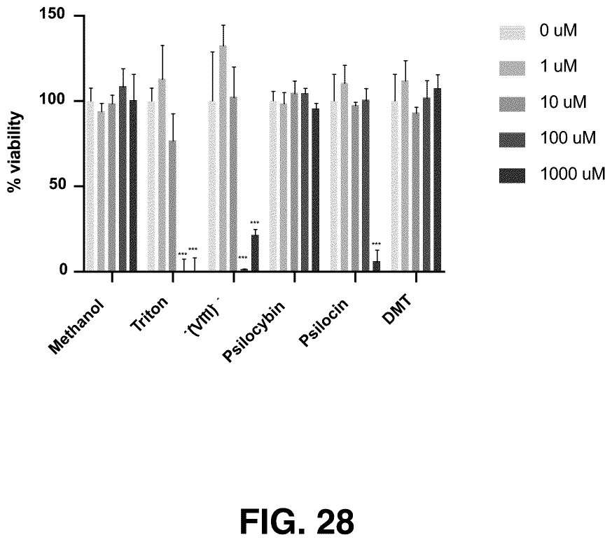

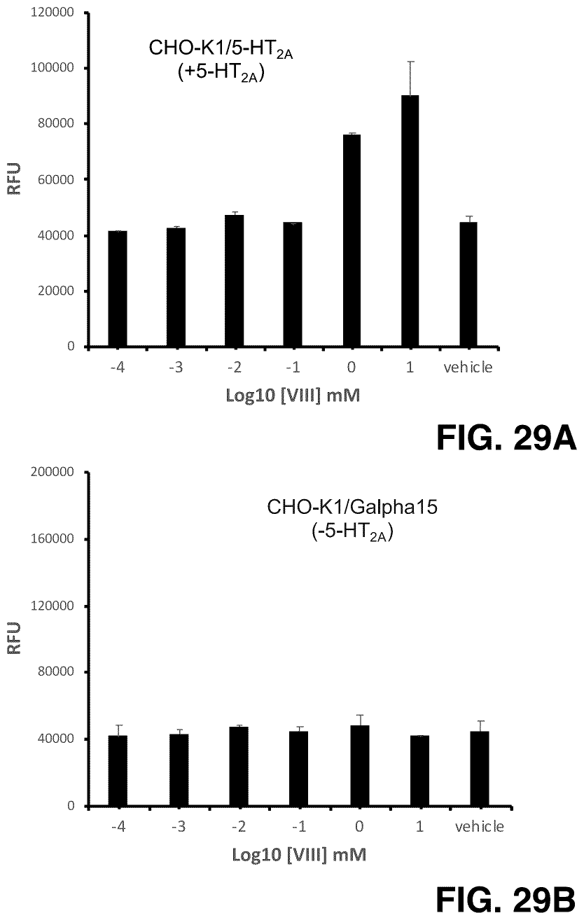

Efficacy testing was carried out as described in Example 5. MTT assay results are shown in FIG. 28, and calcium mobility assay results are shown in FIGS. 29A and 29B. In both cases, the example compound having the chemical formula (VIII) is shown as “(VIII)”.

### Example 17—Process for Making a Seventeenth Halogenated Psilocybin Derivative from Halogenated Indole Feedstock

E. coli strain Ec-3 was constructed as follows. For plasmid cloning, Top10 or XL1-blue strains were used depending on antibiotic markers. Standard LB media was used for culturing. For gene expression and feeding experiments, the parent host strain employed was BL21 (DE3). For this Example, heterologous expression of a non-native or engineered TrpB gene was not necessary, as endogenous tryptophan synthase activity proved sufficient. Plasmids pCDM4-PsiD-FLAG and pETM6-H10-PsiK-V5-PsiM-FLAG are described in Example 10. A third plasmid, pRSM3-ThaI-HIS, was created by cloning an in-frame, C-terminally HIS-tagged (SEQ.ID NO: 89) ThaI gene (SEQ.ID NO: 19), encoding a halogenase having SEQ.ID NO: 20, into the NdeI/XhoI site of pRSM3 (SEQ.ID NO: 98). The three target plasmids pCDM4-PsiD-FLAG, pETM6-H10-PsiK-V5-PsiM-FLAG, and pRSM3-ThaI-HIS were transformed into BL21 (DE3) cells and antibiotics ampicillin plus streptomycin plus kanamycin were used to select for the correct clones containing all three plasmids. Scaled-up culturing of engineered E. coli was conducted as described in Example 10. To assess product, high-resolution LC-HESI-LTQ-Orbitrap-XL MS analysis was conducted as described in Example 1. Singly protonated product with exact m/z and expected elemental formula matching 6-chloro-psilocybin having chemical formula (XVIII):

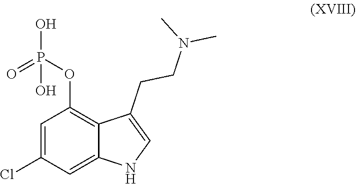 [SMILES: CN(C)CCc1c[nH]c2cc(Cl)cc(OP(=O)(O)O)c12]

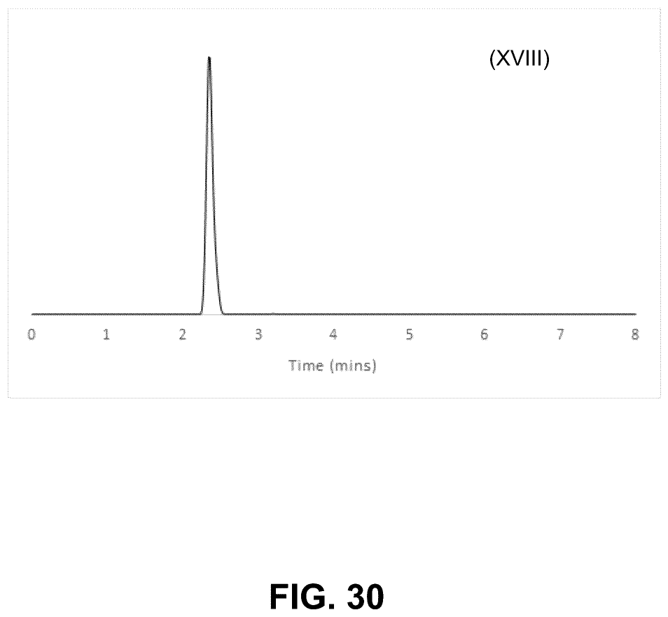

eluted at 2.3 minutes (IC, see: FIG. 30).

1. a chemical compound or salt thereof having the formula (I): 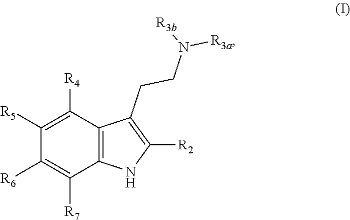
wherein, at least one of R₂, R₄, R₅, R₆ or R₇ is a halogen atom, wherein each non-halogenated R₂, R₅, R₆, or R₇ is a hydrogen atom or an alkyl group or O-alkyl group, wherein R₄ when it is not halogenated is a phosphate group, a hydrogen atom or an alkyl group or O-alkyl group, and wherein R₃ₐ and R_{3b} are a hydrogen atom, an alkyl group, an aryl group, or an acyl group.

2. A chemical compound according to claim 1, wherein one or two of R₄, R₅, R₆, or R₇ are a halogen atom, wherein the halogen atom is selected from fluorine (F), chlorine (Cl), and bromine (Br).

3. A chemical compound according to claim 1, wherein one or two of R₄, R₅, or R₆ are halogenated, wherein the halogen atom is selected from fluorine (F), chlorine (Cl), and bromine (Br).

4. A chemical compound according to claim 1, wherein two of R₄, R₅, and R₆ are halogenated, wherein the halogen atom is selected from fluorine (F), chlorine (Cl), and bromine (Br).

5. A chemical compound according to claim 4, wherein R₄ is halogenated, and wherein one of R₅, and R₆ is halogenated.

6. A chemical compound according to claim 4, wherein R₅ is halogenated, and wherein one of R₄, and R₆ is halogenated.

7. A chemical compound according to claim 5, wherein the halogen atoms are fluorine atoms.

8. A chemical compound according to claim 6, wherein the halogen atoms are fluorine atoms.

9. A chemical compound according to claim 2, wherein R₇ is a fluorine atom, and wherein one atom is halogenated.

10. A chemical compound according to claim 7, wherein each non-halogenated R₂, R₄, R₅, R₆, or R₇, is a hydrogen atom.

11. A chemical compound according to claim 8, wherein each non-halogenated R₂, R₄, R₅, R₆, or R₇, is a hydrogen atom.

12. A chemical compound according to claim 9, wherein each non-halogenated R₂, R₄, R₅, R₆, or R₇, is a hydrogen atom.

13. A chemical compound according to claim 3, wherein at least one of the halogen atoms is a chlorine atom.

14. A chemical compound according to claim 3, wherein R₆ is a chlorine atom.

15. A chemical compound according to claim 14, wherein R₅ is a fluorine atom.

16. A chemical compound according to claim 14, wherein each non-halogenated R₂, R₄, R₅, R₆, or R₇, is a hydrogen atom.

17. A chemical compound according to claim 15, wherein each non-halogenated R₂, R₄, R₅, R₆, or R₇, is a hydrogen atom.

18. A chemical compound according to claim 3, wherein R₅ is a bromine atom, and one atom is halogenated.

19. A chemical compound according to claim 18, wherein each non-halogenated R₂, R₄, R₅, R₆, or R₇, is a hydrogen atom.

20. A chemical compound according to claim 2, wherein R₃ₐ and R_{3b} are each independently selected from a hydrogen atom, a (C₁-C₆)-alkyl group, and a (C₁-C₆)-acyl group.

21. A chemical compound according to claim 10, wherein R₃ₐ and R_{3b} are, respectively, a hydrogen atom and a (C₁-C₆)-acyl group.

22. A chemical compound according to claim 11, wherein R₃ₐ and R_{3b} are, respectively, a hydrogen atom and a (C₁-C₆)-acyl group.

23. A chemical compound according to claim 12, wherein R₃ₐ and R_{3b} are, respectively, a hydrogen atom and a (C₁-C₆)-acyl group.

24. A chemical compound according to claim 16, wherein R₃ₐ and R_{3b} are each a (C₁-C₆)-alkyl group.

25. A chemical compound according to claim 17, wherein R₃ₐ and R_{3b} are each a (C₁-C₆)-alkyl group.

26. A chemical compound according to claim 19, wherein R₃ₐ and R_{3b} are each a (C₁-C₆)-alkyl group.

27. A chemical compound according to claim 1, wherein the chemical compound is selected from the group consisting of compounds having formulas (V); (IX); (X); (XV); (XX); (XXI); (XXII); (XXIII); and (XXIV):

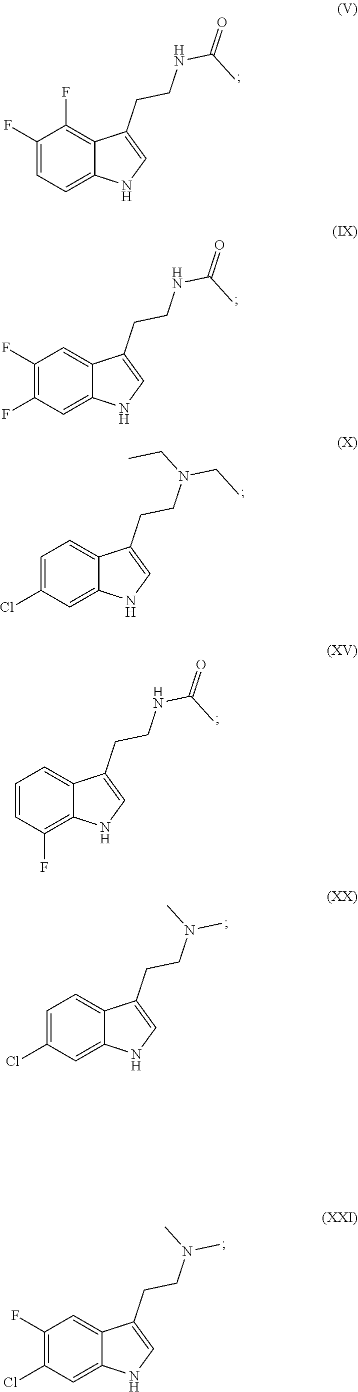 [SMILES: CC(=O)NCCc1c[nH]c2ccc(F)c(F)c12 ; CC(=O)NCCc1c[nH]c2cc(F)c(F)cc12 ; CCN(CC)CCc1c[nH]c2cc(Cl)ccc12 ; CC(=O)NCCc1c[nH]c2c(F)cccc12 ; CN(C)CCc1c[nH]c2cc(Cl)ccc12 ; CN(C)CCc1c[nH]c2cc(Cl)c(F)cc12]

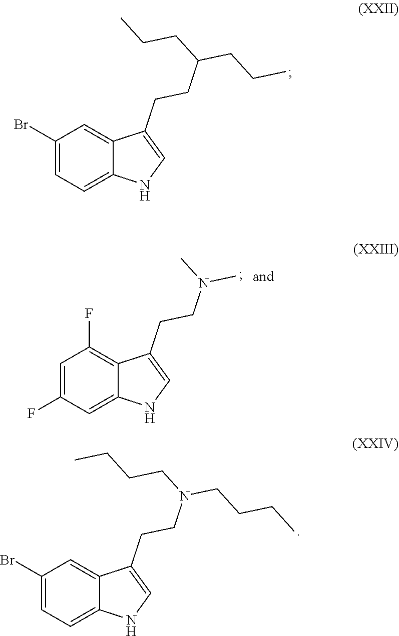

28. A pharmaceutical drug formulation comprising an effective amount of the chemical compound according to claim 1, together with a pharmaceutically acceptable excipient, diluent, or carrier.

29. A pharmaceutical drug formulation comprising an effective amount of the chemical compound according to claim 27, together with a pharmaceutically acceptable excipient, diluent, or carrier.

30. A method for treating a psychiatric disorder, the method comprising administering to a subject in need thereof a pharmaceutical formulation comprising a chemical compound according to claim 1, wherein the pharmaceutical formulation is administered in an effective amount to treat the psychiatric disorder in the subject.
